(19) United States
(12) Patent Application Publication
(10) Pub. No.: US 2011/0040094 A1
(43) Pub. Date: Feb. 17, 2011

(54) 1,3,4-TRISUBSTITUTED BENZENES

(75) Inventors: Gideon Shapiro, Gainesville, FL (US); Richard Chesworth, Boston, MA (US)

Correspondence Address: FISH & RICHARDSON P.C. (BO), P.O. BOX 1022, MINNEAPOLIS, MN, 55440-1022, US

(73) Assignee: ENVIVO PHARMACEUTICALS, INC., Watertown, MA (US)

(21) Appl. No.: 12/678,175

(22) PCT Filed: Sep. 15, 2008
(86) PCT No.: PCT/US08/76408

§ 371 Date: Oct. 29, 2010

Related U.S. Application Data
(60) Provisional application No. 60/972,299, filed on Sep. 14, 2007.

(51) Int. Cl. C07D 471/02; C07C 313/04; C07C 55/00; C07C 59/13; C07C 229/00; C07D 213/55; C07D 231/12; C07D 213/62; C07D 271/12; C07D 285/14

(52) U.S. Cl. 546/121; 558/61; 562/426; 562/469; 562/452; 546/342; 548/377.1; 546/301; 548/126

(57) ABSTRACT

Compounds that are 1,3,4-trisubstituted benzenes which modulate (e.g., inhibit) the activity of γ-secretase. The compounds are expected to reduce the level of Aβ42 in patients and be useful in the treatment of diseases (e.g., Alzheimer's disease) characterized by elevated levels of Aβ42 and/or the formation of Aβ plaques.

### BACKGROUND

Alzheimer's disease (AD) is the most prevalent form of dementia. It is a neurodegenerative disorder that is associated (though not exclusively) with aging. The disorder is clinically characterized by a progressive loss of memory, cognition, reasoning and judgment that leads to an extreme mental deterioration and ultimately death. The disorder is pathologically characterized by the deposition of extracellular plaques and the presence of neurofibrillary tangles. These plaques are considered to play an important role in the pathogenesis of the disease.

These plaques mainly comprise of fibrillar aggregates of β-amyloid peptide (Aβ), which are products of the amyloid precursor protein (APP), a 695 amino-acid protein. APP is initially processed by β-secretase forming a secreted peptide and a membrane bound C99 fragment. The C99 fragment is subsequently processed by the proteolytic activity of γ-secretase. Multiple sites of proteolysis on the C99 fragment lead to the production of a range of smaller peptides (Aβ 37-42 amino acids). N-terminal truncations can also be found e.g. Aβ (4-42) for convenience Aβ40 and Aβ42 as used herein incorporates these N-terminal truncated peptides. Upon secretion, the Aβ peptides initially form soluble aggregates which ultimately lead to the formation of insoluble deposits and plaques. Aβ42 is believed to be the most neurotoxic, the shorter peptides have less propensity to aggregate and form plaques. The Aβ plaques in the brain are also associated with cerebral amyloid angiopathy, hereditary cerebral haemorrage with amyloidosis, multi infarct dementia, dementia pugilistisca and Down's Syndrome.

γ-secretase is an association of proteins, comprising Aph1, Nicastrin, Presenillin and Pen-2 (review De Strooper 2003, Neuron 38, 9). Aβ42 is selectively increased in patients carrying particular mutations in a protein presenilin. These mutations are correlated with early onset a familial AD. Inhibition of γ-secretase resulting in the lowering of Aβ42 is a desirable activity for the pharmaceutical community and numerous inhibitors have been found e.g. Thompson et at (Bio. Org. and Med. Chem. Letters 2006, 16, 2357-63), Shaw et al (Bio. Org. and Med. Chem. Letters 2006, 17, 511-16) and Asberom et at (Bio. Org. and Med. Chem. Letters 2007, 15, 2219-2223). Inhibition of γ-secretase though is not without side-effects, some of which are due to the γ-secretase complex processing substrates other than C99, for e.g. Notch. A more desirable approach is to modulate the proteolytic activity of the γ-secretase complex in a manner that lowers Aβ42 in favor of shorter peptides without affecting the activity of γ-secretase on substrates such as Notch.

Compounds that have shown modulation of γ-secretase include certain non-steroidal, anti-inflammatory drugs (NSAIDs), for example Flurbiprofen, (Stock et at Bio. Org. and Med. Chem. Letters 2006, 16, 2219-2223). Other publications that disclose agents said to reduce Aβ42 through the modulation of γ-secretase include WO 04/074232, WO 05/054193, Perreto et at Journal of Medicinal Chemistry 2005, 48 5705-20, WO05/108362, WO 06/008558, WO 06/021441, WO 06/041874, WO 06/045554, WO 04110350, WO 06/043964, WO 05/115990, WO 07/058,305, WO 05/092002 and U.S. Ser. No. 07/011,7839.

### SUMMARY

Described below are 1,3,4-trisubstituted benzenes of formulas (I) and (II) that modulate (e.g., inhibit) the activity of γ-secretase. The compounds are expected to reduce the level of Aβ42 in patients and be useful in the treatment of diseases characterized by elevated levels of Aβ42 and/or the formation of Aβ plaques (e.g., Alzheimer's disease).

### FIRST GROUP OF EMBODIMENTS

Described herein are compounds of Formula I and II:

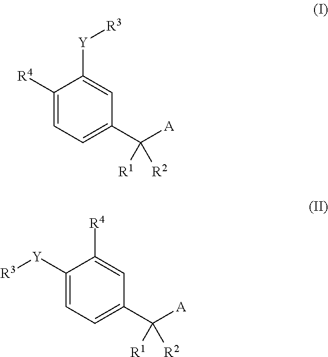

or a pharmaceutically acceptable salts thereof,

wherein:

A is CO₂H;

R₁ and R₂ are independently selected from: H, (C₃-C₆)alkyl, (C₀-C₃)alkyl-(C₃-C₇)cycloalkyl provided that only one of R₁ and R₂ may be H, each alkyl and cycloalkyl is optionally and independently multiply substituted with fluoro, hydroxy, methoxy or CF₃;

or

R₁ and R₂ are taken together with the carbon to which they are attached to form a 3-7 membered cycloalkyl or heterocycloalkyl ring which optionally and independently multiply substituted with C₁-C₄ alkyl hydroxy, fluoro or CF₃;

Y is a bond;

R₃ is aryl or heteroaryl both of which are optionally and independently multiply substituted with R₁₂; wherein each R₁₂ is independently selected from halogen, R₆, CF₃, CN, NH₂, NO₂, OR₆, SR₆CO₂R₆, OCOR₆ and COR₆; wherein the attachment site may be either at a carbon atom or a nitrogen atom of the ring system provided that only three bonds are made to nitrogen;

R₆ is C₁-C₅ alkyl optionally interrupted by —O—, —S—, —S(O)—, or —S(O)₂— groups; —(C₃-C₇)cycloalkyl, (C₁-C₃)alkyl-(C₃-C₇)cycloalkyl each optionally and independently multiply substituted with fluoro, hydroxy, cyano, or CF₃

or

R₆ is (CH₂)ₙAr wherein n=0-2; wherein Ar is a phenyl, napthyl or heteroaryl ring and Ar is optionally substituted with up to 3 groups selected from alkyl, halogen, CF₃, OH, OCF₃, (C₁-C₆) alkoxy, OCH₂CH₂OCH₃, NH₂, (C₁-C₆)alkylamino, (C₁-C₆)(C₁-C₆)dialkylamino, morpholino, CN, NO₂, alkylthio, alkylsulfonyl; or R₁ and R₂ can be taken together to form a 3-7 membered cycloalkyl or heterocycloalkyl ring which optionally bears a C₁-C₄ alkyl substituent; and

R₄ is C₂-C₄ alkyl optionally and multiply independently substituted with fluoro or hydroxy, —OCH₂CF₃, —OCH₂CH₂CF₃, —OCH(CF₃)₂, C₂-C₄ alkoxy optionally and multiply independently substituted with fluoro or hydroxy, —O-cyclopropyl, —O-cyclobutyl, cyclopropyloxymethyl, —SCH(Me)(CF₃), —SCH₂CF₃, —SCH₂CH₂CF₃, —SCH(CF₃)₂, C₂-C₄ thioalkoxy, —S-cyclopropyl, —S-cyclobutyl or S—CH₂-cyclopropyl

In various embodiments of the compounds of Formula I and Formula II:
the compound has formula (I);
the compound has formula (II);
R₁ is H and R₂ is (C₃-C₆)alkyl;
R₁ is H and R₂ is n-propyl;
R₁ is H and R₂ is iso-butyl
R₁ is H and R₂ is (C₀-C₃)alkyl-(C₃-C₇)cycloalkyl
R₁ is H and R₂ is —CH₂-cyclopropyl
R₁ is H and R₂ is —CH₂-cyclobutyl
R₁ and R₂ are taken together with the carbon to which they are attached to form a 3-7 membered cycloalkyl ring
R₁ and R₂ are taken together with the carbon to which they are attached to form a cyclobutyl ring
R₁ and R₂ are taken together with the carbon to which they are attached to form a cyclopentyl ring
R₁ and R₂ are taken together with the carbon to which they are attached to form a cyclohexyl ring
R₃ is aryl
R₃ is phenyl
R₃ is heteroaryl
R₃ is benzothienyl
R₃ is benzofuranyl
R₃ is benzothiazyl
R₃ is benzoxazyl
R₃ is benzo[c][1,2,5]oxadiazyl
R₃ is benzo[c][1,2,5]thiadiazole
R₃ is imidazopyridyl
R₃ is Pyridyl
R₃ is thienyl
R₄ is C₂-C₄ alkyl optionally and multiply independently substituted with fluoro or hydroxyl
R₄ is unsubstituted C₂-C₄ alkyl
R₄ is —OCH₂CF₃
R₄ is —OCH₂CH₂CF₃
R₄ is —OCH(CF₃)₂
R₄ is —OCH(Me)(CF₃)
R₄ is C₂-C₄ alkoxy optionally and multiply independently substituted with fluoro or hydroxyl
R₄ is unsubstituted C₂-C₄ alkoxy
R₄ is —O-cyclopropyl
R₄ is —O-cyclobutyl
R₄ is cyclopropyloxymethyl
R₄ is —SCH(Me)(CF₃)
R₄ is —SCH₂CF₃
R₄ is —SCH₂CH₂CF₃
R₄ is —SCH(CF₃)₂
R₄ is C₂-C₄ thioalkoxy
R₄ is —S-cyclopropyl
R₄ is —S-cyclobutyl
R₄ is S—CH₂-cyclopropyl
each R₁₂ is independently selected from halogen, R₆, CF₃, CN, NO₂, NH₂, OR₆, SR₆CO₂R₆, OCOR₆ and COR₆
each R₁₂ is independently selected from halogen R₆, CF₃, CN, NO₂, OR₆, SR₆ and COR₆
each R₁₂ is independently selected from fluoro, chloro, R₆, CF₃, CN, OR₆, and SR₆
R₆ is C₁-C₅ alkyl optionally interrupted by —O—, —S—, —S(O)—, or —S(O)₂— groups; —(C₃-C₇)cycloalkyl, (C₁-C₃)alkyl-(C₃-C₇)cycloalkyl each optionally and independently multiply substituted with fluoro, hydroxy, cyano, or CF₃
R₆ is C₁-C₅ alkyl where alkyl contains only carbon and hydrogen; —(C₃-C₇)cycloalkyl, (C₁-C₃)alkyl-(C₃-C₇)cycloalkyl each optionally and optionally and independently multiply substituted with fluoro, hydroxy, cyano, or CF₃
R₆ is (CH₂)ₙAr wherein n=0-2, wherein Ar is a phenyl, napthyl or heteroaryl ring and Ar is optionally substituted with up to 3 groups selected from alkyl, halogen, CF₃, OH, OCF₃, alkoxy, OCH₂CH₂OCH₃, NH₂, alkylamino, dialkylamino, morpholino, CN, NO₂, alkylthio, alkylsulfonyl; or R₁ and R₂ can be taken together to form a 3-7 membered cycloalkyl or heterocycloalkyl ring which optionally bears a C₁-C₄ alkyl substituent;
R₆ is (CH₂)ₙAr wherein n=0-2, wherein Ar is a phenyl which is optionally substituted with up to 3 groups selected from alkyl, halogen, CF₃, OH, OCF₃, alkoxy, OCH₂CH₂OCH₃, NH₂, alkylamino, dialkylamino, morpholino, CN, NO₂, alkylthio, alkylsulfonyl; or R₁ and R₂ can be taken together to form a 3-7 membered cycloalkyl or heterocycloalkyl ring which optionally bears a C₁-C₄ alkyl substituent.
R₆ is (CH₂)ₙAr wherein n=0-2 wherein Ar is a napthyl ring which is optionally substituted with up to 3 groups selected from alkyl, halogen, CF₃, OH, OCF₃, alkoxy, OCH₂CH₂OCH₃, NH₂, alkylamino, dialkylamino, morpholino, CN, NO₂, alkylthio, alkylsulfonyl; or R₁ and R₂ can be taken together to form a 3-7 membered cycloalkyl or heterocycloalkyl ring which optionally bears a C₁-C₄ alkyl substituent.
R₆ is (CH₂)ₙAr wherein n=0-2 wherein Ar is a heteroaryl ring which is optionally substituted with up to 3 groups selected from alkyl, halogen, CF₃, OH, OCF₃, alkoxy, OCH₂CH₂OCH₃, NH₂, alkylamino, dialkylamino, morpholino, CN, NO₂, alkylthio, alkylsulfonyl; or R₁ and R₂ can be taken together to form a 3-7 membered cycloalkyl or heterocycloalkyl ring which optionally bears a C₁-C₄ alkyl substituent.

In further embodiments a compound is selected from:
1-(2-(cyclopropylmethoxy)-4′-(trifluoromethyl)biphenyl-4-yl)cyclobutanecarboxylic acid
1-(2-(cyclopropylmethoxy)-4′-fluorobiphenyl-4-yl)cyclobutanecarboxylic acid
1-(4′-chloro-2-(cyclopropylmethoxy)biphenyl-4-yl)cyclobutanecarboxylic acid
2-(2-(cyclopropylmethoxy)-4′-(trifluoromethyl)biphenyl-4-yl)-4-methylpentanoic acid
2-(2-(cyclopropylmethoxy)-4′-fluorobiphenyl-4-yl)-4-methylpentanoic acid
2-(4′-chloro-2-(cyclopropylmethoxy)biphenyl-4-yl)-3-cyclopropylpropanoic acid
2-(4′-chloro-2-(cyclopropylmethoxy)biphenyl-4-yl)-4-methylpentanoic acid
3-cyclopropyl-2-(2-(cyclopropylmethoxy)-4′-(trifluoromethyl)biphenyl-4-yl)propanoic acid
3-cyclopropyl-2-(2-(cyclopropylmethoxy)-4′-fluorobiphenyl-4-yl)propanoic acid
1-(2-(cyclopropylmethoxy)-4′-(methylthio)biphenyl-4-yl)cyclobutanecarboxylic acid
1-(2-(cyclopropylmethoxy)-4′-(trifluoromethoxy)biphenyl-4-yl)cyclobutanecarboxylic acid
1-(3′,4′-dichloro-2-(cyclopropylmethoxy)biphenyl-4-yl)cyclobutanecarboxylic acid
2-(2-(cyclopropylmethoxy)-4′-(methylthio)biphenyl-4-yl)-4-methylpentanoic acid
2-(2-(cyclopropylmethoxy)-4′-(trifluoromethoxy)biphenyl-4-yl)-4-methylpentanoic acid
2-(3′,4′-dichloro-2-(cyclopropylmethoxy)biphenyl-4-yl)-4-methylpentanoic acid
3-cyclopropyl-2-(2-(cyclopropylmethoxy)-4′-(methylthio)biphenyl-4-yl)propanoic acid
3-cyclopropyl-2-(2-(cyclopropylmethoxy)-4′-(trifluoromethoxy)biphenyl-4-yl)propanoic acid
3-cyclopropyl-2-(3′,4′-dichloro-2-(cyclopropylmethoxy)biphenyl-4-yl)propanoic acid
1-(2-(2,2,2-trifluoroethoxy)-4′-(trifluoromethyl)biphenyl-4-yl)cyclobutanecarboxylic acid
1-(4′-chloro-2-(2,2,2-trifluoroethoxy)biphenyl-4-yl)cyclobutanecarboxylic acid
1-(4′-fluoro-2-(2,2,2-trifluoroethoxy)biphenyl-4-yl)cyclobutanecarboxylic acid
2-(4′-chloro-2-(2,2,2-trifluoroethoxy)biphenyl-4-yl)-3-cyclopropylpropanoic acid
2-(4′-chloro-2-(2,2,2-trifluoroethoxy)biphenyl-4-yl)-4-methylpentanoic acid
2-(4′-fluoro-2-(2,2,2-trifluoroethoxy)biphenyl-4-yl)-4-methylpentanoic acid
3-cyclopropyl-2-(2-(2,2,2-trifluoroethoxy)-4′-(trifluoromethyl)biphenyl-4-yl)propanoic acid
3-cyclopropyl-2-(4′-fluoro-2-(2,2,2-trifluoroethoxy)biphenyl-4-yl)propanoic acid
4-methyl-2-(2-(2,2,2-trifluoroethoxy)-4′-(trifluoromethyl)biphenyl-4-yl)pentanoic acid
1-(2-(2,2,2-trifluoroethoxy)-4′-(trifluoromethoxy)biphenyl-4-yl)cyclobutanecarboxylic acid
1-(3′,4′-dichloro-2-(2,2,2-trifluoroethoxy)biphenyl-4-yl)cyclobutanecarboxylic acid
1-(4′-(methylthio)-2-(2,2,2-trifluoroethoxy)biphenyl-4-yl)cyclobutanecarboxylic acid
2-(3′,4′-dichloro-2-(2,2,2-trifluoroethoxy)biphenyl-4-yl)-4-methylpentanoic acid
3-cyclopropyl-2-(2-(2,2,2-trifluoroethoxy)-4′-(trifluoromethoxy)biphenyl-4-yl)propanoic acid
3-cyclopropyl-2-(3′,4′-dichloro-2-(2,2,2-trifluoroethoxy)biphenyl-4-yl)propanoic acid
3-cyclopropyl-2-(4′-(methylthio)-2-(2,2,2-trifluoroethoxy)biphenyl-4-yl)propanoic acid
4-methyl-2-(2-(2,2,2-trifluoroethoxy)-4′-(trifluoromethoxy)biphenyl-4-yl)pentanoic acid
4-methyl-2-(4′-(methylthio)-2-(2,2,2-trifluoroethoxy)biphenyl-4-yl)pentanoic acid
1-(4-(benzo[c][1,2,5]oxadiazol-5-yl)-3-(cyclopropylmethoxy)phenyl)cyclobutanecarboxylic acid
1-(4-(benzo[c][1,2,5]thiadiazol-5-yl)-3-(cyclopropylmethoxy)phenyl)cyclobutanecarboxylic acid
2-(4-(benzo[c][1,2,5]oxadiazol-5-yl)-3-(cyclopropylmethoxy)phenyl)-3-cyclopropylpropanoic acid
2-(4-(benzo[c][1,2,5]oxadiazol-5-yl)-3-(cyclopropylmethoxy)phenyl)-4-methylpentanoic acid
2-(4-(benzo[c][1,2,5]thiadiazol-5-yl)-3-(cyclopropylmethoxy)phenyl)-3-cyclopropylpropanoic acid
2-(4-(benzo[c][1,2,5]thiadiazol-5-yl)-3-(cyclopropylmethoxy)phenyl)-4-methylpentanoic acid
1-(4-(benzo[c][1,2,5]oxadiazol-5-yl)-3-(2,2,2-trifluoroethoxy)phenyl)cyclobutanecarboxylic acid
1-(4-(benzo[c][1,2,5]thiadiazol-5-yl)-3-(2,2,2-trifluoroethoxy)phenyl)cyclobutanecarboxylic acid
2-(4-(benzo[c][1,2,5]oxadiazol-5-yl)-3-(2,2,2-trifluoroethoxy)phenyl)-3-cyclopropylpropanoic acid
2-(4-(benzo[c][1,2,5]oxadiazol-5-yl)-3-(2,2,2-trifluoroethoxy)phenyl)-4-methylpentanoic acid
2-(4-(benzo[c][1,2,5]thiadiazol-5-yl)-3-(2,2,2-trifluoroethoxy)phenyl)-3-cyclopropylpropanoic acid
2-(4-(benzo[c][1,2,5]thiadiazol-5-yl)-3-(2,2,2-trifluoroethoxy)phenyl)-4-methylpentanoic acid
1-(4′-chloro-6-(cyclopropylmethoxy)biphenyl-3-yl)cyclobutanecarboxylic acid
1-(6-(cyclopropylmethoxy)-4′-(trifluoromethyl)biphenyl-3-yl)cyclobutanecarboxylic acid
1-(6-(cyclopropylmethoxy)-4′-fluorobiphenyl-3-yl)cyclobutanecarboxylic acid
2-(4′-chloro-6-(cyclopropylmethoxy)biphenyl-3-yl)-3-cyclopropylpropanoic acid
2-(4′-chloro-6-(cyclopropylmethoxy)biphenyl-3-yl)-4-methylpentanoic acid
2-(6-(cyclopropylmethoxy)-4′-(trifluoromethyl)biphenyl-3-yl)-4-methylpentanoic acid
2-(6-(cyclopropylmethoxy)-4′-fluorobiphenyl-3-yl)-4-methylpentanoic acid
3-cyclopropyl-2-(6-(cyclopropylmethoxy)-4′-(trifluoromethyl)biphenyl-3-yl)propanoic acid
3-cyclopropyl-2-(6-(cyclopropylmethoxy)-4′-fluorobiphenyl-3-yl)propanoic acid
1-(3′,4′-dichloro-6-(cyclopropylmethoxy)biphenyl-3-yl)cyclobutanecarboxylic acid
1-(6-(cyclopropylmethoxy)-4′-(methylthio)biphenyl-3-yl)cyclobutanecarboxylic acid
1-(6-(cyclopropylmethoxy)-4′-(trifluoromethoxy)biphenyl-3-yl)cyclobutanecarboxylic acid
2-(3′,4′-dichloro-6-(cyclopropylmethoxy)biphenyl-3-yl)-4-methylpentanoic acid
2-(6-(cyclopropylmethoxy)-4′-(methylthio)biphenyl-3-yl)-4-methylpentanoic acid
2-(6-(cyclopropylmethoxy)-4′-(trifluoromethoxy)biphenyl-3-yl)-4-methylpentanoic acid
3-cyclopropyl-2-(3′,4′-dichloro-6-(cyclopropylmethoxy)biphenyl-3-yl)propanoic acid
3-cyclopropyl-2-(6-(cyclopropylmethoxy)-4′-(methylthio)biphenyl-3-yl)propanoic acid
3-cyclopropyl-2-(6-(cyclopropylmethoxy)-4′-(trifluoromethoxy)biphenyl-3-yl)propanoic acid
1-(4′-chloro-6-(2,2,2-trifluoroethoxy)biphenyl-3-yl)cyclobutanecarboxylic acid
1-(4′-fluoro-6-(2,2,2-trifluoroethoxy)biphenyl-3-yl)cyclobutanecarboxylic acid
1-(6-(2,2,2-trifluoroethoxy)-4′-(trifluoromethyl)biphenyl-3-yl)cyclobutanecarboxylic acid
2-(4′-chloro-6-(2,2,2-trifluoroethoxy)biphenyl-3-yl)-3-cyclopropylpropanoic acid
2-(4′-chloro-6-(2,2,2-trifluoroethoxy)biphenyl-3-yl)-4-methylpentanoic acid
2-(4′-fluoro-6-(2,2,2-trifluoroethoxy)biphenyl-3-yl)-4-methylpentanoic acid
3-cyclopropyl-2-(4′-fluoro-6-(2,2,2-trifluoroethoxy)biphenyl-3-yl)propanoic acid
3-cyclopropyl-2-(6-(2,2,2-trifluoroethoxy)-4′-(trifluoromethyl)biphenyl-3-yl)propanoic acid
4-methyl-2-(6-(2,2,2-trifluoroethoxy)-4′-(trifluoromethyl)biphenyl-3-yl)pentanoic acid
1-(3′,4′-dichloro-6-(2,2,2-trifluoroethoxy)biphenyl-3-yl)cyclobutanecarboxylic acid
1-(4′-(methylthio)-6-(2,2,2-trifluoroethoxy)biphenyl-3-yl)cyclobutanecarboxylic acid
1-(6-(2,2,2-trifluoroethoxy)-4′-(trifluoromethoxy)biphenyl-3-yl)cyclobutanecarboxylic acid
2-(3′,4′-dichloro-6-(2,2,2-trifluoroethoxy)biphenyl-3-yl)-4-methylpentanoic acid
3-cyclopropyl-2-(3′,4′-dichloro-6-(2,2,2-trifluoroethoxy)biphenyl-3-yl)propanoic acid
3-cyclopropyl-2-(4′-(methylthio)-6-(2,2,2-trifluoroethoxy)biphenyl-3-yl)propanoic acid
3-cyclopropyl-2-(6-(2,2,2-trifluoroethoxy)-4′-(trifluoromethoxy)biphenyl-3-yl)propanoic acid
4-methyl-2-(4′-(methylthio)-6-(2,2,2-trifluoroethoxy)biphenyl-3-yl)pentanoic acid
4-methyl-2-(6-(2,2,2-trifluoroethoxy)-4′-(trifluoromethoxy)biphenyl-3-yl)pentanoic acid
1-(3-(benzo[c][1,2,5]oxadiazol-5-yl)-4-(cyclopropylmethoxy)phenyl)cyclobutanecarboxylic acid
1-(3-(benzo[c][1,2,5]thiadiazol-5-yl)-4-(cyclopropylmethoxy)phenyl)cyclobutanecarboxylic acid
2-(3-(benzo[c][1,2,5]oxadiazol-5-yl)-4-(cyclopropylmethoxy)phenyl)-3-cyclopropylpropanoic acid
2-(3-(benzo[c][1,2,5]oxadiazol-5-yl)-4-(cyclopropylmethoxy)phenyl)-4-methylpentanoic acid
2-(3-(benzo[c][1,2,5]thiadiazol-5-yl)-4-(cyclopropylmethoxy)phenyl)-3-cyclopropylpropanoic acid
2-(3-(benzo[c][1,2,5]thiadiazol-5-yl)-4-(cyclopropylmethoxy)phenyl)-4-methylpentanoic acid
1-(3-(benzo[c][1,2,5]oxadiazol-5-yl)-4-(2,2,2-trifluoroethoxy)phenyl)cyclobutanecarboxylic acid
1-(3-(benzo[c][1,2,5]thiadiazol-5-yl)-4-(2,2,2-trifluoroethoxy)phenyl)cyclobutanecarboxylic acid
2-(3-(benzo[c][1,2,5]oxadiazol-5-yl)-4-(2,2,2-trifluoroethoxy)phenyl)-3-cyclopropylpropanoic acid
2-(3-(benzo[c][1,2,5]oxadiazol-5-yl)-4-(2,2,2-trifluoroethoxy)phenyl)-4-methylpentanoic acid
2-(3-(benzo[c][1,2,5]thiadiazol-5-yl)-4-(2,2,2-trifluoroethoxy)phenyl)-3-cyclopropylpropanoic acid
2-(3-(benzo[c][1,2,5]thiadiazol-5-yl)-4-(2,2,2-trifluoroethoxy)phenyl)-4-methylpentanoic acid

### SECOND GROUP OF EMBODIMENTS

Described herein are of compounds of formulas (I) and (II)

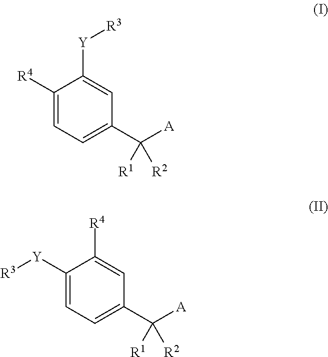

and pharmaceutically acceptable salts thereof
wherein:
A is CO₂H, C(O)NHOH, C(O)NHOCH₃, C(O)NH₂, C(O)NH(R₅), C(O)N(R₅)₂, C(O)NH(SO₂CH₃), COCF₃ or a tetrazole group;
each R₅ is independently chosen from alkyl; cycloalkyl; (CH₂)ₙAr, wherein n=0-2 and wherein Ar is a phenyl, napthyl; and a heteroaryl ring and wherein Ar is optionally substituted with up to 3 groups selected from alkyl, halogen, CF₃, OH, OCF3, alkoxy, OCH₂CH₂OCH₃, NH₂, alkylamino, dialkylamino, morpholino, CN, NO₂, alkylthio, alkylsulfonyl; or in the case when two R₅ are attached to the same N and are both alkyl, they can be taken together to form a 5-membered or 6-membered ring optionally containing O, S, N(H) or N-alkyl.
R₁ and R₂ are independently selected from: H, F, R₆, OH, OR₆, NH₂, NHR₅, N(R₅)₂ wherein R₅ is taken independently and is as defined above, NHC(O)R₅, NHCO₂R₅, SR₆, S(O)R₆, S(O)₂R₆ wherein R₆ is C1-C5 alkyl optionally interrupted by —O—, —S—, —S(O)—, or —S(O)₂— groups; cycloalkyl or (CH₂)ₙAr wherein n=0-2 and wherein Ar is a phenyl, napthyl or heteroaryl ring and wherein Ar is optionally substituted with up to 3 groups selected from alkyl, halogen, CF₃, OH, OCF₃, alkoxy, OCH₂CH₂OCH₃, NH₂, alkylamino, dialkylamino, morpholino, CN, NO₂, alkylthio, alkylsulfonyl; or R₁ and R₂ can be taken together to form a 3-7 membered cycloalkyl or heterocycloalkyl ring which optionally bears a C1-C4 alkyl substituent;
Y is a bond or a divalent linking group selected from —O—, —OCH₂—, —OCH₂CH₂—, —CH₂, —C(O)—, —CH═CH—, —CH₂CH₂—, —CH₂O—, —CH₂OCH₂—, —CH₂CH₂O—, —S—, —SCH₂—, CH₂S—, —CH₂SCH₂—, —C(O)NH—, —C(O)N(R₅)—, —NHC(O)—, —N(R₅)C(O)—, —S(O)—, —S(O₂)—, —S(O)₂N(H)—, —S(O)₂N(R₅)—, —N(H)S(O)₂—, and —N(R₅)S(O)₂—, wherein the point of attachment of divalent linking groups, Y, to R₃ in the Formulas I and II is to the right;
R₃ is a C3-C7 alkyl group optionally interrupted by —O—, —S—, —S(O)—, or —S(O)₂— groups or a mono- or bi-cyclic ring system comprising 5 to 10 ring atoms selected from C, N, O and S, provided that not more than 3 ring atoms in any single ring are other than C, said ring system optionally bearing up to 3 substituents selected from halogen, R₆, CF₃, CN, NO₂, OR₆, SR₆CO₂R₆, OCOR₆ and COR₆; wherein R₆ is as stated above and wherein the attachment site may be either at a carbon atom or a nitrogen atom of the ring system provided that only three bonds are made to nitrogen;
R₄ is C2-C4 alkyl, OCH₂CF₃, C2-C4 alkoxy, cyclopropyloxymethyl, trialkylsilyl, SO₂NH₂, SO₂N(R₇)(R₈), or mono or bicyclic aromatic or heteroaromatic system ring comprising 5 to 10 ring atoms chosen from C, N, O and S, provided that not more than 3 ring atoms in any single ring are other than C, wherein the ring system must be substituted with an R₉ group and is optionally substituted with an R₁₀ groups and wherein the ring attachment may be either at a carbon atom or a nitrogen atom of the ring system provided that only three bonds are made to the attachment nitrogen atom;
R₇ and R₈ are independently selected from H, alkyl, cycloalkyl, (CH₂)ₙAr wherein n=0-2 and wherein Ar is a phenyl, napthyl or heteroaryl ring optionally substituted with up to 3 groups selected from alkyl, halogen, CF₃, OH, OCF₃, alkoxy, OCH₂CH₂OCH₃, NH₂, alkylamino, dialkylamino, morpholino, CN, NO₂, alkylthio, or alkylsulfonyl; and when taken together may form a mono or bicyclic ring system comprising from 5-10 atoms and which may contain up to 3 heteroatoms selected from O, N, or S and which is optionally substituted with halogen, alkyl, alkoxy, CF₃, CN, OH, NO₂, NH₂, alkylamino, dialkylamino, alkylsulfonyl;
R₉ is selected from OH, NH₂, NH(R₅), N(R₅)₂, alkylsulfonyl, arylsulfonyl, SO₂NH₂, SO₂N(R₅)₂, a heteroaryl group optionally substituted with halogen, OH, alkyl, alkoxy, NH₂, CN, NO₂ and a phenyl ring which is substituted with at least one group selected from ethyl, cyclopropyl, OH, alkoxy, CN, NO₂, NH₂, N(R₅)₂, alkylsulfonyl, arylsulfonyl, SO₂NH₂, and is optionally additionally substituted with a halogen group; and
R₁₀ is hydrogen, halogen, alkyl, CF₃, alkoxy, CN, or NO₂.

In one embodiment A is CO₂H or C(O)NHOH and R₁, R₂, Y, R₃ and R₄ are as defined above.

In one embodiment R₁ is hydrogen and R₂ is F, R₆, OH, OR₆, NH₂, NHR₅, N(R₅)₂, NHC(O)R₅, NHCO₂R₅, SR₆, S(O)R₆, S(O)₂R₆ wherein R₅ and R₆ are as defined above.

In other embodiments, R₁ and R₂ are both alkyl, which may, when taken together, form a 3-7 membered cycloalkyl or heterocycloalkyl ring which optionally bears a C1-C4 alkyl substituent. In a further embodiment R₁ is hydrogen and R₂ is a C1-C4 alkyl group optionally interrupted by —O—, —S—, —S(O)—, or —S(O)₂— groups.

In one embodiment Y is a bond. Y comprises a divalent linking group linear chain of 1-2 atoms. In a further embodiment Y is —O—, —OCH₂—, —CH₂O—, —S—, —S(O)₂—, —S(O)₂N(H)—, —S(O)₂N(R₅)—, —C(O)NH— or —C(O)N(R₅)—.

In one embodiment R₃ is a C4-C8 alkyl group optionally interrupted by —O—, —S—, —S(O)—, or —S(O)₂— groups and Y is a bond, —O—, —S—, or —S(O)₂—. In a further embodiment the C4-C8 alkyl group is a saturated linear or branched hydrocarbon examples include but are not limited to butyl, isobutyl, pentyl and isopentyl. R₃ comprises a mono- or bi-cyclic ring system ring system as defined previously that may be saturated or unsaturated including aromatic and heteroaromatic. Examples of monocyclic ring systems include but are not limited to 5-6 membered ring systems such as phenyl, cyclohexyl, cyclopentanyl, pyridyl, piperidinyl, pyrimidyl, pyrrazolyl, thiophene-yl, furanyl, oxadiazolyl, thiadizolyl, triazolyl, oxazolyl and thiazolyl. Examples of bicyclic ring systems include but are not limited to 9-10 membered bicyclic ring systems such as napthyl, quinolinyl, isoquinolinyl, tetrahydroisoquinyl, indolyl, indazolyl, benzimidazolyl, benzthiadiazolyl and imidazopyridinyl. In one embodiment R₃ comprises a fully aromatic ring system including by not limited to phenyl, pyridyl, napthyl, quinolyl, isoquinolyl, oxadiazolyl. R₃ comprises partially aromatic ring system in which an 5-6 membered aromatic ring is fused to a 5-6 membered non-aromatic ring. Examples of partially aromatic ring systems include but are not limited to tetrahydroquinolyl, tetrahydroisoquinolyl, benzimidazolonyl, and indolonyl. In another embodiment R₃ comprises a fully saturated ring system with examples including but not limited to cyclopentyl, cyclohexyl, and piperidinyl. In one embodiment the mono- or bi-cyclic ring system ring system bears 1-2 substitutents. In a further embodiment substitutents are selected from halogen, R₆, CF₃, CN, NO₂, OR₆ and SR₆.

In one embodiment R₄ is C₂-C₄ alkyl. In another embodiment R₄ is C₂-C₄ alkoxy, alkylthio, or alkylsulfonyl group with examples including ethoxy, trifluoroethoxy, isopropoxy, cyclopropyloxymethyl, ethylthio and ethylsulfonyl. In another embodiment R₄ is a sulfonamide selected from SO₂NH₂ or SO₂N(R₇)(R₈) wherein R₇ and R₈ are as previously defined. In another embodiment R₇ and R₈ form a form a 5-7 membered monocyclic ring system optionally containing a single heteroatom selected from O, N, or S wherein when the heteroatom is N it is optionally substituted with an alkyl group. Examples include but are not limited to pyrrolidinyl, piperidinyl, morpholinyl, piperazinyl, and N-alkyl piperazinyl. In another embodiment R₇ and R₈ form a bicyclic ring system comprising from 9-10 atoms which may contain up to 3 heteroatoms selected from O, N, or S and which is optionally substituted with 1-2 groups selected from halogen, alkyl, alkoxy, CF₃, CN, OH, NO₂, NH₂, alkylamino, dialkylamino, alkylsulfonyl. Examples include but are not limited to tetrahydroquinolyl, tetrahydroisoquinolyl, dihydroindolyl and dihyroisoindolyl.

In one embodiment R₄ is a phenyl ring and the ring must be independently substituted with R₉ and R₁₀ groups wherein R₉ and R₁₀ are as previously described. In another embodiment R₄ is a monocyclic heteroaromatic ring system comprising 5-6 atoms chosen from C, N, O and S, wherein not more than 3 ring atoms are other than C. The ring system must be substituted with R₉ and R₁₀ groups which are as described previously. The ring attachment may be either at a carbon atom or a nitrogen atom of the ring system provided that only three bonds are made to the attachment nitrogen atom. Examples of such monocyclic heteroaromatic ring system include but are not limited to 1,2,3-oxadiazyl, 1,2,3-thiadiazyl, 1,2,3-triazyl, 1,2,4-oxadiazyl, 1,2,4-thiadiazyl, 1,2,4-triaziyl, 1,2,5-oxadiazyl, 1,2,5-thiadiazyl, 1,3,4-oxadiazyl, 1,3,4-thiadiazyl, 1,3,5-triazine, 1H-1,2,3-triazyl, 1H-1,2,4-triazyl, 1H-imidazyl, 1H-pyrazyl, 1H-pyrroyl, 1H-tetrazyl, furyl, isothiazyl, isoxazyl, oxazyl, pyrazyl, pyridazyl, pyridyl, pyrimidyl, thiazyl and thienyl.

In another embodiment R₄ is an bicyclic aromatic or heteraromatic ring system comprising 9-10 ring atoms chosen from C, N, O and S, provided that not more than 3 ring atoms in any single ring are other than C. Said ring system must be substituted with R₉ and R₁₀ groups which are as described previously. The ring attachment may be either at a carbon atom or a nitrogen atom of the ring system provided that only three bonds are made to the attachment nitrogen atom. Examples of such bicyclic heteroaromatic ring system include but are not limited to 1,5-naphthyridyl, 6-naphthyridyl, 1,7-naphthyridyl, 1,8-naphthyridyl, 2,6-naphthyridyl, 2,7-naphthyridyl, cinnolyl, isoquinolyl, phthalazyl, quinazolyl, quinolyl, quinoxalyl, benzo[d][1,2,3]triazyl, benzo[e][1,2,4]triazyl, pyrido[2,3-b]pyrazyl, pyrido[2,3-c]pyridazyl, pyrido[2,3-d]pyrimidyl, pyrido[3,2-b]pyrazyl, pyrido[3,2-c]pyridazyl, pyrido[3,2-d]pyrimidyl, pyrido[3,4-b]pyrazyl, pyrido[3,4-c]pyridazyl, pyrido[3,4-d]pyrimidyl, pyrido[4,3-b]pyrazyl, pyrido[4,3-c]pyridazyl, pyrido[4,3-d]pyrimidyl, quinazolyl, 1H-benzo[d][1,2,3]triazoyl, 1H-benzo[d]imidazoyl, 1H-indazoyl, 1H-indoyl, 2H-benzo[d][1,2,3]triazoyl, 2H-pyrazolo[3,4-b]pyridyl, 2H-pyrazolo[4,3-b]pyridyl, [1,2,3]triazolo[1,5-a]pyridyl, [1,2,4]triazolo[1,5-a]pyridyl, [1,2,4]triazolo[4,3-a]pyridyl, benzo[b]thiophenyl, benzo[c][1,2,5]oxadiazyl, benzo[c][1,2,5]thiadiazolyl, benzo[d]isothiazoyl, benzo[d]isoxazoyl, benzo[d]oxazoyl, benzo[d]thiazoyl, benzofuryl, imidazo[1,2-a]pyrazyl, imidazo[1,2-a]pyridyl, imidazo[1,2-a]pyrimidyl, imidazo[1,2-b]pyridazyl, imidazo[1,2-c]pyrimidyl, imidazo[1,5-a]pyrazyl, imidazo[1,5-a]pyridyl, imidazo[1,5-a]pyrimidyl, imidazo[1,5-b]pyridazyl, imidazo[1,5-c]pyrimidyl, indolizyl, pyrazolo[1,5-a]pyrazyl, pyrazolo[1,5-a]pyridyl, pyrazolo[1,5-a]pyrimidyl, pyrazolo[1,5-b]pyridazine, pyrazolo[1,5-c]pyrimidine, pyrrolo[1,2-a]pyrazine, pyrrolo[1,2-a]pyrimidyl, pyrrolo[1,2-b]pyridazyl, pyrrolo[1,2-c]pyrimidyl, 1H-imidazo[4,5-b]pyridyl, 1H-imidazo[4,5-c]pyridyl, 1H-pyrazolo[3,4-b]pyridyl, 1H-pyrazolo[3,4-c]pyridyl, 1H-pyrazolo[4,3-b]pyridyl, 1H-pyrazolo[4,3-c]pyridyl, 1H-pyrrolo[2,3-b]pyridyl, 1H-pyrrolo[2,3-c]pyridyl, 1H-pyrrolo[3,2-b]pyridyl, 1H-pyrrolo[3,2-c]pyridyl, 2H-indazoyl, 3H-imidazo[4,5-b]pyridyl, 3H-imidazo[4,5-c]pyridyl, benzo[c]isothiazyl, benzo[c]isoxazyl, furo[2,3-b]pyridyl, furo[2,3-c]pyridyl, furo[3,2-b]pyridyl, furo[3,2-c]pyridiyl, isothiazolo[4,5-b]pyridyl, isothiazolo[4,5-c]pyridyl, isothiazolo[5,4-b]pyridyl, isothiazolo[5,4-c]pyridyl, isoxazolo[4,5-b]pyridyl, isoxazolo[4,5-c]pyridyl, isoxazolo[5,4-b]pyridyl, isoxazolo[5,4-c]pyridyl, oxazolo[4,5-b]pyridyl, oxazolo[4,5-c]pyridyl, oxazolo[5,4-b]pyridyl, oxazolo[5,4-c]pyridyl, thiazolo[4,5-b]pyridiyl, thiazolo[4,5-c]pyridyl, thiazolo[5,4-b]pyridyl, thiazolo[5,4-c]pyridyl, thieno[2,3-b]pyridyl, thieno[2,3-c]pyridyl, thieno[3,2-b]pyridyl and thieno[3,2-c]pyridyl.

### GENERAL DEFINITIONS

Alkyl is meant to denote a methyl group or a linear or branched saturated or unsaturated aliphatic C2-C7 hydrocarbon. Unsaturation in the form of a double or triple carbon-carbon bond may be internal or terminally located and in the case of a double bond both cis and trans isomers are included. Examples of alkyl groups include but are not limited to methyl, ethyl, isobutyl, neopentyl, cis and trans 2-butenyl, isobutenyl, propargyl. Cycloalkyl is a C3-C7 cyclic hydrocarbon. Examples of cycloalkyl groups include cyclopropyl, cyclobutyl, cyclopentyl, cyclohexyl and cyclopentenyl. Cycloalkylmethyl is a cycloalkyl group attached to a methylene spacer. Examples include cyclopropylmethyl and cyclohexylmethyl.

Alkoxy is alkyl-O— wherein alkyl is as defined above.

Alkylthio is alkyl-S— wherein alkyl is as defined above.

Alkylsulfonyl is alkyl-SO₂— wherein alkyl is as defined above. Alkylamino is alkyl-NH— wherein alkyl is as defined above. Dialkylamino is (alkyl)₂-N—.

Aryl is phenyl or napthyl.

Heteroaryl is a mono- or bi-cyclic ring system, only one ring need be aromatic, comprising 5 to 10 ring atoms selected from C, N, O and S, provided that not more than 3 ring atoms in any single ring are other than C. Examples of heteroaryl groups include but are not limited to 1,2,3-oxadiazyl, 1,2,3-thiadiazyl, 1,2,3-triazyl, 1,2,4-oxadiazyl, 1,2,4-thiadiazyl, 1,2,4-triaziyl, 1,2,5-oxadiazyl, 1,2,5-thiadiazyl, 1,3,4-oxadiazyl, 1,3,4-thiadiazyl, 1,3,5-triazine, 1H-1,2,3-triazyl, 1H-1,2,4-triazyl, 1H-imidazyl, 1H-pyrazyl, 1H-pyrroyl, 1H-tetrazyl, furyl, isothiazyl, isoxazyl, oxazyl, pyrazyl, pyridazyl, pyridyl, pyrimidyl, thiazyl, thiophenyl, 1,5-naphthyridyl, 6-naphthyridyl, 1,7-naphthyridyl, 1,8-naphthyridyl, 2,6-naphthyridyl, 2,7-naphthyridyl, cinnolyl, isoquinolyl, phthalazyl, quinazolyl, quinolyl, quinoxalyl, benzo[d][1,2,3]triazyl, benzo[e][1,2,4]triazyl, pyrido[2,3-b]pyrazyl, pyrido[2,3-c]pyridazyl, pyrido[2,3-d]pyrimidyl, pyrido[3,2-b]pyrazyl, pyrido[3,2-c]pyridazyl, pyrido[3,2-d]pyrimidyl, pyrido[3,4-b]pyrazyl, pyrido[3,4-c]pyridazyl, pyrido[3,4-d]pyrimidyl, pyrido[4,3-b]pyrazyl, pyrido[4,3-c]pyridazyl, pyrido[4,3-d]pyrimidyl, quinazolyl, 1H-benzo[d][1,2,3]triazoyl, 1H-benzo[d]imidazoyl, 1H-indazoyl, 1H-indoyl, 2H-benzo[d][1,2,3]triazoyl, 2H-pyrazolo[3,4-b]pyridyl, 2H-pyrazolo[4,3-b]pyridyl, [1,2,3]triazolo[1,5-a]pyridyl, [1,2,4]triazolo[1,5-a]pyridyl, [1,2,4]triazolo[4,3-a]pyridyl, benzo[b]thiophenyl, benzo[c][1,2,5]oxadiazyl, benzo[c][1,2,5]thiadiazolyl, benzo[d]isothiazoyl, benzo[d]isoxazoyl, benzo[d]oxazoyl, benzo[d]thiazoyl, benzofuryl, imidazo[1,2-a]pyrazyl, imidazo[1,2-a]pyridyl, imidazo[1,2-a]pyrimidyl, imidazo[1,2-b]pyridazyl, imidazo[1,2-c]pyrimidyl, imidazo[1,5-a]pyrazyl, imidazo[1,5-a]pyridyl, imidazo[1,5-a]pyrimidyl, imidazo[1,5-b]pyridazyl, imidazo[1,5-c]pyrimidyl, indolizyl, pyrazolo[1,5-a]pyrazyl, pyrazolo[1,5-a]pyridyl, pyrazolo[1,5-a]pyrimidyl, pyrazolo[1,5-b]pyridazine, pyrazolo[1,5-c]pyrimidine, pyrrolo[1,2-a]pyrazine, pyrrolo[1,2-a]pyrimidyl, pyrrolo[1,2-b]pyridazyl, pyrrolo[1,2-c]pyrimidyl, 1H-imidazo[4,5-b]pyridyl, 1H-imidazo[4,5-c]pyridyl, 1H-pyrazolo[3,4-b]pyridyl, 1H-pyrazolo[3,4-c]pyridyl, 1H-pyrazolo[4,3-b]pyridyl, 1H-pyrazolo[4,3-c]pyridyl, 1H-pyrrolo[2,3-b]pyridyl, 1H-pyrrolo[2,3-c]pyridyl, 1H-pyrrolo[3,2-b]pyridyl, 1H-pyrrolo[3,2-c]pyridyl, 2H-indazoyl, 3H-imidazo[4,5-b]pyridyl, 3H-imidazo[4,5-c]pyridyl, benzo[c]isothiazyl, benzo[c] isoxazyl, furo[2,3-b]pyridyl, furo[2,3-c]pyridyl, furo[3,2-b]pyridyl, furo[3,2-c]pyridiyl, isothiazolo[4,5-b]pyridyl, isothiazolo[4,5-c]pyridyl, isothiazolo[5,4-b]pyridyl, isothiazolo[5,4-c]pyridyl, isoxazolo[4,5-b]pyridyl, isoxazolo[4,5-c]pyridyl, isoxazolo[5,4-b]pyridyl, isoxazolo[5,4-c]pyridyl, oxazolo[4,5-b]pyridyl, oxazolo[4,5-c]pyridyl, oxazolo[5,4-b]pyridyl, oxazolo[5,4-c]pyridyl, thiazolo[4,5-b]pyridiyl, thiazolo[4,5-c]pyridyl, thiazolo[5,4-b]pyridyl, thiazolo[5,4-c]pyridyl, thieno[2,3-b]pyridyl, thieno[2,3-c]pyridyl, thieno[3,2-b]pyridyl and thieno[3,2-c]pyridyl. If a bicyclic heteroaryl ring is substituted, it may be substituted in any ring.

Heterocycloalkyl is a monocyclic saturated or partially unsaturated ring system comprising 5-6 ring atoms C, N, O and S, provided that not more than 2 ring atoms in any single ring are other than C. In the case where the heterocyloalkyl group contains a nitrogen atom the nitrogen may be substituted with an alkyl or acyl group. Heterocycloalkyl groups may be substituted with a hydroxyl group, and alkoxy group and up to two carbonyl groups. Heterocycloalkyl groups may be linked via either carbon or nitrogen ring atoms. Examples of heterocycloalkyl groups include tetrahydrofuranyl, tetrahydropyranyl, pyrrolidinyl, pyrrolidonyl, succinimidyl, piperidinyl, piperazinyl, N-methylpiperazinyl and morpholinyl.

In the case compounds of Formula I and II may contain asymmetric centers and exist as different enantiomers or diastereomers. All enantiomers or diastereomeric forms are embodied herein.

Compounds in the disclosure may be in the form of pharmaceutically acceptable salts. The phrase “pharmaceutically acceptable” refers to salts prepared from pharmaceutically acceptable non-toxic bases and acids, including inorganic and organic bases and inorganic and organic acids. Salts derived from inorganic bases include lithium, sodium, potassium, magnesium, calcium and zinc. Salts derived from organic bases include ammonia, primary, secondary and tertiary amines, and amino acids. Salts derived from inorganic acids include sulfuric, hydrochloric, phosphoric, methanesulphonic, hydrobromic. Salts derived from organic acids include C₁₋₆ alkyl carboxylic acids, di-carboxylic acids and tricarboxylic acids such as acetic acid, propionic acid, fumaric acid, maleic acid, succinic acid, tartaric acid, adipic acid and citric acid, and alkylsulfonic acids such as methanesulphonic, and aryl sulfonic acids such as para-tolouene sulfonic acid and benzene sulfonic acid.

Compounds in the disclosure may be in the form of a solvates. This occurs when a compound of formula (I) or (II) crystallizes in a manner that it incorporates solvent molecules into the crystal lattice. Examples of solvents forming solvates are water (hydrates), MeOH, EtOH, iPrOH, and acetone.

Compounds in the disclosure may exist in different crystal forms known as polymorphs

Practitioners of the art will recognize that certain chemical groups may exist in multiple tautomeric forms. The scope of this disclosure is meant to include all such tautomeric forms. For example, a tetrazole may exist in two tautomeric forms, 1-H tetrazole and a 2-H tetrazole. This is depicted in figure below. This example is not meant to be limiting in the scope of tautomeric forms.

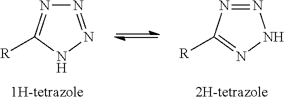

Practitioners of the art will recognize that certain electrophilic ketones, may exist in a hydrated form. The scope of this disclosure is to include all such hydrated forms. For example, a trifluoromethyl ketone may exist in a hydrated form via addition of water to the carbonyl group. This is depicted in figure below. This example is not meant to be limiting in the scope of hydrated forms.

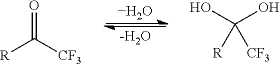

Abbreviations used in the following examples and preparations include:
Aβ Amyloid-beta
ABL Aβ lowering
Ac acyl (Me-C(O)—)
AD Alzheimer's Disease
APP Amyloid Precursor Protein
Bn Benzyl
b/p brain/plasma
BSA Bovine serum Albumin
c Cyclo
calcd. Calculated
cBu Cylcobutyl
c-Bu Cylcobutyl
cₘₐₓ Maximal concentration
cPr Cyclopropyl
c-Pr Cyclopropyl
CHAPS 3-[3-cholamidopropyl)-dimethyl-ammonio]-1-propane sulfonate
CTF Carboxy Terminal Fragment
CSF Cerebrospinal fluid
DCC N.N′, Dicyclohexylcarbodiimide
DEA Di-ethylamine
DIEA Di-isopropylethyl amine
DMAP 4-Dimethylamino Pyridine
DMF Dimethylformamide
DMSO Dimethyl sulfoxide
Dppf 1,4-Bis(diphenylphosphino)ferrocene
EDC 1-(3-Dimethylaminopropyl)-3-ethylcarbodiimide Hydrochloride
EDTA Ethylene Diamine Tetra-acetic Acid
ELISA Enzyme-Linked Immuno Sorbent Assay
Et₃N Triethylamine
Eq. Equivalent
g gram(s)
HOBt 1-Hydroxybenzotriazole
HPLC High Pressure Liquid Chromatography
h Hour(s)
hr Hour(s)
i.v or IV. Intravenous
KHMDS Potassium Hexamethydisilazide
LC-MS Liquid Chromatography-Mass Spectrometry
LDA Lithium Di-isopropylamide
m Multiplet
MeOH Methyl Alcohol or Methanol
m meta
mcpba meta-chloro perbenzoic acid
min Minute(s)
mmol millimoles
mmole millimoles
ul Microliter
μl microliter
Ms Mesylate
MS Mass Spectrometry
MW Molecular Weight (all values are ±0.05)
n normal
NBS N-Bromosuccinamide
NIS N-Iodosuccinamide
NMR Nuclear Magnetic Resonance
NMM N-Methyl Morpholine
NSAIDS Non-Steroidal Anti-Inflammatory Drugs
o ortho
o/n overnight
p para
PBS Phosphate Buffered Saline
PEPPSI 1,3-Bis(2,6-diisopropylphenyl)imidazolidene)(3-chloropyridyl)palladium(II) dichloride
PhNTf₂ 1,1,1-trifluoro-N-phenyl-N-(trifluoromethylsulfonyl)methanesulfonamide
POPd Dihydrogen dichlorobis(di-tert-butylphosphinito-kp)palladate (2-)
p.s.i. Pounds per square inch
PPAA 1-Propanephosphonic Acid Cyclic Anhydride
PyBOP® Benzotriazol-1-yl-oxytripyrrolidinophosphonium hexafluorophosphate
PK Pharmacokinetics
RT (or rt) room temperature (about 20-25° C.)
s Singlet
sat. Saturated
sec secondary
t Triplet
tert tertiary
TBAF Tetra-butyl ammonium fluoride
TFA Trifluoroacetic Acid
THF Tetrahydrofuran
TMB 3,3′5,5′ Tetramethylbenzidine
TMS Trimethylsilyl
Tf Triflate
Ts Tosylate
v/v volume/volume
wt/v weight/volume

### Preparation of Compounds

The 1,3,4-trisubstituted benzene compounds of Formulas I and II may be prepared from known fluoronitrobenzene and chloronitrobenzene starting materials e.g. 2,4-difluoronitrobenzene, 4-fluoro-2-cyano-nitrobenzene, 3-nitro-4-chlorobenzene or alternatively from 4-hydroxyphenyl acetic acid starting materials by one skilled in the art of organic synthesis using established organic synthesis procedures. The 1-position acetic acid moiety common to all compounds of Formulas I and II, as the free acid itself or as an ester derivative thereof, is already present in the case of a 4-hydroxyphenyl acetic acid or 4-hydroxyphenyl acetic acid ester starting material. In the case of a 4-fluoronitrobenzene starting materials or intermediates, the acetic acid moiety is introduced by nucleophilic aromatic substitution of the 4-fluoro group with an unsubstituted malonic ester (e.g. diethyl malonate) or a malonic ester derivative already bearing an R₁ group (e.g. diethyl 2-isobutylmalonate). Introduction of the Y—R₃ group is carried out by substitution or manipulation of suitable 3 or 4-position functional groups in the case of Formulas I and II respectively. In cases where Y is a bond, a 3 or 4-position halogen or triflate group is replaced with an aryl or heteroaryl group by carbon-carbon bond forming reaction typically a Suzuki coupling reaction. In cases where Y is O, S or N, a 3 or 4-position halogen (e.g. the corresponding 2-fluoro group of a 2,4-difluoronitrobenzene starting material) substitution reaction is performed using HO—R₃ or HS—R₃ or H₂N—R₃ and a base (e.g. NaH, K2CO3) in a suitable solvent (e.g. DMF). Compounds where Y is —S(O)— or —S(O₂)— are prepared by oxidation of compounds where Y is S. Compounds where Y is —S(O)₂N(H)—, —S(O)₂N(R₅)— are prepared by conversion of a 3 or 4-position nitro group (e.g. the nitro group of the nitrobenzene starting material) to a sulfonyl chloride via Sandmeyer reaction followed by addition of the corresponding amine. Compounds where Y is N(H)S(O)₂— or —N(R₅)S(O)₂— are prepared by reduction of a 3 or 4-position nitro group to the corresponding aniline followed by reaction with the corresponding sulfonylchloride. Compounds where Y is NHC(O)— or —N(R₅)C(O)— are prepared by reduction of a 3 or 4-position nitro group to the corresponding aniline followed by reaction with the corresponding carboxylic acid chloride. Compounds where Y is a —C(O)— are prepared by addition of an organometallic reagent (e.g. a Grignard reagent or organolithium) to a 3 or 4-position cyano group directly or in a 2-step sequence by addition of an organometallic reagent to a 3 or 4-position carboxaldehyde group followed by oxidation. Compounds where Y is —C(O)NH— or C(O)N(R₅)—)— are prepared by addition of a corresponding amine to a 3 or 4-position carboxylic acid which in turn may be prepared by hydrolysis of a 3 or 4-position cyano group. Either aromatic nucleophilic substitution of a 2-fluoro-1-nitrobenzene intermediate or alkylation of a 3 or 4-hydroxybenzene intermediate with the corresponding alkyl bromide or triflate may be used to prepare compounds of Formulas I and II where the R₄ group is OCH₂CF₃, C2-C4 alkoxy, or cyclopropyloxymethyl. Compounds wherein the R₄ group is an alkyl, aryl or heteroaryl group attached by a carbon-carbon bond may be prepared by a Suzuki coupling reaction. In this process an aryl or heteroaryl boronic acid or borate ester is reacted with an intermediate compound having a 3 or 4-position halogen or triflate group. This method results in replacement of the halogen or triflate group with an aryl or heteroaryl group which is then bonded to the intermediate at the carbon atom previously bearing the boronic acid or ester group. Compounds wherein the R₄ group is a heteroaryl group attached by a carbon-nitrogen bond may be prepared by reacting a 3 or 4-iodo intermediate with a heteroaromatic heterocycle having an acidic N—H group under Ullman reaction or copper catalyzed reaction conditions.

Compounds of Formulas I and II wherein A is C(O)NHOH, C(O)NHOCH₃, C(O)NH₂, C(O)NH(R₅), C(O)N(R₅)₂, C(O)NH(SO₂CH₃) or COCF₃ are readily prepared by from the corresponding carboxylic acid or ethylester compounds, A=CO₂H or CO₂Et. Thus, compounds of formulas I and II where A is C(O)NHOH, C(O)NHOCH₃, C(O)NH₂, C(O)NH(R₅), C(O)N(R₅)₂, C(O)NH(SO₂CH₃), may be prepared by converting the corresponding carboxylic acids first to an active ester or acid chloride according to procedures well established in the art followed by treatment with a corresponding amine of formula NH₂OH, NH₂OCH₃, NH₃, NH₂(R₅), NH(R₅)₂ or NH₂(SO₂CH₃). Compounds of Formulas I and II wherein A=tetrazole may be prepared from their corresponding nitriles A=CN which are available via dehydration of the corresponding primary amides A=C(O)NH₂ whose preparation is described above. Thus, treatment of the nitrile with an azide, such as sodium azide or tributylstanyl azide (Bu₃SnN₃) at a temperature of 20-100° C., optionally with a solvent such as DMF, THF or DMSO. Trifluoromethyl ketone compounds of formulas I and II wherein A=COCF₃ can be prepared from corresponding esters e.g. wherein A=CO₂Et by treatment with TMS-CF₃ in the presence of a fluoride source such as TBAF, KF, or CsF in solvent such as CH₂Cl₂, THF or MeCN. The reaction is run at a temperature of −20 to 100° C.

Compounds of Formula III and IV may be prepared generally as depicted in Scheme 1 or Scheme 2 using ethyl 4-benzyloxyphenylacetate XX as a starting material. The method depicted in Scheme 1 is suited to preparing compounds of Formulas III and IV in which R₁ and/or R₂ are alkyl or aralkyl groups while the method of Scheme 2 is suited to preparing derivatives in which R₁ and/or R₂ are alkoxy or alkylthio groups and amino derivatives. Thus, as depicted in Scheme 1 an R₁ or suitably protected R₁ alkyl or aralkyl group is introduced in the first step by treating an intermediate of Formula XX with one equivalent of a suitable deprotonating base such as sodium hydride in an appropriate organic solvent followed by the addition of the corresponding reactive alkyl bromide R₁Br such as isobutylbromide to yield XXI where R₂ is hydrogen. In cases where a second alkyl or aralkyl group is present this alkylation step is repeated using R₂Br as an alkylating agent.

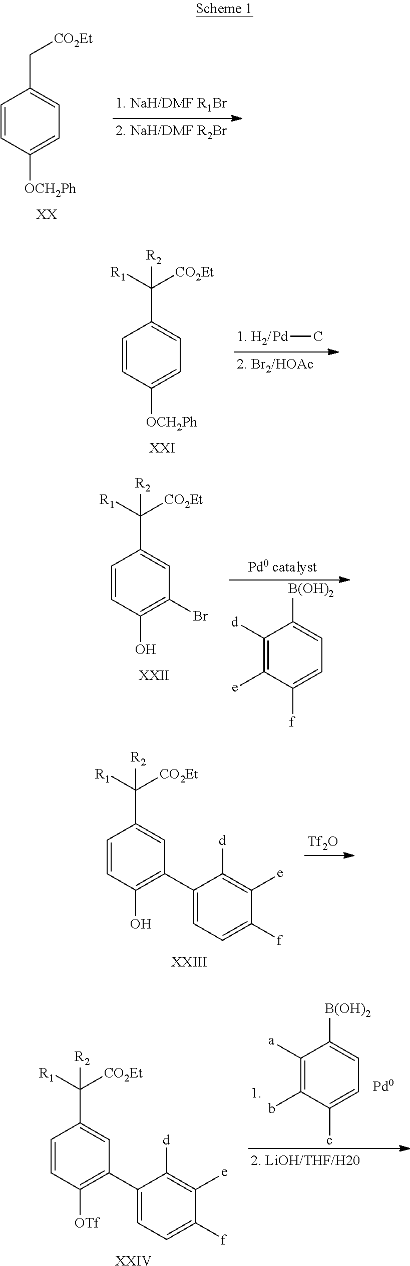

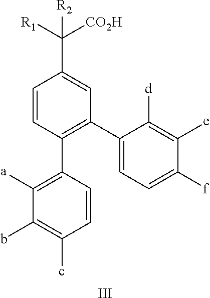

The benzyl group is then removed under standard catalytic hydrogenation conditions and the resulting phenol is treated with bromine in acetic acid to give the bromophenol intermediate XXII. Suzuki coupling of a substituted aryl or heteroaryl boronic acid derivative using a suitable palladium(0) catalyst typically bearing with phosphine ligands (e.g. Pd(PPh₃)₄ or tetrakistriphenylphosphine) gives biphenyl intermediates of general structure XXIII. The phenolic group of XXIII is converted to the corresponding trifluoromethanesulfonate (OSO₂CF₃ or OTf) compounds XXIV. Suzuki coupling of compounds of general formula XXIV with a substituted aryl or heteroaryl boronic acid derivative followed by standard ester hydrolysis yields compounds of Formula III or Formula IV in which R₁ and/or R₂ are alkyl or aralkyl groups.

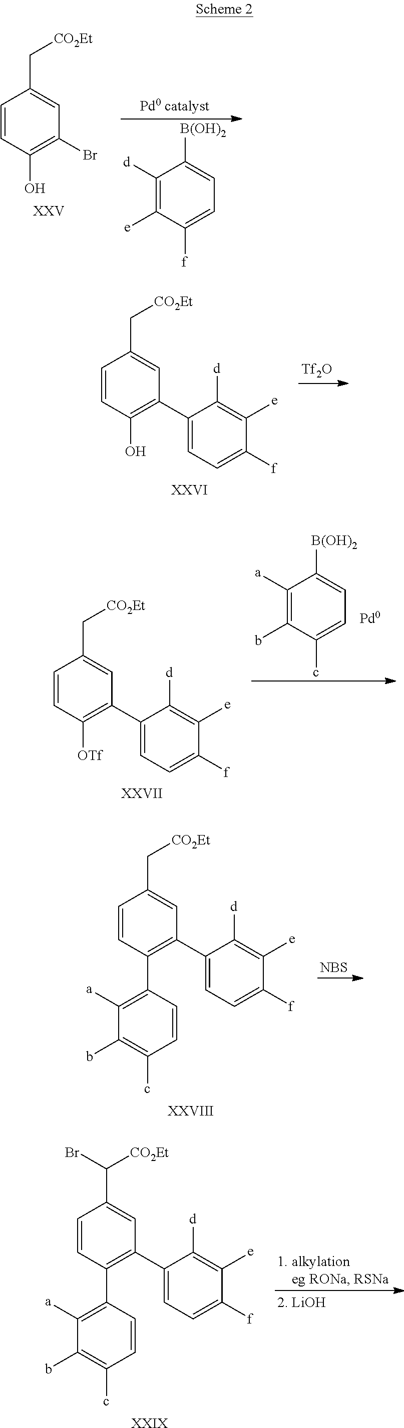

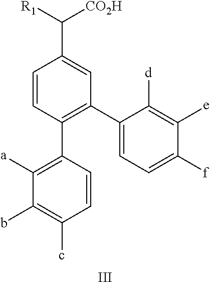

The method depicted in Scheme 2 is suited to preparing compounds of Formulas III and IV in which R₁ and/or R₂ are alkoxy or alkylthio groups or amino derivatives. The synthesis of the key intermediate XXVIII depicted in Scheme 2 in which R₁ and R₂ are both hydrogen is conducted in analogous fashion to the process describe in Scheme 1. Bromination of intermediate XXVIII with N-bromosuccinimide (NBS) yields intermediate XXIX in which R₁=Br. In a subsequent step the Br atom is replaced by a suitable alkoxide, thiolate or masked amine nucleophile (e.g. azide or N₃). The product of the latter reaction is either directly subjected to ester hydrolysis or further processed (e.g. by conversion the masked amine to an amino group followed by reductive amination to give mono or dialkylamine derivatives) and then subjected to final ester hydrolysis to give compounds of Formulas III and IV in which R₁ and/or R₂ are alkoxy or alkylthio groups or amino derivatives.

Compounds of Formula V wherein Y is O, S or SO₂ may be prepared generally as depicted in Scheme 3. Accordingly, the 2-fluoro group of 2,4-difluoronitrobenzene is selectively displaced by reaction with R₃YH (Y=O, S or SO₂) under basic conditions (e.g. NaH/DMF). In a subsequent step the 4-fluoro group undergoes nucleophilic aromatic substitution reaction by treatment with a malonate derivative (e.g. diethylmalonate) under basic conditions (e.g. NaH/DMF). The product of the latter reaction is converted to the corresponding ethyl 4-aminophenylacetate intermediate XXVI by acid hydrolysis, Fischer esterification with ethanol and reduction of the nitro group to the aniline (e.g. with SnCl₂/HCl). Conversion of the aniline amino group of intermediate XXVI to the corresponding iodide group in XXVII is carried out by Sandmeyer reaction via diazotization and treatment of the resulting diazonium salt with copper iodide. Suzuki coupling of compounds of general formula XXVII with a substituted aryl or heteroaryl boronic acid derivative yield intermediates of Formula XXVIII. The latter maybe converted to compounds of Formula V e.g. by alkylation followed by standard ester hydrolysis yields as shown below and previously described in Scheme 1. Alternatively intermediates of Formula XXVIII may be brominated and processed to yield compounds of Formula V in which R₁ and/or R₂ are alkoxy, alkylthio or substituted amino groups.

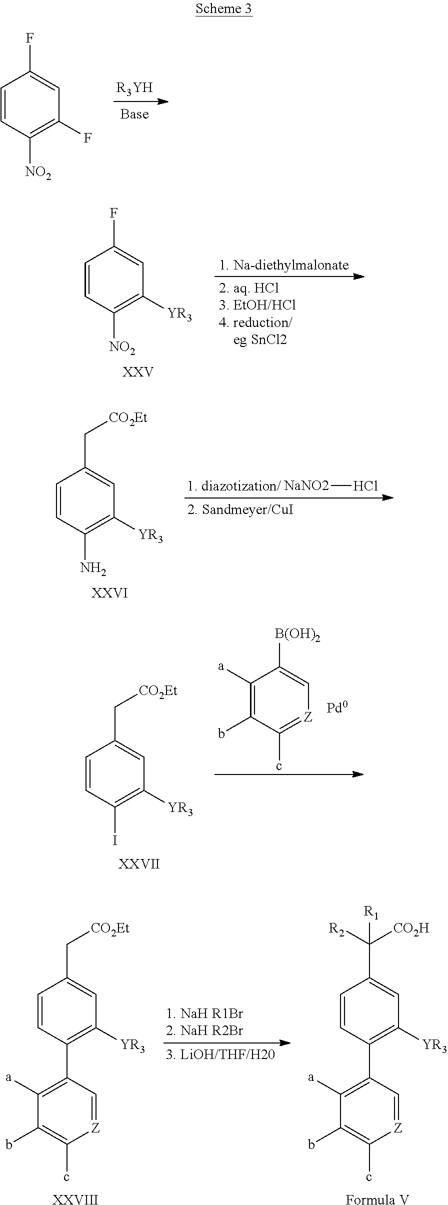

Compounds of Formula VI may be prepared generally as depicted in Scheme 4. Accordingly, the chloro group of 3-nitro-4-chlorobenzaldehyde displaced by reaction with R₃YH (Y=O, S or SO₂) under basic conditions (e.g. NaH/DMF). The resulting intermediate compounds XXIX are converted to XXX by a 4-step sequence. First the carboxaldehyde group is reduced with sodium borohydride to the alcohol which is converted to the corresponding bromide with PBr₃. Bromide displacement with NaCN yields the corresponding cyanide which is converted to the corresponding ester by Pinner reaction with ethanolic hydrochloric acid solution. Reduction of the nitro group (e.g. with SnCl₂) then gives aniline intermediate XXX. Conversion of the aniline amino group of intermediate XXX to the corresponding iodide group in XXXI is carried out by Sandmeyer reaction via diazotization and treatment of the resulting diazonium salt with copper iodide. Suzuki coupling of compounds of general formula XXXI with a substituted aryl or heteroaryl boronic acid derivative yield intermediates of Formula XXXII. The latter maybe converted to compounds of Formula VI e.g., by alkylation followed by standard ester hydrolysis yields as shown below and previously described in Scheme 1. Alternatively intermediates of Formula XXXII may be brominated and processed to yield compounds of Formula VI in which R₁ and/or R₂ are alkoxy, alkylthio or substituted amino groups.

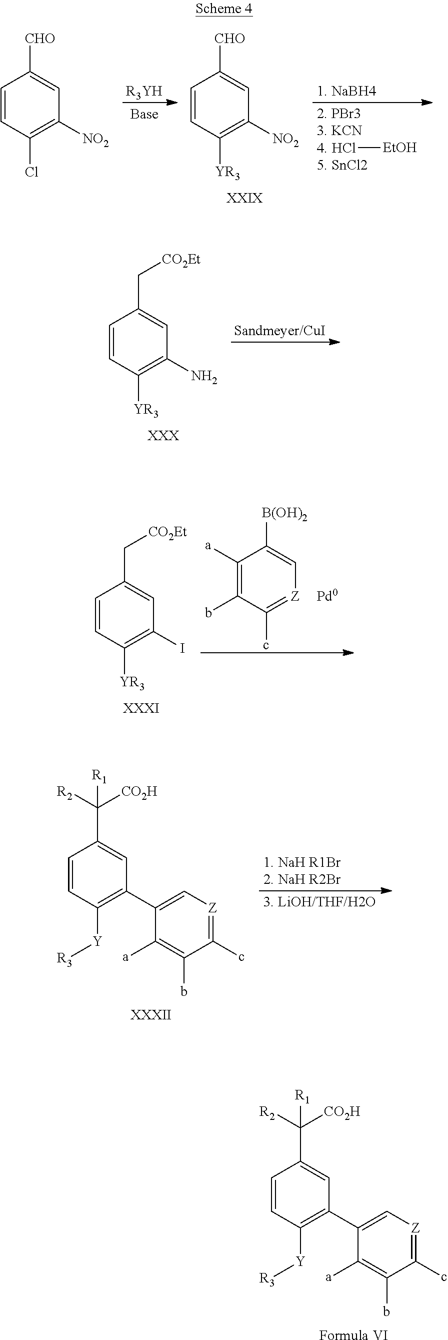

Compounds of Formulas I and II wherein R₄═SO₂N(R₇)(R₈) may be prepared as depicted in Schemes 5 and 6 from intermediates XXX and XXXIII.

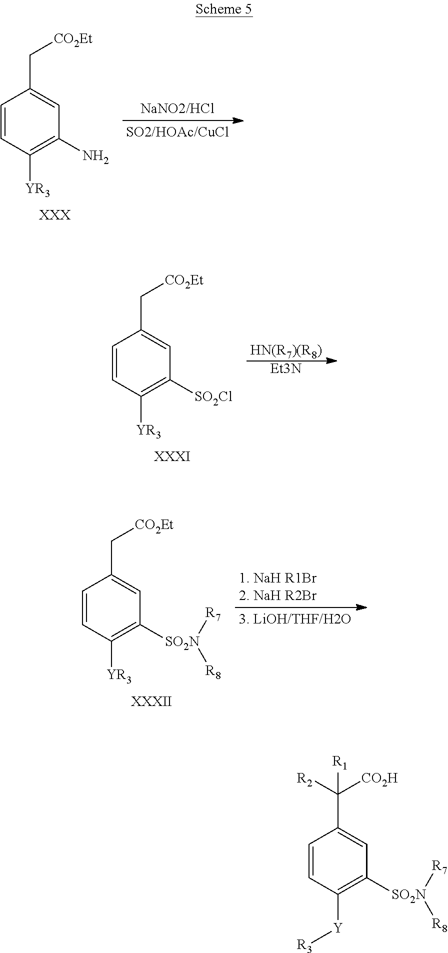

Accordingly, sulfonylchlorides XXXI and XXXIV are prepared by treating the diazonium species formed in situ with SO₂ in acetic acid and CuCl. Subsequent reaction with amines gives sulfonamide intermediates XXXII and XXXV. These intermediates can be processed to compounds of Formula I and II wherein R₄═SO₂N(R₇)(R₈) and A=CO₂H by introduction of R₁ and/or R₂ groups followed by ester hydrolysis under conditions described above.

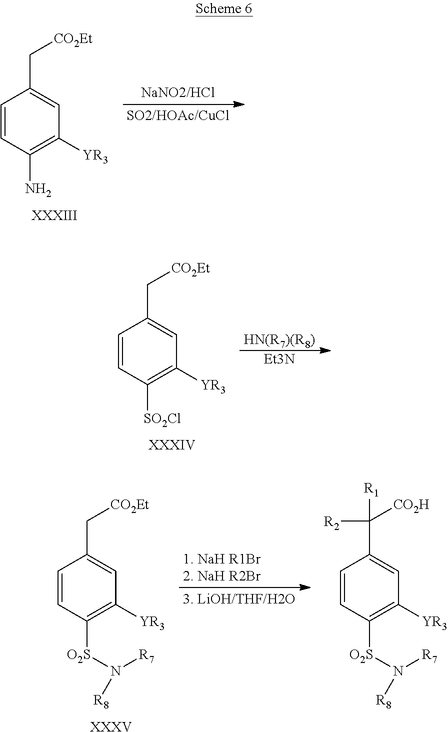

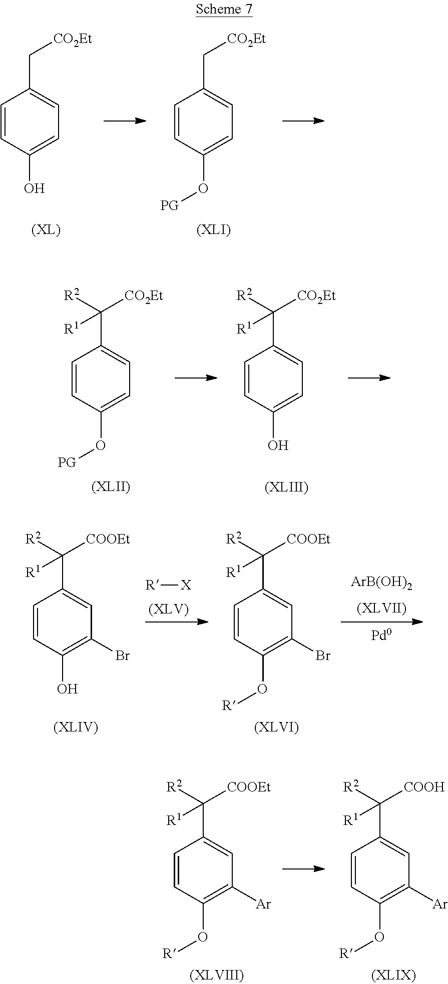

The phenol (XL) may be protected by methods known to those of ordinary skill in the art to give the protected species (XLI). The protecting group of compound (XLII) can be removed by methods known to those of ordinary skill in the art to furnish the phenol (XLIII). The phenol can be transformed into the bromo phenol (XLIV) by treatment with molecular bromine or NBS. The reaction can be performed in an inert solvent such as CCl₄, CHCl₃ or CH₂Cl₂ usually at a temperature of about −20 to 50° C. The phenol (XLIV) is alkylated with the appropriate electrophile R′-X (XLV) in the presence of a base such as K₂CO₃, Cs₂CO₃, LiHMDs, NaH, LDA or KHMDS to give the compounds of formula (XII). The reaction is run in an inert solvent such as DME, THF, toluene, DMF, DMSO, dioxane or a combination of such solvents in a temperature range of −20 to 100° C. The compounds of formula (XLVIII) is synthesized by treating the aromatic compounds of formula (XLVI) with the appropriate boronic acid (XLVII) in the presence of a palladium catalyst such as Pd(PPh₃)₄, PdCl₂(dppf), POPd or PEPPSI and a base such as Cs₂CO₃, KOH, CsF, NaOH or K₂CO₃. The reaction is usually carried out in a solvent such as DME, THF, toluene, water or a mixture of said solvents at a temperature of 0-120° C. The ester (XLVIII) is then hydrolyzed to the corresponding acid (XLIX) by methods known to those of ordinary skill. For example treating the ester with a base such as LiOH, NaOH, KOH or KOTMS in a solvent such as THF, dioxane, MeOH, EtOH, water or a mixture of such solvents.

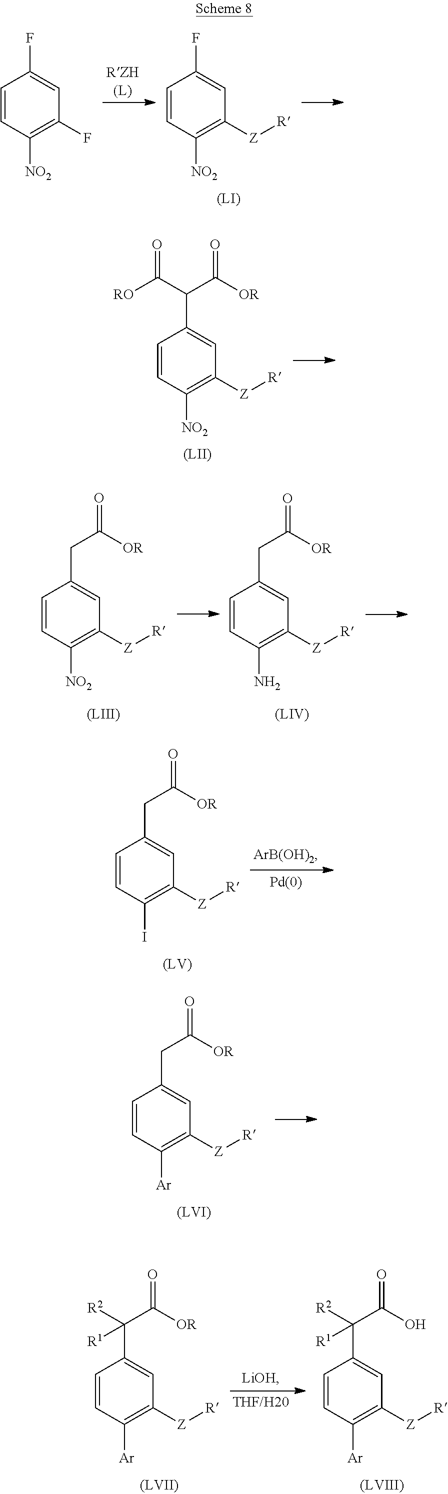

The compounds of formula (LI) are prepared by treating 2,4 difluoronitrobenzene with compound (L), where Z represents an oxygen or sulfur atom. The reaction is usually run in an inert solvent such as TH, dioxane, DMF or DMSO the presence of a base such as K₂CO₃, Cs₂CO₃, LiHMDs, NaH, LDA or KHMDS. The reaction is usually run at a temperature of −10 to 100° C. The malonate derivative is prepared by treating the nitro compound (LI) with a protected malonic acid, such as diethyl malonate or tert-butyl ethyl malonate. The reaction is performed in solvent such as DMSO, DMF, THF or dioxane in the presence of a base such as K₂CO₃, Cs₂CO₃, LiHMDs, NaH, LDA or KHMDS. The reaction is usually performed at a temperature of 0-120° C. The malonate derivative (LII) may be transformed in the phenyl acetic esters of formula (LIII) by different methods depending on which ester groups are present in the malonate derivative. For example if the malonate bears a tert-butyl ester the product may be directly transformed into (LIII) by treatment with an acid such as glacial AcOH and heating under reflux. If the malonate derivate bears only methyl or ethyl esters, the material is treated with a base such as LiOH, NaOH or KOH in a solvent such as MeOH, EtOH, water or a mixture of said solvents. The reaction is usually performed at 20-100° C. These reaction conditions may lead to saponification of the phenyl acetic ester (LIII) and the subsequent acid can be re-esterified via procedures known to those of ordinary skill in the art. The nitrobenzenes of formula (LIII) are reduced to the corresponding anilines (LIV) via reduction using a metal or metal salt such as Zinc, iron or SnCl₂ in the presence of an acid such as HCl or AcOH. The reaction is usually performed at a temperature of 20-100° C. Alternatively the reduction may performed via hydrogenation over a metal such as Pd/C, Pd(OH)₂ or PtO₂ in a solvent such as MeOH, EtOH, THF, dioxane, water or a mixture of said solvents. The reaction is performed under an atmosphere of molecular hydrogen at a pressure of 14-1400 p.s.i. and at a temperature of 20-100° C. The aniline (LIV) is transformed into the aryl iodide (or aryl bromide) via a Sandmeyer reaction. The intermediate diazonium is produced by treating the aniline (LIV) with NaNO₂ in the presence of an acid such as H₂SO₄ or HCl. The reaction is usually performed at −20 to 10° C. using a co-solvent such as EtOH, water, MeOH, THF or a mixture of said solvents. The diazonium intermediate is then treated with an iodide source such as CuI or KI (or CuBr, KBr for aryl bromides), at a temperature of 0-80° C. to give the aryl iodides of formula (LV). The aryl iodides are converted into the compounds of formula (LVI) via a Suzuki reaction. The iodides are treated with the appropriate boronic acid (XLVII) in the presence of a palladium catalyst such as Pd(PPh₃)₄, PdCl₂(dppf), POPd or PEPPSI and a base such as Cs₂CO₃, KOH, CsF, NaOH or K₂CO₃. The reaction is usually carried out in a solvent such as DME, THF, toluene, water or a mixture of said solvents at a temperature of 0-120° C. The phenyl acetic ester of formula (LVI) may be alkylated by treatment with a base such as NaOH, LiHMDs, NaH, ^{t}BuOK, LDA or KHMDs in an inert solvent such as THF, DMSO or DMF at a temperature of −78 to 20° C. followed by the addition of the appropriate alkylating agent(s), such as an alkyl halide. If in the compound of formula (LVII) both R¹ and R² are not hydrogen, a person of ordinary skill in the art will recognize that it may necessary to conduct two separate alkylation reactions in a sequential manner. If R¹ and R² are taken together to form a ring then a di-alkylating agent of such as 1,2 di-bromoethane, 1,3 di-bromopropane, 1,4 di-bromobutane or 1,5 di-bromopentane may be used. The ester (LVII) is then hydrolyzed to the corresponding acid (LVIII) by methods known to those of ordinary skill. For example treating the ester with a base such as LiOH, NaOH, KOH or KOTMS in a solvent such as THF, dioxane, MeOH, EtOH, water or a mixture of such solvents.

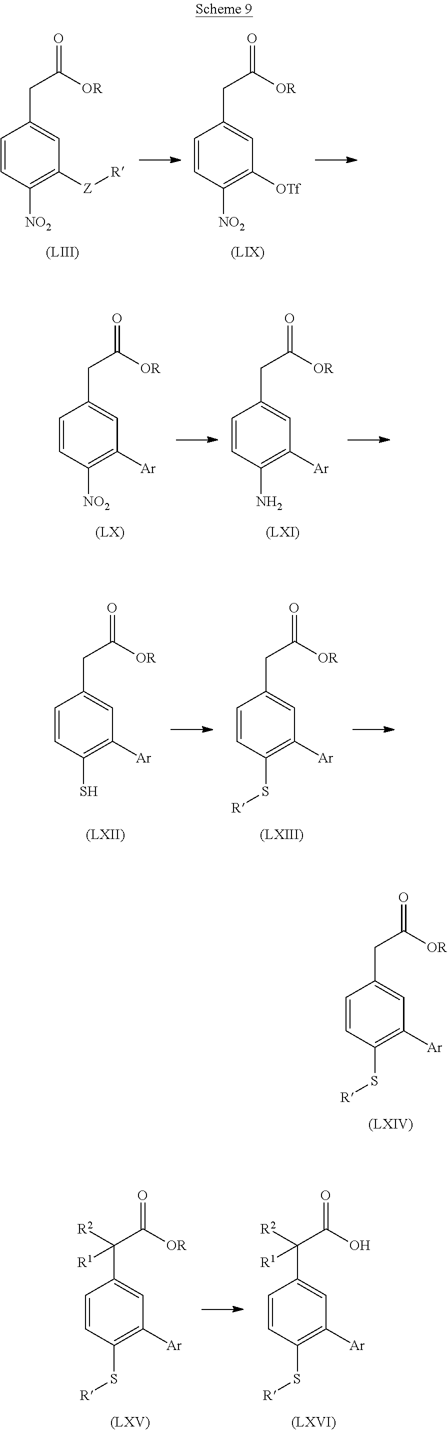

The thioethers of formula (LXVI) may be synthesized according to scheme 9. The nitro phenol (LIII) may be transformed into the triflate (LIX) via a two step protocol. If R′ in this instance represents a protecting group, it may be removed to reveal the corresponding phenol by methods known to those of ordinary skill in the art. The phenol is then converted into the triflate by treatment with a triflating reagent such as triflic anhydride (Tf₂O) or PhNTf₂, in an inert solvent such as THF or CH₂Cl₂ in the presence of a base such as pyridine or lutidine. The reaction is usually run at a temperature of −20 to 40° C.

The resultant triflate (LIX) is transformed into the compound of formula (LX) by treatment with a boronic acid of formula (XLVII) in the presence of a palladium catalyst such as Pd(PPh₃)₄, PdCl₂(dppf), POPd or PEPPSI, a base such as Cs₂CO₃, KOH, CsF, NaOH or K₂CO₃ and a chloride source such as lithium chloride. The reaction is usually carried out in a solvent such as DME, THF, toluene, water or a mixture of said solvents at a temperature of 0-120° C. The nitrobenzenes of formula (LX) are reduced to the corresponding anilines (LXI) via reduction using a metal or metal salt such as Zinc, iron or SnCl₂ in the presence of an acid such as HCl or AcOH. The reaction is usually performed at a temperature of 20-100° C. Alternatively the reduction may performed via hydrogenation over a metal such as Pd/C, Pd(OH)₂ or PtO₂ in a solvent such as MeOH, EtOH, THF, dioxane, water or a mixture of said solvents. The reaction is performed under an atmosphere of molecular hydrogen at a pressure of 14-1400 p.s.i. and at a temperature of 20-100° C. The thiols of formula (LXII) are produced via an intermediate diazonium species. The intermediate diazonium is produced by treating the aniline (LXI) with NaNO₂ in the presence of an acid such as H₂SO₄ or HCl. The reaction is usually performed at −20 to 10° C. using a co-solvent such as EtOH, water, MeOH, THF or a mixture of said solvents. The diazonium intermediate is then treated with a sulfur source such as Na₂S₂. The mixture may have to be reduced using a reagent such as NaBH4 and treated with an acid such as HCl to give the desired thiol (LXII), or alternatively the diazonium ion may be treated with KSCN and the intermediate hydrolyzed to give the thiol (LXII).

The thiol (LXII) is alkylated with an appropriate electrophile R′-X (XLV) in the presence of a base such as K₂CO₃, Cs₂CO₃, LiHMDs, NaH, LDA or KHMDS to give the compounds of formula (XII). The reaction is run in an inert solvent such as DME, THF, toluene, DMF, DMSO, dioxane or a combination of such solvents and is performed at a temperature of 0-100° C. The phenyl acetic ester of formula (LXIII) may be alkylated by treatment with a base such as NaOH, LiHMDs, NaH, ^{t}BuOK, LDA or KHMDs in an inert solvent such as THF or DMF at a temperature of −78 to 20° C. followed by the addition of the appropriate alkylating agent(s), such as an alkyl halide. If in the compound of formula (LXIV) both R¹ and R² are not hydrogen, a person of ordinary skill in the art will recognize that it may necessary to conduct two separate alkylation reactions in a sequential manner. If R¹ and R² are taken together to form a ring then a di-alkylating agent of such as 1,2 di-bromoethane, 1,3 di-bromopropane, 1,4 di-bromobutane 1,5 di-bromopentane may be used. The ester (LXIV) is then hydrolyzed to the corresponding acid (LXV) by methods known to those of ordinary skill. For example treating the ester with a base such as LiOH, NaOH, KOH or KOTMS in a solvent such as THF, dioxane, MeOH, EtOH, water or a mixture of such solvents.

Practitioners of the art will also recognize that the order of certain steps in the above schemes may be altered.

Boronic acids are either commercially available or their synthesis is known to those of ordinary skill in the art.

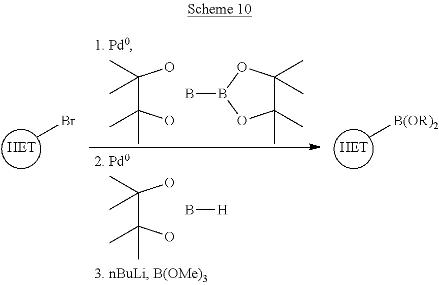

Scheme 10 depicts the synthesis of heteroaromatic and aryl boronic acids/esters.

The bromides are treated in the presence of a Pd catalyst such as Pd(PPh₃)₄, PdCl₂(dppf), POPd or PEPPSI is the presence of a boron transfer reagent such as 4,4,4′,4′,5,5,5′,5′-octamethyl-2,2′-bi(1,3,2-dioxaborolane) or 4,4,5,5-tetramethyl-1,3,2-dioxaborolane. The reaction is performed in the presence of a base such as Et₃N, K₂CO₃ in a solvent such as dioxane, THF or DMF. The reaction is run at a temperature of 20-100° C. Alternatively the boronic acids/esters may be prepared from the bromide via treatment with a BuLi, (n-, sec- or tert-) and trapping the intermediate organolithium with a trialkyl borate such as B(OMe)₃ or B(OEt)₃. N,N,N,N, tetraethylenediamine may also be added to the reaction mixture. The reaction is usually carried out in a solvent such as THF, Et₂O, n-pentane, hexane or a mixture of said solvents.

Reactive groups not involved in the above processes can be protected with standard protecting groups during the reactions and removed by standard procedures (T. W. Greene & P. G. M. Wuts, Protecting Groups in Organic Synthesis, Third Edition, Wiley-Interscience) known to those of ordinary skill in the art. Presently preferred protecting groups include methyl, benzyl, acetate, cyclopropylmethyl and tetrahydropyranyl for the hydroxyl moiety, and BOC, CBz, trifluoroacetamide and benzyl for the amino moiety, methyl, ethyl, tert-butyl and benzyl esters for the carboxylic acid moiety.

### Examples Compounds within Formula I or Formula II

In a further aspect the compounds of the disclosure (Formula I or Formula II) are embodied in general Formula A with distinct examples listed in Table 1 below.

TABLE 1

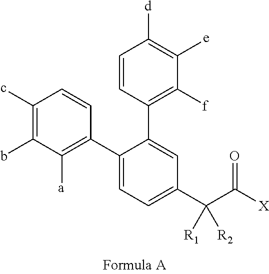

| Ex | X | R₁ | R₂ | A | b | c | d | E | f |
|---|---|---|---|---|---|---|---|---|---|
| 1 | OH | SO₂CH₃ | H | H | H | F | F | H | H |
| 2 | OH | SO₂CH₃ | H | H | H | CF₃ | CF₃ | H | H |
| 3 | OH | SO₂CH₃ | H | H | H | Cl | Cl | H | H |
| 4 | OH | SO₂CH₃ | H | F | H | H | F | H | H |
| 5 | OH | SO₂CH₃ | H | H | F | H | F | H | H |
| 6 | OH | SO₂CH₃ | H | H | Cl | OCH₃ | F | H | H |
| 7 | OH | SO₂CH₃ | H | H | Cl | OH | F | H | H |
| 8 | OH | SCH₂CH₃ | H | H | H | F | F | H | H |
| 9 | OH | SCH₂CH₃ | H | H | H | CF₃ | CF₃ | H | H |
| 10 | OH | SCH₂CH₃ | H | H | H | Cl | Cl | H | H |
| 11 | OH | SCH₂CH₃ | H | F | H | H | F | H | H |
| 12 | OH | SCH₂CH₃ | H | H | F | H | F | H | H |
| 13 | OH | SCH₂CH₃ | H | H | Cl | OCH₃ | F | H | H |
| 14 | OH | SCH₂CH₃ | H | H | Cl | OH | F | H | H |
| 15 | OH | SO₂CH₂CH₃ | H | H | H | F | F | H | H |
| 16 | OH | SO₂CH₂CH₃ | H | H | H | CF₃ | CF₃ | H | H |
| 17 | OH | SO₂CH₂CH₃ | H | H | H | Cl | Cl | H | H |
| 18 | OH | SO₂CH₂CH₃ | H | F | H | H | F | H | H |
| 19 | OH | SO₂CH₂CH₃ | H | H | F | H | F | H | H |
| 20 | OH | SO₂CH₂CH₃ | H | H | Cl | OCH₃ | F | H | H |
| 21 | OH | SO₂CH₂CH₃ | H | H | Cl | OH | F | H | H |
| 22 | OH | OCH₂ | H | H | H | F | F | H | H |
| | | CH₂OCH₃ | | | | | | | |
| 23 | OH | OCH₂ | H | H | H | CF₃ | CF₃ | H | H |
| | | CH₂OCH₃ | | | | | | | |
| 24 | OH | OCH₂ | H | H | H | Cl | Cl | H | H |
| | | CH₂OCH₃ | | | | | | | |
| 25 | OH | OCH₂ | H | F | H | H | F | H | H |
| | | CH₂OCH₃ | | | | | | | |
| 26 | OH | OCH₂ | H | H | F | H | F | H | H |
| | | CH₂OCH₃ | | | | | | | |
| 27 | OH | OCH₂ | H | H | Cl | OCH₃ | F | H | H |
| | | CH₂OCH₃ | | | | | | | |
| 28 | OH | OCH₂ | H | H | H | CF₃ | CF₃ | H | H |
| | | CH₂SCH₃ | | | | | | | |
| 29 | OH | OCH₂CH₂ | H | H | H | CF₃ | CF₃ | H | H |
| | | SO₂CH₃ | | | | | | | |
| 30 | OH | SCH₂CH₂OCH₃ | H | H | H | CF₃ | CF₃ | H | H |
| 31 | OH | NHC(O)CH₃ | H | H | H | CF₃ | CF₃ | H | H |
| 32 | OH | NHC(O) | H | H | H | CF₃ | CF₃ | H | H |
| | | CH₂CH₃ | | | | | | | |
| 33 | OH | NHC(O) | H | H | H | CF₃ | CF₃ | H | H |
| | | CH(CH₃)₂ | | | | | | | |
| 34 | OH | CH₃ | H | H | H | OCH₂ | CF₃ | H | H |
| | | | | | | CH₂OCH₃ | | | |
| 35 | OH | CH₂CH₃ | H | H | H | OCH₂ | CF₃ | H | H |
| | | | | | | CH₂OCH₃ | | | |
| 36 | OH | CH2CH₂CH₃ | H | H | H | OCH₂ | CF₃ | H | H |
| | | | | | | CH₂OCH₃ | | | |
| 37 | OH | CH₂CH(CH₃)₂ | H | H | H | OCH₂ | CF₃ | H | H |
| | | | | | | CH₂OCH₃ | | | |
| 38 | OH | cyclopro- | H | H | H | OCH₂ | CF₃ | H | H |
| | | pylmethyl | | | | CH₂OCH₃ | | | |
| 39 | OH | benzyl | H | H | H | OCH₂ | CF₃ | H | H |
| | | | | | | CH₂OCH₃ | | | |
| 40 | OH | 4-F-benzyl | H | H | H | OCH₂ | CF₃ | H | H |
| | | | | | | CH₂OCH₃ | | | |
| 41 | OH | (CH₂)₂ | | H | H | OCH₂ | CF₃ | H | H |
| | | | | | | CH₂OCH₃ | | | |
| 42 | OH | (CH₂)₄ | | H | H | OCH₂ | CF₃ | H | H |
| | | | | | | CH₂OCH₃ | | | |
| 43 | OH | CH₃ | H | H | H | CF₃ | OCH₂ | H | H |
| | | | | | | | CH₂OCH₃ | | |
| 44 | OH | CH₂CH₃ | H | H | H | CF₃ | OCH₂ | H | H |
| | | | | | | | CH₂OCH₃ | | |
| 45 | OH | CH2CH₂CH₃ | H | H | H | CF₃ | OCH₂ | H | H |
| | | | | | | | CH₂OCH₃ | | |
| 46 | OH | CH₂CH(CH₃)₂ | H | H | H | CF₃ | OCH₂ | H | H |
| | | | | | | | CH₂OCH₃ | | |
| 47 | OH | cyclopro- | H | H | H | CF₃ | OCH₂ | H | H |
| | | pylmethyl | | | | | CH₂OCH₃ | | |
| 48 | OH | benzyl | H | H | H | CF₃ | OCH₂ | H | H |
| | | | | | | | CH₂OCH₃ | | |
| 49 | OH | 4-F-benzyl | H | H | H | CF₃ | OCH₂ | H | H |
| | | | | | | | CH₂OCH₃ | | |
| 50 | OH | (CH₂)₂ | | H | H | CF₃ | OCH₂ | H | H |
| | | | | | | | CH₂OCH₃ | | |
| 51 | OH | (CH₂)₄ | | H | H | CF₃ | OCH₂ | H | H |
| | | | | | | | CH₂OCH₃ | | |
| 52 | OH | CH₃ | H | H | H | SO₂CH₃ | CF₃ | H | H |
| 53 | OH | CH₂CH₃ | H | H | H | SO₂CH₃ | CF₃ | H | H |
| 54 | OH | CH₂CH₂CH₃ | H | H | H | SO₂CH₃ | CF₃ | H | H |
| 55 | OH | CH₂CH(CH₃)₂ | H | H | H | SO₂CH₃ | CF₃ | H | H |
| 56 | OH | cyclopro- | H | H | H | SO₂CH₃ | CF₃ | H | H |
| | | pylmethyl | | | | | | | |
| 57 | OH | benzyl | H | H | H | SO₂CH₃ | CF₃ | H | H |
| 58 | OH | 4-F-benzyl | H | H | H | SO₂CH₃ | CF₃ | H | H |
| 59 | OH | (CH₂)₂ | | H | H | SO₂CH₃ | CF₃ | H | H |
| 60 | OH | (CH₂)₄ | | H | H | SO₂CH₃ | CF₃ | H | H |
| 61 | OH | CH₃ | H | H | H | CF₃ | SO₂CH₃ | H | H |
| 62 | OH | CH₂CH₃ | H | H | H | CF₃ | SO₂CH₃ | H | H |
| 63 | OH | CH2CH₂CH₃ | H | H | H | CF₃ | SO₂CH₃ | H | H |
| 64 | OH | CH₂CH(CH₃)₂ | H | H | H | CF₃ | SO₂CH₃ | H | H |
| 65 | OH | cyclopro- | H | H | H | CF₃ | SO₂CH₃ | H | H |
| | | pylmethyl | | | | | | | |
| 66 | OH | benzyl | H | H | H | CF₃ | SO₂CH₃ | H | H |
| 67 | OH | 4-F-benzyl | H | H | H | CF₃ | SO₂CH₃ | H | H |
| 68 | OH | (CH₂)₂ | | H | H | CF₃ | SO₂CH₃ | H | H |
| 69 | OH | (CH₂)₄ | | H | H | CF₃ | SO₂CH₃ | H | H |

In a further aspect the compounds of the disclosure are embodied in general Formula B with distinct examples listed in Table 2 below.

TABLE 2

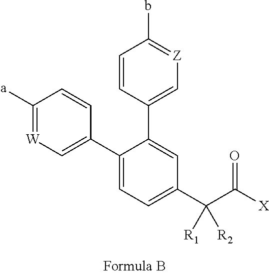

| Ex | X | R₁ | R₂ | W | Z | A | B |
|---|---|---|---|---|---|---|---|
| 70 | OH | SCH₂CH₃ | H | N | CH | H | H |
| 71 | OH | SCH₂CH₃ | H | N | CH | H | F |
| 72 | OH | SCH₂CH₃ | H | N | CH | H | Cl |
| 73 | OH | SCH₂CH₃ | H | N | CH | H | CF₃ |
| 74 | OH | SCH₂CH₃ | H | N | CH | CH₃ | CF₃ |
| 75 | OH | SCH₂CH₃ | H | N | CH | CH₃ | F |
| 76 | OH | SCH₂CH₃ | H | N | CH | OCH₃ | F |
| 77 | OH | SCH₂CH₃ | H | CH | N | H | H |
| 78 | OH | SCH₂CH₃ | H | CH | N | F | H |
| 79 | OH | SCH₂CH₃ | H | CH | N | Cl | H |
| 80 | OH | SCH₂CH₃ | H | CH | N | CF₃ | H |
| 81 | OH | SCH₂CH₃ | H | CH | N | CF₃ | CH₃ |
| 82 | OH | SCH₂CH₃ | H | CH | N | F | OCH₃ |
| 83 | OH | SCH₂CH₃ | H | CH | N | F | CH₃ |
| 84 | OH | SO₂CH₂CH₃ | H | N | CH | CH₃ | CF₃ |
| 85 | OH | SO₂CH₂CH₃ | H | N | CH | H | CF₃ |
| 86 | OH | SO₂CH₂CH₃ | H | CH | N | CF₃ | CH₃ |
| 87 | OH | SO₂CH₂CH₃ | H | CH | N | CF₃ | H |
| 88 | OH | OCH₂ | H | N | CH | CH₃ | CF₃ |
| | | CH₂OCH₃ | | | | | |
| 89 | OH | OCH₂ | H | CH | N | CF₃ | CH₃ |
| | | CH₂OCH₃ | | | | | |
| 90 | OH | CH₃ | H | N | CH | CH₃ | OCH₂ |
| | | | | | | | CH₂OCH₃ |
| 91 | OH | CH₂CH₃ | H | N | CH | CH₃ | OCH₂ |
| | | | | | | | CH₂OCH₃ |
| 92 | OH | CH₂CH₂CH₃ | H | N | CH | CH₃ | OCH₂ |
| | | | | | | | CH₂OCH₃ |
| 93 | OH | CH₂CH(CH₃)₂ | H | N | CH | CH₃ | OCH₂ |
| | | | | | | | CH₂OCH₃ |
| 94 | OH | cyclopro- | H | N | CH | CH₃ | OCH₂ |
| | | pylmethyl | | | | | CH₂OCH₃ |
| 95 | OH | (CH₂)₂ | | N | CH | CH₃ | OCH₂ |
| | | | | | | | CH₂OCH₃ |
| 96 | OH | (CH₂)₄ | | N | CH | CH₃ | OCH₂ |
| | | | | | | | CH₂OCH₃ |
| 97 | OH | CH₃ | H | CH | N | OCH₂ | CH₃ |
| | | | | | | CH₂OCH₃ | |
| 98 | OH | CH₂CH₃ | H | CH | N | OCH₂ | CH₃ |
| | | | | | | CH₂OCH₃ | |
| 99 | OH | CH₂CH₂CH₃ | H | CH | N | OCH₂ | CH₃ |
| | | | | | | CH₂OCH₃ | |
| 100 | OH | CH₂CH(CH₃)₂ | H | CH | N | OCH₂ | CH₃ |
| | | | | | | CH₂OCH₃ | |
| 101 | OH | cyclopro- | H | CH | N | OCH₂ | CH₃ |
| | | pylmethyl | | | | CH₂OCH₃ | |
| 102 | OH | (CH₂)₂ | | CH | N | OCH₂ | CH₃ |
| | | | | | | CH₂OCH₃ | |
| 103 | OH | (CH₂)₄ | | CH | N | OCH₂ | CH₃ |
| | | | | | | CH₂OCH₃ | |
| 104 | OH | CH₃ | H | N | CH | CH₃ | SO₂CH₃ |
| 105 | OH | CH₂CH₃ | H | N | CH | CH₃ | SO₂CH₃ |
| 106 | OH | CH2CH₂CH₃ | H | N | CH | CH₃ | SO₂CH₃ |
| 107 | OH | CH₂CH(CH₃)₂ | H | N | CH | CH₃ | SO₂CH₃ |
| 108 | OH | cyclopro- | H | N | CH | CH₃ | SO₂CH₃ |
| | | pylmethyl | | | | | |
| 109 | | (CH₂)₂ | | N | CH | CH₃ | SO₂CH₃ |
| 110 | OH | (CH₂)₄ | | N | CH | CH₃ | SO₂CH₃ |
| 111 | OH | CH₃ | H | CH | N | SO₂CH₃ | CH₃ |
| 112 | OH | CH₂CH₃ | H | CH | N | SO₂CH₃ | CH₃ |
| 113 | OH | CH₂CH₂CH₃ | H | CH | N | SO₂CH₃ | CH₃ |
| 114 | OH | CH₂CH(CH₃)₂ | H | CH | N | SO₂CH₃ | CH₃ |
| 115 | OH | cyclopro- | H | CH | N | SO₂CH₃ | CH₃ |
| | | pylmethyl | | | | | |
| 116 | | (CH₂)₂ | | CH | N | SO₂CH₃ | CH₃ |
| 117 | OH | (CH₂)₄ | | CH | N | SO₂CH₃ | CH₃ |

In a further aspect the compounds of the disclosure are embodied in general Formula C with distinct examples listed in Table 3 below.

TABLE 3

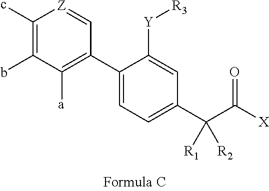

| Ex | X | R₁ | R₂ | Y | R₃ | Z | a | b | c |
|---|---|---|---|---|---|---|---|---|---|
| 118 | OH | CH₃ | H | O | CH₂CF₃ | CH | H | H | CF₃ |
| 119 | OH | CH₂CH₃ | H | O | CH₂CF₃ | CH | H | H | CF₃ |
| 120 | OH | CH₂CH₂CH₃ | H | O | CH₂CF₃ | CH | H | H | CF₃ |
| 121 | OH | CH₂CH(CH₃)₂ | H | O | CH₂CF₃ | CH | H | H | CF₃ |
| 122 | OH | cyclopropylmethyl | H | O | CH₂CF₃ | CH | H | H | CF₃ |
| 123 | OH | benzyl | H | O | CH₂CF₃ | CH | H | H | CF₃ |
| 124 | OH | 4-F-benzyl | H | O | CH₂CF₃ | CH | H | H | CF₃ |
| 125 | OH | (CH₂)₂ | O | CH₂CF₃ | CH | H | H | CF₃ | |
| 126 | OH | (CH₂)₄ | O | CH₂CF₃ | CH | H | H | CF₃ | |
| 127 | OH | CH₂CH₃ | H | O | CH₂CF₃ | CH | H | CF₃ | H |
| 128 | OH | CH₂CH₂CH₃ | H | O | CH₂CF₃ | CH | H | CF₃ | H |
| 129 | OH | CH₂CH(CH₃)₂ | H | O | CH₂CF₃ | CH | H | CF₃ | H |
| 130 | OH | (CH₂)₄ | O | CH₂CF₃ | CH | H | CF₃ | H | |
| 131 | OH | CH₂CH₃ | H | O | CH₂CF₃ | CH | CF₃ | H | H |
| 132 | OH | CH₂CH₂CH₃ | H | O | CH₂CF₃ | CH | CF₃ | H | H |
| 133 | OH | CH₂CH(CH₃)₂ | H | O | CH₂CF₃ | CH | CF₃ | H | H |
| 134 | OH | (CH₂)₄ | O | CH₂CF₃ | CH | CF₃ | H | H | |
| 135 | OH | CH₂CH₃ | H | O | CH₂CF₃ | N | H | H | H |
| 136 | OH | CH₂CH₃ | H | O | CH₂CF₃ | N | H | H | CH₃ |
| 137 | OH | CH₂CH(CH₃)₂ | H | O | CH₂CF₃ | N | H | H | H |
| 138 | OH | CH₂CH(CH₃)₂ | H | O | CH₂CF₃ | N | H | H | CH₃ |
| 139 | OH | CH₃ | H | O | cyclopropylmethyl | CH | H | H | CF₃ |
| 140 | OH | CH₂CH₃ | H | O | cyclopropylmethyl | CH | H | H | CF₃ |
| 141 | OH | CH₂CH₂CH₃ | H | O | cyclopropylmethyl | CH | H | H | CF₃ |
| 142 | OH | CH₂CH(CH₃)₂ | H | O | cyclopropylmethyl | CH | H | H | CF₃ |
| 143 | OH | cyclopropylmethyl | H | O | cyclopropylmethyl | CH | H | H | CF₃ |
| 144 | OH | benzyl | H | O | cyclopropylmethyl | CH | H | H | CF₃ |
| 145 | OH | 4-F-benzyl | H | O | cyclopropylmethyl | CH | H | H | CF₃ |
| 146 | OH | (CH₂)₂ | O | cyclopropylmethyl | CH | H | H | CF₃ | |
| 147 | OH | (CH₂)₄ | O | cyclopropylmethyl | CH | H | H | CF₃ | |
| 148 | OH | CH₂CH₃ | H | O | cyclopropylmethyl | CH | H | CF₃ | H |
| 149 | OH | CH₂CH₃ | H | O | cyclopropylmethyl | CH | CF₃ | H | H |
| 150 | OH | CH₂CH₃ | H | O | cyclopropylmethyl | N | H | H | H |
| 151 | OH | CH₂CH₃ | H | O | cyclopropylmethyl | N | H | H | CH₃ |
| 152 | OH | CH₂CH(CH₃)₂ | H | O | cyclopropylmethyl | CH | H | CF₃ | H |
| 153 | OH | CH₂CH(CH₃)₂ | H | O | cyclopropylmethyl | CH | CF₃ | H | H |
| 154 | OH | CH₂CH(CH₃)₂ | H | O | cyclopropylmethyl | N | H | H | H |
| 155 | OH | CH₂CH(CH₃)₂ | H | O | cyclopropylmethyl | N | H | H | CH₃ |
| 156 | OH | SCH₂CH₃ | H | O | 4-F-phenyl | CH | H | H | CF₃ |
| 157 | OH | SO₂CH₂CH₃ | H | O | 4-F-phenyl | CH | H | H | CF₃ |
| 158 | OH | OCH₂CH₂OCH₃ | H | O | 4-F-phenyl | CH | H | H | CF₃ |
| 159 | OH | OCH₂CH₂SCH₃ | H | O | 4-F-phenyl | CH | H | H | CF₃ |
| 160 | OH | OCH₂CH₂SO₂CH₃ | H | O | 4-F-phenyl | CH | H | H | CF₃ |
| 161 | OH | SCH₂CH₂OCH₃ | H | O | 4-F-phenyl | CH | H | H | CF₃ |
| 162 | OH | NHC(O)CH(CH₃)₂ | H | O | 4-F-phenyl | CH | H | H | CF₃ |
| 163 | OH | SCH₂CH₃ | H | O | 4-F-phenyl | N | H | H | CH₃ |
| 164 | OH | SO₂CH₂CH₃ | H | O | 4-F-phenyl | N | H | H | CH₃ |
| 165 | OH | OCH₂CH₂OCH₃ | H | O | 4-F-phenyl | N | H | H | CH₃ |
| 166 | OH | OCH₂CH₂SCH₃ | H | O | 4-F-phenyl | N | H | H | CH₃ |
| 167 | OH | OCH₂CH₂SO₂CH₃ | H | O | 4-F-phenyl | N | H | H | CH₃ |
| 168 | OH | SCH₂CH₂OCH₃ | H | O | 4-F-phenyl | N | H | H | CH₃ |
| 169 | OH | NHC(O)CH(CH₃)₂ | H | O | 4-F-phenyl | N | H | H | CH₃ |
| 170 | OH | SCH₂CH₃ | H | S | 4-F-phenyl | CH | H | H | CF₃ |
| 171 | OH | SO₂CH₂CH₃ | H | S | 4-F-phenyl | CH | H | H | CF₃ |
| 172 | OH | OCH₂CH₂OCH₃ | H | S | 4-F-phenyl | CH | H | H | CF₃ |
| 173 | OH | OCH₂CH₂SCH₃ | H | S | 4-F-phenyl | CH | H | H | CF₃ |
| 174 | OH | OCH₂CH₂SO₂CH₃ | H | S | 4-F-phenyl | CH | H | H | CF₃ |
| 175 | OH | SCH₂CH₂OCH₃ | H | S | 4-F-phenyl | CH | H | H | CF₃ |
| 176 | OH | NHC(O)CH(CH₃OH)₂ | H | S | 4-F-phenyl | CH | H | H | CF₃ |
| 177 | OH | SCH₂CH₃ | H | S | 4-F-phenyl | N | H | H | CH₃ |
| 178 | OH | SO₂CH₂CH₃ | H | S | 4-F-phenyl | N | H | H | CH₃ |
| 179 | OH | OCH₂CH₂OCH₃ | H | S | 4-F-phenyl | N | H | H | CH₃ |
| 180 | OH | OCH₂CH₂SCH₃ | H | S | 4-F-phenyl | N | H | H | CH₃ |
| 181 | OH | OCH₂CH₂SO₂CH₃ | H | S | 4-F-phenyl | N | H | H | CH₃ |
| 182 | OH | SCH₂CH₂OCH₃ | H | S | 4-F-phenyl | N | H | H | CH₃ |
| 183 | OH | NHC(O)CH(CH₃)₂ | H | S | 4-F-phenyl | N | H | H | CH₃ |
| 184 | OH | CH₂CH₃ | H | O | 4-F-phenyl | CH | H | H | SO₂CH₃ |
| 185 | OH | CH₂CH(CH₃)₂ | H | O | 4-F-phenyl | CH | H | H | SO₂CH₃ |
| 186 | OH | CH₂CH₃ | H | S | 4-F-phenyl | CH | H | H | SO₂CH₃ |
| 187 | OH | CH₂CH(CH₃)₂ | H | S | 4-F-phenyl | CH | H | H | SO₂CH₃ |
| 188 | OH | CH₂CH₃ | H | O | 4-F-phenyl | CH | H | H | OCH₂CH₂OCH₃ |
| 189 | OH | CH₂CH(CH₃)₂ | H | O | 4-F-phenyl | CH | H | H | OCH₂CH₂OCH₃ |
| 190 | OH | CH₂CH₃ | H | S | 4-F-phenyl | CH | H | H | OCH₂CH₂OCH₃ |
| 191 | OH | CH₂CH(CH₃)₂ | H | S | 4-F-phenyl | CH | H | H | OCH₂CH₂OCH₃ |

In a further aspect the compounds of the disclosure are embodied in general Formula D with distinct examples listed in Table 4 below.

TABLE 4
Formula D

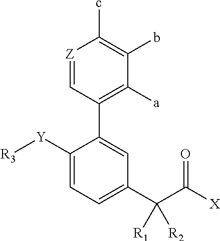

| Ex | X | R1 | R2 | Y | R3 | Z | a | b | c |
|---|---|---|---|---|---|---|---|---|---|
| 192 | OH | CH₃ | H | O | CH₂CF₃ | CH | H | H | CF₃ |
| 193 | OH | CH₂CH₃ | H | O | CH₂CF₃ | CH | H | H | CF₃ |
| 194 | OH | CH₂CH₂CH₃ | H | O | CH₂CF₃ | CH | H | H | CF₃ |
| 195 | OH | CH₂CH(CH₃)₂ | H | O | CH₂CF₃ | CH | H | H | CF₃ |
| 196 | OH | cyclopropylmethyl | H | O | CH₂CF₃ | CH | H | H | CF₃ |
| 197 | OH | benzyl | H | O | CH₂CF₃ | CH | H | H | CF₃ |
| 198 | OH | 4-F-benzyl | H | O | CH₂CF₃ | CH | H | H | CF₃ |
| 199 | OH | (CH₂)₂ | | O | CH₂CF₃ | CH | H | H | CF₃ |
| 200 | OH | (CH₂)₄ | | O | CH₂CF₃ | CH | H | H | CF₃ |
| 201 | OH | CH₂CH₃ | H | O | CH₂CF₃ | CH | H | CF₃ | H |
| 202 | OH | CH₂CH₂CH₃ | H | O | CH₂CF₃ | CH | H | CF₃ | H |
| 203 | OH | CH₂CH(CH₃)₂ | H | O | CH₂CF₃ | CH | H | CF₃ | H |
| 204 | OH | (CH₂)₄ | | O | CH₂CF₃ | CH | H | CF₃ | H |
| 205 | OH | CH₂CH₃ | H | O | CH₂CF₃ | CH | CF₃ | H | H |
| 206 | OH | CH₂CH₂CH₃ | H | O | CH₂CF₃ | CH | CF₃ | H | H |
| 207 | OH | CH₂CH(CH₃)₂ | H | O | CH₂CF₃ | CH | CF₃ | H | H |
| 208 | OH | (CH₂)₄ | | O | CH₂CF₃ | CH | CF₃ | H | H |
| 209 | OH | CH₂CH₃ | H | O | CH₂CF₃ | N | H | H | H |
| 210 | OH | CH₂CH₃ | H | O | CH₂CF₃ | N | H | H | CH₃ |
| 211 | OH | CH₂CH(CH₃)₂ | H | O | CH₂CF₃ | N | H | H | H |
| 212 | OH | CH₂CH(CH₃)₂ | H | O | CH₂CF₃ | N | H | H | CH₃ |
| 213 | OH | CH₃ | H | O | cyclopropylmethyl | CH | H | H | CF₃ |
| 214 | OH | CH₂CH₃ | H | O | cyclopropylmethyl | CH | H | H | CF₃ |
| 215 | OH | CH₂CH₂CH₃ | H | O | cyclopropylmethyl | CH | H | H | CF₃ |
| 216 | OH | CH₂CH(CH₃)₂ | H | O | cyclopropylmethyl | CH | H | H | CF₃ |
| 217 | OH | cyclopropylmethyl | H | O | cyclopropylmethyl | CH | H | H | CF₃ |
| 218 | OH | benzyl | H | O | cyclopropylmethyl | CH | H | H | CF₃ |
| 219 | OH | 4-F-benzyl | H | O | cyclopropylmethyl | CH | H | H | CF₃ |
| 220 | OH | (CH₂)₂ | | O | cyclopropylmethyl | CH | H | H | CF₃ |
| 221 | OH | (CH₂)₄ | | O | cyclopropylmethyl | CH | H | H | CF₃ |
| 222 | OH | CH₂CH₃ | H | O | cyclopropylmethyl | CH | H | CF₃ | H |
| 223 | OH | CH₂CH₃ | H | O | cyclopropylmethyl | CH | CF₃ | H | H |
| 224 | OH | CH₂CH₃ | H | O | cyclopropylmethyl | N | H | H | H |
| 225 | OH | CH₂CH₃ | H | O | cyclopropylmethyl | N | H | H | CH₃ |
| 226 | OH | CH₂CH(CH₃)₂ | H | O | cyclopropylmethyl | CH | H | CF₃ | H |
| 227 | OH | CH₂CH(CH₃)₂ | H | O | cyclopropylmethyl | CH | CF₃ | H | H |
| 228 | OH | CH₂CH(CH₃)₂ | H | O | cyclopropylmethyl | N | H | H | H |
| 229 | OH | CH₂CH(CH₃)₂ | H | O | cyclopropylmethyl | N | H | H | CH₃ |
| 230 | OH | SCH₂CH₃ | H | O | 4-F-phenyl | CH | H | H | CF₃ |
| 231 | OH | SO₂CH₂CH₃ | H | O | 4-F-phenyl | CH | H | H | CF₃ |
| 232 | OH | OCH₂CH₂OCH₃ | H | O | 4-F-phenyl | CH | H | H | CF₃ |
| 233 | OH | OCH₂CH₂SCH₃ | H | O | 4-F-phenyl | CH | H | H | CF₃ |
| 234 | OH | OCH₂CH₂SO₂CH₃ | H | O | 4-F-phenyl | CH | H | H | CF₃ |
| 235 | OH | SCH₂CH₂OCH₃ | H | O | 4-F-phenyl | CH | H | H | CF₃ |
| 236 | OH | NHC(O)CH(CH₃)₂ | H | O | 4-F-phenyl | CH | H | H | CF₃ |
| 237 | OH | SCH₂CH₃ | H | O | 4-F-phenyl | N | H | H | CH₃ |
| 238 | OH | SO₂CH₂CH₃ | H | O | 4-F-phenyl | N | H | H | CH₃ |
| 239 | OH | OCH₂CH₂OCH₃ | H | O | 4-F-phenyl | N | H | H | CH₃ |
| 240 | OH | OCH₂CH₂SCH₃ | H | O | 4-F-phenyl | N | H | H | CH₃ |
| 241 | OH | OCH₂CH₂SO₂CH₃ | H | O | 4-F-phenyl | N | H | H | CH₃ |
| 242 | OH | SCH₂CH₂OCH₃ | H | O | 4-F-phenyl | N | H | H | CH₃ |
| 243 | OH | NHC(O)CH(CH₃)₂ | H | O | 4-F-phenyl | N | H | H | CH₃ |
| 244 | OH | SCH₂CH₃ | H | S | 4-F-phenyl | CH | H | H | CF₃ |
| 245 | OH | SO₂CH₂CH₃ | H | S | 4-F-phenyl | CH | H | H | CF₃ |
| 246 | OH | OCH₂CH₂OCH₃ | H | S | 4-F-phenyl | CH | H | H | CF₃ |
| 247 | OH | OCH₂CH₂SCH₃ | H | S | 4-F-phenyl | CH | H | H | CF₃ |
| 248 | OH | OCH₂CH₂SO₂CH₃ | H | S | 4-F-phenyl | CH | H | H | CF₃ |
| 249 | OH | SCH₂CH₂OCH₃ | H | S | 4-F-phenyl | CH | H | H | CF₃ |
| 250 | OH | NHC(O)CH(CH₃)₂ | H | S | 4-F-phenyl | CH | H | H | CF₃ |
| 251 | OH | SCH₂CH₃ | H | S | 4-F-phenyl | N | H | H | CH₃ |
| 252 | OH | SO₂CH₂CH₃ | H | S | 4-F-phenyl | N | H | H | CH₃ |
| 253 | OH | OCH₂CH₂OCH₃ | H | S | 4-F-phenyl | N | H | H | CH₃ |
| 254 | OH | OCH₂CH₂SCH₃ | H | S | 4-F-phenyl | N | H | H | CH₃ |
| 255 | OH | OCH₂CH₂SO₂CH₃ | H | S | 4-F-phenyl | N | H | H | CH₃ |
| 256 | OH | SCH₂CH₂OCH₃ | H | S | 4-F-phenyl | N | H | H | CH₃ |
| 257 | OH | NHC(O)CH(CH₃)₂ | H | S | 4-F-phenyl | N | H | H | CH₃ |
| 258 | OH | CH₂CH₃ | H | O | 4-F-phenyl | CH | H | H | SO₂CH₃ |
| 259 | OH | CH₂CH(CH₃)₂ | H | O | 4-F-phenyl | CH | H | H | SO₂CH₃ |
| 260 | OH | CH₂CH₃ | H | S | 4-F-phenyl | CH | H | H | SO₂CH₃ |
| 261 | OH | CH₂CH(CH₃)₂ | H | S | 4-F-phenyl | CH | H | H | SO₂CH₃ |
| 262 | OH | CH₂CH₃ | H | O | 4-F-phenyl | CH | H | H | OCH₂CH₂OCH₃ |
| 263 | OH | CH₂CH(CH₃)₂ | H | O | 4-F-phenyl | CH | H | H | OCH₂CH₂OCH₃ |
| 264 | OH | CH₂CH₃ | H | S | 4-F-phenyl | CH | H | H | OCH₂CH₂OCH₃ |
| 265 | OH | CH₂CH(CH₃)₂ | H | S | 4-F-phenyl | CH | H | H | OCH₂CH₂OCH₃ |

In a further aspect the compounds of the disclosure are embodied in general Formula E with distinct examples listed in Table 5 below.

TABLE 5
Formula E

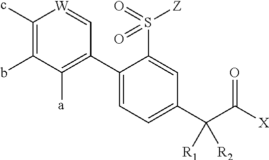

| Ex | X | R1 | R2 | W | a | b | c | Z |
|---|---|---|---|---|---|---|---|---|
| 266 | OH | CH₃ | H | CH | H | H | CF₃ | tetrahydro- |
| | | | | | | | | isoquinolyl |
| 267 | OH | CH₂CH₃ | H | CH | H | H | CF₃ | tetrahydro- |
| | | | | | | | | isoquinolyl |
| 268 | OH | CH₂CH₂CH₃ | H | CH | H | H | CF₃ | Tetrahydro- |
| | | | | | | | | isoquinolyl |
| 269 | OH | CH₂CH(CH₃)₂ | H | CH | H | H | CF₃ | Tetrahydro- |
| | | | | | | | | isoquinolyl |
| 270 | OH | cyclopropylmethyl | H | CH | H | H | CF₃ | Tetrahydro- |
| | | | | | | | | isoquinolyl |
| 271 | OH | benzyl | H | CH | H | H | CF₃ | Tetrahydro- |
| | | | | | | | | isoquinolyl |
| 272 | OH | 4-F-benzyl | H | CH | H | H | CF₃ | Tetrahydro- |
| | | | | | | | | isoquinolyl |
| 273 | OH | (CH₂)₂ | | CH | H | H | CF₃ | Tetrahydro- |
| | | | | | | | | isoquinolyl |
| 274 | OH | (CH₂)₄ | | CH | H | H | CF₃ | Tetrahydro- |
| | | | | | | | | isoquinolyl |
| 275 | OH | CH₂CH₃ | H | CH | H | CF₃ | H | Tetrahydro- |
| | | | | | | | | isoquinolyl |
| 276 | OH | CH₂CH₃ | H | CH | CF₃ | H | H | Tetrahydro- |
| | | | | | | | | isoquinolyl |
| 277 | OH | CH₂CH(CH₃)₂ | H | N | H | H | H | Tetrahydro- |
| | | | | | | | | isoquinolyl |
| 278 | OH | CH₂CH(CH₃)₂ | H | N | H | H | CH₃ | Tetrahydro- |
| | | | | | | | | isoquinolyl |
| 279 | OH | SCH₂CH₃ | H | CH | H | H | CF₃ | Tetrahydro- |
| | | | | | | | | isoquinolyl |
| 280 | OH | SO₂CH₂CH₃ | H | CH | H | H | CF₃ | Tetrahydro- |
| | | | | | | | | isoquinolyl |
| 281 | OH | OCH₂CH₂OCH₃ | H | CH | H | H | CF₃ | Tetrahydro- |
| | | | | | | | | isoquinolyl |
| 282 | OH | OCH₂CH₂SCH₃ | H | CH | H | H | CF₃ | Tetrahydro- |
| | | | | | | | | isoquinolyl |
| 283 | OH | OCH₂CH₂SO₂CH₃ | H | CH | H | H | CF₃ | Tetrahydro- |
| | | | | | | | | isoquinolyl |
| 284 | OH | SCH₂CH₂OCH₃ | H | CH | H | H | CF₃ | Tetrahydro- |
| | | | | | | | | isoquinolyl |
| 285 | OH | CH₃ | | CH | H | H | CF₃ | Piperidinyl |
| 286 | OH | CH₂CH₃ | H | CH | H | H | CF₃ | Piperidinyl |
| 287 | OH | CH₂CH₂CH₃ | H | CH | H | H | CF₃ | Piperidinyl |
| 288 | OH | CH₂CH(CH₃)₂ | H | CH | H | H | CF₃ | Piperidinyl |
| 289 | OH | cyclopropylmethyl | H | CH | H | H | CF₃ | Piperidinyl |
| 290 | OH | benzyl | H | CH | H | H | CF₃ | Piperidinyl |
| 291 | OH | 4-F-benzyl | H | CH | H | H | CF₃ | Piperidinyl |
| 292 | OH | (CH₂)₂ | | CH | H | H | CF₃ | Piperidinyl |
| 293 | OH | (CH₂)₄ | | CH | H | H | CF₃ | Piperidinyl |
| 294 | OH | CH₂CH₃ | H | CH | H | CF₃ | H | Piperidinyl |
| 295 | OH | CH₂CH₃ | H | CH | CF₃ | H | H | Piperidinyl |
| 296 | OH | CH₂CH(CH₃)₂ | H | N | H | H | H | Piperidinyl |
| 297 | OH | CH₂CH(CH₃)₂ | H | N | H | H | CH₃ | Piperidinyl |
| 298 | OH | SCH₂CH₃ | H | CH | H | H | CF₃ | Piperidinyl |
| 299 | OH | SO₂CH₂CH₃ | H | CH | H | H | CF₃ | Piperidinyl |
| 300 | OH | OCH₂CH₂OCH₃ | H | CH | H | H | CF₃ | Piperidinyl |
| 301 | OH | OCH₂CH₂SCH₃ | H | CH | H | H | CF₃ | Piperidinyl |
| 302 | OH | OCH₂CH₂SO₂CH₃ | H | CH | H | H | CF₃ | Piperidinyl |
| 303 | OH | SCH₂CH₂OCH₃ | H | CH | H | H | CF₃ | Piperidinyl |

In a further aspect the compounds of the disclosure are embodied in general Formula F with distinct examples listed in Table 6 below.

TABLE 6
Formula F

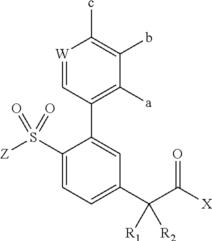

| Ex | X | R1 | R2 | W | a | b | c | Z |
|---|---|---|---|---|---|---|---|---|
| 304 | OH | CH₃ | H | CH | H | H | CF₃ | Tetrahydro- |
| | | | | | | | | isoquinolyl |
| 305 | OH | CH₂CH₃ | H | CH | H | H | CF₃ | Tetrahydro- |
| | | | | | | | | isoquinolyl |
| 306 | OH | CH₂CH₂CH₃ | H | CH | H | H | CF₃ | Tetrahydro- |
| | | | | | | | | isoquinolyl |
| 307 | OH | CH₂CH(CH₃)₂ | H | CH | H | H | CF₃ | Tetrahydro- |
| | | | | | | | | isoquinolyl |
| 308 | OH | cyclopropylmethyl | H | CH | H | H | CF₃ | Tetrahydro- |
| | | | | | | | | isoquinolyl |
| 309 | OH | benzyl | H | CH | H | H | CF₃ | Tetrahydro- |
| | | | | | | | | isoquinolyl |
| 310 | OH | 4-F-benzyl | H | CH | H | H | CF₃ | Tetrahydro- |
| | | | | | | | | isoquinolyl |
| 311 | OH | (CH₂)₂ | — | CH | H | H | CF₃ | Tetrahydro- |
| | | | | | | | | isoquinolyl |
| 312 | OH | (CH₂)₄ | — | CH | H | H | CF₃ | Tetrahydro- |
| | | | | | | | | isoquinolyl |
| 313 | OH | CH₂CH₃ | H | CH | H | CF₃ | H | Tetrahydro- |
| | | | | | | | | isoquinolyl |
| 314 | OH | CH₂CH₃ | H | CH | CF₃ | H | H | Tetrahydro- |
| | | | | | | | | isoquinolyl |
| 315 | OH | CH₂CH(CH₃)₂ | H | N | H | H | H | Tetrahydro- |
| | | | | | | | | isoquinolyl |
| 316 | OH | CH₂CH(CH₃)₂ | H | N | H | H | CH₃ | Tetrahydro- |
| | | | | | | | | isoquinolyl |
| 317 | OH | SCH₂CH₃ | H | CH | H | H | CF₃ | Tetrahydro- |
| | | | | | | | | isoquinolyl |
| 318 | OH | SO₂CH₂CH₃ | H | CH | H | H | CF₃ | Tetrahydro- |
| | | | | | | | | isoquinolyl |
| 319 | OH | OCH₂CH₂OCH₃ | H | CH | H | H | CF₃ | Tetrahydro- |
| | | | | | | | | isoquinolyl |
| 320 | OH | OCH₂CH₂SCH₃ | H | CH | H | H | CF₃ | Tetrahydro- |
| | | | | | | | | isoquinolyl |
| 321 | OH | OCH₂CH₂SO₂CH₃ | H | CH | H | H | CF₃ | Tetrahydro- |
| | | | | | | | | isoquinolyl |
| 322 | OH | SCH₂CH₂OCH₃ | H | CH | H | H | CF₃ | Tetrahydro- |
| | | | | | | | | isoquinolyl |
| 323 | OH | CH₃ | H | CH | H | H | CF₃ | Piperidinyl |
| 324 | OH | CH₂CH₃ | H | CH | H | H | CF₃ | Piperidinyl |
| 325 | OH | CH₂CH₂CH₃ | H | CH | H | H | CF₃ | Piperidinyl |
| 326 | OH | CH₂CH(CH₃)₂ | H | CH | H | H | CF₃ | Piperidinyl |
| 327 | OH | cyclopropylmethyl | H | CH | H | H | CF₃ | Piperidinyl |
| 328 | OH | benzyl | H | CH | H | H | CF₃ | Piperidinyl |
| 329 | OH | 4-F-benzyl | H | CH | H | H | CF₃ | Piperidinyl |
| 330 | OH | (CH₂)₂ | — | CH | H | H | CF₃ | Piperidinyl |
| 331 | OH | (CH₂)₄ | — | CH | H | H | CF₃ | Piperidinyl |
| 332 | OH | CH₂CH₃ | H | CH | H | CF₃ | H | Piperidinyl |
| 333 | OH | CH₂CH₃ | H | CH | CF₃ | H | H | Piperidinyl |
| 334 | OH | CH₂CH(CH₃)₂ | H | N | H | H | H | Piperidinyl |
| 335 | OH | CH₂CH(CH₃)₂ | H | N | H | H | CH₃ | Piperidinyl |
| 336 | OH | SCH₂CH₃ | H | CH | H | H | CF₃ | Piperidinyl |
| 337 | OH | SO₂CH₂CH₃ | H | CH | H | H | CF₃ | Piperidinyl |
| 338 | OH | OCH₂CH₂OCH₃ | H | CH | H | H | CF₃ | Piperidinyl |
| 339 | OH | OCH₂CH₂SCH₃ | H | CH | H | H | CF₃ | Piperidinyl |
| 340 | OH | OCH₂CH₂SO₂CH₃ | H | CH | H | H | CF₃ | Piperidinyl |
| 341 | OH | SCH₂CH₂OCH₃ | H | CH | H | H | CF₃ | Piperidinyl |

In a further aspect the compounds of the disclosure are embodied in general Formula G with distinct examples listed in Table 7 below.

TABLE 7
Formula G

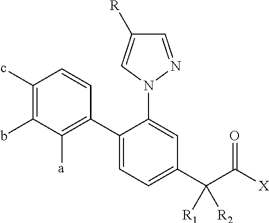

| Ex | X | R1 | R2 | R | a | B | C |
|---|---|---|---|---|---|---|---|
| 342 | OH | CH₃ | H | 4-ethylphenyl | H | H | CF₃ |
| 343 | OH | CH₂CH₃ | H | 4-ethylphenyl | H | H | CF₃ |
| 344 | OH | CH₂CH₂CH₃ | H | 4-ethylphenyl | H | H | CF₃ |
| 345 | OH | CH₂CH(CH₃)₂ | H | 4-ethylphenyl | H | H | CF₃ |
| 346 | OH | (CH₂)₄ | — | 4-ethylphenyl | H | H | CF₃ |
| 347 | OH | CH₃ | H | 4-methoxy- | H | H | CF₃ |
| | | | | phenyl | | | |
| 348 | OH | CH₂CH₃ | H | 4-methoxy- | H | H | CF₃ |
| | | | | phenyl | | | |
| 349 | OH | CH₂CH₂CH₃ | H | 4-methoxy- | H | H | CF₃ |
| | | | | phenyl | | | |
| 350 | OH | CH₂CH(CH₃)₂ | H | 4-methoxy- | H | H | CF₃ |
| | | | | phenyl | | | |
| 351 | OH | (CH₂)₄ | — | 4-methoxy- | H | H | CF₃ |
| | | | | phenyl | | | |
| 352 | OH | CH₂CH(CH₃)₂ | H | 4-ethylphenyl | H | CF₃ | H |
| 353 | OH | CH₂CH(CH₃)₂ | H | 4-ethylphenyl | CF₃ | H | H |
| 354 | OH | SCH₂CH₃ | H | 4-fluorophenyl | H | H | CF₃ |
| 355 | OH | SO₂CH₂CH₃ | H | 4-fluorophenyl | H | H | CF₃ |
| 356 | OH | OCH₂CH₂OCH₃ | H | 4-fluorophenyl | H | H | CF₃ |
| 357 | OH | OCH₂CH₂SCH₃ | H | 4-fluorophenyl | H | H | CF₃ |
| 358 | OH | OCH₂CH₂SO₂CH₃ | H | 4-fluorophenyl | H | H | CF₃ |
| 359 | OH | SCH₂CH₂OCH₃ | H | 4-fluorophenyl | H | H | CF₃ |
| 360 | OH | CH₂CH₃ | H | 4-fluorophenyl | H | H | SO₂CH₃ |
| 361 | OH | CH₂CH₂CH₃ | H | 4-fluorophenyl | H | H | SO₂CH₃ |
| 362 | OH | CH₂CH(CH₃)₂ | H | 4-fluorophenyl | H | H | SO₂CH₃ |
| 363 | OH | (CH₂)₄ | — | 4-fluorophenyl | H | H | SO₂CH₃ |

In a further aspect the compounds of the disclosure are embodied in general Formula H with distinct examples listed in Table 8 below.

TABLE 8
Formula H

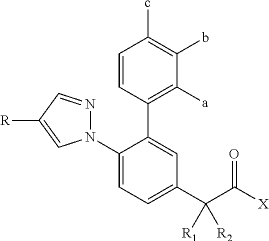

| Ex | X | R1 | R2 | R | a | b | c |
|---|---|---|---|---|---|---|---|
| 364 | OH | CH₃ | H | 4-ethylphenyl | H | H | CF₃ |
| 365 | OH | CH₂CH₃ | H | 4-ethylphenyl | H | H | CF₃ |
| 366 | OH | CH₂CH₂CH₃ | H | 4-ethylphenyl | H | H | CF₃ |
| 367 | OH | CH₂CH(CH₃)₂ | H | 4-ethylphenyl | H | H | CF₃ |
| 368 | OH | (CH₂)₄ | — | 4-ethylphenyl | H | H | CF₃ |
| 369 | OH | CH₃ | H | 4-methoxy- | H | H | CF₃ |
| | | | | phenyl | | | |
| 370 | OH | CH₂CH₃ | H | 4-methoxy- | H | H | CF₃ |
| | | | | phenyl | | | |
| 371 | OH | CH₂CH₂CH₃ | H | 4-methoxy- | H | H | CF₃ |
| | | | | phenyl | | | |
| 372 | OH | CH₂CH(CH₃)₂ | H | 4-methoxy- | H | H | CF₃ |
| | | | | phenyl | | | |
| 373 | OH | (CH₂)₄ | — | 4-methoxy- | H | H | CF₃ |
| | | | | phenyl | | | |
| 374 | OH | CH₂CH(CH₃)₂ | H | 4-ethylphenyl | H | CF₃ | H |
| 375 | OH | CH₂CH(CH₃)₂ | H | 4-ethylphenyl | CF₃ | H | H |
| 376 | OH | SCH₂CH₃ | H | 4-fluorophenyl | H | H | CF₃ |
| 377 | OH | SO₂CH₂CH₃ | H | 4-fluorophenyl | H | H | CF₃ |
| 378 | OH | OCH₂CH₂OCH₃ | H | 4-fluorophenyl | H | H | CF₃ |
| 379 | OH | OCH₂CH₂SCH₃ | H | 4-fluorophenyl | H | H | CF₃ |
| 380 | OH | OCH₂CH₂SO₂CH₃ | H | 4-fluorophenyl | H | H | CF₃ |
| 381 | OH | SCH₂CH₂OCH₃ | H | 4-fluorophenyl | H | H | CF₃ |
| 382 | OH | CH₂CH₃ | H | 4-fluorophenyl | H | H | SO₂CH₃ |
| 383 | OH | CH₂CH₂CH₃ | H | 4-fluorophenyl | H | H | SO₂CH₃ |
| 384 | OH | CH₂CH(CH₃)₂ | H | 4-fluorophenyl | H | H | SO₂CH₃ |
| 385 | OH | (CH₂)₄ | — | 4-fluorophenyl | H | H | SO₂CH₃ |

In a further aspect the compounds of the disclosure are embodied in general Formula I with distinct examples listed in Table 9 below.

TABLE 9
Formula I

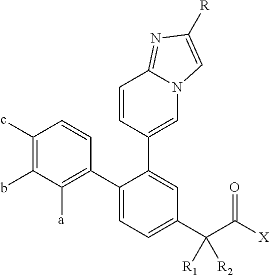

| Ex | X | R1 | R2 | R | a | b | c |
|---|---|---|---|---|---|---|---|
| 386 | OH | SCH₂CH₃ | H | CF₃ | H | H | CF₃ |
| 387 | OH | SO₂CH₂CH₃ | H | CF₃ | H | H | CF₃ |
| 388 | OH | OCH₂CH₂OCH₃ | H | CF₃ | H | H | CF₃ |
| 389 | OH | OCH₂CH₂SCH₃ | H | CF₃ | H | H | CF₃ |
| 390 | OH | OCH₂CH₂SO₂CH₃ | H | CF₃ | H | H | CF₃ |
| 391 | OH | SCH₂CH₂OCH₃ | H | CF₃ | H | H | CF₃ |
| 392 | OH | CH₂CH₃ | H | CF₃ | H | H | SO₂CH₃ |
| 393 | OH | CH₂CH₂CH₃ | H | CF₃ | H | H | SO₂CH₃ |
| 394 | OH | CH₂CH(CH₃)₂ | H | CF₃ | H | H | SO₂CH₃ |
| 395 | OH | (CH₂)₄ | — | CF₃ | H | H | SO₂CH₃ |

In a further aspect the compounds of the disclosure are embodied in general Formula J with distinct examples listed in Table 10 below.

TABLE 10
Formula J

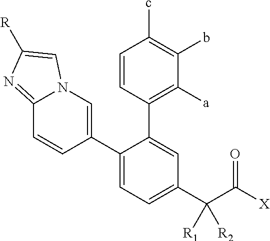

| Ex | X | R1 | R2 | R | a | b | c |
|---|---|---|---|---|---|---|---|
| 396 | OH | SCH₂CH₃ | H | CF₃ | H | H | CF₃ |
| 397 | OH | SO₂CH₂CH₃ | H | CF₃ | H | H | CF₃ |
| 398 | OH | OCH₂CH₂OCH₃ | H | CF₃ | H | H | CF₃ |
| 399 | OH | OCH₂CH₂SCH₃ | H | CF₃ | H | H | CF₃ |
| 400 | OH | OCH₂CH₂SO₂CH₃ | H | CF₃ | H | H | CF₃ |
| 401 | OH | SCH₂CH₂OCH₃ | H | CF₃ | H | H | CF₃ |
| 402 | OH | CH₂CH₃ | H | CF₃ | H | H | SO₂CH₃ |
| 403 | OH | CH₂CH₂CH₃ | H | CF₃ | H | H | SO₂CH₃ |
| 404 | OH | CH₂CH(CH₃)₂ | H | CF₃ | H | H | SO₂CH₃ |
| 405 | OH | (CH₂)₄ | — | CF₃ | H | H | SO₂CH₃ |

### Example 406

### 2-(6-(cyclopropylmethoxy)-4′-(trifluoromethyl)biphenyl-3-yl)acetic acid

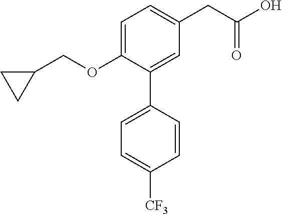

Further examples are demonstrated in Table 11.

Compounds of formula (I)

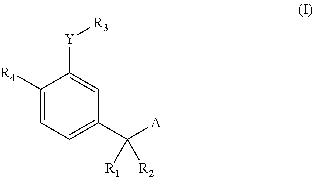

where A is CO₂H and Y is a bond

TABLE 11

| Example # | Formula | R¹ | R² | R³ | R⁴ |
|---|---|---|---|---|---|
| 440 | (I) | H | CH₂-cPr | p-C₆H₄—CF₃ | O—CH₂CF₃ |
| 441 | (I) | Cyclobutyl | | p-C₆H₄—CF₃ | O—CH₂CF₃ |
| 442 | (I) | Cyclopentyl | | p-C₆H₄—CF₃ | O—CH₂CF₃ |
| 203 | (I) | H | Isobutyl | p-C₆H₄—CF₃ | O—CH₂CF₃ |
| 443 | (I) | H | CH₂-cPr | p-C₆H₄—CF₃ | O—CH₂CH₂CF₃ |
| 444 | (I) | Cyclobutyl | | p-C₆H₄—CF₃ | O—CH₂CH₂CF₃ |
| 445 | (I) | Cyclopentyl | | p-C₆H₄—CF₃ | O—CH₂CH₂CF₃ |
| 446 | (I) | H | Isobutyl | p-C₆H₄—CF₃ | O—CH₂CH₂CF₃ |
| 447 | (I) | H | CH₂-cPr | p-C₆H₄—CF₃ | O—CH(CF₃)₂ |
| 448 | (I) | Cyclobutyl | | p-C₆H₄—CF₃ | O—CH(CF₃)₂ |
| 449 | (I) | Cyclopentyl | | p-C₆H₄—CF₃ | O—CH(CF₃)₂ |
| 450 | (I) | H | Isobutyl | p-C₆H₄—CF₃ | O—CH(CF₃)₂ |
| 451 | (I) | H | CH₂-cPr | p-C₆H₄—CF₃ | O—CH(Me)(CF₃) |
| 452 | (I) | Cyclobutyl | | p-C₆H₄—CF₃ | O—CH(Me)(CF₃) |
| 453 | (I) | Cyclopentyl | | p-C₆H₄—CF₃ | O—CH(Me)(CF₃) |
| 454 | (I) | H | Isobutyl | p-C₆H₄—CF₃ | O—CH(Me)(CF₃) |
| 455 | (I) | H | CH₂-cPr | p-C₆H₄—CF₃ | O-cPr |
| 456 | (I) | Cyclobutyl | | p-C₆H₄—CF₃ | O-cPr |
| 457 | (I) | Cyclopentyl | | p-C₆H₄—CF₃ | O-cPr |
| 458 | (I) | H | Isobutyl | p-C₆H₄—CF₃ | O-cPr |
| 459 | (I) | H | CH₂-cPr | p-C₆H₄—CF₃ | O—CH₂-cPr |
| 460 | (I) | Cyclobutyl | | p-C₆H₄—CF₃ | O—CH₂-cPr |
| 461 | (I) | Cyclopentyl | | p-C₆H₄—CF₃ | O—CH₂-cPr |
| 216 | (I) | H | Isobutyl | p-C₆H₄—CF₃ | O—CH₂-cPr |
| 462 | (I) | H | CH₂-cPr | p-C₆H₄—Cl | O—CH₂CF₃ |
| 463 | (I) | Cyclobutyl | | p-C₆H₄—Cl | O—CH₂CF₃ |
| 464 | (I) | Cyclopentyl | | p-C₆H₄—Cl | O—CH₂CF₃ |
| 465 | (I) | H | Isobutyl | p-C₆H₄—Cl | O—CH₂CF₃ |
| 466 | (I) | H | CH₂-cPr | p-C₆H₄—Cl | O—CH₂CH₂CF₃ |
| 467 | (I) | Cyclobutyl | | p-C₆H₄—Cl | O—CH₂CH₂CF₃ |
| 468 | (I) | Cyclopentyl | | p-C₆H₄—Cl | O—CH₂CH₂CF₃ |
| 469 | (I) | H | Isobutyl | p-C₆H₄—Cl | O—CH₂CH₂CF₃ |
| 470 | (I) | H | CH₂-cPr | p-C₆H₄—Cl | O—CH(CF₃)₂ |
| 471 | (I) | Cyclobutyl | | p-C₆H₄—Cl | O—CH(CF₃)₂ |
| 472 | (I) | Cyclopentyl | | p-C₆H₄—Cl | O—CH(CF₃)₂ |
| 473 | (I) | H | Isobutyl | p-C₆H₄—Cl | O—CH(CF₃)₂ |
| 474 | (I) | H | CH₂-cPr | p-C₆H₄—Cl | O—CH(Me)(CF₃) |
| 475 | (I) | Cyclobutyl | | p-C₆H₄—Cl | O—CH(Me)(CF₃) |
| 476 | (I) | Cyclopentyl | | p-C₆H₄—Cl | O—CH(Me)(CF₃) |
| 477 | (I) | H | Isobutyl | p-C₆H₄—Cl | O—CH(Me)(CF₃) |
| 478 | (I) | H | CH₂-cPr | p-C₆H₄—Cl | O-cPr |
| 479 | (I) | Cyclobutyl | | p-C₆H₄—Cl | O-cPr |
| 480 | (I) | Cyclopentyl | | p-C₆H₄—Cl | O-cPr |
| 481 | (I) | H | Isobutyl | p-C₆H₄—Cl | O-cPr |
| 482 | (I) | H | CH₂-cPr | p-C₆H₄—Cl | O—CH₂-cPr |
| 483 | (I) | Cyclobutyl | | p-C₆H₄—Cl | O—CH₂-cPr |
| 484 | (I) | Cyclopentyl | | p-C₆H₄—Cl | O—CH₂-cPr |
| 428 | (I) | H | Isobutyl | p-C₆H₄—Cl | O—CH₂-cPr |
| 485 | (I) | H | CH₂-cPr | p-C₆H₄—F | O—CH₂CF₃ |
| 486 | (I) | Cyclobutyl | | p-C₆H₄—F | O—CH₂CF₃ |
| 487 | (I) | Cyclopentyl | | p-C₆H₄—F | O—CH₂CF₃ |
| 488 | (I) | H | Isobutyl | p-C₆H₄—F | O—CH₂CF₃ |
| 489 | (I) | H | CH₂-cPr | p-C₆H₄—F | O—CH₂CH₂CF₃ |
| 490 | (I) | Cyclobutyl | | p-C₆H₄—F | O—CH₂CH₂CF₃ |
| 491 | (I) | Cyclopentyl | | p-C₆H₄—F | O—CH₂CH₂CF₃ |
| 492 | (I) | H | Isobutyl | p-C₆H₄—F | O—CH₂CH₂CF₃ |
| 493 | (I) | H | CH₂-cPr | p-C₆H₄—F | O—CH(CF₃)₂ |
| 494 | (I) | Cyclobutyl | | p-C₆H₄—F | O—CH(CF₃)₂ |
| 495 | (I) | Cyclopentyl | | p-C₆H₄—F | O—CH(CF₃)₂ |
| 496 | (I) | H | Isobutyl | p-C₆H₄—F | O—CH(CF₃)₂ |
| 497 | (I) | H | CH₂-cPr | p-C₆H₄—F | O—CH(Me)(CF₃) |
| 498 | (I) | Cyclobutyl | | p-C₆H₄—F | O—CH(Me)(CF₃) |
| 499 | (I) | Cyclopentyl | | p-C₆H₄—F | O—CH(Me)(CF₃) |
| 500 | (I) | H | Isobutyl | p-C₆H₄—F | O—CH(Me)(CF₃) |
| 501 | (I) | H | CH₂-cPr | p-C₆H₄—F | O-cPr |
| 502 | (I) | Cyclobutyl | | p-C₆H₄—F | O-cPr |
| 503 | (I) | Cyclopentyl | | p-C₆H₄—F | O-cPr |
| 504 | (I) | H | Isobutyl | p-C₆H₄—F | O-cPr |
| 505 | (I) | H | CH₂-cPr | p-C₆H₄—F | O—CH₂-cPr |
| 506 | (I) | Cyclobutyl | | p-C₆H₄—F | O—CH₂-cPr |
| 507 | (I) | Cyclopentyl | | p-C₆H₄—F | O—CH₂-cPr |
| 427 | (I) | H | Isobutyl | p-C₆H₄—F | O—CH₂-cPr |
| 508 | (I) | H | CH₂-cPr | p-C₆H₄—SMe | O—CH₂CF₃ |
| 509 | (I) | Cyclobutyl | | p-C₆H₄—SMe | O—CH₂CF₃ |
| 510 | (I) | Cyclopentyl | | p-C₆H₄—SMe | O—CH₂CF₃ |
| 511 | (I) | H | Isobutyl | p-C₆H₄—SMe | O—CH₂CF₃ |
| 512 | (I) | H | CH₂-cPr | p-C₆H₄—SMe | O—CH₂CH₂CF₃ |
| 513 | (I) | Cyclobutyl | | p-C₆H₄—SMe | O—CH₂CH₂CF₃ |
| 514 | (I) | Cyclopentyl | | p-C₆H₄—SMe | O—CH₂CH₂CF₃ |
| 515 | (I) | H | Isobutyl | p-C₆H₄—SMe | O—CH₂CH₂CF₃ |
| 516 | (I) | H | CH₂-cPr | p-C₆H₄—SMe | O—CH(CF₃)₂ |
| 517 | (I) | Cyclobutyl | | p-C₆H₄—SMe | O—CH(CF₃)₂ |
| 518 | (I) | Cyclopentyl | | p-C₆H₄—SMe | O—CH(CF₃)₂ |
| 519 | (I) | H | Isobutyl | p-C₆H₄—SMe | O—CH(CF₃)₂ |
| 520 | (I) | H | CH₂-cPr | p-C₆H₄—SMe | O—CH(Me)(CF₃) |
| 521 | (I) | Cyclobutyl | | p-C₆H₄—SMe | O—CH(Me)(CF₃) |
| 522 | (I) | Cyclopentyl | | p-C₆H₄—SMe | O—CH(Me)(CF₃) |
| 523 | (I) | H | Isobutyl | | |
| 524 | (I) | H | CH₂-cPr | p-C₆H₄—SMe | O-cPr |
| 525 | (I) | Cyclobutyl | | p-C₆H₄—SMe | O-cPr |
| 526 | (I) | Cyclopentyl | | p-C₆H₄—SMe | O-cPr |
| 527 | (I) | H | Isobutyl | p-C₆H₄—SMe | O-cPr |
| 528 | (I) | H | CH₂-cPr | p-C₆H₄—SMe | O—CH₂-cPr |
| 529 | (I) | Cyclobutyl | | p-C₆H₄—SMe | O—CH₂-cPr |
| 530 | (I) | Cyclopentyl | | p-C₆H₄—SMe | O—CH₂-cPr |
| 416 | (I) | H | Isobutyl | p-C₆H₄—SMe | O—CH₂-cPr |
| 531 | (I) | H | CH₂-cPr | p-C₆H₄—OMe | O—CH₂CF₃ |
| 532 | (I) | Cyclobutyl | | p-C₆H₄—OMe | O—CH₂CF₃ |
| 533 | (I) | Cyclopentyl | | p-C₆H₄—OMe | O—CH₂CF₃ |
| 534 | (I) | H | Isobutyl | p-C₆H₄—OMe | O—CH₂CF₃ |
| 535 | (I) | H | CH₂-cPr | p-C₆H₄—OMe | O—CH₂CH₂CF₃ |
| 536 | (I) | Cyclobutyl | | p-C₆H₄—OMe | O—CH₂CH₂CF₃ |
| 537 | (I) | Cyclopentyl | | p-C₆H₄—OMe | O—CH₂CH₂CF₃ |
| 538 | (I) | H | Isobutyl | p-C₆H₄—OMe | O—CH₂CH₂CF₃ |
| 539 | (I) | H | CH₂-cPr | p-C₆H₄—OMe | O—CH(CF₃)₂ |
| 540 | (I) | Cyclobutyl | | p-C₆H₄—OMe | O—CH(CF₃)₂ |
| 541 | (I) | Cyclopentyl | | p-C₆H₄—OMe | O—CH(CF₃)₂ |
| 542 | (I) | H | Isobutyl | p-C₆H₄—OMe | O—CH(CF₃)₂ |
| 543 | (I) | H | CH₂-cPr | p-C₆H₄—OMe | O—CH(Me)(CF₃) |
| 544 | (I) | Cyclobutyl | | p-C₆H₄—OMe | O—CH(Me)(CF₃) |
| 545 | (I) | Cyclopentyl | | p-C₆H₄—OMe | O—CH(Me)(CF₃) |
| 546 | (I) | H | Isobutyl | p-C₆H₄—OMe | O—CH(Me)(CF₃) |
| 547 | (I) | H | CH₂-cPr | p-C₆H₄—OMe | O-cPr |
| 548 | (I) | Cyclobutyl | | p-C₆H₄—OMe | O-cPr |
| 549 | (I) | Cyclopentyl | | p-C₆H₄—OMe | O-cPr |
| 550 | (I) | H | Isobutyl | p-C₆H₄—OMe | O-cPr |
| 551 | (I) | H | CH₂-cPr | p-C₆H₄—OMe | O—CH₂-cPr |
| 552 | (I) | Cyclobutyl | | p-C₆H₄—OMe | O—CH₂-cPr |
| 553 | (I) | Cyclopentyl | | p-C₆H₄—OMe | O—CH₂-cPr |
| 554 | (I) | H | Isobutyl | p-C₆H₄—OMe | O—CH₂-cPr |
| 555 | (I) | H | CH₂-cPr | 3,4-di-chloro phenyl | O—CH₂CF₃ |
| 556 | (I) | Cyclobutyl | | 3,4-di-chloro phenyl | O—CH₂CF₃ |
| 557 | (I) | Cyclopentyl | | 3,4-di-chloro phenyl | O—CH₂CF₃ |
| 558 | (I) | H | Isobutyl | | |
| 559 | (I) | H | CH₂-cPr | 3,4-di-chloro phenyl | O—CH₂CH₂CF₃ |
| 560 | (I) | Cyclobutyl | | 3,4-di-chloro phenyl | O—CH₂CH₂CF₃ |
| 561 | (I) | Cyclopentyl | | 3,4-di-chloro phenyl | O—CH₂CH₂CF₃ |
| 562 | (I) | H | Isobutyl | 3,4-di-chloro phenyl | O—CH₂CH₂CF₃ |
| 563 | (I) | H | CH₂-cPr | 3,4-di-chloro phenyl | O—CH(CF₃)₂ |
| 564 | (I) | Cyclobutyl | | 3,4-di-chloro phenyl | O—CH(CF₃)₂ |
| 565 | (I) | Cyclopentyl | | 3,4-di-chloro phenyl | O—CH(CF₃)₂ |
| 566 | (I) | H | Isobutyl | 3,4-di-chloro phenyl | O—CH(CF₃)₂ |
| 567 | (I) | H | CH₂-cPr | 3,4-di-chloro phenyl | O—CH(Me)(CF₃) |
| 568 | (I) | Cyclobutyl | | 3,4-di-chloro phenyl | O—CH(Me)(CF₃) |
| 569 | (I) | Cyclopentyl | | 3,4-di-chloro phenyl | O—CH(Me)(CF₃) |
| 570 | (I) | H | Isobutyl | 3,4-di-chloro phenyl | O—CH(Me)(CF₃) |
| 571 | (I) | H | CH₂-cPr | 3,4-di-chloro phenyl | O-cPr |
| 572 | (I) | Cyclobutyl | | 3,4-di-chloro phenyl | O-cPr |
| 573 | (I) | Cyclopentyl | | 3,4-di-chloro phenyl | O-cPr |
| 574 | (I) | H | Isobutyl | 3,4-di-chloro phenyl | O-cPr |
| 575 | (I) | H | CH2-cPr | 3,4-di-chloro phenyl | O—CH2-cPr |
| 576 | (I) | Cyclobutyl | | 3,4-di-chloro phenyl | O—CH2-cPr |
| 577 | (I) | Cyclopentyl | | 3,4-di-chloro phenyl | O—CH2-cPr |
| 578 | (I) | H | Isobutyl | 3,4-di-chloro phenyl | O—CH2-cPr |
| 579 | (I) | H | CH2-cPr | 5- | O—CH2CF3 |
| | | | | benzo[c][1,2,5]oxadiazyl | |
| 580 | (I) | Cyclobutyl | | 5- | O—CH2CF3 |
| | | | | benzo[c][1,2,5]oxadiazyl | |
| 581 | (I) | Cyclopentyl | | 5- | O—CH2CF3 |
| | | | | benzo[c][1,2,5]oxadiazyl | |
| 582 | (I) | H | Isobutyl | 5- | O—CH2CF3 |
| | | | | benzo[c][1,2,5]oxadiazyl | |
| 583 | (I) | H | CH2-cPr | 5- | O—CH2CH2CF3 |
| | | | | benzo[c][1,2,5]oxadiazyl | |
| 584 | (I) | Cyclobutyl | | 5- | O—CH2CH2CF3 |
| | | | | benzo[c][1,2,5]oxadiazyl | |
| 585 | (I) | Cyclopentyl | | 5- | O—CH2CH2CF3 |
| | | | | benzo[c][1,2,5]oxadiazyl | |
| 586 | (I) | H | Isobutyl | 5- | O—CH2CH2CF3 |
| | | | | benzo[c][1,2,5]oxadiazyl | |
| 587 | (I) | H | CH2-cPr | 5- | O—CH(CF3)2 |
| | | | | benzo[c][1,2,5]oxadiazyl | |
| 588 | (I) | Cyclobutyl | | 5- | O—CH(CF3)2 |
| | | | | benzo[c][1,2,5]oxadiazyl | |
| 589 | (I) | Cyclopentyl | | 5- | O—CH(CF3)2 |
| | | | | benzo[c][1,2,5]oxadiazyl | |
| 590 | (I) | H | Isobutyl | 5- | O—CH(CF3)2 |
| | | | | benzo[c][1,2,5]oxadiazyl | |
| 591 | (I) | H | CH2-cPr | 5- | O—CH(Me)(CF3) |
| | | | | benzo[c][1,2,5]oxadiazyl | |
| 592 | (I) | Cyclobutyl | | 5- | O—CH(Me)(CF3) |
| | | | | benzo[c][1,2,5]oxadiazyl | |
| 593 | (I) | Cyclopentyl | | 5- | O—CH(Me)(CF3) |
| | | | | benzo[c][1,2,5]oxadiazyl | |
| 594 | (I) | H | Isobutyl | 5- | O—CH(Me)(CF3) |
| | | | | benzo[c][1,2,5]oxadiazyl | |
| 595 | (I) | H | CH2-cPr | 5- | O-cPr |
| | | | | benzo[c][1,2,5]oxadiazyl | |
| 596 | (I) | Cyclobutyl | | 5- | O-cPr |
| | | | | benzo[c][1,2,5]oxadiazyl | |
| 597 | (I) | Cyclopentyl | | 5- | O-cPr |
| | | | | benzo[c][1,2,5]oxadiazyl | |
| 598 | (I) | H | Isobutyl | | |
| 599 | (I) | H | CH2-cPr | 5- | O—CH2-cPr |
| | | | | benzo[c][1,2,5]oxadiazyl | |
| 600 | (I) | Cyclobutyl | | 5- | O—CH2-cPr |
| | | | | benzo[c][1,2,5]oxadiazyl | |
| 601 | (I) | Cyclopentyl | | 5- | O—CH2-cPr |
| | | | | benzo[c][1,2,5]oxadiazyl | |
| 418 | (I) | H | Isobutyl | 5- | O—CH2-cPr |
| | | | | benzo[c][1,2,5]oxadiazyl | |
| 602 | (I) | H | CH2-cPr | 5- | O—CH2CF3 |
| | | | | benzo[c][1,2,5]thiadiazyl | |
| 603 | (I) | Cyclobutyl | | 5- | O—CH2CF3 |
| | | | | benzo[c][1,2,5]thiadiazyl | |
| 604 | (I) | Cyclopentyl | | 5- | O—CH2CF3 |
| | | | | benzo[c][1,2,5]thiadiazyl | |
| 605 | (I) | H | Isobutyl | 5- | O—CH2CF3 |
| | | | | benzo[c][1,2,5]thiadiazyl | |
| 606 | (I) | H | CH2-cPr | 5- | O—CH2CH2CF3 |
| | | | | benzo[c][1,2,5]thiadiazyl | |
| 607 | (I) | Cyclobutyl | | 5- | O—CH2CH2CF3 |
| | | | | benzo[c][1,2,5]thiadiazyl | |
| 608 | (I) | Cyclopentyl | | 5- | O—CH2CH2CF3 |
| | | | | benzo[c][1,2,5]thiadiazyl | |
| 609 | (I) | H | Isobutyl | 5- | O—CH2CH2CF3 |
| | | | | benzo[c][1,2,5]thiadiazyl | |
| 610 | (I) | H | CH2-cPr | 5- | O—CH(CF3)2 |
| | | | | benzo[c][1,2,5]thiadiazyl | |
| 611 | (I) | Cyclobutyl | | 5- | O—CH(CF3)2 |
| | | | | benzo[c][1,2,5]thiadiazyl | |
| 612 | (I) | Cyclopentyl | | 5- | O—CH(CF3)2 |
| | | | | benzo[c][1,2,5]thiadiazyl | |
| 613 | (I) | H | Isobutyl | 5- | O—CH(CF3)2 |
| | | | | benzo[c][1,2,5]thiadiazyl | |
| 614 | (I) | H | CH2-cPr | 5- | O—CH(Me)(CF3) |
| | | | | benzo[c][1,2,5]thiadiazyl | |
| 615 | (I) | Cyclobutyl | | 5- | O—CH(Me)(CF3) |
| | | | | benzo[c][1,2,5]thiadiazyl | |
| 616 | (I) | Cyclopentyl | | 5- | O—CH(Me)(CF3) |
| | | | | benzo[c][1,2,5]thiadiazyl | |
| 617 | (I) | H | Isobutyl | 5- | O—CH(Me)(CF3) |
| | | | | benzo[c][1,2,5]thiadiazyl | |
| 618 | (I) | H | CH2-cPr | 5- | O-cPr |
| | | | | benzo[c][1,2,5]thiadiazyl | |
| 619 | (I) | Cyclobutyl | | 5- | O-cPr |
| | | | | benzo[c][1,2,5]thiadiazyl | |
| 620 | (I) | Cyclopentyl | | 5- | O-cPr |
| | | | | benzo[c][1,2,5]thiadiazyl | |
| 621 | (I) | H | Isobutyl | 5- | O-cPr |
| | | | | benzo[c][1,2,5]thiadiazyl | |
| 622 | (I) | H | CH2-cPr | 5- | O—CH2-cPr |
| | | | | benzo[c][1,2,5]thiadiazyl | |
| 623 | (I) | Cyclobutyl | | 5- | O—CH2-cPr |
| | | | | benzo[c][1,2,5]thiadiazyl | |
| 624 | (I) | Cyclopentyl | | 5- | O—CH2-cPr |
| | | | | benzo[c][1,2,5]thiadiazyl | |
| 419 | (I) | H | Isobutyl | 5- | O—CH2-cPr |
| | | | | benzo[c][1,2,5]thiadiazyl | |
| 625 | (I) | H | CH2-cPr | 5-benzo[d]thiazolyl | O—CH2CF3 |
| 626 | (I) | Cyclobutyl | | 5-benzo[d]thiazolyl | O—CH2CF3 |
| 627 | (I) | Cyclopentyl | | 5-benzo[d]thiazolyl | O—CH2CF3 |
| 628 | (I) | H | Isobutyl | 5-benzo[d]thiazolyl | O—CH2CF3 |
| 629 | (I) | H | CH2-cPr | 5-benzo[d]thiazolyl | O—CH2CH2CF3 |
| 630 | (I) | Cyclobutyl | | 5-benzo[d]thiazolyl | O—CH2CH2CF3 |
| 631 | (I) | Cyclopentyl | | 5-benzo[d]thiazolyl | O—CH2CH2CF3 |
| 632 | (I) | H | Isobutyl | 5-benzo[d]thiazolyl | O—CH2CH2CF3 |
| 633 | (I) | H | CH2-cPr | 5-benzo[d]thiazolyl | O—CH(CF3)2 |
| 634 | (I) | Cyclobutyl | | 5-benzo[d]thiazolyl | O—CH(CF3)2 |
| 635 | (I) | Cyclopentyl | | 5-benzo[d]thiazolyl | O—CH(CF3)2 |
| 636 | (I) | H | Isobutyl | 5-benzo[d]thiazolyl | O—CH(CF3)2 |
| 637 | (I) | H | CH2-cPr | 5-benzo[d]thiazolyl | O—CH(Me)(CF3) |
| 638 | (I) | Cyclobutyl | | 5-benzo[d]thiazolyl | O—CH(Me)(CF3) |
| 639 | (I) | Cyclopentyl | | 5-benzo[d]thiazolyl | O—CH(Me)(CF3) |
| 640 | (I) | H | Isobutyl | 5-benzo[d]thiazolyl | O—CH(Me)(CF3) |
| 641 | (I) | H | CH2-cPr | 5-benzo[d]thiazolyl | O-cPr |
| 642 | (I) | Cyclobutyl | | 5-benzo[d]thiazolyl | O-cPr |
| 643 | (I) | Cyclopentyl | | 5-benzo[d]thiazolyl | O-cPr |
| 644 | (I) | H | Isobutyl | 5-benzo[d]thiazolyl | O-cPr |
| 645 | (I) | H | CH2-cPr | 5-benzo[d]thiazolyl | O—CH2-cPr |
| 646 | (I) | Cyclobutyl | | 5-benzo[d]thiazolyl | O—CH2-cPr |
| 647 | (I) | Cyclopentyl | | 5-benzo[d]thiazolyl | O—CH2-cPr |
| 648 | (I) | H | Isobutyl | 5-benzo[d]thiazolyl | O—CH2-cPr |
| 649 | (I) | H | CH2-cPr | 6-benzo[d]thiazolyl | O—CH2CF3 |
| 650 | (I) | Cyclobutyl | | 6-benzo[d]thiazolyl | O—CH2CF3 |
| 651 | (I) | Cyclopentyl | | 6-benzo[d]thiazolyl | O—CH2CF3 |
| 652 | (I) | H | Isobutyl | 6-benzo[d]thiazolyl | O—CH2CF3 |
| 653 | (I) | H | CH2-cPr | 6-benzo[d]thiazolyl | O—CH2CH2CF3 |
| 654 | (I) | Cyclobutyl | | 6-benzo[d]thiazolyl | O—CH2CH2CF3 |
| 655 | (I) | Cyclopentyl | | 6-benzo[d]thiazolyl | O—CH2CH2CF3 |
| 656 | (I) | H | Isobutyl | 6-benzo[d]thiazolyl | O—CH2CH2CF3 |
| 657 | (I) | H | CH2-cPr | 6-benzo[d]thiazolyl | O—CH(CF3)2 |
| 658 | (I) | Cyclobutyl | | 6-benzo[d]thiazolyl | O—CH(CF3)2 |
| 659 | (I) | Cyclopentyl | | 6-benzo[d]thiazolyl | O—CH(CF3)2 |
| 660 | (I) | H | Isobutyl | 6-benzo[d]thiazolyl | O—CH(CF3)2 |
| 661 | (I) | H | CH2-cPr | 6-benzo[d]thiazolyl | O—CH(Me)(CF3) |
| 662 | (I) | Cyclobutyl | | 6-benzo[d]thiazolyl | O—CH(Me)(CF3) |
| 663 | (I) | Cyclopentyl | | 6-benzo[d]thiazolyl | O—CH(Me)(CF3) |
| 664 | (I) | H | Isobutyl | 6-benzo[d]thiazolyl | O—CH(Me)(CF3) |
| 665 | (I) | H | CH2-cPr | 6-benzo[d]thiazolyl | O-cPr |
| 666 | (I) | Cyclobutyl | | 6-benzo[d]thiazolyl | O-cPr |
| 667 | (I) | Cyclopentyl | | 6-benzo[d]thiazolyl | O-cPr |
| 668 | (I) | H | Isobutyl | 6-benzo[d]thiazolyl | O-cPr |
| 669 | (I) | H | CH2-cPr | 6-benzo[d]thiazolyl | O—CH2-cPr |
| 670 | (I) | Cyclobutyl | | 6-benzo[d]thiazolyl | O—CH2-cPr |
| 671 | (I) | Cyclopentyl | | 6-benzo[d]thiazolyl | O—CH2-cPr |

Further examples are demonstrated in table 12

Compounds of formula (II)

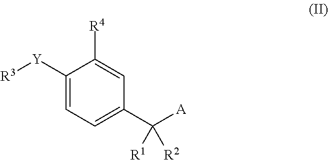

where A is CO₂H and Y is a bond

TABLE 12

| Example # | Formula | R¹ | R² | R³ | R⁴ |
|---|---|---|---|---|---|
| 700 | (II) | H | CH₂-cPr | p-C₆H₄—CF₃ | O—CH₂CF₃ |
| 701 | (II) | Cyclobutyl | | p-C₆H₄—CF₃ | O—CH₂CF₃ |
| 702 | (II) | Cyclopentyl | | p-C₆H₄—CF₃ | O—CH₂CF₃ |
| 133 | (II) | H | Isobutyl | p-C₆H₄—CF₃ | O—CH₂CF₃ |
| 703 | (II) | H | CH₂-cPr | p-C₆H₄—CF₃ | O—CH₂CH₂CF₃ |
| 704 | (II) | Cyclobutyl | | p-C₆H₄—CF₃ | O—CH₂CH₂CF₃ |
| 705 | (II) | Cyclopentyl | | p-C₆H₄—CF₃ | O—CH₂CH₂CF₃ |
| 706 | (II) | H | Isobutyl | p-C₆H₄—CF₃ | O—CH₂CH₂CF₃ |
| 707 | (II) | H | CH₂-cPr | p-C₆H₄—CF₃ | O—CH(CF₃)₂ |
| 708 | (II) | Cyclobutyl | | p-C₆H₄—CF₃ | O—CH(CF₃)₂ |
| 709 | (II) | Cyclopentyl | | p-C₆H₄—CF₃ | O—CH(CF₃)₂ |
| 710 | (II) | H | Isobutyl | p-C₆H₄—CF₃ | O—CH(CF₃)₂ |
| 711 | (II) | H | CH₂-cPr | p-C₆H₄—CF₃ | O—CH(Me)(CF₃) |
| 712 | (II) | Cyclobutyl | | p-C₆H₄—CF₃ | O—CH(Me)(CF₃) |
| 713 | (II) | Cyclopentyl | | p-C₆H₄—CF₃ | O—CH(Me)(CF₃) |
| 714 | (II) | H | Isobutyl | p-C₆H₄—CF₃ | O—CH(Me)(CF₃) |
| 715 | (II) | H | CH₂-cPr | p-C₆H₄—CF₃ | O-cPr |
| 716 | (II) | Cyclobutyl | | p-C₆H₄—CF₃ | O-cPr |
| 717 | (II) | Cyclopentyl | | p-C₆H₄—CF₃ | O-cPr |
| 718 | (II) | H | Isobutyl | p-C₆H₄—CF₃ | O-cPr |
| 414 | (II) | H | CH₂-cPr | p-C₆H₄—CF₃ | O—CH₂-cPr |
| 415 | (II) | Cyclobutyl | | p-C₆H₄—CF₃ | O—CH₂-cPr |
| 719 | (II) | Cyclopentyl | | p-C₆H₄—CF₃ | O—CH₂-cPr |
| 142 | (II) | H | Isobutyl | p-C₆H₄—CF₃ | O—CH₂-cPr |
| 720 | (II) | H | CH₂-cPr | p-C₆H₄—Cl | O—CH₂CF₃ |
| 721 | (II) | Cyclobutyl | | p-C₆H₄—Cl | O—CH₂CF₃ |
| 722 | (II) | Cyclopentyl | | p-C₆H₄—Cl | O—CH₂CF₃ |
| 723 | (II) | H | Isobutyl | p-C₆H₄—Cl | O—CH₂CF₃ |
| 724 | (II) | H | CH₂-cPr | p-C₆H₄—Cl | O—CH₂CH₂CF₃ |
| 725 | (II) | Cyclobutyl | | p-C₆H₄—Cl | O—CH₂CH₂CF₃ |
| 726 | (II) | Cyclopentyl | | p-C₆H₄—Cl | O—CH₂CH₂CF₃ |
| 727 | (II) | H | Isobutyl | p-C₆H₄—Cl | O—CH₂CH₂CF₃ |
| 728 | (II) | H | CH₂-cPr | p-C₆H₄—Cl | O—CH(CF₃)₂ |
| 729 | (II) | Cyclobutyl | | p-C₆H₄—Cl | O—CH(CF₃)₂ |
| 730 | (II) | Cyclopentyl | | p-C₆H₄—Cl | O—CH(CF₃)₂ |
| 731 | (II) | H | Isobutyl | p-C₆H₄—Cl | O—CH(CF₃)₂ |
| 732 | (II) | H | CH₂-cPr | p-C₆H₄—Cl | O—CH(Me)(CF₃) |
| 733 | (II) | Cyclobutyl | | p-C₆H₄—Cl | O—CH(Me)(CF₃) |
| 734 | (II) | Cyclopentyl | | p-C₆H₄—Cl | O—CH(Me)(CF₃) |
| 735 | (II) | H | Isobutyl | p-C₆H₄—Cl | O—CH(Me)(CF₃) |
| 736 | (II) | H | CH₂-cPr | p-C₆H₄—Cl | O-cPr |
| 737 | (II) | Cyclobutyl | | p-C₆H₄—Cl | O-cPr |
| 738 | (II) | Cyclopentyl | | p-C₆H₄—Cl | O-cPr |
| 739 | (II) | H | Isobutyl | p-C₆H₄—Cl | O-cPr |
| 740 | (II) | H | CH₂-cPr | p-C₆H₄—Cl | O—CH₂-cPr |
| 741 | (II) | Cyclobutyl | | p-C₆H₄—Cl | O—CH₂-cPr |
| 742 | (II) | Cyclopentyl | | p-C₆H₄—Cl | O—CH₂-cPr |
| 743 | (II) | H | Isobutyl | p-C₆H₄—Cl | O—CH₂-cPr |
| 744 | (II) | H | CH₂-cPr | p-C₆H₄—F | O—CH₂CF₃ |
| 745 | (II) | Cyclobutyl | | p-C₆H₄—F | O—CH₂CF₃ |
| 746 | (II) | Cyclopentyl | | p-C₆H₄—F | O—CH₂CF₃ |
| 747 | (II) | H | Isobutyl | p-C₆H₄—F | O—CH₂CF₃ |
| 748 | (II) | H | CH₂-cPr | p-C₆H₄—F | O—CH₂CH₂CF₃ |
| 749 | (II) | Cyclobutyl | | p-C₆H₄—F | O—CH₂CH₂CF₃ |
| 750 | (II) | Cyclopentyl | | p-C₆H₄—F | O—CH₂CH₂CF₃ |
| 751 | (II) | H | Isobutyl | p-C₆H₄—F | O—CH₂CH₂CF₃ |
| 752 | (II) | H | CH₂-cPr | p-C₆H₄—F | O—CH(CF₃)₂ |
| 753 | (II) | Cyclobutyl | | p-C₆H₄—F | O—CH(CF₃)₂ |
| 754 | (II) | Cyclopentyl | | p-C₆H₄—F | O—CH(CF₃)₂ |
| 755 | (II) | H | Isobutyl | p-C₆H₄—F | O—CH(CF₃)₂ |
| 756 | (II) | H | CH₂-cPr | p-C₆H₄—F | O—CH(Me)(CF₃) |
| 757 | (II) | Cyclobutyl | | p-C₆H₄—F | O—CH(Me)(CF₃) |
| 758 | (II) | Cyclopentyl | | p-C₆H₄—F | O—CH(Me)(CF₃) |
| 759 | (II) | H | Isobutyl | p-C₆H₄—F | O—CH(Me)(CF₃) |
| 760 | (II) | H | CH₂-cPr | p-C₆H₄—F | O-cPr |
| 761 | (II) | Cyclobutyl | | p-C₆H₄—F | O-cPr |
| 762 | (II) | Cyclopentyl | | p-C₆H₄—F | O-cPr |
| 763 | (II) | H | Isobutyl | p-C₆H₄—F | O-cPr |
| 764 | (II) | H | CH₂-cPr | p-C₆H₄—F | O—CH₂-cPr |
| 765 | (II) | Cyclobutyl | | p-C₆H₄—F | O—CH₂-cPr |
| 766 | (II) | Cyclopentyl | | p-C₆H₄—F | O—CH₂-cPr |
| 767 | (II) | H | Isobutyl | p-C₆H₄—F | O—CH₂-cPr |
| 768 | (II) | H | CH₂-cPr | p-C₆H₄—SMe | O—CH₂CF₃ |
| 769 | (II) | Cyclobutyl | | p-C₆H₄—SMe | O—CH₂CF₃ |
| 770 | (II) | Cyclopentyl | | p-C₆H₄—SMe | O—CH₂CF₃ |
| 771 | (II) | H | Isobutyl | p-C₆H₄—SMe | O—CH₂CF₃ |
| 772 | (II) | H | CH₂-cPr | p-C₆H₄—SMe | O—CH₂CH₂CF₃ |
| 773 | (II) | Cyclobutyl | | p-C₆H₄—SMe | O—CH₂CH₂CF₃ |
| 774 | (II) | Cyclopentyl | | p-C₆H₄—SMe | O—CH₂CH₂CF₃ |
| 775 | (II) | H | Isobutyl | p-C₆H₄—SMe | O—CH₂CH₂CF₃ |
| 776 | (II) | H | CH₂-cPr | p-C₆H₄—SMe | O—CH(CF₃)₂ |
| 777 | (II) | Cyclobutyl | | p-C₆H₄—SMe | O—CH(CF₃)₂ |
| 778 | (II) | Cyclopentyl | | p-C₆H₄—SMe | O—CH(CF₃)₂ |
| 779 | (II) | H | Isobutyl | p-C₆H₄—SMe | O—CH(CF₃)₂ |
| 780 | (II) | H | CH₂-cPr | p-C₆H₄—SMe | O—CH(Me)(CF₃) |
| 781 | (II) | Cyclobutyl | | p-C₆H₄—SMe | O—CH(Me)(CF₃) |
| 782 | (II) | Cyclopentyl | | p-C₆H₄—SMe | O—CH(Me)(CF₃) |
| 783 | (II) | H | Isobutyl | | |
| 784 | (II) | H | CH₂-cPr | p-C₆H₄—SMe | O-cPr |
| 785 | (II) | Cyclobutyl | | p-C₆H₄—SMe | O-cPr |
| 786 | (II) | Cyclopentyl | | p-C₆H₄—SMe | O-cPr |
| 787 | (II) | H | Isobutyl | p-C₆H₄—SMe | O-cPr |
| 788 | (II) | H | CH₂-cPr | p-C₆H₄—SMe | O—CH₂-cPr |
| 789 | (II) | Cyclobutyl | | p-C₆H₄—SMe | O—CH₂-cPr |
| 790 | (II) | Cyclopentyl | | p-C₆H₄—SMe | O—CH₂-cPr |
| 412 | (II) | H | Isobutyl | p-C₆H₄—SMe | O—CH₂-cPr |
| 791 | (II) | H | CH₂-cPr | p-C₆H₄—OMe | O—CH₂CF₃ |
| 792 | (II) | Cyclobutyl | | p-C₆H₄—OMe | O—CH₂CF₃ |
| 793 | (II) | Cyclopentyl | | p-C₆H₄—OMe | O—CH₂CF₃ |
| 794 | (II) | H | Isobutyl | p-C₆H₄—OMe | O—CH₂CF₃ |
| 795 | (II) | H | CH₂-cPr | p-C₆H₄—OMe | O—CH₂CH₂CF₃ |
| 796 | (II) | Cyclobutyl | | p-C₆H₄—OMe | O—CH₂CH₂CF₃ |
| 797 | (II) | Cyclopentyl | | p-C₆H₄—OMe | O—CH₂CH₂CF₃ |
| 798 | (II) | H | Isobutyl | p-C₆H₄—OMe | O—CH₂CH₂CF₃ |
| 799 | (II) | H | CH₂-cPr | p-C₆H₄—OMe | O—CH(CF₃)₂ |
| 800 | (II) | Cyclobutyl | | p-C₆H₄—OMe | O—CH(CF₃)₂ |
| 801 | (II) | Cyclopentyl | | p-C₆H₄—OMe | O—CH(CF₃)₂ |
| 802 | (II) | H | Isobutyl | p-C₆H₄—OMe | O—CH(CF₃)₂ |
| 803 | (II) | H | CH₂-cPr | p-C₆H₄—OMe | O—CH(Me)(CF₃) |
| 804 | (II) | Cyclobutyl | | p-C₆H₄—OMe | O—CH(Me)(CF₃) |
| 805 | (II) | Cyclopentyl | | p-C₆H₄—OMe | O—CH(Me)(CF₃) |
| 806 | (II) | H | Isobutyl | p-C₆H₄—OMe | O—CH(Me)(CF₃) |
| 807 | (II) | H | CH₂-cPr | p-C₆H₄—OMe | O-cPr |
| 808 | (II) | Cyclobutyl | | p-C₆H₄—OMe | O-cPr |
| 809 | (II) | Cyclopentyl | | p-C₆H₄—OMe | O-cPr |
| 810 | (II) | H | Isobutyl | p-C₆H₄—OMe | O-cPr |
| 811 | (II) | H | CH₂-cPr | p-C₆H₄—OMe | O—CH₂-cPr |
| 812 | (II) | Cyclobutyl | | p-C₆H₄—OMe | O—CH₂-cPr |
| 813 | (II) | Cyclopentyl | | p-C₆H₄—OMe | O—CH₂-cPr |
| 814 | (II) | H | Isobutyl | p-C₆H₄—OMe | O—CH₂-cPr |
| 815 | (II) | H | CH₂-cPr | 3,4-di-chloro phenyl | O—CH₂CF₃ |
| 816 | (II) | Cyclobutyl | | 3,4-di-chloro phenyl | O—CH₂CF₃ |
| 817 | (II) | Cyclopentyl | | 3,4-di-chloro phenyl | O—CH₂CF₃ |
| 818 | (II) | H | Isobutyl | | |
| 819 | (II) | H | CH₂-cPr | 3,4-di-chloro phenyl | O—CH₂CH₂CF₃ |
| 820 | (II) | Cyclobutyl | | 3,4-di-chloro phenyl | O—CH₂CH₂CF₃ |
| 821 | (II) | Cyclopentyl | | 3,4-di-chloro phenyl | O—CH₂CH₂CF₃ |
| 822 | (II) | H | Isobutyl | 3,4-di-chloro phenyl | O—CH₂CH₂CF₃ |
| 823 | (II) | H | CH₂-cPr | 3,4-di-chloro phenyl | O—CH(CF₃)₂ |
| 824 | (II) | Cyclobutyl | | 3,4-di-chloro phenyl | O—CH(CF₃)₂ |
| 825 | (II) | Cyclopentyl | | 3,4-di-chloro phenyl | O—CH(CF₃)₂ |
| 826 | (II) | H | Isobutyl | 3,4-di-chloro phenyl | O—CH(CF₃)₂ |
| 827 | (II) | H | CH₂-cPr | 3,4-di-chloro phenyl | O—CH(Me)(CF₃) |
| 828 | (II) | Cyclobutyl | | 3,4-di-chloro phenyl | O—CH(Me)(CF₃) |
| 829 | (II) | Cyclopentyl | | 3,4-di-chloro phenyl | O—CH(Me)(CF₃) |
| 830 | (II) | H | Isobutyl | 3,4-di-chloro phenyl | O—CH(Me)(CF₃) |
| 831 | (II) | H | CH₂-cPr | 3,4-di-chloro phenyl | O-cPr |
| 832 | (II) | Cyclobutyl | | 3,4-di-chloro phenyl | O-cPr |
| 833 | (II) | Cyclopentyl | | 3,4-di-chloro phenyl | O-cPr |
| 834 | (II) | H | Isobutyl | 3,4-di-chloro phenyl | O-cPr |
| 835 | (II) | H | CH₂-cPr | 3,4-di-chloro phenyl | O—CH₂-cPr |
| 836 | (II) | Cyclobutyl | | 3,4-di-chloro phenyl | O—CH₂-cPr |
| 837 | (II) | Cyclopentyl | | 3,4-di-chloro phenyl | O—CH₂-cPr |
| 838 | (II) | H | Isobutyl | 3,4-di-chloro phenyl | O—CH₂-cPr |
| 839 | (II) | H | CH₂-cPr | 5- | O—CH₂CF₃ |
| | | | | benzo[c][1,2,5]oxadiazyl | |
| 840 | (II) | Cyclobutyl | | 5- | O—CH₂CF₃ |
| | | | | benzo[c][1,2,5]oxadiazyl | |
| 841 | (II) | Cyclopentyl | | 5- | O—CH₂CF₃ |
| | | | | benzo[c][1,2,5]oxadiazyl | |
| 842 | (II) | H | Isobutyl | 5- | O—CH₂CF₃ |
| | | | | benzo[c][1,2,5]oxadiazyl | |
| 843 | (II) | H | CH₂-cPr | 5- | O—CH₂CH₂CF₃ |
| | | | | benzo[c][1,2,5]oxadiazyl | |
| 844 | (II) | Cyclobutyl | | 5- | O—CH₂CH₂CF₃ |
| | | | | benzo[c][1,2,5]oxadiazyl | |
| 845 | (II) | Cyclopentyl | | 5- | O—CH₂CH₂CF₃ |
| | | | | benzo[c][1,2,5]oxadiazyl | |
| 846 | (II) | H | Isobutyl | 5- | O—CH₂CH₂CF₃ |
| | | | | benzo[c][1,2,5]oxadiazyl | |
| 847 | (II) | H | CH₂-cPr | 5- | O—CH(CF₃)₂ |
| | | | | benzo[c][1,2,5]oxadiazyl | |
| 848 | (II) | Cyclobutyl | | 5- | O—CH(CF₃)₂ |
| | | | | benzo[c][1,2,5]oxadiazyl | |
| 849 | (II) | Cyclopentyl | | 5- | O—CH(CF₃)₂ |
| | | | | benzo[c][1,2,5]oxadiazyl | |
| 850 | (II) | H | Isobutyl | 5- | O—CH(CF₃)₂ |
| | | | | benzo[c][1,2,5]oxadiazyl | |
| 851 | (II) | H | CH₂-cPr | 5- | O—CH(Me)(CF₃) |
| | | | | benzo[c][1,2,5]oxadiazyl | |
| 852 | (II) | Cyclobutyl | | 5- | O—CH(Me)(CF₃) |
| | | | | benzo[c][1,2,5]oxadiazyl | |
| 853 | (II) | Cyclopentyl | | 5- | O—CH(Me)(CF₃) |
| | | | | benzo[c][1,2,5]oxadiazyl | |
| 854 | (II) | H | Isobutyl | 5- | O—CH(Me)(CF₃) |
| | | | | benzo[c][1,2,5]oxadiazyl | |
| 855 | (II) | H | CH₂-cPr | 5- | O-cPr |
| | | | | benzo[c][1,2,5]oxadiazyl | |
| 856 | (II) | Cyclobutyl | | 5- | O-cPr |
| | | | | benzo[c][1,2,5]oxadiazyl | |
| 857 | (II) | Cyclopentyl | | 5- | O-cPr |
| | | | | benzo[c][1,2,5]oxadiazyl | |
| 858 | (II) | H | Isobutyl | | |
| 859 | (II) | H | CH₂-cPr | 5- | O—CH₂-cPr |
| | | | | benzo[c][1,2,5]oxadiazyl | |
| 860 | (II) | Cyclobutyl | | 5- | O—CH₂-cPr |
| | | | | benzo[c][1,2,5]oxadiazyl | |
| 861 | (II) | Cyclopentyl | | 5- | O—CH₂-cPr |
| | | | | benzo[c][1,2,5]oxadiazyl | |
| 862 | (II) | H | Isobutyl | 5- | O—CH₂-cPr |
| | | | | benzo[c][1,2,5]oxadiazyl | |
| 863 | (II) | H | CH₂-cPr | 5- | O—CH₂CF₃ |
| | | | | benzo[c][1,2,5]thiadiazyl | |
| 864 | (II) | Cyclobutyl | | 5- | O—CH₂CF₃ |
| | | | | benzo[c][1,2,5]thiadiazyl | |
| 865 | (II) | Cyclopentyl | | 5- | O—CH₂CF₃ |
| | | | | benzo[c][1,2,5]thiadiazyl | |
| 866 | (II) | H | Isobutyl | 5- | O—CH₂CF₃ |
| | | | | benzo[c][1,2,5]thiadiazyl | |
| 867 | (II) | H | CH₂-cPr | 5- | O—CH₂CH₂CF₃ |
| | | | | benzo[c][1,2,5]thiadiazyl | |
| 868 | (II) | Cyclobutyl | | 5- | O—CH₂CH₂CF₃ |
| | | | | benzo[c][1,2,5]thiadiazyl | |
| 869 | (II) | Cyclopentyl | | 5- | O—CH₂CH₂CF₃ |
| | | | | benzo[c][1,2,5]thiadiazyl | |
| 870 | (II) | H | Isobutyl | 5- | O—CH₂CH₂CF₃ |
| | | | | benzo[c][1,2,5]thiadiazyl | |
| 871 | (II) | H | CH₂-cPr | 5- | O—CH(CF₃)₂ |
| | | | | benzo[c][1,2,5]thiadiazyl | |
| 872 | (II) | Cyclobutyl | | 5- | O—CH(CF₃)₂ |
| | | | | benzo[c][1,2,5]thiadiazyl | |
| 873 | (II) | Cyclopentyl | | 5- | O—CH(CF₃)₂ |
| | | | | benzo[c][1,2,5]thiadiazyl | |
| 874 | (II) | H | Isobutyl | 5- | O—CH(CF₃)₂ |
| | | | | benzo[c][1,2,5]thiadiazyl | |
| 875 | (II) | H | CH₂-cPr | 5- | O—CH(Me)(CF₃) |
| | | | | benzo[c][1,2,5]thiadiazyl | |
| 876 | (II) | Cyclobutyl | | 5- | O—CH(Me)(CF₃) |
| | | | | benzo[c][1,2,5]thiadiazyl | |
| 877 | (II) | Cyclopentyl | | 5- | O—CH(Me)(CF₃) |
| | | | | benzo[c][1,2,5]thiadiazyl | |
| 878 | (II) | H | Isobutyl | 5- | O—CH(Me)(CF₃) |
| | | | | benzo[c][1,2,5]thiadiazyl | |
| 879 | (II) | H | CH₂-cPr | 5- | O-cPr |
| | | | | benzo[c][1,2,5]thiadiazyl | |
| 880 | (II) | Cyclobutyl | | 5- | O-cPr |
| | | | | benzo[c][1,2,5]thiadiazyl | |
| 881 | (II) | Cyclopentyl | | 5- | O-cPr |
| | | | | benzo[c][1,2,5]thiadiazyl | |
| 882 | (II) | H | Isobutyl | 5- | O-cPr |
| | | | | benzo[c][1,2,5]thiadiazyl | |
| 883 | (II) | H | CH₂-cPr | 5- | O—CH₂-cPr |
| | | | | benzo[c][1,2,5]thiadiazyl | |
| 884 | (II) | Cyclobutyl | | 5- | O—CH₂-cPr |
| | | | | benzo[c][1,2,5]thiadiazyl | |
| 885 | (II) | Cyclopentyl | | 5- | O—CH₂-cPr |
| | | | | benzo[c][1,2,5]thiadiazyl | |
| 886 | (II) | H | Isobutyl | 5- | O—CH₂-cPr |
| | | | | benzo[c][1,2,5]thiadiazyl | |
| 887 | (II) | H | CH₂-cPr | 5-benzo[d]thiazolyl | O—CH₂CF₃ |
| 888 | (II) | Cyclobutyl | | 5-benzo[d]thiazolyl | O—CH₂CF₃ |
| 889 | (II) | Cyclopentyl | | 5-benzo[d]thiazolyl | O—CH₂CF₃ |
| 890 | (II) | H | Isobutyl | 5-benzo[d]thiazolyl | O—CH₂CF₃ |
| 891 | (II) | H | CH₂-cPr | 5-benzo[d]thiazolyl | O—CH₂CH₂CF₃ |
| 892 | (II) | Cyclobutyl | | 5-benzo[d]thiazolyl | O—CH₂CH₂CF₃ |
| 893 | (II) | Cyclopentyl | | 5-benzo[d]thiazolyl | O—CH₂CH₂CF₃ |
| 894 | (II) | H | Isobutyl | 5-benzo[d]thiazolyl | O—CH₂CH₂CF₃ |
| 895 | (II) | H | CH₂-cPr | 5-benzo[d]thiazolyl | O—CH(CF₃)₂ |
| 896 | (II) | Cyclobutyl | | 5-benzo[d]thiazolyl | O—CH(CF₃)₂ |
| 897 | (II) | Cyclopentyl | | 5-benzo[d]thiazolyl | O—CH(CF₃)₂ |
| 898 | (II) | H | Isobutyl | 5-benzo[d]thiazolyl | O—CH(CF₃)₂ |
| 899 | (II) | H | CH₂-cPr | 5-benzo[d]thiazolyl | O—CH(Me)(CF₃) |
| 900 | (II) | Cyclobutyl | | 5-benzo[d]thiazolyl | O—CH(Me)(CF₃) |
| 901 | (II) | Cyclopentyl | | 5-benzo[d]thiazolyl | O—CH(Me)(CF₃) |
| 902 | (II) | H | Isobutyl | 5-benzo[d]thiazolyl | O—CH(Me)(CF₃) |
| 903 | (II) | H | CH₂-cPr | 5-benzo[d]thiazolyl | O-cPr |
| 904 | (II) | Cyclobutyl | | 5-benzo[d]thiazolyl | O-cPr |
| 905 | (II) | Cyclopentyl | | 5-benzo[d]thiazolyl | O-cPr |
| 906 | (II) | H | Isobutyl | 5-benzo[d]thiazolyl | O-cPr |
| 907 | (II) | H | CH₂-cPr | 5-benzo[d]thiazolyl | O—CH₂-cPr |
| 908 | (II) | Cyclobutyl | | 5-benzo[d]thiazolyl | O—CH₂-cPr |
| 909 | (II) | Cyclopentyl | | 5-benzo[d]thiazolyl | O—CH₂-cPr |
| 910 | (II) | H | Isobutyl | 5-benzo[d]thiazolyl | O—CH₂-cPr |
| 911 | (II) | H | CH₂-cPr | 6-benzo[d]thiazolyl | O—CH₂CF₃ |
| 912 | (II) | Cyclobutyl | | 6-benzo[d]thiazolyl | O—CH₂CF₃ |
| 913 | (II) | Cyclopentyl | | 6-benzo[d]thiazolyl | O—CH₂CF₃ |
| 914 | (II) | H | Isobutyl | 6-benzo[d]thiazolyl | O—CH₂CF₃ |
| 915 | (II) | H | CH₂-cPr | 6-benzo[d]thiazolyl | O—CH₂CH₂CF₃ |
| 916 | (II) | Cyclobutyl | | 6-benzo[d]thiazolyl | O—CH₂CH₂CF₃ |
| 917 | (II) | Cyclopentyl | | 6-benzo[d]thiazolyl | O—CH₂CH₂CF₃ |
| 918 | (II) | H | Isobutyl | 6-benzo[d]thiazolyl | O—CH₂CH₂CF₃ |
| 919 | (II) | H | CH₂-cPr | 6-benzo[d]thiazolyl | O—CH(CF₃)₂ |
| 920 | (II) | Cyclobutyl | | 6-benzo[d]thiazolyl | O—CH(CF₃)₂ |
| 921 | (II) | Cyclopentyl | | 6-benzo[d]thiazolyl | O—CH(CF₃)₂ |
| 922 | (II) | H | Isobutyl | 6-benzo[d]thiazolyl | O—CH(CF₃)₂ |
| 923 | (II) | H | CH₂-cPr | 6-benzo[d]thiazolyl | O—CH(Me)(CF₃) |
| 924 | (II) | Cyclobutyl | | 6-benzo[d]thiazolyl | O—CH(Me)(CF₃) |
| 925 | (II) | Cyclopentyl | | 6-benzo[d]thiazolyl | O—CH(Me)(CF₃) |
| 926 | (II) | H | Isobutyl | 6-benzo[d]thiazolyl | O—CH(Me)(CF₃) |
| 927 | (II) | H | CH₂-cPr | 6-benzo[d]thiazolyl | O-cPr |
| 928 | (II) | Cyclobutyl | | 6-benzo[d]thiazolyl | O-cPr |
| 929 | (II) | Cyclopentyl | | 6-benzo[d]thiazolyl | O-cPr |
| 930 | (II) | H | Isobutyl | 6-benzo[d]thiazolyl | O-cPr |
| 931 | (II) | H | CH₂-cPr | 6-benzo[d]thiazolyl | O—CH₂-cPr |
| 932 | (II) | Cyclobutyl | | 6-benzo[d]thiazolyl | O—CH₂-cPr |
| 933 | (II) | Cyclopentyl | | 6-benzo[d]thiazolyl | O—CH₂-cPr |

### Experimental Procedures and Further Examples

### Example 55

### 2-(6-(4-methylsulfonylphenyl)-4′-(trifluoromethyl)biphenyl-3-yl)-4-methylpentanoic acid

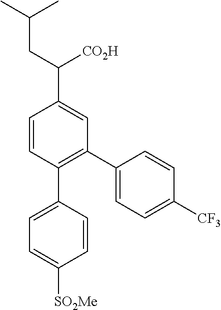

Step-1: ethyl-2-(3-bromo-4-benzyloxyphenyl)-4-methylpentanoate. Benzyl chloride (2.17 g, 17 mmol) was slowly added to a mixture of ethyl-2-(3-bromo-4-hydroxyphenyl)-4-methylpentanoate (4.5 g, 14.28 mmol), K₂CO₃ (2.95 g, 21.42 mmol) in 50 ml of DMSO at 60° C. for 3 h. The reaction mixture was poured into 200 ml of water and extracted with (2×300 ml) of hexane. The combined organic layers were washed with 100 ml of water, dried over sodium sulfate, filtered and concentrated under vacuum to give 5.0 g of ethyl 2-(3-bromo-4-benzyloxyphenyl)-4-methylpentanoate (86% yield). ¹H NMR (CDCl₃): δ 7.35 (m, 5H), 7.20 (d, J=7.9 Hz, 2H), 6.80 (s, 1H), 4.90 (s, 2H), 4.15 (q, 2H), 3.60 (t, 1H), 1.95-2.00 (m, 1H), 1.75-1.80 (m, 1H), 1.45-1.50 (m, 1H), 1.20 (t, 3H), 1.00 (m, 6H). Mass: (406, M+1, 100%).

Step-2: ethyl 2-(6-benzyloxy)-4′-(trifluoromethyl)biphenyl-3-yl)-4-methylpentanoate. A mixture of 2-(3-bromo-4-benzyloxyphenyl)-4-methylpenatoate (2.55 g, 6.3 mmol), 4-trifluoromethylbenzene boronic acid (1.43 g, 7.5 mmol), Pd(PPh₃)₄ (730 mg, 0.63 mmol), and Cs₂CO₃ (7.2 g, 2.2 mmol), was stirred overnight in DMF/Water (40 ml/10 ml) at 90° C. The reaction was filtered and the filter pad was washed with 100 ml of ethyl acetate. The combined filtrates were poured into 200 ml of water and extracted with (2×250 ml) of ethyl acetate. The combined organic layers were washed with 100 ml of brine solution followed by 100 ml of water. The organic layer was dried over sodium sulfate, filtered and concentrated. The residue was purified by flash column chromatography to give 1.6 g of ethyl 2-(6-benzyloxy)-4′-(trifluoromethyl)biphenyl-3-yl)-4-methylpentanoate (54% yield). ¹H NMR (CDCl₃): δ 7.45-7.50 (d, J=7.9 Hz, 2H), 7.32-7.38 (d, J=7.9 Hz, 2H) 7.30 (m, 5H), 7.20 (d, J=7.9 Hz, 2H), 6.80 (s, 1H), 4.90 (s, 2H), 4.15 (q, 2H), 3.60 (t, 1H), 1.95-2.00 (m, 1H), 1.75-1.80 (m, 1H), 1.45-1.50 (m, 1H), 1.20 (t, 3H), 1.00 (m, 6H). Mass: (471, M+1, 100%).

Step-3: ethyl 2-(6-hydroxy-4′-(trifluoromethyl)biphenyl-3-yl)-4-methylpentanoate. Ethyl 2-(6-benzyloxy)-4′-(trifluoromethyl)biphenyl-3-yl)-4-methylpentanoate (1.6 g) was hydrogenated overnight in 100 ml of MeOH at room temperature over 100 mg of Pd(OH)₂/C. The reaction mixture was filtered under nitrogen atmosphere and was washed with 100 ml of MeOH. The combined solution was concentrated under vacuum to yield 830 mg (64%) of the crude product ethyl 2-(6-hydroxy-4′-(trifluoromethyl)biphenyl-3-yl)-4-methylpentanoate which was used directly in the next step.

Step-4: ethyl 2-(6-trifluoromethylsulfonyloxy)-4′-(trifluoromethyl)biphenyl-3-yl)-4-methylpentanoate. A mixture of ethyl 2-(6-hydroxy)-4′-(trifluoromethyl)biphenyl-3-yl)-4-methylpentanoate (830 mg, 2.18 mmol) and diisopropylethylamine (370 mg, 2.85 mmol) in 25 ml dichloromethane was stirred for 30 minutes at 0° C. Triflic anhydride (740 mg, 2.62 mmol) was slowly added to the reaction mixture at 0° C. followed by stirring for 2 h at 0° C. The mixture was poured into 150 ml of water and extracted with dichloromethane (2×150 ml). The combined organic layers were washed with 10% NaHCO3 solution (150 ml) and 100 ml of water, dried over sodium sulfate, filtered and concentrated under vacuum to yield 1 g (89%). of crude product ethyl 2-(6-trifluoromethylsulfonyloxy)-4′-(trifluoromethyl)biphenyl-3-yl)-4-methylpentanoate as an oil that was used directly I the next step without further purification.

Step-5: ethyl 2-(6-(4-methylthiophenyl)-4′-(trifluoromethyl)biphenyl-3-yl)-4-methylpentanoate. A mixture of ethyl 2-(6-trifluoromethylsulfonyloxy)-4′-(trifluoromethyl)biphenyl-3-yl)-4-methylpentanoate (660 mg, 1.29 mmol), 4-thiomethyl benzene boronic acid (238 mg, 1.42 mmol), Pd(PPh₃)₄ (150 mg, 0.13 mmol) and Cs₂CO₃ 1.47 g, 4.511 mmol) in DMF/Water (25 ml/5 ml) was stirred overnight at 90° C. The reaction mixture was filtered and filter pad was washed with 100 ml of ethyl acetate. The combined filtrates were poured into 200 ml of water and extracted with ethyl acetate (2×150 ml). The combined organic layers were washed successively with brine (100 ml) and water (100 ml), dried over sodium sulfate, filtered and concentrated under reduced pressure. The resulting residue was purified by flash column chromatography to give 350 mg of the product ethyl 2-(6-(4-methylthiophenyl)-4′-(trifluoromethyl)biphenyl-3-yl)-4-methylpentanoate in 56% yield. ¹H NMR (CDCl₃): δ 7.45-7.50 (d, J=7.9 Hz, 2H), 7.32-7.38 (d, J=7.9 Hz, 2H) 7.30 (d, 2H), 7.20 (d, J=7.9 Hz, 2H), 7.00 (d, 2H) 6.80 (d, 1H), 4.15 (q, 2H), 3.60 (t, 1H), 2.35 (s, 3H), 2.00 (m, 1H), 1.75-1.80 (m, 1H), 1.45-1.50 (m, 1H), 1.20 (t, 3H), 1.00 (m, 6H). Mass: (487, M+1, 100%).

Step-6: ethyl 2-(6-(4-methylsulfonylphenyl)-4′-(trifluoromethyl)biphenyl-3-yl)-4-methylpentanoate: A mixture of ethyl 2-(6-(4-methylthiophenyl)-4′-(trifluoromethyl)biphenyl-3-yl)-4-methylpentanoate (240 mg, 0.49 mmol) and MCPBA (707 mg, 2.47 mmol) in 25 ml of dichloromethane was stirred for 5 h at room temperature. The reaction mixture was poured into 150 ml of water and extracted with dichloromethane (2×150 ml). The combined organic layers were washed with washed with 10% NaHCO3 solution (150 ml) and 100 ml of water, dried over sodium sulfate, filtered and concentrated under vacuum to an oil. The oil was purified by flash column chromatography to provide 140 mg of product ethyl 2-(6-(4-methylsulfonylphenyl)-4′-(trifluoromethyl)biphenyl-3-yl)-4-methylpentanoate (55%). ¹H NMR (CDCl₃): δ 7.45-7.50 (d, J=7.9 Hz, 2H), 7.32-7.38 (d, J=7.9 Hz, 2H) 7.30 (d, 2H), 7.20 (d, J=7.9 Hz, 2H), 7.00 (d, 2H) 6.80 (d, 1H), 4.15 (q, 2H), 3.60 (t, 1H), 3.25 (s, 3H), 2.00 (m, 1H), 1.75-1.80 (m, 1H), 1.45-1.50 (m, 1H), 1.20 (t, 3H), 1.00 (m, 6H). Mass: (519, M+1, 100%).

Step-7: 2-(6-(4-methylsulfonylphenyl)-4′-(trifluoromethyl)biphenyl-3-yl)-4-methyl-pentanoic acid: A mixture of 2-(6-(4-methylsulfonylphenyl)-4′-(trifluoromethyl)-biphenyl-3-yl)-4-methylpentanoate (100 mg, 0.21 mmol) and LiOH.H₂O (26 mg, 0.62 mmol) in MeOH/THF/Water mixture (10 ml/10 ml/5 ml) was stirred for 5 h at room temperature. The mixture was concentrated under reduced pressure and 50 ml of water was added. The solution was acidified to (pH 2) with 10% HCl solution and extracted with (2×100 ml) of ethyl acetate. The combined organic layers were washed with 50 ml of water, dried over sodium sulfate and filtered. The organic layer was concentrated under vacuum and residue was purified by flash column chromatography to give 44 mg of product 2-(6-(4-methylsulfonylphenyl)-4′-(trifluoromethyl)biphenyl-3-yl)-4-methyl-pentanoic acid (36% yield. ¹H NMR (CDCl₃): δ 7.45-7.50 (d, J=7.9 Hz, 2H), 7.32-7.38 (d, J=7.9 Hz, 2H) 7.30 (d, 2H), 7.20 (d, J=7.9 Hz, 2H), 7.00 (d, 2H) 6.80 (d, 1H), 3.60 (t, 1H), 3.25 (s, 3H), 2.00 (m, 1H), 1.75-1.80 (m, 1H), 1.45-1.50 (m, 1H), 1.00 (m, 6H). Mass: (491, M+1, 100%).

### Example 142

### 2-(2-cyclopropylmethoxy-4′-trifluoromethyl-biphenyl-4-yl)-4-methyl-pentanoic acid

### Step 1

### 2-cyclopropylmethoxy-4-fluoro-1-nitrobenzene

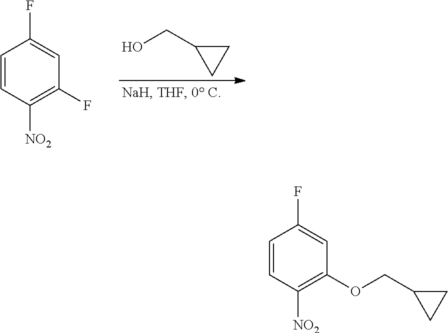

Cyclopropylmethanol (12 g, 167 mmol,) was added to a stirred suspension of NaH (60% in mineral oil, 6.7 g) in 200 mL THF over 15 min. at 0° C. under nitrogen. The reaction mixture was allowed to warm to room temperature and stirred for 1 h. A solution of 2,4-difluoro-1-nitrobenzene (24 g, 151 mmol) in 200 mL THF was added dropwise at 0° C. The reaction mixture was stirred at 0° C. for 2 h and then poured into ice water. The reaction mixture was extracted with diethyl ether (3×100 mL). The combined organic layers were dried over MgSO₄ and concentrated under reduced pressure to give 22.0 g of product as an orange oil that was used in the next step without purification.

### Step 2

### Ethyl-2-(3-(cyclopropylmethoxy)-4-nitrophenyl)-acetate

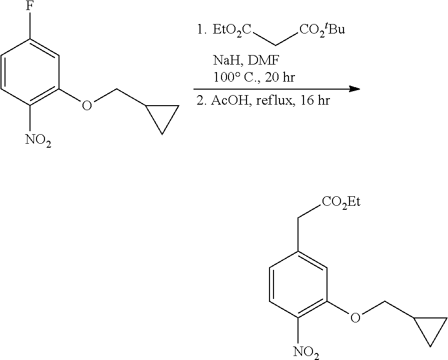

tert-Butyl ethyl malonate (21.6 g, 114.9 mmol) was added to a stirred suspension of sodium hydride (60% in mineral oil, 4.6 g) in 100 mL DMF over 15 min. at 0° C. under nitrogen. The reaction mixture was allowed to warm to room temperature and stirred for 1 h. A solution of 2-cyclopropylmethoxy-4-fluoro-1-nitrobenzene (22.0 g, 104.3 mmol) in DMF (100 mL) was added dropwise at 0° C., and the reaction mixture was heated 100° C. for 20 h. The reaction mixture was allowed to cool to room temperature, poured into ice water and extracted with EtOAc (3×100 mL). The combined organic phases were washed with water (3×100 mL), brine (100 mL) and dried (MgSO₄). Evaporation of solvent under reduced pressure gave 35.0 g of crude product as brown oil that was used for the next step without purification. The crude product (30.0 g, 79 mmol) was dissolved in glacial AcOH (100 mL) and the reaction mixture was heated under reflux for 16 h. After cooling a solvent evaporated to give crude brown oil 20 g, which was purified by chromatography over silica gel using a heptane-EtOAc solvent gradient (9:1-4:1) to give 14.0 g of product ethyl-2-(3-(cyclopropylmethoxy)-4-nitrophenyl)-acetate as a yellow oil (64%).

### Step 3

### Ethyl-(4-amino-3-cyclopropylmethoxy-phenyl)-acetate

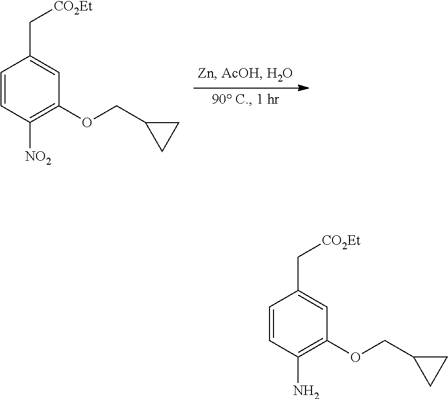

Ethyl-2-(3-(cyclopropylmethoxy)-4-nitrophenyl)-acetate (12.5 g, 45 mmol) was dissolved in a mixture of glacial AcOH (300 mL) and H₂O (60 mL) and zinc powder was added. The reaction mixture was heated at 90° C. for 1 h, cooled to room temperature and filtered. The filtrate was concentrated under reduced pressure and extracted with EtOAc (3×100 mL). The combined organic phases were dried over MgSO₄ and concentrated under reduced pressure to a dark oil. The crude product was purified by chromatography over silica gel using a heptane-EtOAc solvent gradient (4:1-1:1) to give the product ethyl-(4-amino-3-cyclopropylmethoxyphenyl)-acetate as an oil 5.6 g, (50%).

### Step 4

### Ethyl-(4-iodo-3-cyclopropylmethoxy-phenyl)-acetate

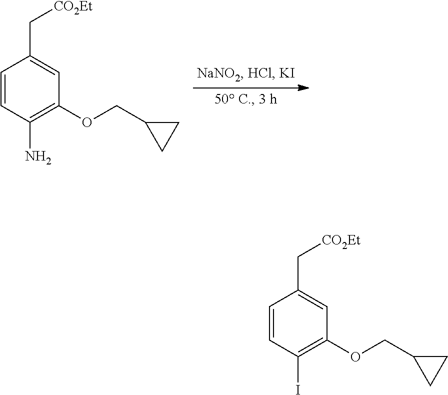

Ethyl-(4-amino-3-cyclopropylmethoxy-phenyl)-acetate (2.5 g, 10.0 mmol) was dissolved in a mixture of EtOH/H₂O/H₂SO₄ (96%) 50 mL/100 mL/2.5 mL at 0° C. A solution of NaNO₂ (0.8 g, 11.6 mmol) in water (10 mL) was added dropwise at 0° C., and the reaction mixture was stirred for 40 min. at the same temperature. A solution of KI (5.0 g, 30.1 mmol) in water (20 mL) was added dropwise at 0° C. The reaction mixture was heated 50-60° C. for 2.5 h and the solvent was evaporated. The reaction mixture was extracted with EtOAc (3×50 mL), and the combined organic layers were washed with 10% sodium thiosulfate (1×30 mL) followed by brine (30 mL). The solution was dried over MgSO₄ and concentrated to give crude black oil 24.3 g, which was purified which was purified by chromatography over silica gel using a heptane-EtOAc solvent gradient (9:1-4:1) to give the product ethyl-(4-iodo-3-cyclopropylmethoxy-phenyl)-acetate as a yellow oil 1.2 g, (33%).

### Step 5

### Ethyl-(2-cyclopropylmethoxy-4′-trifluoromethyl-biphenyl-4-yl)-acetate

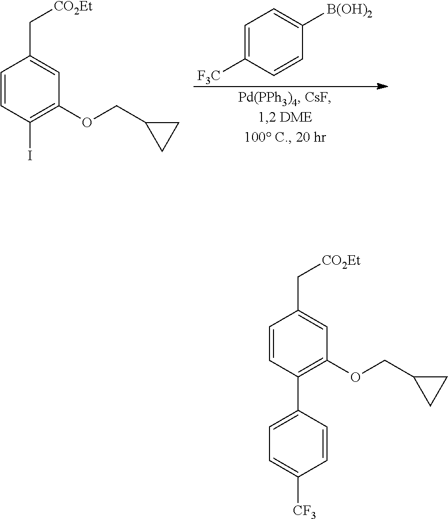

A mixture of ethyl-(4-iodo-3-cyclopropylmethoxy-phenyl)-acetate (0.28 g, 0.77 mmol), 4-trifluoromethylphenylboronic acid (0.18 g, 0.95 mmol), CsF (0.28 g, 1.84 mmol) and Pd(PPh₃)₄ (0.036 g, 0.026 mmol) in 10 mL anhydrous DME was refluxed for 20 h under argon. The reaction mixture was cooled, and 15 mL of EtOAc and 15 mL or water were added. The organic phase was separated, dried over MgSO₄ and concentrated under reduced pressure to a yellow oil. The oil was purified by chromatography over silica gel using a heptane-EtOAc solvent gradient (9:1-4:1) to give 0.2 g of product ethyl-(2-cyclopropylmethoxy-4′-trifluoromethyl-biphenyl-4-yl)-acetate (69%) as a yellow oil.

### Example 142

### 2-(2-cyclopropylmethoxy-4′-trifluoromethyl-biphenyl-4-yl)-4-methyl-pentanoic acid

### 2-(2-cyclopropylmethoxy-4′-trifluoromethyl-biphenyl-4-yl)-4-methyl-pentanoic acid

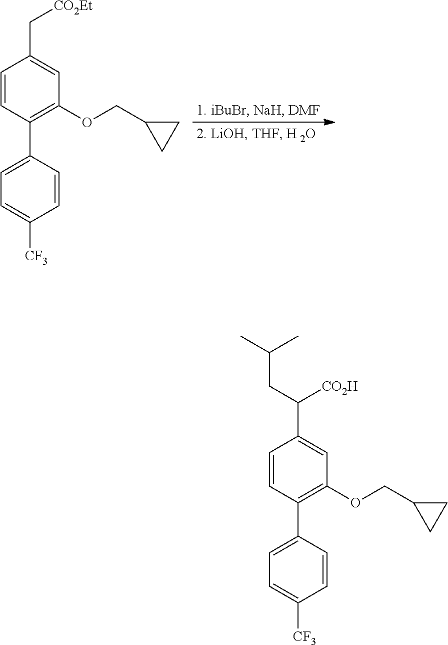

Ethyl-(2-cyclopropylmethoxy-4′-trifluoromethyl-biphenyl-4-yl)-acetate (0.1 g, 0.26 mmol) was dissolved in 10 mL anhydrous DMF and NaH (60% wt. in oil, 0.013 g, 0.33 mmol) was added at 0° C. The reaction mixture was stirred for 0.5 h at 25° C. and i-BuBr (0.04 g, 0.031 mL, 0.29 mmol) was added dropwise at 0° C. The reaction mixture was stirred an additional 1 h at 0° C. and saturated NH₄Cl solution (10 mL) was added. The reaction mixture was extracted with EtOAc (3×10 mL) and the organic combined organic phases were washed with water (3×20 mL) and brine (10 mL). The organic phase was dried over MgSO₄ and evaporated under reduced pressure to give 0.08 g of a colorless oil. The oil was dissolved in 10 mL of EtOH/H₂O (9:1, vvl) and 0.2 g KOH added. The reaction mixture was refluxed for 18 h and concentrated under reduced pressure. Water (10 mL) was added and the reaction mixture was extracted with EtOAc (3×10 mL). The combined organic phases were dried over MgSO₄ and evaporated under reduced pressure to give 0.065 g of crude product as a colorless oil. Purification by gradient column chromatography over silica gel (heptane-EtOAc 9:1-1:1) gave the 0.03 g of the product 2-(2-cyclopropylmethoxy-4′-trifluoromethyl-biphenyl-4-yl)-4-methyl-pentanoic acid. as a white solid (40%). M.P.=142-143° C., ¹H NMR (300 MHz, CDCl₃/TMS): δ 7.69-7.62 (m, 4H), 7.27 (d, J=7.8 Hz, 1H), 7.00 (d, J=7.8 Hz, 1H), 6.95 (s, 1H), 3.83 (d, J=6.6 Hz, 2H), 3.68 (t, J=6.6 Hz, 1H), 2.01-1.94 (m, 1H), 1.77-1.67 (m, 1H), 1.60-1.51 (m, 1H), 1.17 (m, 1H), 0.94 (d, J=6.6 Hz, 6H), 0.60-0.50 (m, 2H), 0.30-0.22 (m, 2H). ¹³C NMR (75 MHz, CDCl₃/TMS): δ 179.2, 155.9, 141.8, 140.0, 130.7, 129.6, 128.7 (q, ²J_{CF}=33 Hz), 128.6, 124.6 (q, ³J_{CF}=4 Hz), 124.3 (q, ¹J_{CF}=271 Hz), 120.7, 112.8, 73.2, 49.5, 42.1, 25.9, 22.7, 22.3, 10.2, 3.2.

### Example 216

### 2-(6-cyclopropylmethoxy)-4′-(trifluoromethyl)biphenyl-3-yl)-4-methylpentanoic acid

### Step-1

### Ethyl 2-(3-bromo-4-cyclopropylmethoxyphenyl)-4-methylpenatoate

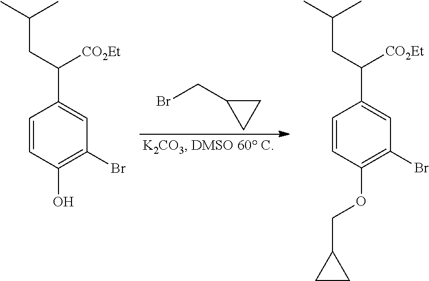

Cyclopropylmethyl bromide (235 mg, 1.74 mmol) was added to a stirred mixture of ethyl 2-(3-bromo-4-hydroxyphenyl)-4-methylpentanoate (500 mg, 1.59 mmol) and K₂CO₃ (438 mg, 3.17 mmol) in DMSO (15 ml) at room temperature. The resulting reaction mixture was allowed to stir at 60° C. for 3 h, poured into 100 ml of water and extracted with ethyl acetate (2×200 ml). The combined organic extracts were washed with 100 ml of water, dried over sodium sulfate, filtered and concentrated under vacuum to give 530 mg of ethyl 2-(3-bromo-4-cyclopropylmethoxyphenyl)-4-methylpentanoate (91%) which was used directly in the next step. ¹H NMR (CDCl₃): δ 7.20 (d, J=7.9 Hz, 2H), 6.80 (s, 1H), 4.15 (q, 2H), 3.80 (m, 2H), 3.60 (t, 1H), 1.95-2.00 (m, 1H), 1.75-1.80 (m, 2H), 1.45-1.50 (m, 1H), 1.20 (t, 3H), 1.00 (m, 6H), 0.65-0.75 (m, 2H), 0.55-0.62 (m, 2H). Mass: (370, M+1, 100%).

### Step 2

### Ethyl 2-(6-cyclopropylmethoxy)-4′-(trifluoromethyl)biphenyl-3-yl)-4-methylpentanoate

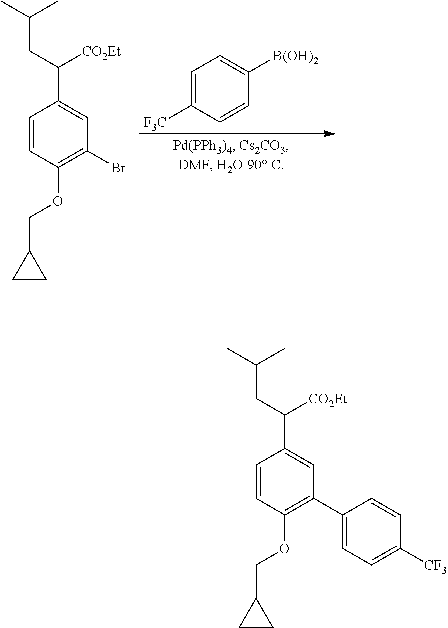

A mixture of ethyl 2-(3-bromo-4-cyclopropylmethoxyphenyl)-4-methylpentanoate (500 mg, 1.35 mmol), 4-trifluoromethylbenzene boronic acid (283 mg, 1.49 mmol), Pd(PPh₃)₄ (156.5 mg, 0.135 mmol) and Cs₂CO₃ (1.546 g, 4.74 mmol) in DMF/Water (25 ml/5 ml) was stirred overnight at 90° C. under argon. The reaction mixture was filtered and the filtrate extracted with 100 ml of ethyl acetate. Water (200 mL) was added to the combined filtrates followed by extraction with ethyl acetate (2×250 ml). The combined organic extracts were washed with 100 ml of brine solution followed by 100 ml of water and dried over sodium sulfate. The organic phase was concentrated under reduced pressure to a residue which was purified by column chromatography over silica gel (Ethyl acetate:Hexane, 1:4) to give 234 mg of ethyl 2-(6-cyclopropylmethoxy)-4′-(trifluoromethyl)biphenyl-3-yl)-4-methylpentanoate product (40%). ¹HNMR: (CDCl₃): δ7.45-7.50 (d, J=7.9 Hz, 2H), 7.32-7.38 (d, J=7.9 Hz, 2H), 7.20 (d, J=7.9 Hz, 2H), 6.80 (s, 1H), 4.15 (q, 2H), 3.80 (m, 2H), 3.60 (t, 1H), 1.95-2.00 (m, 1H), 1.75-1.80 (m, 2H), 1.45-1.50 (m, 1H), 1.20 (t, 3H), 1.00 (m, 6H), 0.65-0.75 (m, 2H), 0.55-0.62 (m, 2H). Mass: (436, M+1, 100%).

### Step 4

### 2-(6-cyclopropylmethoxy)-4′-(trifluoromethyl)biphenyl-3-yl)-4-methylpentanoic acid

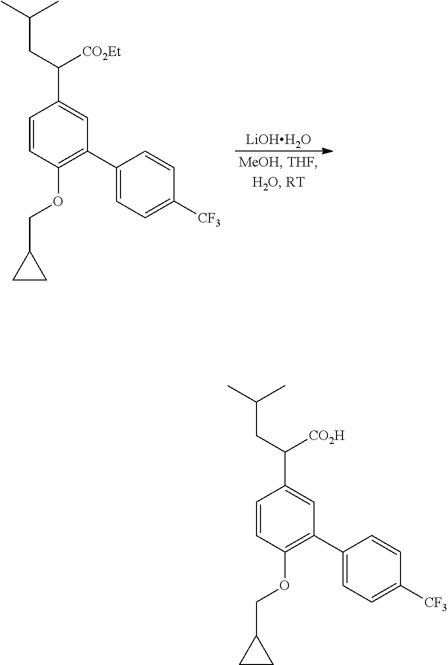

A mixture of 2-(6-cyclopropylmethoxy)-4′-(trifluoromethyl)biphenyl-3-yl)-4-methylpentanoate (110 mg, 0.253 mmol) and lithium hydroxide monohydrate (32 mg, 0.76 mmol), in 25 ml MeOH/THF/Water (10 v/10 v/5 v) was stirred for 5 h at RT. The mixture was concentrated under vacuum, 50 ml of water were added and the solution acidified to ca. pH 2 with 10% HCl solution. This mixture was extracted with ethyl acetate (2×100 ml) and the combined organic layers were washed with 50 ml of water, dried over sodium sulfate and filtered. The filtrate was concentrated under vacuum to a residue which was purified by column chromatography over silica gel (ethyl acetate:hexane, 2:3) to give 54 mg of 2-(6-cyclopropylmethoxy)-4′-(trifluoromethyl)biphenyl-3-yl)-4-methylpentanoic acid (51%). ¹HNMR: (CDCl₃): δ7.45-7.50 (d, J=7.9 Hz, 2H), 7.32-7.38 (d, J=7.9 Hz, 2H), 7.20 (d, J=7.9 Hz, 2H), 6.80 (s, 1H), 3.80 (m, 2H), 3.60 (t, 1H), 1.95-2.00 (m, 1H), 1.75-1.80 (m, 2H), 1.45-1.50 (m, 1H), 1.00 (m, 6H), 0.65-0.75 (m, 2H), 0.55-0.62 (m, 2H). Mass: (407, M+1, 100%).

### Synthesis of Example 406

### (6-Cyclopropylmethoxy-4′-trifluoromethylbiphenyl-3-yl)-acetic Acid

### Step 1

### Cyclopropylmethyl Mesylate

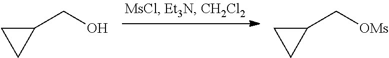

To a stirred solution of cyclopropanemethanol (1.00 g, 13.9 mmol) in dry CH₂Cl₂ (6 mL) cooled at −30° C. was added MsCl (2.38 g, 20.8 mmol) in one portion. Et₃N (2.25 g, 22.2 mmol) was then added dropwise via syringe over 5 min. The resulting mixture was stirred at −20 to 0° C. for 45 min. Cold 2 N aqueous HCl solution (3 mL) and brine (2.5 mL) were added dropwise via pipettes at 0° C. The resulting biphasic mixture was separated. The aqueous layer was diluted with cold brine (5 mL) and extracted with CH₂Cl₂ (2×7 mL). The combined organic phases were washed with cold brine (5 mL), dried over MgSO₄, filtered and concentrated under reduced pressure. The resulting yellow liquid was dried under high vacuum to afford the title compound (1.84 g, 88%). ¹H NMR (300 MHz, CDCl₃/TMS) δ 4.08 (d, J=7.5 Hz, 2H), 3.04 (s, 3H), 1.30-1.16 (m, 1H), 0.74-0.62 (m, 2H), 0.42-0.34 (m, 2H); ¹³C NMR (CDCl₃) δ 75.4, 37.8, 10.1, 3.9.

### Step 2

### (3-Bromo-4-cyclopropylmethoxy-phenyl)-acetic acid methyl ester

Method A (from Mesylate)

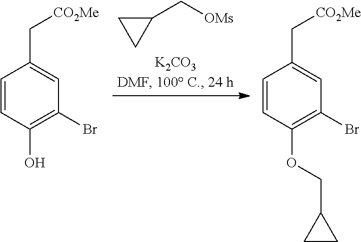

A mixture of methyl (3-bromo-4-hydroxyphenyl)-acetate (1.00 g, 4.08 mmol), cyclopropanemethyl mesylate (2, 0.92 g, 6.12 mmol) and K₂CO₃ (1.69 g, 12.24 mmol) in dry DMF (10 mL) was stirred and heated to 100° C. for 24 h. The mixture was then cooled to room temperature, water (100 mL) was added, and the mixture was extracted with EtOAc (3×30 mL). The combined organic phases were washed with brine (15 mL), dried over MgSO₄, filtered and concentrated under reduced pressure. The resulting black oil (1.00 g) was purified by chromatography (heptane/EtOAc, 10:1) to afford the title compound (0.55 g, 45%). ¹H NMR (300 MHz, CDCl₃/TMS) δ 7.46 (d, J=2.1 Hz, 1H), 7.14 (dd, J=8.4, 2.1 Hz, 1H), 6.82 (d, J=8.4 Hz, 1H), 3.86 (d, J=6.6 Hz, 2H), 3.68 (s, 3H), 3.53 (s, 2H), 1.36-1.22 (m, 1H), 0.66-0.58 (m, 2H), 0.42-0.34 (m, 2H); ¹³C NMR (75 MHz, CDCl₃/TMS) δ 171.5, 154.3, 133.8, 129.0, 127.3, 113.6, 112.3, 73.7, 52.0, 39.7, 10.1, 3.2.

Method B (from Cyclopropylmethyl Bromide)

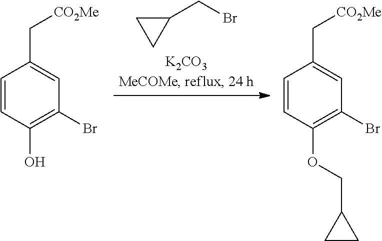

To a solution of methyl (3-bromo-4-hydroxyphenyl)-acetate (5.00 g, 20.4 mmol) in acetone (55 mL) was added K₂CO₃ (8.46 g, 61.2 mmol), followed by cyclopropanemethyl bromide (4.13 g, 30.6 mmol). The mixture was stirred and heated to reflux for 24 h under argon. It was cooled to room temperature and the solvent was removed under reduced pressure. The residue was suspended in water (100 mL) and washed with water (2×20 mL) and brine (10 mL), dried over MgSO₄, filtered, and concentrated under reduced pressure. The resulting yellow oil (8.00 g) was purified by chromatography (heptane/EtOAc, 15:1, 10:1) to afford the title compound (5.34 g, 87%).

### Step 3

### (6-Cyclopropylmethoxy-4′-trifluoromethyl-biphenyl-3-yl)-acetic acid methyl ester

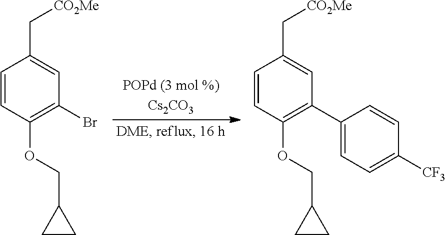

A mixture of (3-bromo-4-cyclopropylmethoxy-phenyl)-acetic acid methyl ester (500 mg, 1.67 mmol), 4-trifluoromethylphenylboronic acid (476 mg, 2.51 mmol), Cs₂CO₃ (1634 mg, 5.01 mmol) and DME (10 mL) was purged with argon. POPd catalyst (42 mg, 0.08 mmol) was added and the mixture was purged with argon again. The mixture was heated to reflux under argon for 16 h. The reaction mixture was cooled to room temperature and passed through a Celite™ pad, which was washed with EtOAc (15 mL). The filtrate was evaporated to dryness. The residue (750 mg) was purified by chromatography (silica gel, heptane/EtOAc, 15:1, 10:1) to afford the title compound (500 mg, 96%) as a yellow oil. HPLC purity: 72.5%. ¹H NMR (300 MHz, CDCl₃/TMS) δ 7.67 (m, 4H), 7.24 (m, 2H), 6.93 (m, 1H), 3.81 (d, J=6.9 Hz, 2H), 3.70 (s, 3H), 3.61 (s, 2H), 1.25-1.10 (m, 1H), 0.57-0.54 (m, 2H), 0.29-0.22 (m, 2H); ¹³C NMR (75 MHz, CDCl₃/TMS) δ 171.9, 154.8, 141.8, 131.5, 129.9, 129.6, 129.4, 128.6 (q, J=32 Hz), 126.3, 124.5 (q, J=4 Hz), 124.2 (q, J=270 Hz), 113.1, 73.2, 52.0, 40.2, 10.2, 3.1; ¹⁹F NMR (282 MHz, CDCl₃/CFCl₃) δ −62.2.

### Step 4

### (6-Cyclopropylmethoxy-4′-trifluoromethylbiphenyl-3-yl)-acetic acid

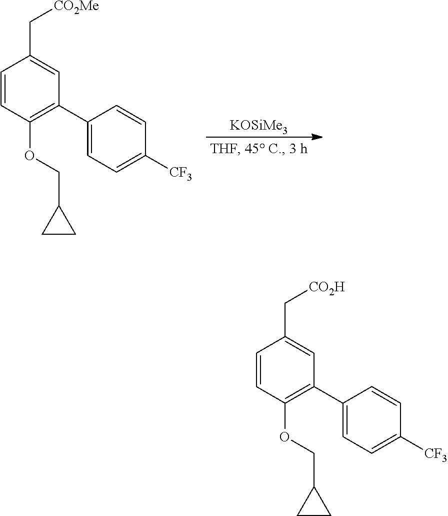

To a solution of (6-Cyclopropylmethoxy-4′-trifluoromethyl-biphenyl-3-yl)-acetic acid methyl ester (500 mg, 1.61 mmol) in dry THF (8 mL) was added potassium trimethylsilanolate (90% technical grade; 230 mg, 1.61 mmol) and the solution was heated to 45° C. for 2 h. An additional 230 mg of potassium trimethylsilanolate was added and heating was continued for 1 h longer. The solution was allowed to cool to room temperature and the solvent was evaporated. The residue was suspended in EtOAc (20 mL) and acidified with 1 N HCl to pH 2. The organic layer was separated from the aqueous layer, washed with brine (10 mL), and dried over MgSO₄. After evaporation of the solvent, the residue (520 mg) was purified by chromatography (normal phase, heptane/EtOAc, 3:1 followed by reverse phase C-18 column, ACN/H₂O, 60:40) to afford 260 mg of title compound (54%) as a white solid. HPLC purity: 91.1%; ¹H NMR (300 MHz, CDCl₃/TMS) δ 9.02 (br s, 1H), 7.74-7.50 (m, 4H), 7.28-7.10 (m, 2H), 6.92 (d, J=8.4 Hz, 1H), 3.80 (d, J=6.6 Hz, 2H), 3.61 (s, 3H), 3.61 (s, 2H), 1.35-1.05 (m, 1H), 0.60-0.42 (m, 2H), 0.30-0.14 (m, 2H); ¹³C NMR (75 MHz, CDCl₃/TMS) δ 177.8, 155.0, 141.6, 131.6, 130.0, 129.6, 129.5, 128.7 (q, J=32 Hz), 125.6, 124.6 (q, J=4 Hz), 124.2 (q, J=270 Hz), 113.1, 73.2, 40.1, 10.1, 3.1; ¹⁹F NMR (282 MHz, CDCl₃/CFCl₃) δ −62.2.

### Example 410

### (2-(2-Isopropylsulfanyl-4′-trifluoromethyl-biphenyl-4-yl)-butyric Acid

### Step 1

### 2-(3-Isopropylsulfanyl-4-nitro-phenyl)-butyric acid ethyl ester

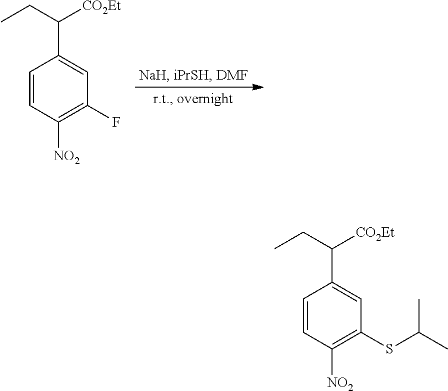

To a solution of 2-propanethiol (2.15 g, 28.2 mmol) in DMF (60 mL) cooled in an ice bath was added NaH (60%, 1.35 g, 33.8 mmol) and the mixture was stirred at room temperature for 30 min. 2-(3-Fluoro-4-nitro-phenyl)-butyric acid ethyl ester (6.00 g, 23.5 mmol) in DMF (10 mL) was added dropwise and the mixture was stirred at room temperature overnight. Water (600 mL) was added carefully to quench the reaction and the mixture was adjusted by addition of NH₄Cl (3.0 g) to neutral, followed by extraction with ethyl acetate (600 mL). The organic layer was washed with brine (600 mL), dried over sodium sulfate, concentrated under reduced pressure. The residue was purified by a silica gel flash chromatography eluted with heptane/ethyl acetate (20:1) to give the desired product (4.70 g, 64%) as a yellow oil: ¹H NMR (300 MHz, CDCl₃) δ 8.10 (d, 1 H, J=8.7 Hz), 7.45 (s, 1H), 7.18 (dd, 1H, J=8.7, 1.5 Hz), 4.15 (m, 2H), 3.62 (m, 1H), 3.53 (t, 1H, J=7.8 Hz), 2.13 (m, 1H), 1.82 (m, 1H), 1.40 (m, 6H), 1.23 (t, 3H, J=6.9 Hz), 0.92 (t, 3H, J=7.2 Hz); ¹³C NMR (75 MHz, CDCl₃) δ 172.51, 145.71, 144.71, 136.77, 127.29, 126.04, 124.37, 61.13, 53.22, 35.06, 26.89, 22.25, 14.16, 12.06.

### Step 2

### 2-(4-Amino-3isopropylsulfanyl-phenyl_-butyric acid ethyl ester

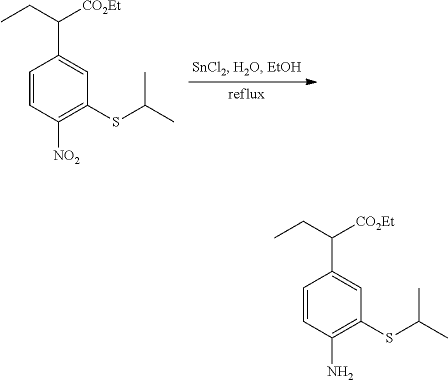

A suspension of 2-(3-isopropylsulfanyl-4-nitro-phenyl)-butyric acid ethyl ester (0.31 g, 1.0 mmol) and SnCl₂ (0.77 g, 4.07 mmol) and water (0.147 g, 8.14 mmol) in ethanol (7.0 mL) was heated at reflux for 3.5 h. The reaction mixture was concentrated under reduced pressure. To the residue were added ethyl acetate (50 mL) and 1 N NaOH (30 mL). The organic layer was washed with water (30 mL), brine (30 mL), dried over sodium sulfate and concentrated under reduced pressure to give the desired product (0.29 g, 100%) as a yellowish oil: ¹H NMR (300 MHz, CDCl₃) δ 7.30 (s, 1H), 7.07 (d, 1H, J=8.1 Hz), 6.68 (d, 1H, J=8.1 Hz), 4.35 (s, 2H, br), 4.10 (m, 2H), 3.29 (t, 1H, J=7.8 Hz), 3.19 (m, 1 H), 2.03 (m, 1H), 1.74 (m, 1H), 1.20 (m, 9H), 0.87 (t, 3H, J=7.2 Hz); ¹³C NMR (75 MHz, CDCl₃) δ 174.11, 147.71, 136.38, 129.25, 128.69, 117.13, 114.71, 60.42, 52.42, 38.55, 26.61, 23.28, 14.22, 12.14.

### Step 3

### 2-(4-Iodo-3-isopropylsulfanyl-phenyl)-butyric acid ethyl ester

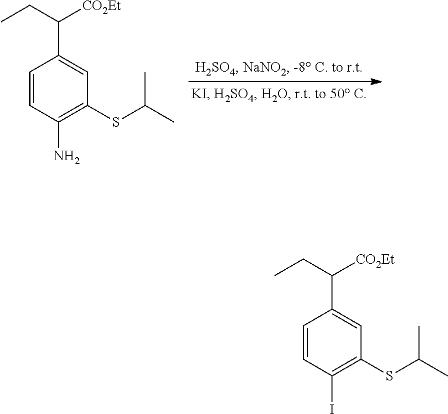

To sulfuric acid (95-98%, 12.3 mL) cooled at −8° C. was added 2-(4-amino-3-isopropylsulfanyl-phenyl)-butyric acid ethyl ester (3.45 g, 12.3 mmol), and then water (18.5 mL) was added carefully. A solution of sodium nitrite (1.02 g, 14.8 mmol) in water (12.3 mL) was added dropwise. After the reaction was stirred for additional 10 min, the solution of the diazonium salt was added at room temperature to a mixture of KI (6.13 g, 36.9 mmol), water (9.8 mL) and sulfuric acid (1.41 mL). The mixture was stirred at room temperature for 60 min and then at 50° C. for 2 h. After the mixture was cooled to room temperature, dichloromethane (100 mL) was added. The organic layer was washed with 5% NaHSO₃ solution (100 mL), brine, dried over sodium sulfate, concentrated under reduced pressure, followed by purification through a silica-gel flash chromatography eluting with heptane/ethyl acetate (20:1) to give the desired product (2.90 g, 60%) as a yellow oil: ¹H NMR (300 MHz, CDCl₃) δ 7.77 (d, 1H, J=8.1 Hz), 7.30 (d, 1H, J=1.8 Hz), 6.83 (dd, 1H, J=8.1, 1.8 Hz), 4.12 (m, 2H), 3.48 (m, 1H), 3.37 (t, 1H, J=7.8 Hz), 2.07 (m, 1H), 1.76 (m, 1H), 1.36 (d, 6H, J=6.6 Hz), 1.21 (t, 3H, J=7.2 Hz), 0.89 (t, 3H, J=7.5 Hz); ¹³C NMR (75 MHz, CDCl₃) δ 173.18, 140.98, 139.75, 139.54, 129.37, 127.01, 100.36, 60.82, 52.99, 38.28, 26.71, 22.64, 14.22, 12.13.

### Step 4

### 2-(2-Isopropylsulfanyl-4′-trifluoromethyl-biphenyl-4-yl)-butyric acid ethyl ester

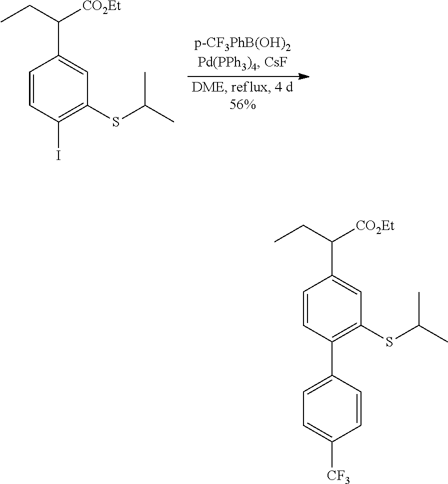

A mixture of 2-(4-iodo-3-isopropylsulfanyl-phenyl)-butyric acid ethyl ester (0.50 g, 1.3 mmol), CsF (0.46 g, 3.0 mmol), (4-trifluoromethyl)benzeneboronic acid (0.30 g, 1.6 mmol) and Pd(PPh₃)₄ (0.075 g, 0.065 mmol) in 1,2-dimethoxyethane (25 mL) was heated at 100° C. for 4 days. After the mixture was concentrated under reduced pressure to remove the solvent, the residue was treated with ethyl acetate (30 mL) and filtered through Celite 521®. The filtrate was washed with a 5% Na₂CO₃ solution (20 mL), brine (20 mL), dried over Na₂SO₄, concentrated under reduced pressure, followed by purification with a silica-gel flash chromatography eluted with heptane/ethyl acetate (50:1) to give the desired product (0.30 g, 56%) as a light yellow oil: ¹H NMR (300 MHz, CDCl₃) δ 7.65 (d, 2H, J=8.1 Hz), 7.52 (d, 2H, J=8.1 Hz), 7.45 (s, 1H), 7.20 (m, 2H), 4.15 (m, 2H), 3.48 (t, 1H, J=7.5 Hz), 3.24 (m, 1H), 2.14 (m, 1H), 1.84 (m, 1H), 1.24 (t, 3H, J=6.9 Hz), 1.19 (m, 6H), 0.94 (t, 3H, J=7.2 Hz); ¹³C NMR (75 MHz, CDCl₃) δ 173.51, 144.15, 140.57, 139.26, 134.48, 130.30, 130.16, 129.81, 129.10 (q, J=32 Hz), 125.79, 124.67 (q, J=4 Hz), 124.16 (q, J=270 Hz), 60.78, 53.14, 37.52, 26.87, 22.74, 14.22, 12.22.

### Step 5

### 2-(2-Isopropylsulfanyl-4′-trifluoromethyl-biphenyl-4-yl)-butyric acid

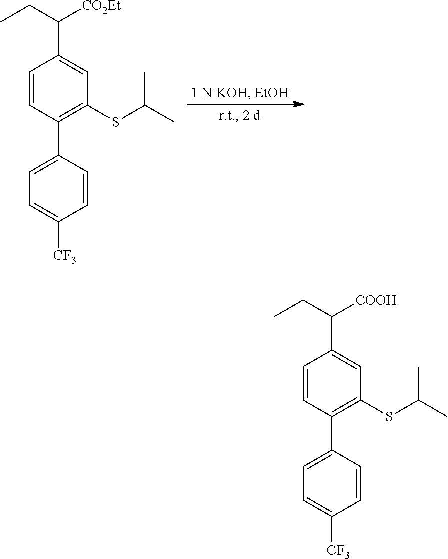

A mixture of 2-(2-isopropylsulfanyl-4′-trifluoromethyl-biphenyl-4-yl)-butyric acid ethyl ester (0.10 g, 0.24 mmol) and aqueous KOH (1.0 M, 2.4 mL) in ethanol (10 mL) was stirred at room temperature for two days. After the reaction mixture was concentrated under reduced pressure, the residue was diluted with water (30 mL), acidified with a 1 N HCl solution to pH 1, and then extracted with ethyl acetate (30 mL). The organic layer was dried over sodium sulfate, concentrated under reduced pressure, followed by lyophilization to give the desired product (0.07 g) as a light yellow solid; HRMS (DIP-CI-MS): calcd. for C₂₀H₂₁O₂F₃S M⁺ 382.1177, found 382.1214; ¹H NMR (300 MHz, CDCl₃/TMS): δ 11.48 (s, 1H, br), 7.65 (d, 2H, J=8.1 Hz), 7.51 (d, 2H, J=8.1 Hz), 7.43 (s, 1H), 7.20 (m, 2H), 3.50 (t, 1H, J=7.5 Hz), 3.22 (m, 1H), 2.13 (m, 1H), 1.86 (m, 1H), 1.18 (d, 6H, J=6.6 Hz), 0.97 (t, 3H, J=7.2 Hz); ¹³C NMR (75 MHz, CDCl₃/TMS): δ 179.53, 144.04, 140.96, 138.36, 134.84, 130.45, 130.27, 129.82, 129.27 (q, J=32 Hz), 125.85, 124.78 (q, J=4 Hz), 124.18 (q, J=271 Hz), 52.95, 37.57, 26.43, 22.79, 12.21; HPLC purity: 96.9%, retention time=11.07 min.

### Example 411

### 2-[2-(Propane-2-sulfonyl)-4′-trifluoromethyl-biphenyl-4-yl]-butyric Acid

### Step 1

### 2-[2-(Propane-2-sulfonyl)-4′-trifluoromethyl-biphenyl-4-yl]-butyric acid ethyl ester

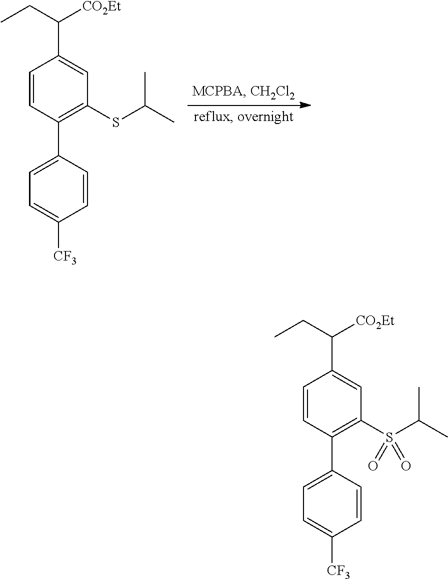

A mixture of 2-(2-isopropylsulfanyl-4′-trifluoromethyl-biphenyl-4-yl)-butyric acid ethyl ester (0.20 g, 0.49 mmol) and 3-chloroperbenzoic acid (77% max, 0.38 g, 1.7 mmol) in dichloromethane (60 mL) was heated at reflux overnight. The reaction mixture was cooled to room temperature, treated with a solution of 5% NaHCO₃ (30 mL), washed with brine (30 mL), dried over sodium sulfate, concentrated under reduced pressure; the residue was purified through a silica gel flash chromatography eluted with heptane/ethyl acetate (5:1) to give the desired product (0.20 g, 92%) as a colorless oil: ¹H NMR (300 MHz, CDCl₃/TMS): δ 8.13 (s, 1H), 7.69 (d, 3H, J=7.8 Hz), 7.58 (d, 2H, J=7.8 Hz), 7.32 (d, 1H, J=7.8 Hz), 4.18 (m, 2H), 3.64 (t, 1H, J=7.5 Hz), 2.64 (m, 1H), 2.19 (m, 1 H), 1.90 (m, 1H), 1.26 (t, 3H, J=7.2 Hz), 1.11 (d, 6H, J=7.2 Hz), 0.95 (t, 3H, J=7.2 Hz); ¹³C NMR (75 MHz, CDCl₃/TMS): δ 172.70, 141.98, 139.84, 139.02, 135.58, 132.64, 132.27, 130.15, 130.09, 129.97 (q, J=32 Hz), 124.37 (q, J=4 Hz), 123.78 (q, J=270 Hz), 60.90, 54.07, 52.80, 26.72, 14.84, 14.75, 14.05, 12.01.

### Step 2

### Example 411

### 2-[2-(Propane-2-sulfonyl)-4′-trifluoromethyl-biphenyl-4-yl]-butyric acid

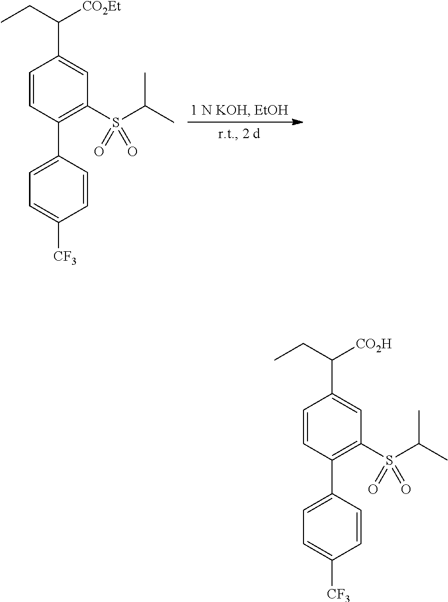

A mixture of 2-[2-(propane-2-sulfonyl)-4′-trifluoromethyl-biphenyl-4-yl]-butyric acid ethyl ester (0.20 g, 0.45 mmol) and aqueous KOH (1.4 M, 2.5 mL) in ethanol (20 mL) was stirred at room temperature for two days. After the solvent was removed under reduced pressure, the residue was diluted with water (30 mL), acidified with 1 N HCl to pH 1, and then extracted with ethyl acetate (30 mL). The organic layer was dried over sodium sulfate, concentrated under reduced pressure, followed by lyophilization to give the desired product (0.16 g) as a white solid; HRMS (DIP-CI-MS): calcd for C₂₀H₂₂O₄F₃S (M+H)⁺ 415.1228, found 415.1191; ¹H NMR (300 MHz, CDCl₃/TMS): δ 10.09 (s, 1H, br), 8.12 (s, 1H), 7.67 (m, 3H), 7.55 (d, 2H, J=8.1 Hz), 7.31 (d, 1H, J=7.8 Hz), 3.65 (t, 1H, J=7.5 Hz), 2.60 (m, 1H), 2.21 (m, 1H), 1.91 (m, 1H), 1.09 (d, 6H, J=6.9 Hz), 0.97 (t, 3H, J=7.5 Hz); ¹³C NMR (75 MHz, CDCl₃/TMS): δ 178.61, 141.92, 139.51, 139.09, 136.00, 132.86, 132.54, 130.34 (q, J=32 Hz), 130.33, 130.20, 124.62 (q, J=4 Hz), 123.89 (q, J=270 Hz), 54.21, 52.69, 26.56, 15.06, 14.95, 12.16; HPLC purity: 99.0%, retention time=12.19 min.

### 2-(3-Cyclopropylmethoxy-4-iodo-phenyl)-4-methyl-pentanoic acid ethyl ester

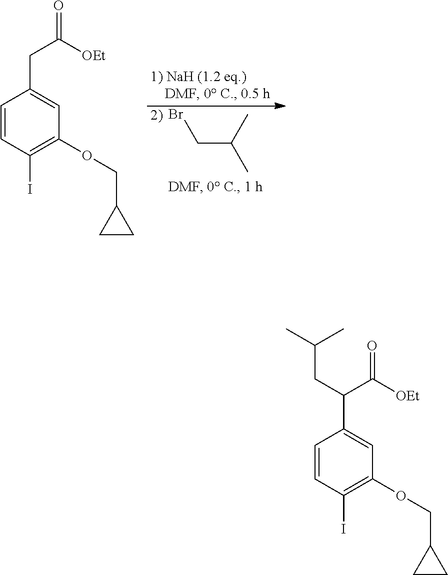

(3-Cyclopropylmethoxy-4-iodo-phenyl)-acetic acid ethyl ester (1.3 g, 3.61 mmol) was dissolved in anhydrous DMF (20 mL) and sodium hydride (60% in oil, 0.17 g, 4.33 mmol) was added at 0° C. The reaction mixture was stirred at 0° C. for 20 min and isobutyl bromide (0.54 g, 3.94 mmol) was added. The reaction mixture was stirred for 1 h at the same temperature and saturated ammonium chloride (10 mL) was added. The reaction mixture was extracted with ethyl acetate (2×20 mL) and the combined organics were washed with water (3×20 mL), saturated sodium chloride solution (10 mL) and dried over magnesium sulfate. Evaporation gave the crude yellow oil (1.26 g, 84%), which was used for the next steps without purification.

### Procedure for the Synthesis of Example 412

### 2-(2-Cyclopropylmethoxy-4′-methylsulfanyl-biphenyl-4-yl)-4-methyl-pentanoic acid

### Step 1

### 2-(2-cyclopropylmethoxy-4′-methylsulfanyl-biphenyl-4-yl)-4-methyl-pentanoic acid ethyl ester

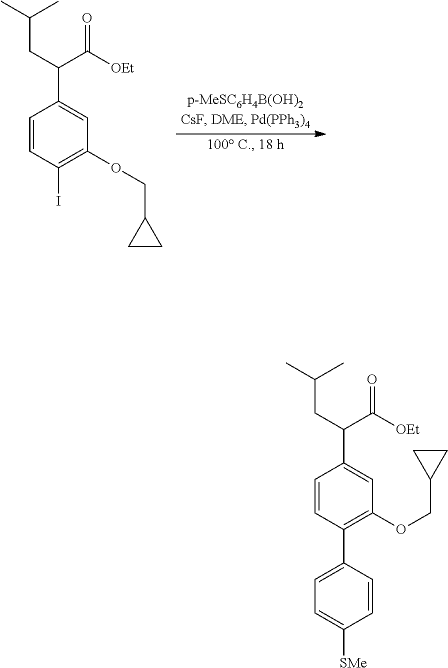

A mixture of the 2-(3-cyclopropylmethoxy-4-iodo-phenyl)-4-methyl-pentanoic acid ethyl ester (0.26 g, 0.63 mmol), 4-(methylthio)phenyl boronic acid (0.13 g, 0.77 mmol), CsF (0.23 g, 1.50 mmol) and Pd(PPh₃)₄ (22 mg, 0.02 mmol) in anhydrous DME (15 mL) was heated at 100° C. for 18 h under argon atmosphere. A mixture of water/EtOAc (15 mL/15 mL) was added and the layers were separated. The aqueous layer was extracted with EtOAc (3×20 mL). The combined organics washed with saturated sodium chloride solution (10 mL) and dried over magnesium sulfate. The solvent was evaporated to give the crude product, which was purified by column chromatography (silica gel, heptane:EtOAc, 9:1-4:1) to produce the 2-(2-cyclopropylmethoxy-4′-methylsulfanyl-biphenyl-4-yl)-4-methyl-pentanoic acid ethyl ester as a yellow oil 0.23 g, (90%).

### Example 412

### Step 2

### 2-(2-Cyclopropylmethoxy-4′-methylsulfanyl-biphenyl-4-yl)-4-methyl-pentanoic acid

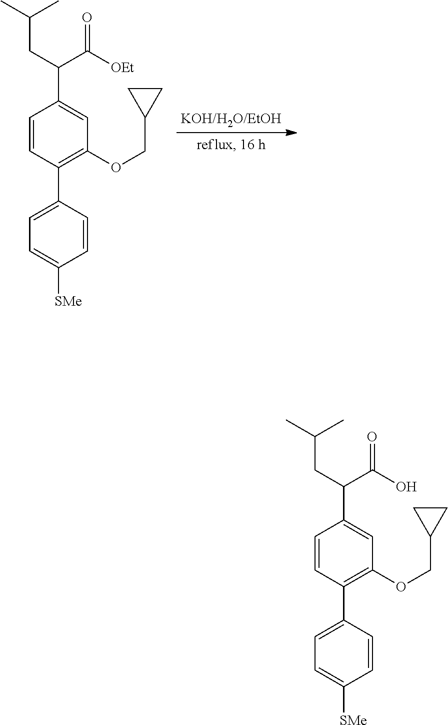

2-(2-Cyclopropylmethoxy-4′-methylsulfanyl-biphenyl-4-yl)-4-methyl-pentanoic acid ethyl ester (0.07 g, 0.17 mmol) was dissolved in a mixture of EtOH/H₂O (9 mL/1 mL) and KOH (0.1 g, 1.80 mmol) was added. The reaction mixture was refluxed for 16 h and after cooling the solvent was evaporated. Then, 6 N aqueous HCl was added to pH 5 and the reaction mixture was extracted with EtOAc (3×10 mL). The organic phase was dried over MgSO₄ and evaporated under reduced pressure to give the crude product as a colorless oil. Purification by gradient column chromatography on silica gel (heptanes/EtOAc=9:1-4:1) gave the desired product as a white solid 0.06 g, (92%), HPLC purity 96.1%.

2-(2-Cyclopropylmethoxy-4′-methylsulfanyl-biphenyl-4-yl)-4-methyl-pentanoic acid: ¹H NMR (300 MHz, CDCl₃/TMS): δ 8.80 (br s, 1H), 7.50 (d, J=8.2 Hz, 2H), 7.28-7.24 (m, 3H), 6.97 (d, J=8.0 Hz, 1H), 6.92 (s, 1H), 3.80 (d, J=6.6 Hz, 2H), 3.66 (t, J=7.6 Hz, 1H), 2.50 (s, 3H), 2.02-1.93 (m, 1H), 1.75-1.66 (m, 1H), 1.58-1.50 (m, 1H), 1.16-1.10 (m, 1H), 0.93 (d, J=6.6 Hz, 6H), 0.57-0.53 (m, 2H), 0.32-0.26 (m, 2H). ¹³C NMR (75 MHz, CDCl₃/TMS): δ 180.1, 155.8, 138.9, 136.6, 134.9, 130.5, 129.8, 129.5, 125.9, 120.7, 113.0, 73.2, 49.5, 42.0, 25.9, 22.6, 22.3, 15.9, 10.3, 3.2.

### Procedure for the Preparation of Example 413

### 2-[3-Cyclopropylmethoxy-4-(2-methoxy-pyridin-4-yl)-phenyl]-4-methyl-pentanoic acid

### Step 1

### 2-[3-Cyclopropylmethoxy-4-(2-methoxy-pyridin-4-yl)-phenyl]-4-methyl-pentanoic acid ethyl ester

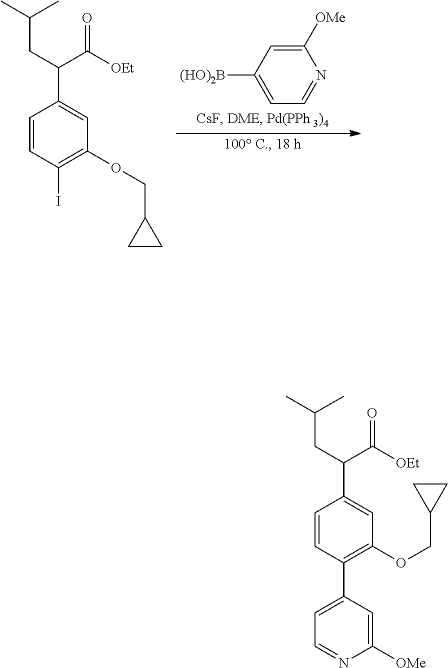

A mixture of the 2-(3-cyclopropylmethoxy-4-iodo-phenyl)-4-methyl-pentanoic acid ethyl ester (0.1 g, 0.24 mmol), 2-methoxypyridine-4-boronic acid (0.05 g, 0.33 mmol), CsF (0.09 g, 0.6 mmol) and Pd(PPh₃)₄ (8.0 mg, 0.007 mmol) in anhydrous DME (15 mL) was heated at 100° C. for 18 h under argon atmosphere. A mixture of water/EtOAc (15 mL/15 mL) was added and the layers were separated. The aqueous layer was extracted with EtOAc (3×20 mL). The combined organic layers were washed with saturated sodium chloride solution (10 mL) and dried over magnesium sulfate. The solvent was evaporated to give a crude product, which was purified by column chromatography (silica gel, heptane:EtOAc, 9:1-4:1) to produce the 2-[3-cyclopropylmethoxy-4-(2-methoxy-pyridin-4-yl)-phenyl]-4-methyl-pentanoic acid ethyl ester as a yellow oil (0.08 g, 84%).

### Step 2

### Example 413

### Cyclopropylmethoxy-4-(2-methoxy-pyridin-4-yl)-phenyl]-4-methyl-pentanoic acid

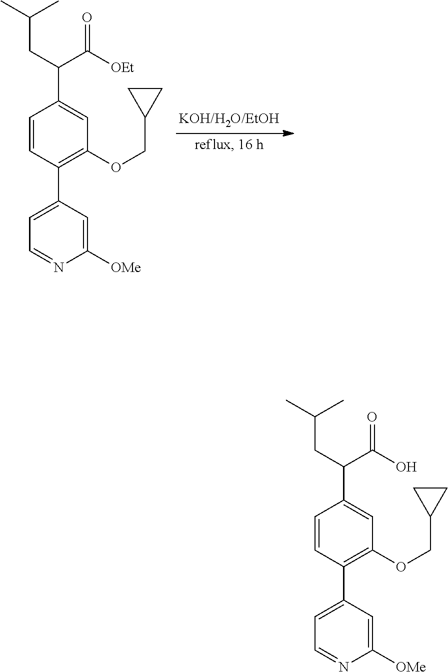

2-[3-Cyclopropylmethoxy-4-(2-methoxy-pyridin-4-yl)-phenyl]-4-methyl-pentanoic acid ethyl ester (0.08 g, 0.2 mmol) was dissolved in a mixture of EtOH/H₂O (9 mL/1 mL) and KOH (0.1 g, 1.80 mmol) was added. The reaction mixture was refluxed for 16 h and after cooling the solvent was evaporated. Then, 6 N aqueous HCl was added to adjust to pH 5 and the reaction mixture was extracted with EtOAc (3×10 mL). The organic phase was dried over MgSO₄ and evaporated under reduced pressure to give a crude product. Purification by gradient column chromatography on silica gel (heptanes/EtOAc=9:1-4:1) gave 2-[3-Cyclopropylmethoxy-4-(2-methoxy-pyridin-4-yl)-phenyl]-4-methyl-pentanoic acid as a brown oil (0.035 g, 50%). HPLC purity 95.3%.

2-[3-Cyclopropylmethoxy-4-(2-methoxy-pyridin-4-yl)-phenyl]-4-methyl-pentanoic acid: ¹H NMR (300 MHz, CDCl₃/TMS): δ 8.42 (br s, 1H), 8.31 (d, J=5.0 Hz, 1H), 7.40-7.36 (m, 2H), 7.25 (s, 1H), 7.10 (d, J=7.7 Hz, 1H), 7.04 (s, 1H), 4.16 (s, 3H), 3.94 (d, J=6.8 Hz, 2H), 3.77 (t, J=7.7 Hz, 1H), 2.11-2.01 (m, 1H), 1.82-1.72 (m, 1H), 1.67-1.56 (m, 1H), 1.32-1.27 (m, 1H), 1.00 (d, J=6.6 Hz, 6H), 0.72-0.60 (m, 2H), 0.42-0.32 (m, 2H). ¹³C NMR (75 MHz, CDCl₃/TMS): δ 177.9, 162.3, 156.1, 152.5, 142.4, 142.3, 130.3, 125.2, 120.9, 118.2, 112.7, 110.9, 73.3, 55.8, 49.6, 42.1, 25.9, 22.6, 22.3, 10.1, 3.2.

### Example 203

### 4-Methyl-2-(6-(2,2,2-trifluoroethoxy)-4′-(trifluoromethyl)biphenyl-3-yl)pentanoic acid

### Step 1

### Ethyl 2-(4-(benzyloxy)phenyl)acetate

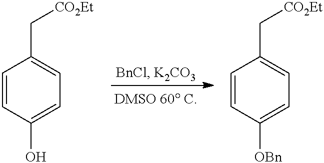

To a stirred solution of ethyl 2-(4-hydroxyphenyl)acetate (200 g, 1.19 mol) and K₂CO₃ (246.4 g, 1.785 mol) in 1 L of DMSO was slowly added benzyl chloride (165 ml, 1.428 mol) at room temperature. After addition was complete the reaction mixture was heated to 60° C. and heating was continued for 4 h, upon which the reaction mixture was poured into water and extracted with ethyl acetate twice. The combined organic layers were washed with water, dried over Na₂SO₄ and evaporated to give the desired product in 78% yield (234 g).

¹HNMR (CDCl3): 7.4 (bs, 5H); 7.21 (d, 2H); 6.92 (d, 2H); 5.08 (s. 2H); 4.14 (q, 2H); 3.56 (s, 2H); 1.26 (t, 3H).

### Step 2

### Ethyl 2-(4-(benzyloxy)phenyl)-4-methylpentanoate

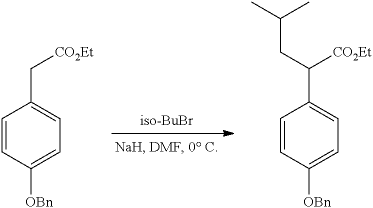

To a suspension of NaH (22.39 g, 50% suspension, 0.46 mol) in 300 ml of DMF slowly added a mixture of ethyl 2-(4-(benzyloxy)phenyl)acetate (120 g, 0.44 mol) and iso-butylbromide (64 g, 0.46 mol) in 700 ml of DMF at 0° C. under nitrogen atmosphere for 15 min. The reaction mixture was stirred for 15 min at 0° C., upon which the reaction mixture was poured on to crushed ice and extracted with ethyl acetate twice. The combined organic layers were washed with water, dried over Na₂SO₄ and evaporated to give the desired product in 60% yield, (87 g).

¹HNMR (CDCl₃): 7.42 (bs, 5H); 7.25 (d, 2H); 6.91 (d, 2H); 5.06 (s, 2H); 4.11 (q, 2H); 3.58 (t, 1H); 1.9 (m, 1H); 1.66 (m, 1H); 1.45 (m, 1H); 1.22 (t, 3H); 0.91 (d, 6H).

### Step 3

### Ethyl 2-(4-hydroxyphenyl)-4-methylpentanoate

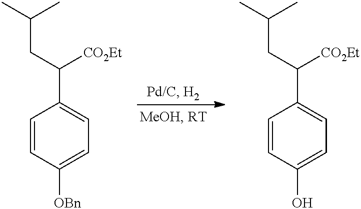

Pd/C (5 g) was slowly added to a stirred mass of ethyl 2-(4-(benzyloxy)phenyl)-4-methylpentanoate (50 g, 0.15 mol) in 700 ml of MeOH under nitrogen atmosphere. The mixture was hydrogenated at ˜40 p.s.i. of H₂ for 2 h. Upon which the reaction catalyst was removed by filtration through a pad of Celite™ and was further washed with MeOH (100 ml×2). The volatiles were evaporated from the filtrate to give the desired product in 85% yield, (30.7 g).

¹HNMR (CDCl3): 7.15 (d, 2H); 6.45 (d, 2H); 4.11 (q, 2H); 3.59 (t, 1H); 1.88 (m, 1H); 1.64 (m, 1H); 1.42 (m, 1H); 1.21 (t, 3H); 0.88 (d, 6H).

### Step 4

### Ethyl 2-(3-bromo-4-hydroxyphenyl)-4-methylpentanoate

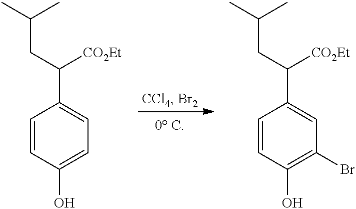

To a stirred solution of ethyl 2-(4-hydroxyphenyl)-4-methylpentanoate (35 g, 0.14 mol) in 200 ml of CCl₄, was slowly added bromine (24.9 g, 0.15 mol) as a solution CCl₄ (100 mL) at 0° C. over a period of 30 min. The reaction mixture was stirred for a further 30 min at 0° C., upon which the reaction mixture was poured onto crushed ice and extracted with DCM twice. The combined organic layers were washed with water and 10% sodium bi-sulfite solution, dried over Na₂SO₄ and evaporated to give the desired product in 65% yield. (30.3 g).

¹HNMR (CDCl3): 7.43 (s, 1H); 7.18 (d, 1H); 6.97 (d, 1H); 5.47 (bs, 1H); 4.11 (q, 2H); 3.52 (t, 1H); 1.91 (m, 1H); 1.69 to 1.37 (m, 2H); 1.21 (t, 3H); 0.88 (d, 6H).

### Step 5

### Ethyl 2-(3-bromo-4-(2,2,2-trifluoroethoxy)phenyl)-4-methyl pentanoate

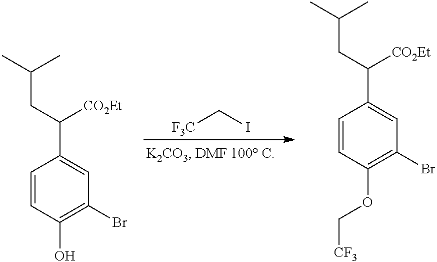

To a stirred mixture of ethyl 2-(3-bromo-4-hydroxyphenyl)-4-methylpentanoate (1 g, 3.12 mmol) and K₂CO₃ (2.19 g, 15.87 mmol) in 10 ml of DMF was slowly added trifluoroethyl iodide (1.99 g, 9.52 mmol) at 0° C. over a period of 10 min. The reaction mixture was stirred for another 30 min at 0° C. and was then heated at 100° C. for 4 h. The reaction mixture was poured into water and extracted with ethyl acetate (×2). The combined organic layers were washed with water, dried over Na₂SO₄ and evaporated. The residue was purified by flash column chromatography to give the desired product in 55% yield. (693 mg).

¹HNMR (CDCl3): 7.45 (s, 1H); 7.16 (d, 1H); 6.96 (d, 1H); 5.48 (bs, 1H); 4.1 (m, 4H); 3.53 (t, 1H); 1.88 (m, 1H); 1.68 to 1.38 (m, 2H); 1.22 (t, 3H); 0.91 (d, 6H).

### Step 6

### Ethyl 4-methyl-2-(6-(2,2,2-trifluoroethoxy)-4′-(trifluoromethyl)biphenyl-3-yl)pentanoate

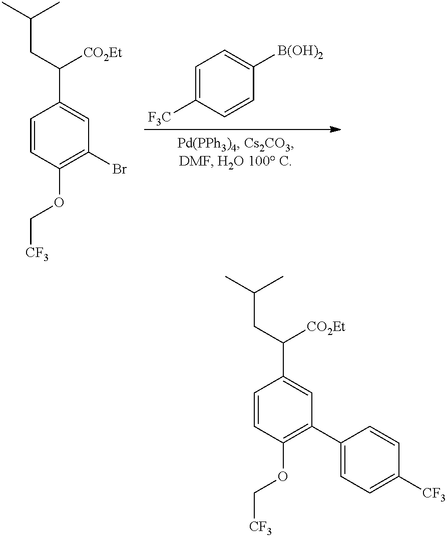

A mixture of ethyl 2-(3-bromo-4-(2,2,2-trifluoroethoxy)phenyl)-4-methyl pentanoate (480 mg, 1.20 mmol), 4-trifluoromethyl phenylboronic acid (252.6 mg, 1.33 mmol), Palladium Tetrakis(triphenylphosphine) (140 mg, 0.12 mmol), cesium carbonate (1.38 g, 4.23 mmol) in DMF/water mixture (50 ml/5 ml) was stirred for overnight at 100° C. The reaction mixture was filtered, the filtrate was diluted with water and extracted with ethyl acetate (×2). The combined organic layers were washed with water and brine, dried over Na₂SO₄ and evaporated. The residue was purified by flash column chromatography to give the desired product in 52% yield. (290 mg).

¹HNMR (CDCl₃): 7.64 (bs, 4H); 7.34 (bs, 2H); 6.92 (d, 1H); 4.31 (q, 2H); 4.12 (q. 2H); 3.65 (t, 1H); 1.96 (m, 1H); 1.67 (m, 1H); 1.38 (m, 1H); 1.23 (t, 3H); 0.88 (d, 6H).

### Step 7

### Example 20

### 4-Methyl-2-(6-(2,2,2-trifluoroethoxy)-4′-(trifluoromethyl)biphenyl-3-yl)pentanoic acid

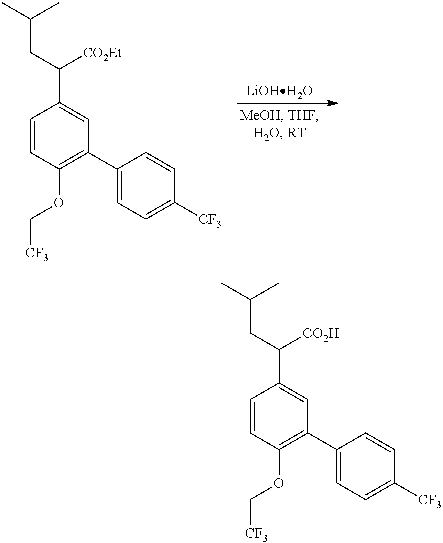

A mixture of ethyl 4-methyl-2-(6-(2,2,2-trifluoroethoxy)-4′-(trifluoromethyl)biphenyl-3-yl)pentanoate (100 mg, 0.21 mmol) and lithium hydroxide monohydrate (27.2 mg, 0.648 mmol) in a MeOH/THF/Water solvent mixture (5 ml/5 ml/5 ml) was stirred for 3 h at room temperature. The reaction volatiles were removed under reduced pressure, the residue was diluted with water, acidified with 5% HCl solution and extracted with ethyl acetate (×2). The combined organic layers were washed with water, dried over Na₂SO₄, filtered and evaporated. The residue was purified by flash column chromatography to give 4-methyl-2-(6-(2,2,2-trifluoroethoxy)-4′-(trifluoromethyl)biphenyl-3-yl)pentanoic acid in 50% yield, (46 mg).

¹HNMR (CDCl3): 7.66 (s, 4H); 7.36 (d, 1H); 7.35 (s, 1H); 6.94 (d, 1H); 4.28 (q, 2H); 3.69 (t, 1H); 1.98 (m, 1H); 1.71 (m, 1H); 1.56 (m, 1H); 0.93 (d, 6H).

### Example 414

### 3-cyclopropyl-2-(2-(cyclopropylmethoxy)-4′-(trifluoromethyl)biphenyl-4-yl)propanoic acid

### Step 1

### 2-(Cyclopropylmethoxy)-4-fluoro-1-nitrobenzene

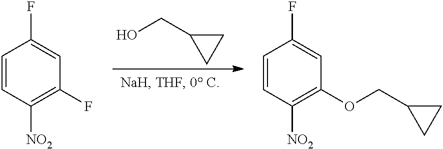

Cyclopropylmethanol (15 g, 207 mmol) was added to a stirred suspension of NaH (60% in mineral oil, 8.37 g) in 200 mL THF over 15 min at 0° C. under nitrogen. The reaction mixture was allowed to warm to room temperature and stirred for 1 h. A solution of 2,4-difluoro-1-nitrobenzene (30 g, 187 mmol) in 200 mL THF was added drop wise at 0° C. The reaction mixture was stirred at 0° C. for 2 h and then poured into ice water. The reaction mixture was extracted with ethyl acetate (3×100 mL). The combined organic layers were dried over MgSO₄ and concentrated under reduced pressure to give 22.0 g of product as orange oil (86%).

### Step 2

### Diethyl 2-(3-(cyclopropylmethoxy)-4-nitrophenyl)malonate

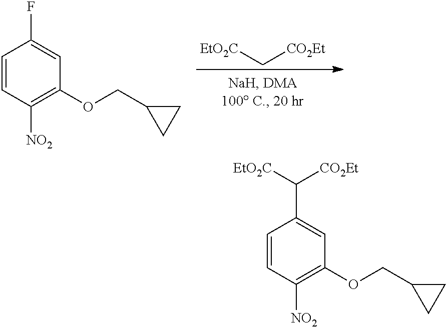

Diethyl malonate (9.8 g, 1.1 eq.) was added to a stirred suspension of sodium hydride (60% in mineral oil, 2.09 g) in 88 mL DMF over 15 min. at 0° C. under nitrogen. The reaction mixture was allowed to warm to room temperature and stirred for 1 h. A solution of 2-cyclopropylmethoxy-4-fluoro-1-nitrobenzene (10 g, 1 eq.) in DMF (88 mL) was added drop wise at 0° C., and the reaction mixture was heated to 100° C. for 3 h. The reaction mixture was allowed to cool to room temperature, poured into ice water and extracted with EtOAc (3×100 mL). The combined organic phases were washed with water (3×100 mL), brine (100 mL) and dried (MgSO₄). Evaporation of solvent under reduced pressure gave 10.0 g of crude product which was purified by silica gel chromatography (hexane/EtOAc) gave 7.0 g of the desired product (42%)

¹H-NMR (CDCl3, 200 MHz): 0.4 (m, 2H), 0.71 (m, 2H), 1.3 (m, 1H), 1.3 (t, 6H), 3.96 (d, 2H), 4.25 (q, 4H), 4.5 (s, 1H), 7.02 (d, 1H), 7.18 (s, 1H), 7.81 (d, 2H).

### Step 3

### Ethyl 2-(3-(cyclopropylmethoxy)-4-nitrophenyl)acetate

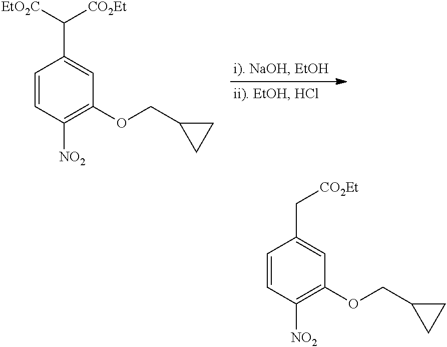

i) Diethyl 2-(3-(cyclopropylmethoxy)-4-nitrophenyl)malonate (10 g) was dissolved in 100 mL ethanol and cooled to 0° C., NaOH solution (4 eq) was added slowly to the reaction mixture for about 15 min. The reaction mixture was heated gently up to 60° C. for 5 h. Progress of the reaction was monitored by TLC analysis. After complete conversion of starting material solvent was evaporated under reduced pressure, the residue dissolved in H₂O, acidified with 6N HCl to pH-2. The solid material was collected via filtration, washed with water, dried under reduced pressure to yield 6.5 g (90%) of 2-(3-(cyclopropylmethoxy)-4-nitrophenyl)acetic acid as a yellow solid.

¹H-NMR (CDCl3, 200 MHz): 0.36 (m, 2H), 0.58 (m, 2H), 1.28 (m, 1H), 3.71 (s, 2H), 4.01 (d, 2H), 7.02 (d, 1H), 7.23 (s, 1H), 7.81 (d, 1H).

ii) 2-(3-(Cyclopropylmethoxy)-4-nitrophenyl)acetic acid (6.5 g) was taken up in an ethanolic HCl solution (50 mL, 25%) and refluxed for 4 h, monitored by TLC. The reaction mixture was concentrated in vacuo to dryness and dissolved in ethyl acetate. The mixture was washed with NaHCO₃ solution, dried over NaSO₄ and concentrated in vacuo to give crude yellow solid which was purified by recrystallization to give the desired product (4.2 g).

¹H-NMR (CDCl3, 200 MHz): 0.36 (m, 2H), 0.58 (m, 2H), 1.12 (t, 3H), 1.28 (m, 1H), 3.71 (s, 2H), 4.01 (d, 2H), 4.21 (q, 2H), 7.02 (d, 1H), 7.23 (s, 1H), 7.81 (d, 1H).

### Step 4

### Ethyl 2-(4-amino-3-(cyclopropylmethoxy)phenyl)acetate

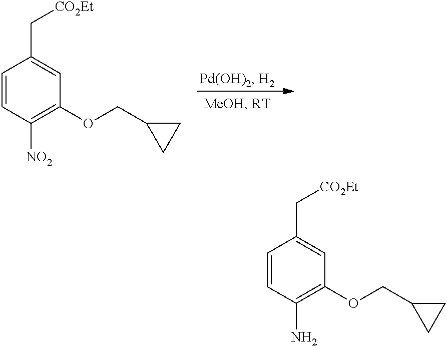

To a stirred solution of ethyl 2-(3-(cyclopropylmethoxy)-4-nitrophenyl)acetate (10 g), in dry MeOH (100 mL) was added Pd(OH)₂ (2 g). The mixture was hydrogenated under a H₂ atmosphere for 6 h at room temperature. The reaction mixture was filtered through a pad of Celite™, washing with MeOH. The combined filtrates were concentrated under reduced pressure to yield 7.5 g of the desired product as an oil.

¹H-NMR (CDCl3, 200 MHz): 0.38 (m, 2H), 0.61 (m, 2H), 1.23 (m, 1H), 1.23 (t, 3H), 3.51 (s, 2H), 3.80 (d, 2H), 4.16 (q, 2H), 6.72 (m, 3H).

### Step 5

### Ethyl 2-(3-(cyclopropylmethoxy)-4-iodophenyl)acetate

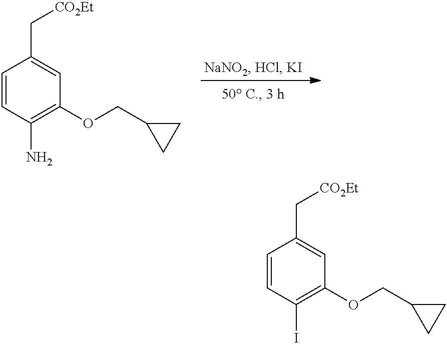

Ethyl-(4-amino-3-cyclopropylmethoxy-phenyl)-acetate (2.5 g, 10.0 mmol) was dissolved in a mixture of EtOH/H₂O/H₂SO₄ (96%) 200 mL/400 mL/10 mL at 0° C. A solution of NaNO₂ (3.2 g, 1.16 eq.) in water (40 mL) was added drop wise at 0° C., and the reaction mixture was stirred for 40 min, at the same temperature. A solution of KI (30 g, 30.1 mmol) in water (80 mL) was added drop wise at 0° C. The reaction mixture was heated to 50° C. for 2.5 h and the solvent was evaporated. The reaction mixture was extracted with EtOAc (3×50 mL), and the combined organic layers were washed with 10% sodium thiosulfate (2×50 mL), water (300 mL) followed by brine (300 mL). The solution was dried over NaSO₄ and concentrated to give crude black oil which was purified by chromatography over silica gel (hexane/EtOAc) to give the product ethyl-(4-iodo-3-cyclopropylmethoxy-phenyl)-acetate as yellow oil 8.7 g, (60%).

¹H-NMR (CDCl3, 200 MHz): 0.41 (m, 2H), 0.62 (m, 2H), 1.22 (t, 3H), 1.23 (m, 1H), 3.58 (s, 2H), 3.89 (d, 2H), 4.17 (q, 2H), 6.60 (d, 1H), 6.74 (s, 1H), 7.73 (d, 1H).

### Step 6

### Ethyl 2-(2-(cyclopropylmethoxy)-4′-(trifluoromethyl)biphenyl-4-yl)acetate

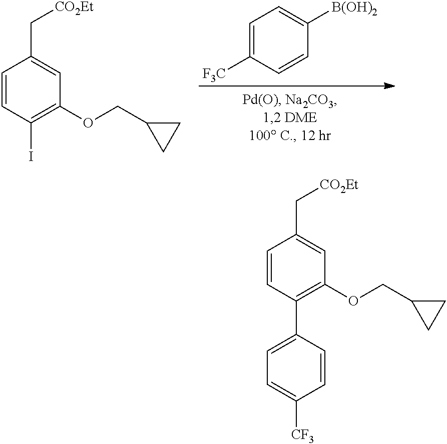

A mixture of ethyl-(4-iodo-3-cyclopropylmethoxy-phenyl)-acetate (5.1 g, 14 mmol), 4-trifluoromethylphenylboronic acid (3.36 g, 17 mmol), CsF (0.28 g, 1.84 mmol) and Pd (PPh₃)₄ (0.410 g, 0.4 mmol) in 75 mL anhydrous 1,2-dimethoxy ethane was refluxed for 8 h under an atmosphere of argon. The reaction mixture was cooled, and 75 mL of EtOAc and 75 mL of water were added. The organic phase was separated, dried over NaSO₄ and concentrated under reduced pressure to yellow oil. The oil was purified by chromatography over silica gel (hexane/EtOAc) to give 4.6 g of product ethyl-(2-cyclopropylmethoxy-4′-trifluoromethyl-biphenyl-4-yl)-acetate (85% yield) as a yellow oil.

¹H-NMR (CDCl3, 200 MHz): 0.41 (m, 2H), 0.62 (m, 2H), 1.22 (t, 3H), 1.23 (m, 1H), 3.58 (s, 2H), 3.89 (d, 2H), 4.17 (q, 2H), 6.96 (m, 2H), 7.31 (s, 1H), 7.64 (m, 4H).

### Step 7

### Example 414

### 3-cyclopropyl-2-(2-(cyclopropylmethoxy)-4′-(trifluoromethyl)biphenyl-4-yl)propanoic acid

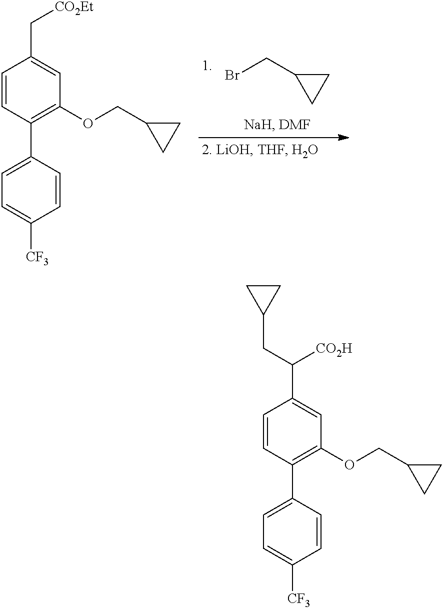

Ethyl-(2-cyclopropylmethoxy-4′-trifluoromethyl-biphenyl-4-yl)-acetate (0.5 g, mmol) was dissolved in 10 mL anhydrous DMF and NaH (60% wt. in oil, 0.13 g, mmol) was added at 0° C. The reaction mixture was stirred for 0.5 h at 25° C. and cyclopropyl methyl bromide (0.24 g, mmol) was added drop wise at 0° C. The reaction mixture was stirred an additional 1 h at 0° C. and saturated NH₄Cl solution (10 mL) was added. The reaction mixture was extracted with EtOAc (3×20 mL) and the combined organic phases were washed with water (3×20 mL) and brine (20 mL), and dried over MgSO₄. Filtered and evaporated under reduced pressure to give a colorless oil (380 mg). The crude oil was dissolved in 10 mL of EtOH/H₂O (9:1, v/v) and 1.0 g LiOH added. The reaction mixture was refluxed for 5 h upon which it was concentrated under reduced pressure. Water (10 mL) was added and the mixture was extracted with EtOAc (3×10 mL). The combined organic phases were dried over MgSO₄ and evaporated under reduced pressure. Purification by column chromatography over silica gel (hexane/EtOAc 9:1) gave the desired product 3-cyclopropyl-2-(2-(cyclopropylmethoxy)-4′-(trifluoromethyl)biphenyl-4-yl) propanoic acid (0.210 g, 60%).

¹H NMR (500 MHz, CDCl₃/TMS): δ 7.79-7.61 (m, 4H), 7.33 (d, 1H), 7.00 (s, 1H), 6.95 (s, 1H), 3.84 (d, 2H), 3.75 (t, 1H), 1.96 (m, 1H), 1.81 (m, 1H), 1.19 (m, 1H), 0.72 (m, 1H), 0.59 (m, 2H), 0.46 (m, 2H), 0.28 (m, 2H), 0.11 (m, 2H).

### Example 415

### 1-(2-(cyclopropylmethoxy)-4′-(trifluoromethyl)biphenyl-4-yl)cyclobutane carboxylic acid

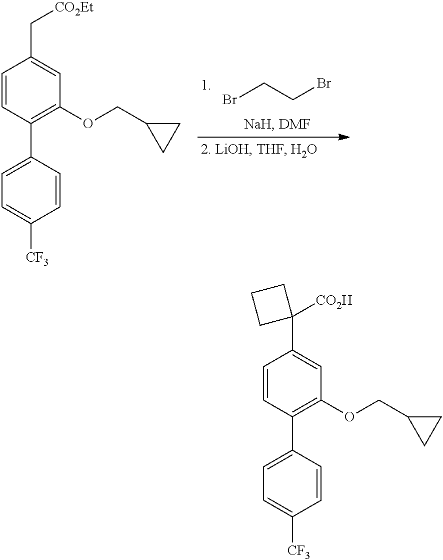

Ethyl-(2-cyclopropylmethoxy-4′-trifluoromethyl-biphenyl-4-yl)-acetate (0.5 g, mmol) was dissolved in 10 mL anhydrous DMF and NaH (60% wt. in oil, 0.13 g, mmol) was added at 0° C. The reaction mixture was stirred for 0.5 h at 25° C. and 1.3 dibromo propane (0.24 g, mmol) was added drop wise at 0° C. The reaction mixture was stirred for an additional 5 h at 0° C. and saturated NH₄Cl solution (10 mL) was added. The reaction mixture was extracted with EtOAc (3×20 mL) and the combined organic phases were washed with water (3×20 mL) and brine (20 mL), and dried over MgSO₄ and evaporated under reduced pressure to give 380 mg of a colorless oil. The oil was dissolved in 10 mL of EtOH/H₂O (9:1) and KOH (1.0 g) added. The reaction mixture was refluxed for 5 h upon which it was concentrated under reduced pressure. Water (10 mL) was added and the reaction mixture was extracted with EtOAc (3×10 mL). The combined organic phases were dried over MgSO₄ and evaporated under reduced pressure. Purification by column chromatography over silica gel (hexane/EtOAc 9:1) gave the desired product 1-(2-(cyclopropylmethoxy)-4′-(trifluoromethyl)biphenyl-4-yl)cyclobutane carboxylic acid (0.210 g, 60%).

¹H NMR (400 MHz, CDCl₃/TMS): δ 7.79-7.61 (m, 4H), 7.33 (d, 1H), 7.00 (s, 1H), 6.85 (s, 1H), 3.82 (d, 2H), 2.91 (m, 1H), 2.58 (m, 2H), 1.91-2.15 (m, 2H), 1.21 (m, 1H), 0.59 (m, 2H), 0.31 (m, 2H).

### Example 416

### 2-(6-(cyclopropylmethoxy)-4′-(methylthio)biphenyl-3-yl)-4-methylpentanoic acid

### Step 1

### Ethyl-2-(6-(cyclopropylmethoxy)-4′-(methylthio)biphenyl-3-yl)-4-methyl pentanoate

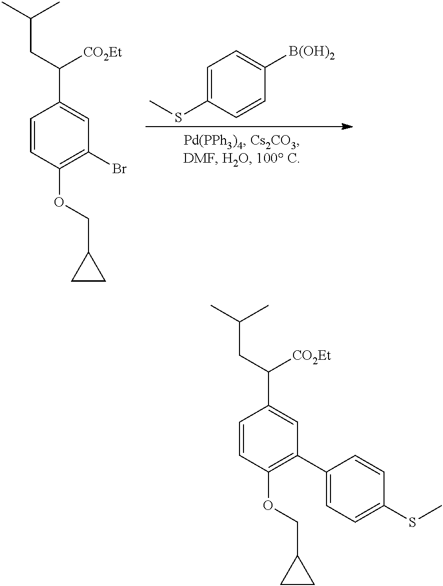

A mixture of ethyl 2-(3-bromo-4-(cyclopropyl methyl)phenyl)-4-methyl pentanoate (500 mg. 1.58 mmol), 4-methylthiophenylboronic acid (295 mg, 1.746 mmol), palladium tetrakis(triphenylphosphine) (18.5 mg, 0.158 mmol), cesium carbonate (1.8 mg, 5.55 mmol) in DMF/water mixture (18 ml/4 ml) was stirred for overnight at 100° C. After completion of reaction, the solids were removed by filtration. The filtrate was diluted with water and extracted with ethyl acetate (×2). The combined organic layers were washed with water followed by brine, dried over Na₂SO₄ and evaporated. The residue was purified by flash column chromatography to give the desired product in 48% yield (290 mg).

### Step 2

### 2-(6-(cyclopropylmethoxy)-4′-(methylthio)biphenyl-3-yl)-4-methylpentanoic acid

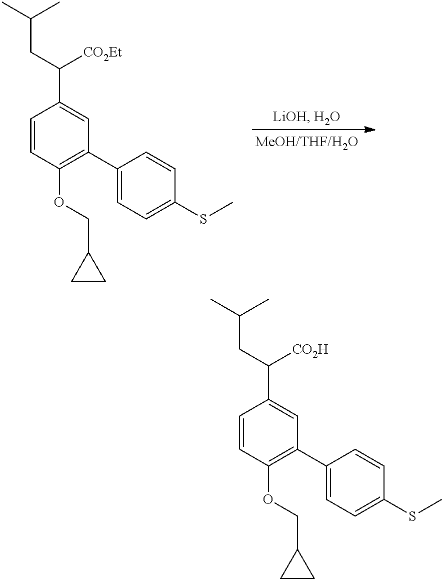

A mixture of ethyl-2-(6-(cyclopropylmethoxy)-4′-(methylthio)biphenyl-3-yl)-4-methyl pentanoate (118 mg, 0.00029 mol) and lithium hydroxide monohydrate (36 mg, 0.000873 mol) in MeOH/THF/Water solvent mixture (5 mL) was stirred for 3 h at room temperature. After completion of reaction volatiles were removed under reduced pressure. Residue was diluted with water, acidified with 5% HCl solution and extracted with ethyl acetate (×2). The combined organic layers were washed with water, dried over Na₂SO₄, filtered and evaporated. The residue was purified by flash column chromatography to give the desired product in 42% yield (46 mg).

¹HNMR (CDCl3): 7.54 (d, 2H); 7.32 (d, 2H); 7.31 (s, 1H); 7.18 (d, 1H), 6.91 (d, 1H); 3.78 (d, 2H); 3.64 (t, 1H); 2.52 (s, 3H), 1.98 (m, 1H); 1.71 (m, 1H); 1.56 (m, 1H); 1.20 (m, 1H), 0.93 (d, 6H), 0.56 (d, 2H), 0.24 (d, 2H).

### Example 417

### Ethyl-2-(6-(cyclopropylmethoxy)-4′-(methylsulfonyl)biphenyl-3-yl)-4-methylpentanoate

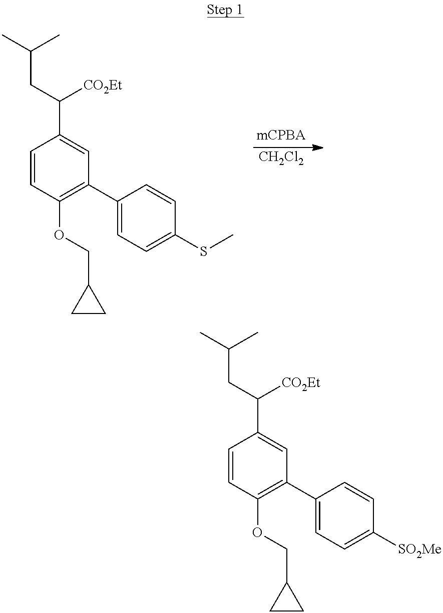

To ethyl-2-(6-(cyclopropylmethoxy)-4′-(methylthio)biphenyl-3-yl)-4-methyl pentanoate (150 mg. 0.872 mmol) in dry DCM (5 mL), at 0° C. was added mCPBA (313 mg, 4.36 mmol) and the mixture was allowed to stir overnight. After completion of reaction, the mixture was diluted with water and extracted with DCM (2×10 mL), the organic extracts washed with water, brine, dried over Na₂SO₄ and evaporated. The crude mixture was purified by Flash Column Chromatography to give the desired product in 34% yield. (55 mg).

### Step 2

### 2-(6-(cyclopropyl methoxy)-4′-methylsulfonyl)biphenyl-3-yl)-4-methylpentanoic acid

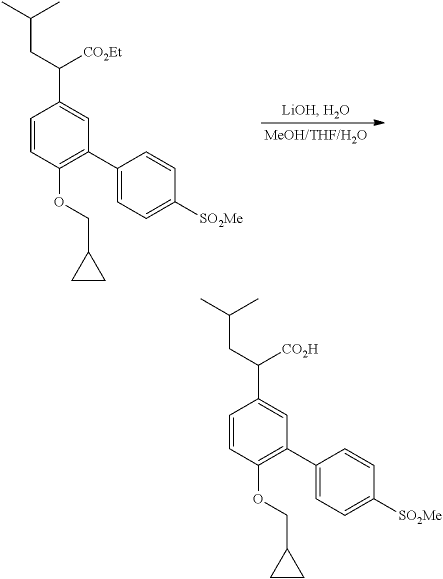

A mixture of ethyl-2-(6-(cyclopropylmethoxy)-4′-(methylsulfonyl)biphenyl-3-yl)-4-methylpentanoate (55 mg, 1.238 mmol) and lithium hydroxide monohydrate (18 mg, 3.71 mmol) in MeOH/THF/Water (5 mL) was stirred for 3 h at room temperature. After completion of the reaction, the volatiles were removed under reduced pressure. The residue was diluted with water, acidified with 5% HCl solution and extracted with ethyl acetate (2×30 mL). The combined organic layers were washed with water, dried over Na₂SO₄, filtered and evaporated. The residue was purified by flash column Chromatography to give the desired product in 88% yield (45 mg).

¹HNMR (CDCl3): 7.98 (d, 2H), 7.78 (d, 2H); 7.35 (d, 1H); 7.28 (d, 1H); 6.95 (d, 1H), 3.83 (d, 2H), 3.68 (t, 1H); 3.14 (s, 3H), 1.98 (m, 1H); 1.71 (m, 1H); 1.56 (m, 1H); 1.18 (m, 1H), 0.93 (d, 6H), 0.59 (d, 2H), 0.28 (d, 2H).

### Example 418

### 2-(3-(benzo[c][1,2,5]oxadiazol-5-yl)-4-(cyclopropylmethoxy)phenyl)-4-pentanoic acid

### Step 1

### Ethyl-2-(3-(benzo[c][1,2,5]oxadiazol-5-yl)-4-(cyclopropylmethoxy)phenyl)-4-methylpentanoate

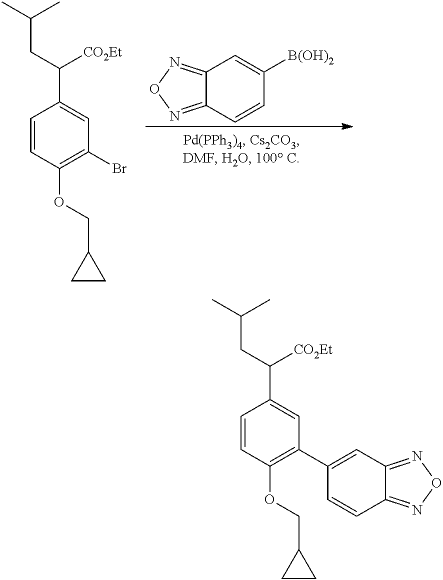

A mixture of 2-(3-bromo-4-(cyclopropyl methyl)phenyl)-4-methyl pentanoate (500 mg, 0.00135 mol), 5-benzaoxadiazol boronic acid (366 mg, 0.00149 mol), Palladium Tetrakis(triphenylphosphine) (156 mg, 0.000135 mol), cesium carbonate (1.54 g, 0.0047 mol) in DMF/water mixture (20 ml/5 ml) was stirred for overnight at 100° C. After completion of reaction, the solids were removed by filtration. The filtrate was diluted with water and extracted with ethyl acetate (2×50 mL). The organic extracts were washed with water, brine, dried over Na₂SO₄ and evaporated. The crude mixture was purified by clash column chromatography to give the desired product in 64% yield (350 mg).

### Step 2

### 2-(3-(benzo[c][1,2,5]oxadiazol-5-yl)-4-(cyclopropylmethoxy)phenyl)-4-pentanoic acid

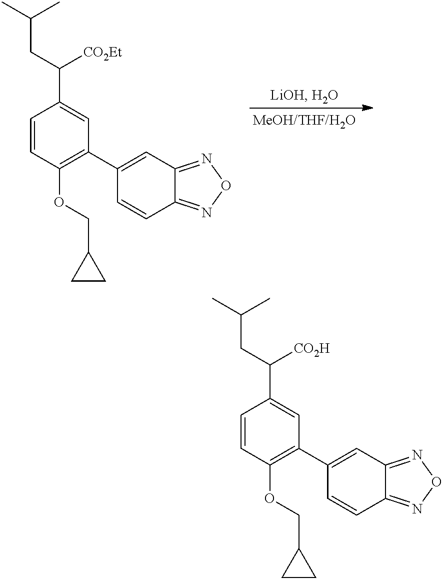

A mixture of ethyl-2-(3-(benzo[c][1,2,5]oxadiazol-5-yl)-4-(cyclopropylmethoxy)phenyl)-4-methylpentanoate (347 mg, 0.84 mmol) and lithium hydroxide monohydrate (107 mg, 2.5 mmol) in MeOH/THF/Water (10 ml/10 ml/5 ml) was stirred for 3 h at room temperature. After completion of the reaction, the volatiles were removed under reduced pressure. The residue was diluted with water, acidified with 5% HCl solution and extracted with ethyl acetate (2×50 mL). The combined organic layers were washed with water, dried over Na₂SO₄, filtered and evaporated. The residue was purified by flash column Chromatography to give the desired product in 34% yield (110 mg).

¹HNMR (CDCl3): 10.25 (bs, 1H), 7.92 (s, 1H), 7.81, (d, 1H), 7.72 (d, 1H), 7.38 (m, 2H); 6.97 (d, 1H), 3.85 (d, 2H), 3.68 (t, 1H); 1.98 (m, 1H); 1.73 (m, 1H); 1.56 (m, 1H); 1.18 (m, 1H), 0.94 (d, 6H), 0.58 (d, 2H), 0.24 (d, 2H).

### Example 419

### 2-(3-(benzo[c][1,2,5]thiadiazol-5-yl)-4-(cyclopropylmethoxy)phenyl)-4-pentanic acid

### Step 1

### Ethyl-2-(3-(benzo[c][1,2,5]thiadiazol-5-yl)-4-(cyclopropylmethoxy)phenyl)-4-methylpentanoate

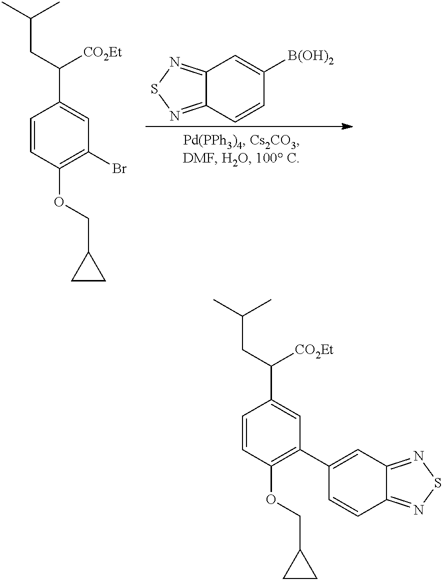

A mixture of 2-(3-bromo-4-(cyclopropyl methyl)phenyl)-4-methyl pentanoate (500 mg. 0.00135 mol), 5-benzothiadiazol boronic acid (390 mg, 0.00149 mol), Palladium Tetrakis(triphenylphosphine) (156 mg, 0.00013 mmol), cesium carbonate (1.54 gm, 0.0047 mol) in DMF/water mixture (20 ml/5 ml) was stirred for overnight at 100° C. After completion of the reaction, the solids were removed by filtration. The filtrate was diluted with water and extracted with ethyl acetate (2×50 mL), the organic extracts were washed with water, brine, dried over Na₂SO₄ and evaporated. The crude mixture was purified by Flash Column Chromatography to give the desired product in 64% yield. (347 mg).

### Step 2

### 2-(3-(benzo[c][1,2,5]thiadiazol-5-yl)-4-(cyclopropylmethoxy)phenyl)-4-pentanic acid

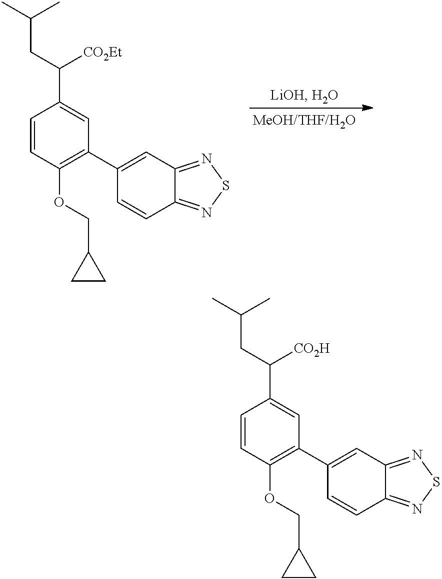

A mixture of ethyl-2-(3-(benzo[c][1,2,5]thiadiazol-5-yl)-4-(cyclopropylmethoxy)phenyl)-4-methylpentanoate (312 mg, 0.00074 mol) and lithium hydroxide monohydrate (92 mg, 0.0022 mol) in MeOH/THF/Water (10 ml/10 ml/5 ml) was stirred for 3 h at room temperature. After completion of reaction volatiles were removed under reduced pressure. Residue was diluted with water, acidified with 5% HCl solution and extracted with ethyl acetate (2×50 mL). Combined organic layer was washed with water, dried over Na₂SO₄, filtered and evaporated. Residue was purified by flash column chromatography to the desired product in 48% yield (140 mg).

¹HNMR (CDCl3): 10.25 (bs, 1H), 7.89 (s, 1H), 7.81, (d, 1H), 7.72 (d, 1H), 7.38 (d, 2H); 6.97 (d, 1H), 3.88 (d, 2H), 3.65 (t, 1H); 1.98 (m, 1H); 1.73 (m, 1H); 1.56 (m, 1H); 1.18 (m, 1H), 0.96 (d, 6H), 0.58 (d, 2H), 0.24 (d, 2H).

### Example 420

### 2-(6-(cyclopropylmethoxy)-3′-methylbiphenyl-3-yl)-4-methylpentanic acid

### Step 1

### Ethyl-2-(6-(cyclopropylmethoxy)-3′-methylbiphenyl-3-yl)-4-methylpentanoate

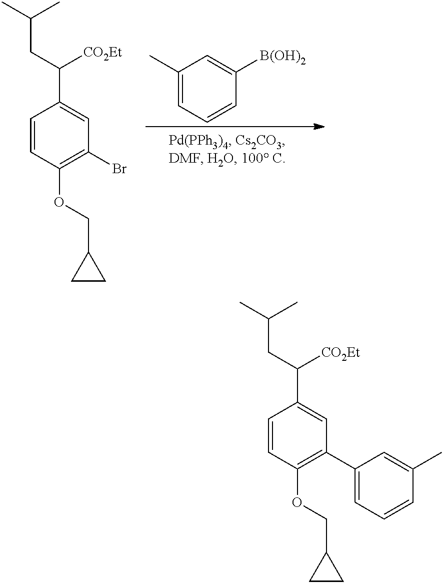

A mixture of ethyl 2-(3-bromo-4-(cyclopropyl methyl)phenyl)-4-methyl pentanoate (500 mg. 0.0013 mol), 3-methylphenyl boronic acid (200 mg, 0.0014 mol), Palladium Tetrakis(triphenylphosphine) (156 mg, 0.00013 mol), cesium carbonate (1.54 g, 0.0047 mol) in DMF/water mixture (20 ml/5 ml) was stirred for overnight at 100° C. After completion of the reaction, the solids were removed by filtration. The filtrate was diluted with water and extracted with ethyl acetate (2×50 mL), the organic extracts were washed with water, brine, dried over Na₂SO₄ and evaporated. The crude mixture was purified by Flash Column Chromatography to give the desired product in 67% yield (347 mg).

### Step 2

### 2-(6-(cyclopropylmethoxy)-3′-methylbiphenyl-3-yl)-4-methylpentanic acid

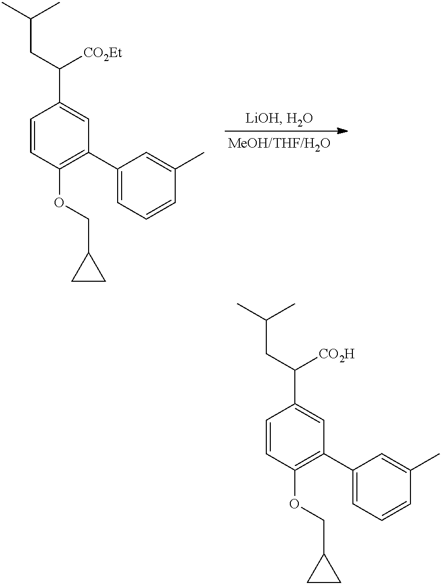

A mixture of ethyl-2-(6-(cyclopropylmethoxy)-3′-methylbiphenyl-3-yl)-4-methylpentanoate (347 mg, 0.91 mmol) and lithium hydroxide monohydrate (115 mg, 2.7 mmol) in MeOH/THF/Water (10 ml/10 ml/5 ml) was stirred for 3 h at room temperature. After completion of the reaction, the volatiles were removed under reduced pressure. The residue was diluted with water, acidified with 5% HCl solution and extracted with ethyl acetate (2×30 mL). The combined organic extracts were washed with water, dried over Na₂SO₄, filtered and evaporated. The residue was purified by flash column Chromatography to give compound the desired product in 62% yield (325 mg).

¹HNMR (CDCl3, 500 MHz): 7.39 (d, 2H), 7.32, (m, 2H), 7.22 (d, 1H), 7.18 (d, 1H); 6.93 (d, 1H), 3.81 (d, 2H), 3.63 (t, 1H); 2.40 (s, 3H), 1.98 (m, 1H); 1.73 (m, 1H); 1.56 (m, 1H); 1.18 (m, 1H), 0.93 (d, 6H), 0.54 (d, 2H), 0.24 (d, 2H).

### Example 421

### 2-(4-(cyclopropylmethoxy-3-(pyridine-4-yl)phenyl)-4-methylpentanoic acid

### Step 1

### Ethyl-2-(4-(cyclopropylmethoxy)-3-(pyridine-4-yl)phenyl)-4-methylpentanoate

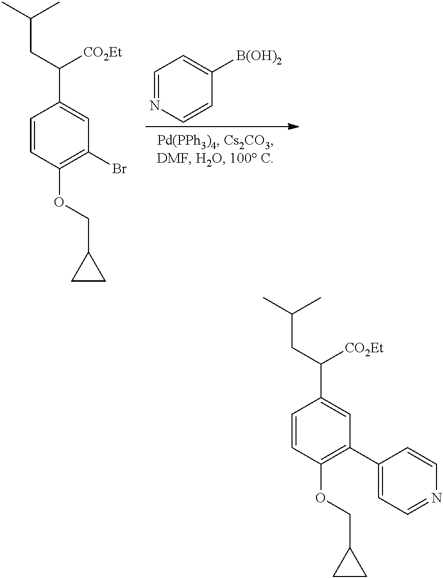

A mixture of ethyl 2-(3-bromo-4-(cyclopropyl methyl)phenyl)-4-methyl pentanoate (500 mg. 0.00135 mol), 4-pyridineboronic acid (200 mg, 0.00162 mol), Palladium Tetrakis(triphenylphosphine) (156 mg, 0.000135 mol), cesium carbonate (1.54 g, 0.0047 mol) in DMF/water mixture (20 ml/5 ml) was stirred for overnight at 100° C. After completion of the reaction, solids were removed by filtration. The filtrate was diluted with water and extracted with ethyl acetate (×2). The combined organic extracts were washed with water followed by brine, dried over Na₂SO₄ and evaporated. The residue was purified by flash column chromatography to give the desired product in 40% yield (200 mg).

### Step 2

### 2-(4-(cyclopropylmethoxy-3-(pyridine-4-yl)phenyl)-4-methylpentanoic acid

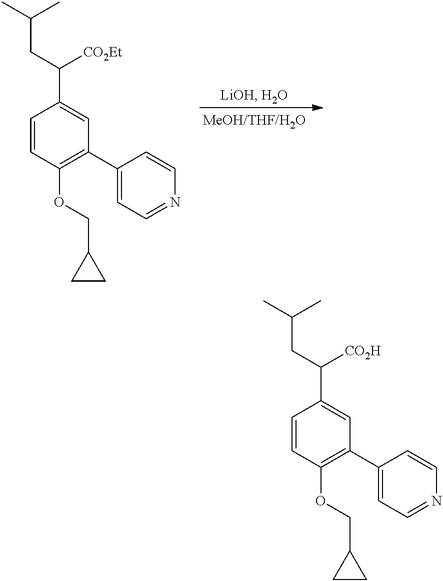

A mixture of ethyl-2-(4-(cyclopropylmethoxy)-3-(pyridine-4-yl)phenyl)-4-methylpentanoate (200 mg) and lithium hydroxide monohydrate (67 mg, 0.00159 mol) in MeOH/THF/Water solvent mixture (25 ml) was stirred for 3 h at room temperature. After completion of the reaction, the volatiles were removed under reduced pressure. The residue was diluted with water, acidified with 5% HCl solution and extracted with ethyl acetate (×2). The combined organic extracts were washed with water, dried over Na₂SO₄, filtered and evaporated. The residue was purified by Flash Column Chromatography to give the desired product in 40% yield (72 mg).

¹HNMR (CDCl₃, 500 MHz): 10.45 (bs, 1H), 7.58 (d, 2H); 7.41 (d, 2H), 7.25 (m, 3H), 6.94 (d, 1H); 3.81 (d, 2H); 3.62 (t, 1H); 1.98 (m, 1H); 1.75 (m, 1H); 1.56 (m, 1H); 1.18 (m, 1H), 0.93 (d, 6H), 0.58 (d, 2H), 0.23 (d, 2H).

### Example 422

### 2-(4′-Cyano-6-(cyclopropylmethoxy)biphenyl-3-yl)-4-methylpentanic acid

### Step 1

### Ethyl-2-(4′cyano-6-(cyclopropylmethoxy)biphenyl-3-yl)-4-methylpentanoate

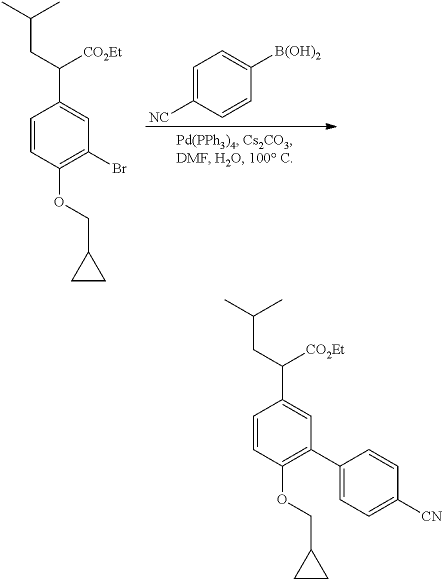

A mixture of ethyl 2-(3-bromo-4-(cyclopropyl methyl)phenyl)-4-methyl pentanoate (500 mg. 0.00135 mol), 4-cyanophenyl boronic acid (218 mg, 0.00149 mol), Palladium Tetrakis(triphenylphosphine) (156 mg, 0.000135 mol), cesium carbonate (1.56 g, 0.00149 mol) in DMF/water mixture (20 ml/5 ml) was stirred for overnight at 100° C. After completion of the reaction, the solids were removed by filtration. The filtrate was diluted with water and extracted with ethyl acetate (2×50 mL), organic extracts washed with water, brine, dried over Na₂SO₄ and evaporated. The crude mixture was purified by Flash Column Chromatography to give the desired product in 66% yield. (350 mg).

### Step 2

### 2-(4′-Cyano-6-(cyclopropylmethoxy)biphenyl-3-yl)-4-methylpentanic acid

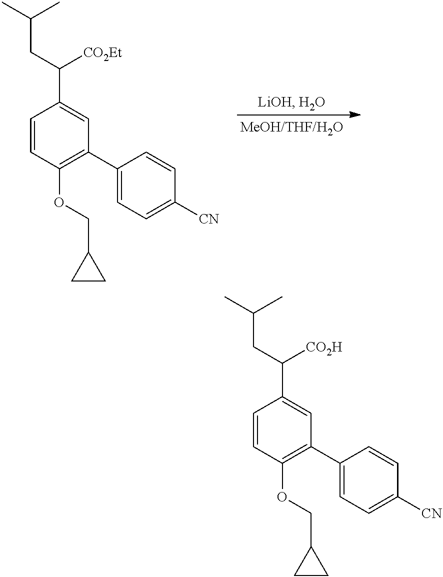

A mixture of ethyl-2-(4′cyano-6-(cyclopropylmethoxy)biphenyl-3-yl)-4-methylpentanoate (346 mg, 0.884 mmol) and lithium hydroxide monohydrate (112 mg, 2.64 mmol) in MeOH/THF/Water (10 ml/10 ml/5 ml) was stirred for 3 h at room temperature. After completion of the reaction, the volatiles were removed under reduced pressure. The residue was diluted with water, acidified with 5% HCl solution and extracted with ethyl acetate (2×50 mL). The combined organic extracts were washed with water, dried over Na₂SO₄, filtered and evaporated. The residue was purified by flash column Chromatography to give the desired product in 40% yield (130 mg).

¹HNMR (CDCl₃, 500 MHz): 7.65 (m, 4H), 7.28, (m, 2H), 6.97 (d, 1H), 3.81 (d, 2H), 3.67 (t, 1H); 1.98 (m, 1H); 1.73 (m, 1H); 1.56 (m, 1H); 1.18 (m, 1H), 0.93 (d, 6H), 0.58 (d, 2H), 0.24 (d, 2H).

### Example 423

### 2-(6-(cyclopropylmethoxy)-3′-nitrobiphenyl-3-yl)-4-methylpentanoic acid

### Step 1

### Ethyl-2-(6-(cyclopropylmethoxy)-3′-nitrobiphenyl-3-yl)-4-methylpentanoate

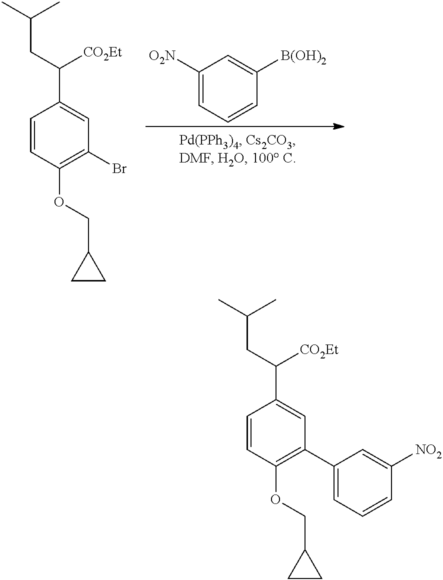

A mixture of ethyl 2-(3-bromo-4-(cyclopropyl methyl)phenyl)-4-methyl pentanoate (500 mg. 0.00135 mol), 3-nitrophenyl boronic acid (280 mg, 0.00167 mol), Palladium Tetrakis(triphenylphosphine) (155 mg, 0.000134 mol), cesium carbonate (1.54 g, 0.0047 mol) in DMF/water mixture (20 ml/5 ml) was stirred for overnight at 100° C. After completion of the reaction, the solids were removed by filtration. The filtrate was diluted with water and extracted with ethyl acetate (2×50 mL), organic extracts washed with water, brine, dried over Na₂SO₄ and evaporated. The crude mixture was purified by Flash Column Chromatography to give the desired product in 48.7% yield (300 mg).

### Step 2

### 2-(6-(cyclopropylmethoxy)-3′-nitrobiphenyl-3-yl)-4-methylpentanoic acid

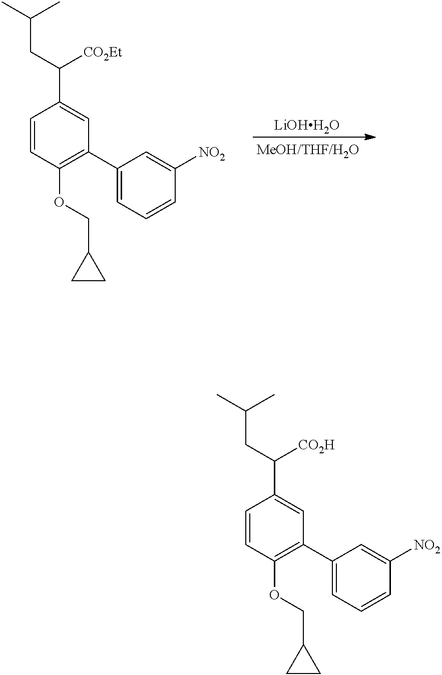

A mixture of ethyl-2-(6-(cyclopropylmethoxy)-3′-nitrobiphenyl-3-yl)-4-methylpentanoate (200 mg, 0.00044 mol) and lithium hydroxide monohydrate (140 mg, 0.00132 mol) in MeOH/THF/Water (10 ml/10 ml/5 ml) was stirred for 3 h at room temperature. After completion of the reaction, the volatiles were removed under reduced pressure. The residue was diluted with water, acidified with 5% HCl solution and extracted with ethyl acetate (2×30 mL). The combined organic extracts were washed with water, dried over Na₂SO₄, filtered and evaporated. The residue was purified by flash column Chromatography to give the desired product in 42% yield (80 mg).

¹HNMR (CDCl₃): 8.57 (s, 1H), 8.19 (d, 1H), 7.88 (d, 1H), 7.58, (t, 1H), 7.31 (d, 1H), 7.37 (d, 2H); 6.97 (d, 1H), 3.85 (d, 2H), 3.67 (t, 1H); 1.98 (m, 1H); 1.73 (m, 1H); 1.56 (m, 1H); 1.18 (m, 1H), 0.95 (d, 6H), 0.58 (d, 2H), 0.24 (d, 2H).

### Example 424

### 2-(6-(cyclopropylmethoxy)-4′-methoxy biphenyl-3-yl)-4-methylpentanoic acid

### Step 1

### Ethyl-2-(6-(cyclopropylmethoxy)-4′-methoxybiphenyl-3-yl)-4-methylpentanoate

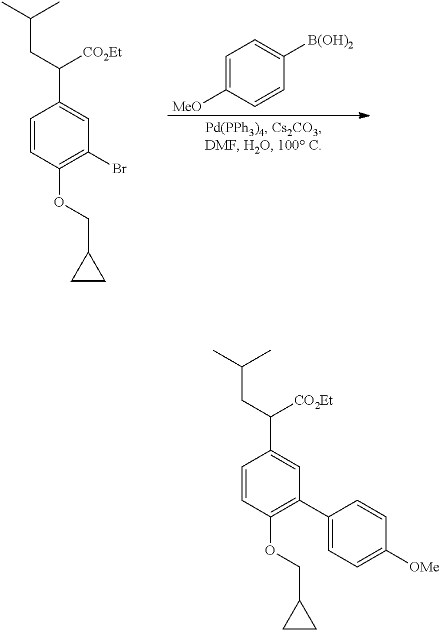

A mixture of ethyl 2-(3-bromo-4-(cyclopropyl methyl)phenyl)-4-methyl pentanoate (500 mg. 0.00129 mol), 4-methoxy phenylboronic acid (196 mg, 0.0014 mol), Palladium Tetrakis(triphenylphosphine) (156 mg, 0.00013 mol), cesium carbonate (1.54 g, 0.0047 mol) in DMF/water mixture (20 ml/5 ml) was stirred for overnight at 100° C. After completion of the reaction, the solids were removed by filtration. The filtrate was diluted with water and extracted with ethyl acetate (×2). The combined organic extracts were washed with water followed by brine, dried over Na₂SO₄ and evaporated. The residue was purified by Flash Column Chromatography to give the desired product in 67% yield (345 mg).

### Step 2

### 2-(6-(cyclopropylmethoxy)-4′-methoxy biphenyl-3-yl)-4-methylpentanoic acid

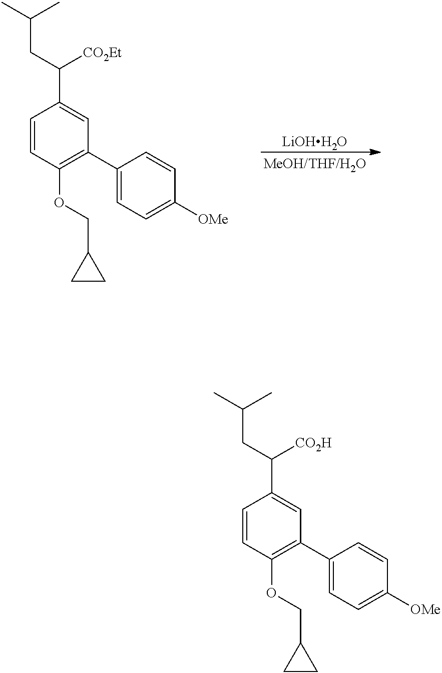

A mixture of ethyl-2-(6-(cyclopropylmethoxy)-4′-methoxybiphenyl-3-yl)-4-methylpentanoate (300 mg, 0.68 mmol) and lithium hydroxide monohydrate (150 mg, 2.04 mmol) in MeOH/THF/Water solvent mixture (10 ml/10 ml/5 ml) was stirred for 3 h at room temperature. After completion of the reaction, the volatiles were removed under reduced pressure. The residue was diluted with water, acidified with 5% HCl solution and extracted with ethyl acetate (×2). The combined organic extracts were washed with water, dried over Na₂SO₄, filtered and evaporated. The residue was purified by Flash Column Chromatography to give the desired product in 71% yield. (200 mg).

¹HNMR (CDCl₃, 500 MHz): 10.1 (bs, 1H), 7.54 (d, 2H); 7.23 (s, 1H), 7.20 (d, 1H); 6.98 (d, 2H); 6.91 (d, 1H); 3.88 (s, 3H), 3.79 (d, 2H); 3.64 (t, 1H); 1.98 (m, 1H); 1.71 (m, 1H); 1.56 (m, 1H); 1.20 (m, 1H), 0.93 (d, 6H), 0.56 (d, 2H), 0.24 (d, 2H).

### Example 425

### 2-(6-(cyclopropylmethoxy)-6′-isopropylbiphenyl-3-yl)-4-methylpentanoic acid

### Step 1

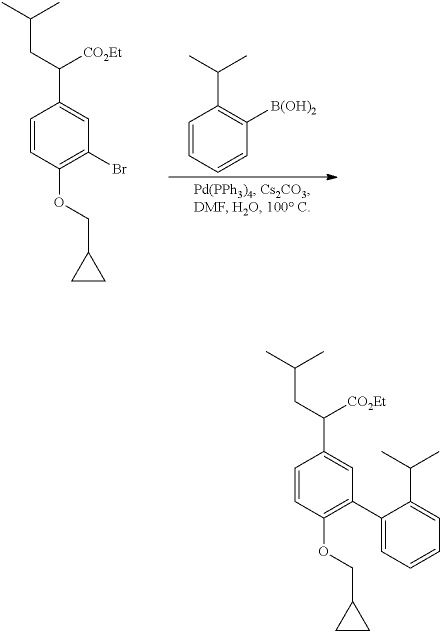

### Ethyl-2-(6-(cyclopropylmethoxy)-6′-isopropylbiphenyl-3-yl)-4-methylpentanoate

A mixture of ethyl 2-(3-bromo-4-(cyclopropyl methyl)phenyl)-4-methyl pentanoate (500 mg. 0.00135 mol), 2-isopropyl phenylboronic acid (260 mg, 0.00153), Palladium Tetrakis(triphenylphosphine) (156 mg, 0.00013 mol), cesium carbonate (1.54 g, 0.0047 mol) in DMF/water mixture (20 ml/5 ml) was stirred for overnight at 100° C. After completion of the reaction, the solids were removed by filtration. The filtrate was diluted with water and extracted with ethyl acetate (×2). The combined organic extracts were washed with water followed by brine, dried over Na₂SO₄ and evaporated. The residue was purified by flash column chromatography to give the desired product in 45% yield (250 mg).

### Step 2

### 2-(6-(cyclopropylmethoxy)-6′-isopropylbiphenyl-3-yl)-4-methylpentanoic acid

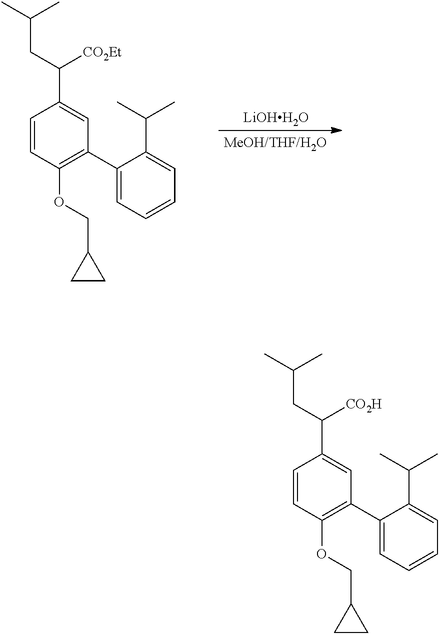

A mixture of ethyl-2-(6-(cyclopropylmethoxy)-6′-isopropylbiphenyl-3-yl)-4-methylpentanoate (230 mg, 0.00056 mol) and lithium hydroxide monohydrate (71 mg, 0.00166 mol) in MeOH/THF/Water solvent mixture (25 mL) was stirred for 3 h at room temperature. After completion of the reaction, the volatiles were removed under reduced pressure. The residue was diluted with water, acidified with 5% HCl solution and extracted with ethyl acetate (×2). The combined organic layers were washed with water, dried with Na₂SO₄, filtered and evaporated. The crude mixture was purified by column chromatography to give the desired product in 14% yield (38 mg).

¹HNMR (CDCl3, 500 MHz): 7.38 (m, 4H); 7.21 (m, 1H), 7.14 (m, 2H); 6.98 (d, 1H); 3.76 (d, 2H), 3.62 (t, 1H); 1.98 (m, 1H); 1.42-171 (m, 3H); 1.2 (s, 3H); 1.11 (m, 1H), 1.05 (s, 3H), 0.93 (d, 6H), 0.44 (d, 2H), 0.18 (d, 2H).

### Example 426

### 2-(6-(cyclopropylmethoxy)-3′-fluorobiphenyl-3-yl)-4-methylpentanoic acid

### Step 1

### Ethyl-2-(6-(cyclopropylmethoxy)-3′-fluorobiphenyl-3-yl)-4-methylpentanoate

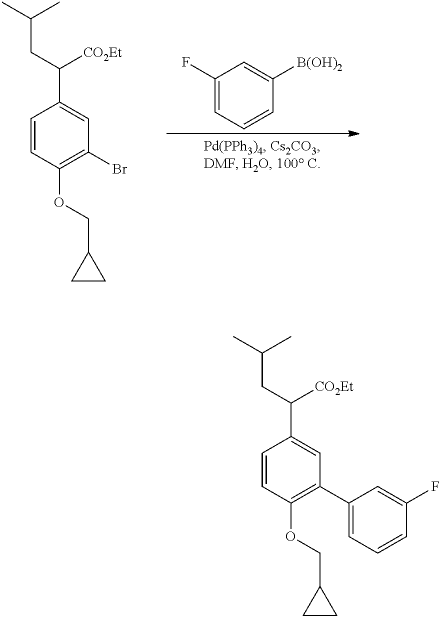

A mixture of ethyl 2-(3-bromo-4-(cyclopropyl methyl)phenyl)-4-methyl pentanoate (500 mg. 0.00135 mol), 3-fluoro benzeneboronic acid (208 mg, 0.00149 mol), Palladium Tetrakis(triphenylphosphine) (156 mg, 0.000135 mol), cesium carbonate (1.54 g, 0.0047 mol) in DMF/water mixture (20 ml/5 ml) was stirred for overnight at 100° C. After completion of the reaction, the solids were removed by filtration. The filtrate was diluted with water and extracted with ethyl acetate (×2). The combined organic extracts were washed with water followed by brine, dried over Na₂SO₄ and evaporated. The residue was purified by Flash Column Chromatography to give the desired product in 50% yield (250 mg).

### Step 2

### 2-(6-(cyclopropylmethoxy)-3′-fluorobiphenyl-3-yl)-4-methylpentanoic acid

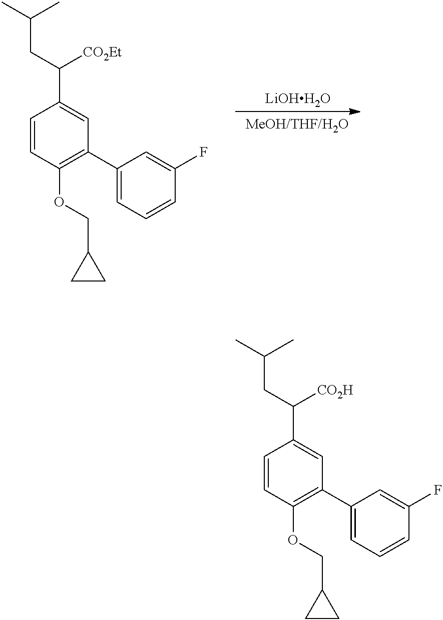

A mixture of ethyl-2-(6-(cyclopropylmethoxy)-3′-fluorobiphenyl-3-yl)-4-methylpentanoate (310 mg) and lithium hydroxide monohydrate (112 mg, 0.266 mmol) in MeOH/THF/Water solvent mixture (10 ml/10 ml/5 ml) was stirred for 3 h at room temperature. After completion of the reaction, the volatiles were removed under reduced pressure. The residue was diluted with water, acidified with 5% HCl solution and extracted with ethyl acetate (×2). The combined organic extracts were washed with water, dried over Na₂SO₄, filtered and evaporated. The residue was purified by Flash Column Chromatography to give the desired product in 75% yield. (210 mg).

¹HNMR (CDCl₃, 500 MHz): 7.31 (m, 3H); 7.18 (s, 1H), 7.03 (d, 1H); 6.96 (m, 1H); 6.63 (d, 1H); 3.62 (d, 2H); 3.41 (t, 1H); 1.78 (m, 1H); 1.54 (m, 1H); 1.31 (m, 1H); 1.12 (m, 1H), 0.79 (d, 6H), 0.52 (d, 2H), 0.21 (d, 2H).

### Example 427

### 2-(6-(cyclopropylmethoxy)-4′-fluorobiphenyl-3-yl)-4-methylpentanoic acid

### Step 1

### Ethyl-2-(6-(cyclopropylmethoxy)-4′-fluorobiphenyl-3-yl)-4-methylpentanoate

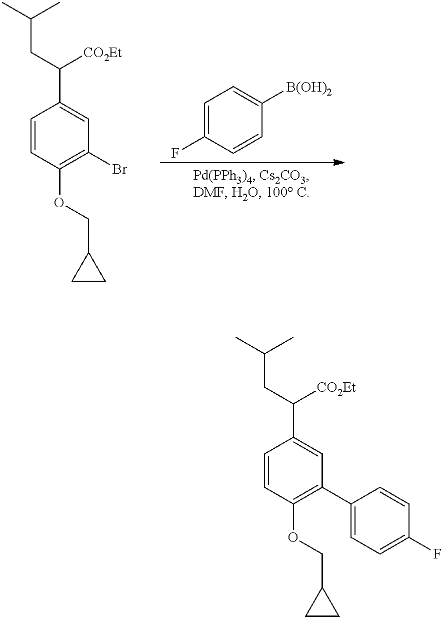

A mixture of ethyl 2-(3-bromo-4-(cyclopropyl methyl)phenyl)-4-methyl pentanoate (500 mg. 0.00135 mol), 4-fluoro phenylboronic acid (304 mg, 0.00149 mol), Palladium Tetrakis(triphenylphosphine) (156 mg, 0.000135 mol), Cesium carbonate (1.54 g, 0.0047 mol) in DMF/water mixture (20 ml/5 ml) was stirred for overnight at 100° C. After completion of the reaction, the solids were removed by filtration. The filtrate was diluted with water and extracted with ethyl acetate (×2). The combined organic extracts were washed with water followed by brine, dried over Na₂SO₄ and evaporated. The residue was purified by Flash Column Chromatography to give the desired product in 67% yield (350 mg).

### Step 2

### 2-(6-(cyclopropylmethoxy)-4′-fluorobiphenyl-3-yl)-4-methylpentanoic acid

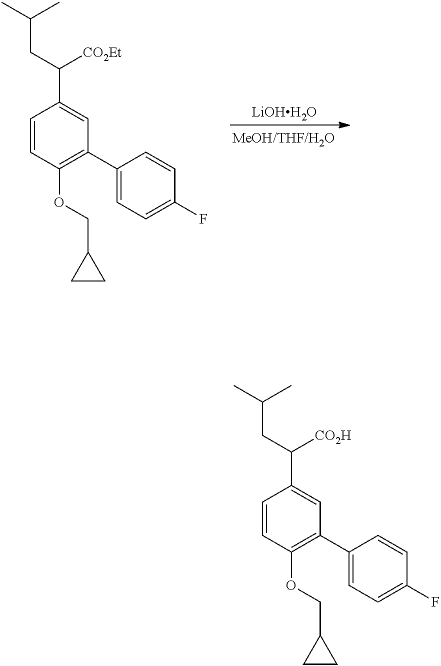

A mixture of ethyl-2-(6-(cyclopropylmethoxy)-4′-fluorobiphenyl-3-yl)-4-methylpentanoate (350 mg, 0.00091 mol) and lithium hydroxide monohydrate (114 mg, 0.0027 mol) in MeOH/THF/Water solvent mixture (10 ml/10 ml/10 ml) was stirred for 3 h at room temperature. After completion of the reaction, the volatiles were removed under reduced pressure. The residue was diluted with water, acidified with 5% HCl solution and extracted with ethyl acetate (×2). The combined organic extracts were washed with water, dried with Na₂SO₄, filtered and evaporated. The residue was purified by Flash Column Chromatography to give the desired product in 62% yield (200 mg).

¹HNMR (CDCl₃, 500 MHz): 7.57 (d, 2H); 7.22 (m, 2H), 7.08 (d, 2H); 6.92 (d, 1H); 3.81 (d, 2H); 3.62 (t, 1H); 1.98 (m, 1H); 1.75 (m, 1H); 1.56 (m, 1H); 1.18 (m, 1H), 0.93 (d, 6H), 0.58 (d, 2H), 0.23 (d, 2H).

### Example 428

### 2-(6-(cyclopropylmethoxy)-4′-chlorobiphenyl-3-yl)-4-methylpentanoic acid

### Step 1

### Ethyl-2-(6-(cyclopropylmethoxy)-4′-chlorobiphenyl-3-yl)-4-methylpentanoate

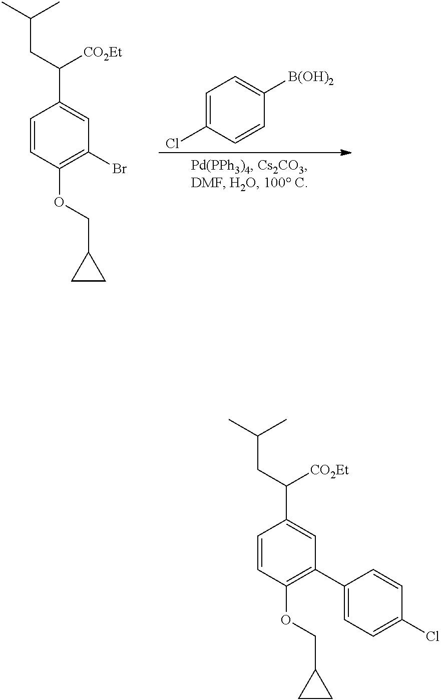

A mixture of ethyl 2-(3-bromo-4-(cyclopropyl methyl)phenyl)-4-methyl pentanoate (500 mg. 0.00135 mg), 4-chloro phenylboronic acid (240 mg, 0.00153 mol), Palladium Tetrakis(triphenylphosphine) (156 mg, 0.00013 mol), cesium carbonate (1.54 g, 0.0047 mol) in DMF/water mixture (20 ml/5 ml) was stirred for overnight at 100° C. After completion of the reaction, the solids were removed by filtration. The filtrate was diluted with water and extracted with ethyl acetate (×2). The combined organic extracts were washed with water followed by brine, dried over Na₂SO₄ and evaporated. The residue was purified by Flash Column Chromatography to give the desired product in 37% yield (200 mg).

### Step 2

### 2-(6-(cyclopropylmethoxy)-4′-chlorobiphenyl-3-yl)-4-methylpentanoic acid

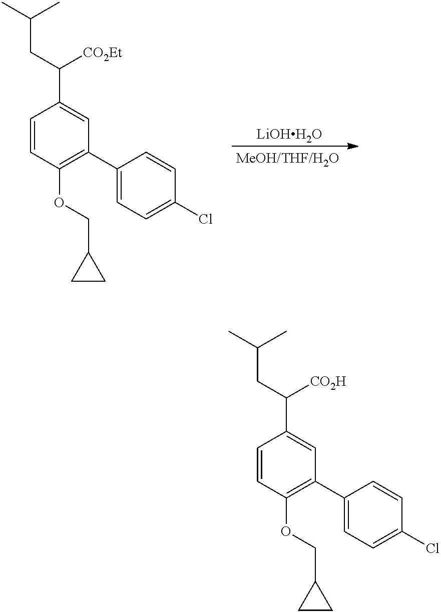

A mixture of ethyl-2-(6-(cyclopropylmethoxy)-4′-chlorobiphenyl-3-yl)-4-methylpentanoate (200 mg, 0.0005 mol) and lithium hydroxide monohydrate (63 mg, 0.0015 mol) in MeOH/THF/Water solvent mixture (10 ml/10 ml/10 ml) was stirred for 3 h at room temperature. After completion of the reaction, the volatiles were removed under reduced pressure. The residue was diluted with water, acidified with 5% HCl solution and extracted with ethyl acetate (×2). The combined organic extracts were washed with water, dried with Na₂SO₄, filtered and evaporated. The residue was purified by flash column chromatography to give the desired product in 27% yield (50 mg).

¹HNMR (CDCl₃, 500 MHz): 7.57 (d, 2H); 7.38 (d, 2H), 7.22 (d, 1H); 6.92 (d, 1H); 3.79 (d, 2H); 3.63 (t, 1H); 1.98 (m, 1H); 1.75 (m, 1H); 1.56 (m, 1H); 1.18 (m, 1H), 0.93 (d, 6H), 0.58 (d, 2H), 0.23 (d, 2H).

### Example 429

### 2-(6-(cyclopropylmethoxy)biphenyl-3-yl)-4-methylpentanoic acid

### Step 1

### Ethyl-2-(6-(cyclopropylmethoxy)biphenyl-3-yl)-4-methylpentanoate

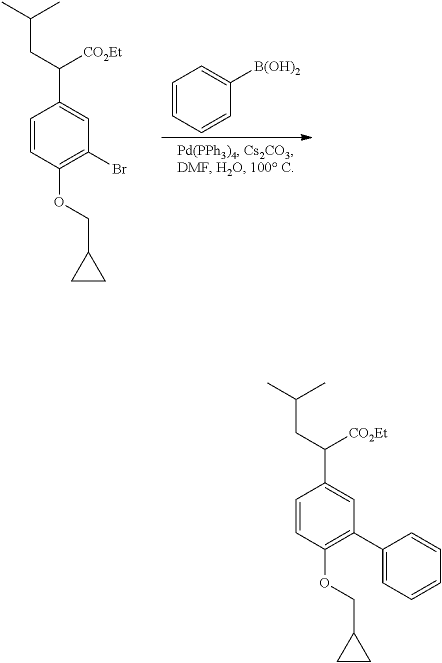

A mixture of ethyl 2-(3-bromo-4-(cyclopropyl methyl)phenyl)-4-methyl pentanoate (500 mg. 0.00135 mol), phenylboronic acid (180 mg, 0.00149 mol), Palladium Tetrakis(triphenylphosphine) (156 mg, 0.000139 mol), cesium carbonate (1.54 g, 0.0047 mol) in DMF/water mixture (20 ml/5 ml) was stirred for overnight at 100° C. After completion of the reaction, the solids were removed by filtration. The filtrate was diluted with water and extracted with ethyl acetate (×2). The combined organic extracts were washed with water followed by brine, dried over Na₂SO₄ and evaporated. The residue was purified by Flash Column Chromatography to give the desired product in 60% yield (300 mg).

### Step 2

### 2-(6-(cyclopropylmethoxy)biphenyl-3-yl)-4-methylpentanoic acid

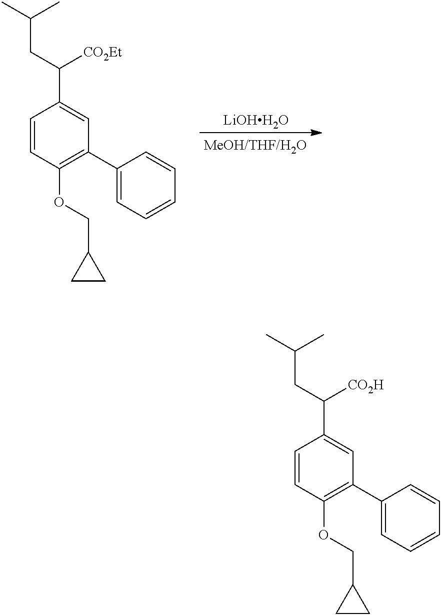

A mixture of 2-(6-(cyclopropylmethoxy)biphenyl-3-yl)-4-methylpentanoic acid (300 mg, 0.818 mmol) and lithium hydroxide monohydrate (103 mg, 2.4 mmol) in MeOH/THF/Water solvent mixture (10 ml/10 ml/10 ml) was stirred for 3 h at room temperature. After completion of the reaction, the volatiles were removed under reduced pressure. The residue was diluted with water, acidified with 5% HCl solution and extracted with ethyl acetate (×2). The combined organic extracts were washed with water, dried with Na₂SO₄, filtered and evaporated. The residue was purified by flash column chromatography to give the desired product in 59% yield (160 mg).

¹HNMR (CDCl₃, 500 MHz): 10.45 (bs, 1H), 7.58 (d, 2H); 7.41 (d, 2H), 7.25 (m, 3H), 6.94 (d, 1H); 3.81 (d, 2H); 3.62 (t, 1H); 1.98 (m, 1H); 1.75 (m, 1H); 1.56 (m, 1H); 1.18 (m, 1H), 0.93 (d, 6H), 0.58 (d, 2H), 0.23 (d, 2H).

### Example 430

### 2-(4-(cyclopropylmethoxy)-3-(2-trifluoromethyl)imidazo[1,2-α]pyridine-7-yl)phenyl)-4-methylpentanoic acid

### Step 1

### Ethyl-2-(4-(cyclopropylmethoxy)-3-(2-trifluoromethyl)imidazo[1,2-α]pyridine-7-yl)phenyl)-4-methylpentanoate

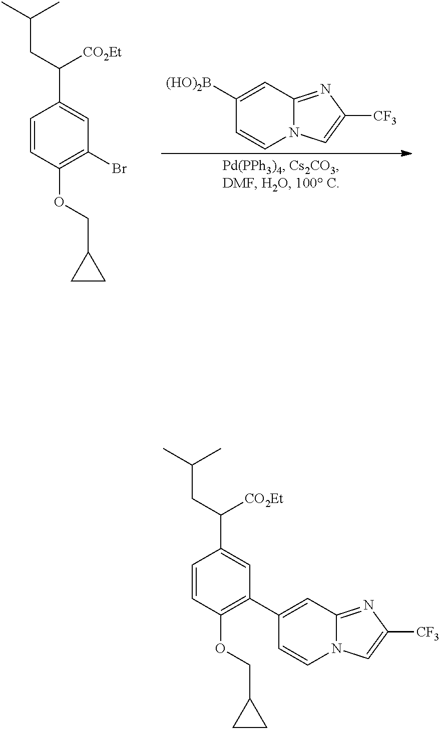

A mixture of ethyl 2-(3-bromo-4-(cyclopropyl methyl)phenyl)-4-methyl pentanoate (100 mg. 0.27 mmol), 2-(trifluoromethyl)imidazo[1,2-α]pyridine-7-ylboronic acid (93 mg, 0.298 mmol), Palladium Tetrakis(triphenylphosphine) (62 mg, 0.054 mmol), cesium carbonate (310 mg, 0.948 mmol) in DMF/water mixture (10 ml/2 ml) was stirred for overnight at 100° C. After completion of the reaction, the solids were removed by filtration, the filtrate was diluted with water and extracted with ethyl acetate (×2). The combined organic extracts were washed with water followed by brine, dried over Na₂SO₄ and evaporated. The residue was purified by flash column chromatography to give the desired product in 47% yield (60 mg).

### Step 2

### 2-(4-(cyclopropylmethoxy)-3-(2-trifluoromethyl)imidazo[1,2-α]pyridine-7-yl)phenyl)-4-methylpentanoic acid

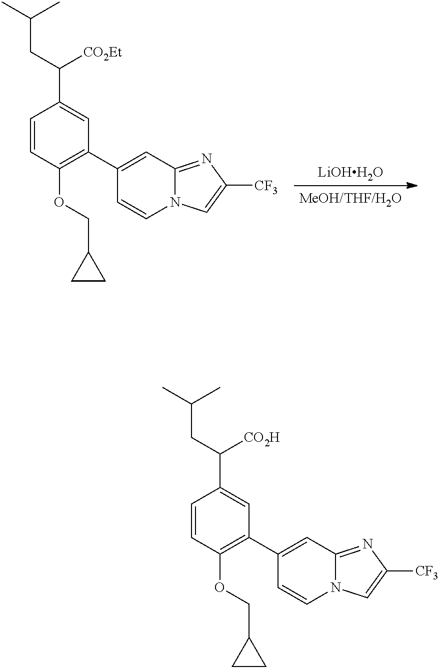

A mixture of ethyl-2-(4-(cyclopropylmethoxy)-3-(2-trifluoromethyl)imidazo[1,2-α]pyridine-7-yl)phenyl)-4-methylpentanoate (55 mg, 0.915 mmol) and lithium hydroxide monohydrate (11.5 mg, 0.274 mmol) in a MeOH/THF/Water solvent mixture (5 ml/5 ml/5 ml) was stirred for 3 h at room temperature. After completion of the reaction the volatiles were removed under reduced pressure. The residue was diluted with water, acidified with 5% HCl solution and extracted with ethyl acetate (×2). The combined organic extracts were washed with water, dried over Na₂SO₄, filtered and evaporated. The residue was purified by flash column chromatography to give the desired product in 87% yield (45 mg).

¹HNMR (CDCl₃, 500 MHz): 8.14 (d, 1H); 7.88 (s, 2H), 7.40 (s, 1H); 7.25 (m, 2H), 6.94 (d, 1H); 3.85 (d, 2H); 3.68 (t, 1H); 1.98 (m, 1H); 1.75 (m, 1H); 1.56 (m, 1H); 1.18 (m, 1H), 0.93 (d, 6H), 0.58 (d, 2H), 0.26 (d, 2H).

### Example 431

### 2-(6-(ethoxy-4′-(trifluoromethyl)biphenyl-3-yl)-4-methylpentanoic acid

### Step 1

### Ethyl 2-(3-bromo-4-ethoxyphenyl)-4-methylpentanoate

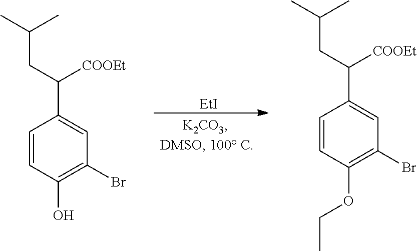

To a stirred solution of ethyl 2-(3-bromo-4-hydroxyphenyl)-4-methylpentanoate (1 g, 3.174 mmol) and K₂CO₃ (657 mg, 4.76 mmol) in 15 ml of DMSO slowly added ethyl iodide (643 mg, 4.126 mmol) at 0° C., allowed to stir at 60° C. for 4 h. After completion of the reaction, the mixture was poured into water and extracted with ethyl acetate (2×100 mL). The combined organic extracts were washed with water, dried over Na₂SO₄ and evaporated to give the desired product in 55% yield (600 mg).

### Step 2

### Ethyl 2-(6-ethoxy-4′-(trifluoromethyl)biphenyl-3-yl)-4-methylpentanoate

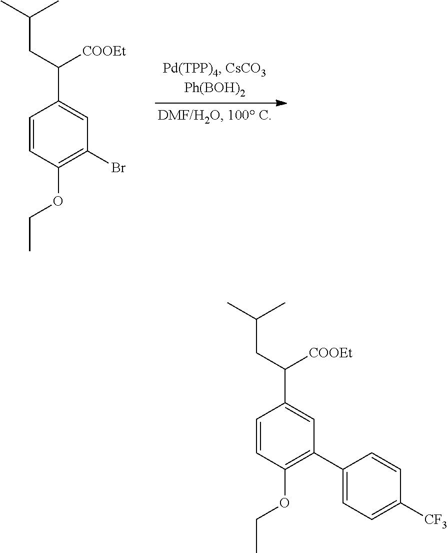

A mixture of compound ethyl 2-(3-bromo-4-ethoxyphenyl)-4-methylpentanoate (580 mg, 1.69 mmol), 4-Trifluoromethyl phenylboronic acid (360 mg, 1.86 mmol), Palladium Tetrakis(triphenylphosphine) (190 mg, 0.169 mmol), Cesium carbonate (1.93 g, 5.98 mmol) in DMF/water mixture (20 ml/2 ml) was stirred for overnight at 100° C. After completion of the reaction, the solids were removed by filtration. The filtrate was diluted with water and extracted with ethyl acetate (×2). The combined organic extracts were washed with water followed by brine, dried over Na₂SO₄ and evaporated. The residue was purified by Flash Column Chromatography to give the desired product 72% yield (500 mg).

### Step 3

### 2-(6-(ethoxy-4′-(trifluoromethyl)biphenyl-3-yl)-4-methylpentanoic acid

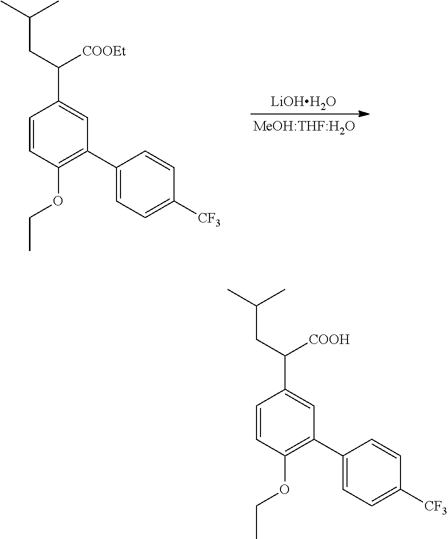

A mixture of ethyl 2-(6-ethoxy-4′-(trifluoromethyl)biphenyl-3-yl)-4-methylpentanoate (500 mg, 1.22 mmol) and lithium hydroxide monohydrate (154 mg, 3.67 mmol) in MeOH/THF/Water solvent mixture (20 ml) was stirred for 3 h at room temperature. After completion of the reaction, the volatiles were removed under reduced pressure. The residue was diluted with water, acidified with 5% HCl solution and extracted with ethyl acetate (×2). The combined organic extracts were washed with water, dried with Na₂SO₄, filtered and evaporated. The residue was purified by flash column chromatography to give the desired product in 66% yield (300 mg).

¹HNMR (CDCl3, 500 MHz): 7.64 (m, 4H); 7.26 (m, 2H), 6.94 (d, 1H); 4.04 (q, 2H); 3.64 (t, 1H); 1.98 (m, 1H); 1.75 (m, 1H); 1.56 (m, 1H); 1.35 (t, 3H), 0.93 (d, 6H).

### Example 140

### 2-(cyclopropylmethoxy)-4′-trifluoromethyl)biphenyl-4-yl)butanoic acid

### Step 1

### Ethyl-(2-(cyclopropylmethoxy)-4′-trifluoromethyl)biphenyl-4-yl)butanoate

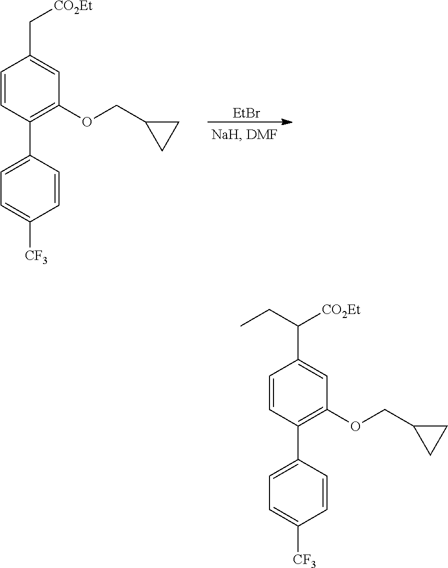

Ethyl-(2-cyclopropylmethoxy-4′-trifluoromethyl-biphenyl-4-yl)-acetate (0.5 g, 0.13 mol) was dissolved in 10 mL anhydrous DMF and NaH (60% wt. in oil, 0.053 g) was added at 0° C. The reaction mixture was stirred for 0.5 h at 25° C. and ethyl bromide (0.144 g) was added drop wise at 0° C. The reaction mixture was stirred an additional 1 h at 0° C. and saturated NH₄Cl solution (10 mL) was added. The reaction mixture was extracted with EtOAc (3×20 mL) and the combined organic phases were washed with water (3×20 mL) and brine (20 mL), dried over MgSO₄, filtered and evaporated under reduced pressure to give 380 mg of the desired product (76% yield).

### Step 2

### 2-(cyclopropylmethoxy)-4′-trifluoromethyl)biphenyl-4-yl)butanoic acid

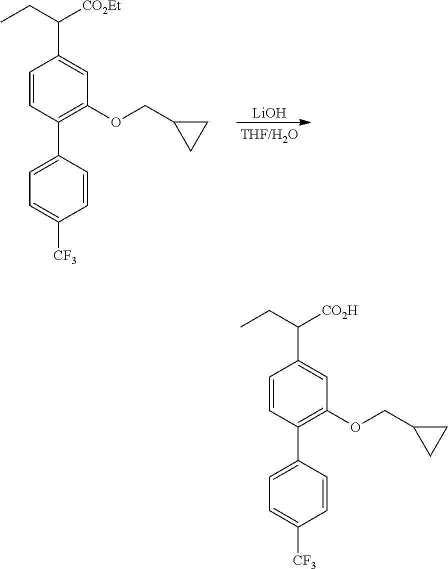

A mixture of ethyl-(2-(cyclopropylmethoxy)-4′-trifluoromethyl)biphenyl-4-yl)butanoate (300 mg) and lithium hydroxide monohydrate (45 mg) in MeOH/THF/Water solvent mixture (5 ml/5 ml/5 ml) was stirred for 3 h at room temperature. After completion of the reaction, the volatiles were removed under reduced pressure. The residue was diluted with water, acidified with 5% HCl solution and extracted with ethyl acetate (×2). The combined organic extracts were washed with water, dried with Na₂SO₄, filtered and evaporated. The residue was purified by flash column chromatography to give the desired product in 43% yield (120 mg).

¹HNMR (CDCl₃, 500 MHz): 7.68 (dd, 4H); 7.31 (d, 1H), 7.01 (d, 3H), 6.94 (s, 1H); 3.82 (d, 2H); 3.62 (t, 1H); 2.18 (m, 1H); 1.87 (m, 1H), 1.18 (m, 1H), 0.98 (d, 6H), 0.58 (d, 2H), 0.24 (d, 2H).

### Example 133

### 4-Methyl-2-[2-(2,2,2-trifluoro-ethoxy)-4′-trifluoromethyl-biphenyl-4-yl]-pentanoic Acid

### Step 1

### 4-Fluoro-1-nitro-2-(2,2,2-trifluoroethoxy)benzene

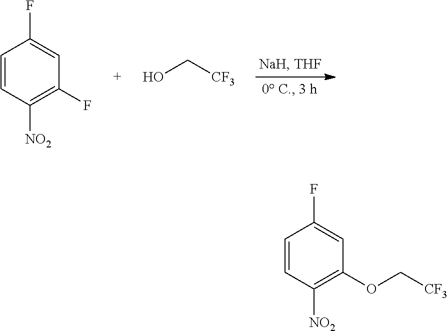

To a solution of 2,2,2-trifluoroethanol (1.10 g, 11.0 mmol) in anhydrous THF (10 mL) was added NaH (60%, 0.44 g, 11.0 mmol) in portions. Twenty minutes later, the reaction mixture was cooled in an ice bath and 2,4-difluoronitrobenzene (1.59 g, 10.0 mmol) in anhydrous THF (5 mL) was added dropwise. The reaction mixture was continued to stir at 0° C. for 3 h, carefully quenched with water (50 mL), and then extracted with diethyl ether (100 mL). The organic layer was washed with brine (50 mL), dried over sodium sulfate, and concentrated under reduced pressure, followed by silica gel flash chromatography eluting with heptane/ethyl acetate (20:1) to give the desired product (1.73 g, 72%) as a white solid: mp 44-45° C.; ¹H NMR (300 MHz, CDCl₃) δ 8.01 (dd, 1H, J=9.0, 5.4 Hz), 6.90 (ddd, 1H, J=9.0, 6.9, 2.4 Hz), 6.83 (dd, 1H, J=9.0, 2.4 Hz), 4.48 (q, 2H, J=7.8 Hz); ¹³C NMR (75 MHz, CDCl₃) δ 165.23 (d, J=256 Hz), 152.50 (d, J=11 Hz), 137.15, 128.22 (d, J=11 Hz), 122.50 (q, J=276 Hz), 110.18 (d, J=23 Hz), 104.62 (d, J=26 Hz), 67.95 (q, J=37 Hz).

### Step 2

### 2-[4-Nitro-3-(2,2,2-trifluoro-ethoxy)-phenyl]-malonic acid tert-butyl ester ethyl ester

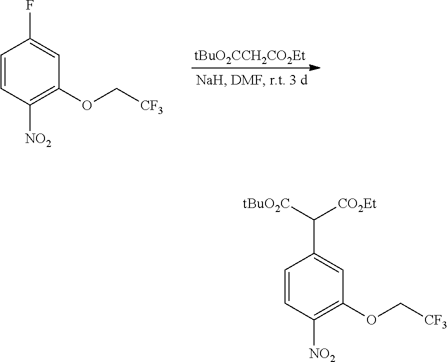

To a solution of tert-butyl ethyl malonate (17.8 g, 94.8 mmol) in anhydrous DMF (400 mL) at room temperature was added NaH (60%, 4.16 g, 104 mmol) in portions and the mixture was stirred at room temperature for 30 min. 4-Fluoro-1-nitro-2-(2,2,2-trifluoroethoxy)benzene (19.7 g, 82.4 mmol) was added and the mixture was stirred at room temperature overnight. Additional NaH (60%, 4.16 g, 104 mmol) was added in portions and the reaction mixture was stirred for two more days. The reaction was slowly quenched with water (1.0 L) and the pH was adjusted to 7 with ammonium chloride. The mixture was extracted with ethyl acetate (1.0 L). The organic layer was washed with water (1.0 L), brine (1.0 L), dried over sodium sulfate, and concentrated under reduced pressure, followed by silica gel flash chromatography eluting with a gradient of heptane/ethyl acetate (from 10:1 to 5:1) to give the desired product (23.9 g, 71%) as a yellow oil: ¹H NMR (300 MHz, CDCl₃) δ 7.88 (d, 1H, J=8.4 Hz), 7.29 (s, 1H), 7.20 (dd, 1H, J=8.4, 1.2 Hz), 4.60 (s, 1H), 4.52 (q, 2H, J=8.1 Hz), 4.24 (m, 2H), 1.47 (s, 9H), 1.29 (t, 3H, J=7.2 Hz); ¹³C NMR (75 MHz, CDCl₃) δ 166.91, 165.56, 150.17, 140.01, 139.81, 125.66, 124.06, 122.61 (q, J=276 Hz), 117.46, 83.43, 67.57 (q, J=36 Hz), 62.25, 58.41, 27.77, 14.01.

### Step 3

### [4-Nitro-3-(2,2,2-trifluoro-ethoxy)-phenyl]-acetic acid ethyl ester

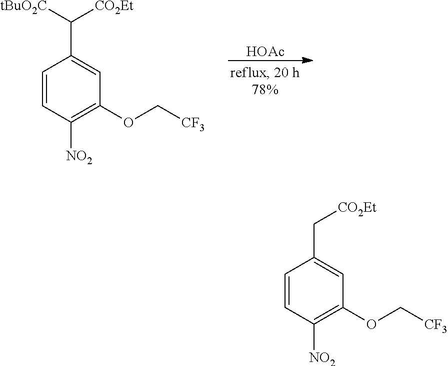

A solution of 2-[4-nitro-3-(2,2,2-trifluoro-ethoxy)-phenyl]-malonic acid tert-butyl ester ethyl ester (23.8 g, 58.4 mmol) in acetic acid (500 mL) was heated at reflux overnight and then concentrated under reduced pressure. The residue was purified by silica gel flash chromatography eluting with a gradient of heptane/ethyl acetate (from 10:1 to 5:1) to give the desired product (14.9 g, 78%) as a light yellow solid: mp 76-77° C.; ¹H NMR (300 MHz, CDCl₃) δ 7.87 (d, 1H, J=8.4 Hz), 7.10 (m, 2H), 4.50 (q, 2H, J=7.8 Hz), 4.18 (q, 2H, J=7.2 Hz), 3.68 (s, 2H), 1.28 (t, 3H, J=7.2 Hz); ¹³C NMR (75 MHz, CDCl₃) δ 169.73, 150.45, 141.33, 139.45, 125.98, 123.96, 122.64 (q, J=276 Hz), 117.61, 67.72 (q, J=36 Hz), 61.50, 41.05, 14.15.

### Step 4

### [4-Amino-3-(2,2,2-trifluoro-ethoxy)-phenyl]-acetic acid ethyl ester

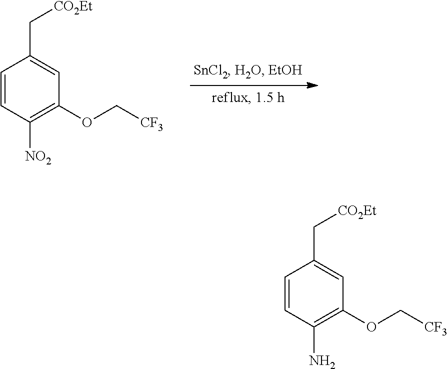

A suspension of [4-nitro-3-(2,2,2-trifluoro-ethoxy)-phenyl]-acetic acid ethyl ester (13.3 g, 43.2 mmol) and tin (II) chloride (32.8 g, 173 mmol) in water (6.23 g, 346 mmol) and ethanol (300 mL) was heated at reflux for 1.5 h. The reaction mixture was concentrated under reduced pressure. Ethyl acetate (300 mL) and NaOH (1 N, 600 mL) were added. The aqueous layer was extracted with ethyl acetate (300 mL) again. The combined organic layers were washed with water (300 mL), brine (300 mL), dried over sodium sulfate, and concentrated under reduced pressure, followed by silica gel flash chromatography eluting with heptane/ethyl acetate (3:1) to give the desired product (10.6 g, 88%) as a yellow oil: ¹H NMR (300 MHz, CDCl₃) δ 6.78 (d, 1H, J=7.8 Hz), 6.73 (s, 1H), 6.68 (d, 1H, J=7.8 Hz), 4.36 (q, 2H, J=8.4 Hz), 4.13 (q, 2H, J=7.2 Hz), 3.81 (s, 2H, br), 3.49 (s, 2H), 1.25 (t, 3H, J=7.2 Hz); ¹³C NMR (75 MHz, CDCl₃) δ 171.74, 144.69, 135.69, 123.96, 123.26 (q, J=276 Hz), 115.59, 113.83, 66.48 (q, J=35 Hz), 60.80, 40.73, 14.24.

### Step 5

### [4-Iodo-3-(2,2,2-trifluoro-ethoxy)-phenyl]-acetic acid

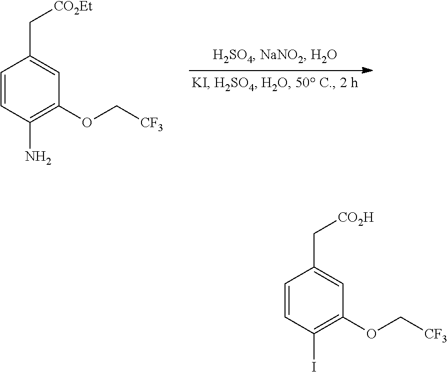

To sulfuric acid (95-98%, 36 mL) cooled at −8° C. was added [4-Amino-3-(2,2,2-trifluoro-ethoxy)-phenyl]-acetic acid ethyl ester (10.0 g, 36.1 mmol), and water (CARE!, 54 mL). A solution of sodium nitrite (2.99 g, 43.3 mmol) in water (36 mL) was added dropwise and the reaction mixture was stirred for additional 25 min. Subsequently, the solution of the diazonium salt was added dropwise at room temperature to a mixture of KI (17.9 g, 108 mmol), water (29 mL) and sulfuric acid (4.13 mL). The mixture was stirred for one hour at room temperature and then at 50-65° C. for two hours. The reaction mixture was cooled to room temperature and diluted with dichloromethane (200 mL). The organic layer was washed with 6% Na₂S₂O₃ solution (200 mL), water (200 mL), brine (200 mL), dried over sodium sulfate, and concentrated under reduced pressure, followed by recrystallization from ethanol (20 mL) and water (40 mL) to give the desired product (5.86 g, 45%) as a brown solid: mp 153-154° C.; ¹H NMR (300 MHz, DMSO-d₆) δ 12.41 (s, 1H, br), 7.72 (d, 1H, J=7.8 Hz), 7.05 (s, 1H), 6.75 (d, 1H, J=7.8 Hz), 4.78 (q, 2H, J=8.7 Hz), 3.55 (s, 2H); ¹³C NMR (75 MHz, DMSO-d₆) δ 171.84, 155.33, 138.67, 137.09, 125.26, 122.67 (q, J=276 Hz), 114.74, 83.92, 65.51 (q, J=34 Hz), 40.23.

### Step 6

### [4-Iodo-3-(2,2,2-trifluoro-ethoxy)-phenyl]-acetic acid ethyl ester

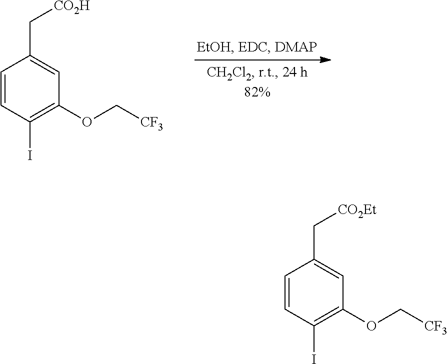

To a mixture of [4-Iodo-3-(2,2,2-trifluoro-ethoxy)-phenyl]-acetic acid (5.10 g, 14.2 mmol), EDC.HCl (4.06 g, 21.2 mmol) and 4-dimethylaminopyridine (0.256 g, 2.12 mmol) cooled in an ice bath were added anhydrous dichloromethane (200 mL) and ethanol (6.54 g, 142 mmol), and the resulting solution was stirred at room temperature for two days. More EDC.HCl (0.70 g, 3.6 mmol) was added and the reaction mixture was stirred at room temperature for one more day. The reaction mixture was washed with water (2×200 mL), brine (200 mL), dried over anhydrous sodium sulfate, and concentrated under reduced pressure, followed by silica gel flash chromatography eluting with a gradient of heptane/ethyl acetate (from 20:1 to 10:1) to give the desired product (4.52 g, 82%) as a yellow oil: ¹H NMR (300 MHz, CDCl₃) δ 7.72 (d, 1H, J=7.8 Hz), 6.80 (s, 1H), 6.74 (d, 1H, J=7.8 Hz), 4.39 (q, 2H, J=8.1 Hz), 4.15 (q, 2H, J=7.2 Hz), 3.56 (s, 2H), 1.25 (t, 3H, J=7.2 Hz); ¹³C NMR (75 MHz, CDCl₃) δ 170.51, 156.15, 139.66, 136.01, 125.37, 122.87 (q, J=276 Hz), 114.37, 84.86, 66.92 (q, J=36 Hz), 61.11, 40.89, 14.15.

### Step 7

### [2-(2,2,2-Trifluoro-ethoxy)-4′-trifluoromethyl-biphenyl-4-yl]-acetic acid ethyl ester

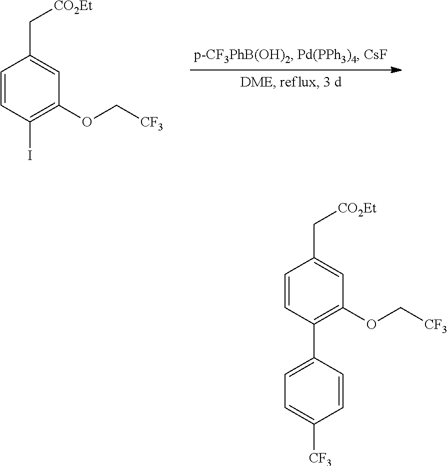

A mixture of [4-Iodo-3-(2,2,2-trifluoro-ethoxy)-phenyl]-acetic acid ethyl ester (2.00 g, 5.15 mmol), CsF (1.79 g, 11.8 mmol), (4-trifluoromethyl)benzeneboronic acid (1.23 g, 6.50 mmol) and Pd(PPh₃)₄ (0.298 g, 0.258 mmol) in 1,2-dimethoxyethane (60 mL) was heated at reflux for four days. The reaction mixture was concentrated under reduced pressure, and the residue was extracted with ethyl acetate (100 mL) and filtered. The filtrate was washed with 5% Na₂CO₃ (100 mL), brine (100 mL), dried over sodium sulfate, and concentrated under reduced pressure, followed by recrystallization from ethanol (12 mL) and water (4 mL) to give the desired product (1.05 g, 50%) as an off-white solid: ¹H NMR (300 MHz, CDCl₃) δ 7.64 (m, 4H), 7.33 (d, 1H, J=7.8 Hz), 7.08 (d, 1H, J=7.8 Hz), 6.94 (s, 1H), 4.31 (q, 2H, J=7.8 Hz), 4.19 (q, 2H, J=7.2 Hz), 3.66 (s, 2H), 1.29 (t, 3H, J=7.2 Hz); ¹³C NMR (75 MHz, CDCl₃) δ 170.93, 154.04, 140.68, 135.72, 131.20, 129.54, 128.89, 127.78 (q, J=270 Hz), 124.89 (q, J=4 Hz), 123.92, 123.08 (q, J=277 Hz), 114.44, 66.38 (q, J=35 Hz), 61.18, 41.14, 14.26.

### Step 8

### 4-Methyl-2-[2-(2,2,2-trifluoro-ethoxy)-4′-trifluoromethyl-biphenyl-4-yl]-pentanoic acid ethyl ester

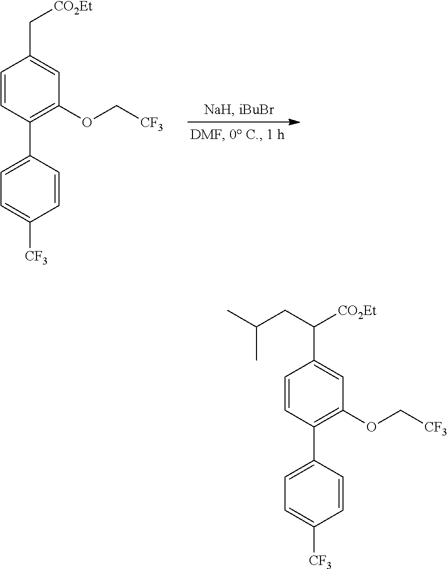

To a solution of [2-(2,2,2-trifluoro-ethoxy)-4′-trifluoromethyl-biphenyl-4-yl]-acetic acid ethyl ester (0.20 g, 0.49 mmol) in anhydrous DMF (2 mL) cooled in an ice bath was added NaH (60%, 0.024 g, 0.60 mmol). After the reaction mixture was stirred for 30 min, isobutyl bromide (0.069 g, 0.50 mmol) in anhydrous DMF (1 mL) was added dropwise. After at 0° C. for one hour, water (30 mL) was added and then ammonium chloride (0.10 g) to neutralize the reaction mixture, followed by extraction with ethyl acetate (30 mL). The organic layer was washed with brine (30 mL), dried over sodium sulfate, concentrated under reduced pressure to give a yellow oil, which was purified by silica gel flash chromatography eluting with heptane/ethyl acetate (25:1) to generate the desired product (0.05 g, 22%) as a colorless oil: ¹H NMR (300 MHz, CDCl₃) δ 7.63 (m, 4 H), 7.31 (d, 1H, J=7.8 Hz), 7.12 (dd, 1H, J=7.8, 1.5 Hz), 6.98 (d, 1H, J=1.5 Hz), 4.32 (q, 2H, J=7.8 Hz), 4.14 (m, 2H), 3.69 (t, 1H, J=7.8 Hz), 2.00 (m, 1H), 1.69 (m, 1H), 1.52 (m, 1H), 1.25 (t, 3H, J=7.2 Hz), 0.95 (d, 6H, J=6.3 Hz); ¹³C NMR (75 MHz, CDCl₃) δ 173.84, 154.50, 141.63, 141.07, 131.38, 130.99, 129.81, 129.32, 125.49 (q, J=270 Hz), 125.15 (q, J=4 Hz), 123.40 (q, J=277 Hz), 123.13, 113.55, 66.85 (q, J=36 Hz), 61.17, 49.98, 43.11, 26.40, 22.84, 22.63, 14.46.

### Step 9

### Example 133

### 4-Methyl-2-[2-(2,2,2-trifluoro-ethoxy)-4′-trifluoromethyl-biphenyl-4-yl]-pentanoic acid

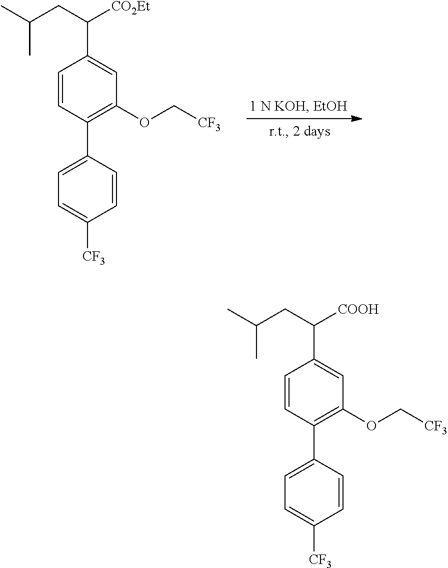

A mixture of 4-methyl-2-[2-(2,2,2-trifluoro-ethoxy)-4′-trifluoromethyl-biphenyl-4-yl]-pentanoic acid ethyl ester (0.050 g, 0.11 mmol) and aqueous KOH (1.1 M, 1.0 mL) in ethanol (10 mL) was stirred at room temperature for two days. After the solvent was removed under reduced pressure, the residue was diluted with water (30 mL), acidified with 1 N HCl solution to pH 1.0 and extracted with ethyl acetate (30 mL). The organic extract was dried over sodium sulfate, concentrated under reduced pressure, followed by lyophilization overnight to give the desired product (0.030 g, 63%) as a white solid; HRMS (DIP-CI): calcd for C₂₁H₂₀O₃F₆ (M⁺) 434.1317, found 434.1328; ¹H NMR (300 MHz, CDCl₃) δ 9.20 (s, 1H, br), 7.63 (m, 4H), 7.33 (d, 1H, J=7.5 Hz), 7.13 (d, 1H, J=7.5 Hz), 6.96 (s, 1H), 4.31 (q, 2H, J=8.1 Hz), 3.71 (t, 1H, J=7.5 Hz), 2.00 (m, 1H), 1.73 (m, 1H), 1.55 (m, 1H), 0.95 (d, 6H, J=6.3 Hz); ¹³C NMR (75 MHz, CDCl₃) δ 178.85, 154.39, 140.67, 140.44, 131.30, 129.57, 129.26, 124.98 (q, J=4 Hz), 124.24 (q, J=270 Hz), 123.14 (q, J=277 Hz), 123.02, 113.63, 113.29, 66.72 (q, J=35 Hz), 49.45, 42.32, 26.07, 22.64, 22.31; HPLC purity: 99.6%, retention time=7.46 min.

### Example 433

### 2-(2-(cyclopropylmethoxy)-4′-(trifluoromethyl)biphenyl-4-yl)acetic acid

Example 433 may be synthesized via analogous procedures to those described above

### Screening of Compounds of the Disclosure for Aβ 42 Inhibition

Aβ42 and Aβ40 were measured in the culture medium of HEK293/Sw cells, human embryonic kidney 293 (HEK 293) stably expressing the APP₆₉₅ isoform carrying the double Swedish mutations (K595N/M596L) (1). The cells were cultured in Dulbecco's modified Eagle's medium (DMEM) supplemented with 10% or 2% fetal bovine serum and 200 μg/ml G418. Cells were seeded onto 24-well plates and allowed to grow to sub-confluence overnight. Compounds were then added in a final volume of 0.5 ml culture medium. After overnight incubation (15-16 hours), 100 μl of supernatants were removed from each well for Aβ measurement using ELISA (Wako). Then 80 μl MTS reagent (Promega) was added to each well for determining cytotoxicity.

### Table

### Experimental Procedures for Rat Primary Cortical Culture-Based Abeta₁_{_{→}}_{42/1}_{_{→}}ₓ ELISAs

Rat primary neocortical cultures are established through the dissection of the neocortices from 10-12 E17 embryos harvested from time-pregnant CD (Sprague Dawley) rats (Charles River Laboratories). Following dissection, the combined neocortical tissue specimen volume is brought up to 5 mL with dissection medium (DM; 1×HBSS (Invitrogen Corp., cat#14185-052)/10 mM HEPES (Invitrogen Corp., cat#15630-080)/1 mM Sodium Pyruvate (Invitrogen Corp., cat#11360-070)) supplemented with 100 uL Trypsin (0.25%; Invitrogen Corp., cat#15090-046) and 100 uL DNase I (0.1% stock solution in DM, Roche Diagnostics Corp., cat#0104159), undergoing digestion via incubation at 37° C. for 10 minutes. Digested tissue is washed once in plating medium (PM; NeuroBasal (Invitrogen Corp., cat#21103-049)/10% Horse Serum (Sigma-Aldrich Co., cat# H1138)/0.5 mM L-Glutamine (Invitrogen Corp., cat#25030-081)), then resuspended in a fresh 10 mL PM volume for trituration. Trituration consists of 18 cycles with a 5 mL-serological pipet, followed by 18 cycles with a flame-polished glass Pasteur pipet. The volume is elevated to 50 mL with PM, the contents then passed over a 70 um cell-strainer (BD Biosciences, cat#352350) and transferred directly to a wet-ice bath. The cell-density is quantified using a hemacytometer, and diluted to allow for the plating of 50000 cells/well/100 uL in pre-coated 96-well PDL-coated plates (Corning, Inc., cat#3665). Cells are incubated for 4-5 hours at 37° C./5% CO₂, after which time the entire volume is exchanged to feeding medium (FM; NeuroBasal/2% B-27 Serum-free supplement (Invitrogen Corp., cat#17504-044)/0.5 mM L-Glutamine/1% Penicillin-Streptomycin (Invitrogen Corp., cat#15140-122)). The cultures undergo two 50% fresh FM exchanges, after 3 days in vitro (DIV3), and again at DIV7.

Human C-terminal recognition-site Abeta₁_{_{→}}₄₂ and Rat N-terminal recognition-site Abeta₁_{_{→}}ₓ capture-antibodies, diluted 1:300 in 0.05M Carbonate-Bicarbonate buffer (Sigma-Aldrich Co., C-3041), are plated at 100 uL/well on flat-bottomed F96 MicroWell™ (MaxiSorp™ surface) plates (Nalge Nunc International, cat#439454), and incubated overnight at 4° C. Compounds to be screened are solubilized in dimethyl sulphoxide (DMSO, Sigma-Aldrich Co., cat#15493-8), and further diluted in DMSO in an eight-point dose-response format. Into 96-well plates, dose-response compound dilutions (1000× the desired final concentration) are stamped out at 2 uL/well, in duplicate (up to 3 compounds/plate), as a daughter plate. In addition, DMSO and N—[N-(3,5-difluorophenacetyl)-L-alanyl]-S-phenylglycine t-butyl ester (DAPT), a gamma-secretase inhibitor (GSI), are incorporated as solvent and positive controls, respectively. With the assistance of liquid-handling automation, the compound daughter plate is diluted 1:500 with warmed FM, and two DIV8 culture plates are leveled to 60 uL/well, and immediately overlaid with 60 uL/well of the 2× diluted daughter plate. The plates are returned to the 37° C./5% CO₂-incubator for 24 hours.

Each capture-antibody ELISA plate undergoes 4×250 uL/well Phosphate-buffered saline with 0.05% Tween®-20 SigmaUltra (PBS-T; Fluka, cat#79383/Sigma-Aldrich Co., cat# P7949) washes. The ELISA plates are then overlaid with 120 uL/well PBS-T supplemented with 1% Bovine Serum Albumin Diluent/Blocking solution (BSA; Kirkegaard & Perry Laboratories (KPL), Inc., cat#50-61-01) and incubate at room-temperature on an orbital shaker for a minimum of 2 hours.

Rat Abeta₁_{_{→}}₄₂ and rat Abeta₁_{_{→}}₄₀ peptide (American Peptide Co., cat#62-0-84/62-0-86A) DMSO stock solutions are serially-diluted 1:2 in FM yielding a final concentration range of 0-500 pg/mL, to be plated on the respective ELISA plates for determination of the corresponding standard curve, from which concentrations of specific or total Abeta peptides in the presence of a particular drug concentration can be calculated. The conditioned medium from the duplicate culture plates are collected and combined into one round-bottom 96-well transfer plate which is incubated on wet-ice. The culture plates are rinsed once with 120 ul/well FM, and replenished immediately with 100 uL/well FM, being returned to the incubator for 10 minutes. Cell-viability is evaluated by adding 20 uL/well of warmed CellTiter 96® Aqᵤₑₒᵤₛ One Solution (MTS/PES; Promega Corp., cat# G3581), and returning the plates to the incubator for 30-90 minutes. Plate absorbance at 492 nm is read on a spectrophotometer, and from which, the ratio of absorbance of compound-treated cells to absorbance of solvent (DMSO)-treated control cells is calculated. The calculation of the corresponding EC₅₀ values is performed following non-linear curve-fitting using GraphPad Prism® software.

For each ELISA plate, a corresponding transfer-plate is created containing 120 uL/well of either the rat Abeta₁_{_{→}}₄₂ or rat Abeta₁_{_{→}}₄₀ peptide standard solutions, in duplicate, and 110-115 uL/well of the collected conditioned-medium plate, half designated for the Abeta₁_{_{→}}₄₂ ELISA, and the other half for the Abeta₁_{_{→}}ₓ ELISA. The ELISA plates undergo a second set of 4×250 uL/well PBS-T washes, immediately followed by being overlaid with their designated transfer-plate. The ELISA plates incubate on an orbital-shaker for 16-18 hours at 4° C.

Detection antibody solution is prepared by diluting beta-Amyloid 17-24 (4G8) biotinylated monoclonal antibody (Covance, Inc., cat# SIG-39240-200) 1:1500 in PBS-T supplemented with 0.67% BSA. The ELISA plates undergo 4×250 uL/well PBS-T washes, and are overlaid with 100 uL/well of 4G8 diluted detection-antibody solution. The Abeta₁_{_{→}}₄₂ ELISA plates are incubated on an orbital-shaker at room-temperature for 90 minutes, the Abeta₁_{_{→}}ₓ ELISA plates for 60 minutes.

In order to conjugate the biotinylated monoclonal 4G8 antibody, following 4×250 uL/well PBS-T washes, the ELISA plates undergo a one-hour incubation at 100 ul/well with a 1:15000 dilution of Streptavidin-HRP conjugate (Jackson ImmunoResearch Laboratories, Inc., cat#016-030-0840) on an orbital-shaker at room temperature.

Following a final set of 4×250 uL/well PBS-T washes, the ELISA plates are overlaid with 100 ul/well SureBlue 3,3′,5, 5′-Tetramethylbenzidine (TMB) Microwell Peroxidase substrate solution (Kirkegaard & Perry Laboratories, Inc., cat#52-00-02), protected from light, and incubate for 20-45 minutes at room temperature. At the point the desired level of development is attained, 100 ul/well of TMB Stop solution (Kirkegaard & Perry Laboratories, Inc., cat#50-85-05) is added, and the plate thoroughly shaken in preparation for reading on a spectrophotometer. SureBlue TMB Microwell Substrate develops a deep blue color in the presence of a peroxidase-labeled conjugate, and turns yellow when stopped by acidification, allowing for plate absorbance at 450 nm to be read. From the calculation of the standard curve, the compound dose-response curves, normalized to DAPT performance, are plotted as % DMSO using GraphPad Prism® software, and the corresponding IC₅₀ values calculated.

### In Vivo Studies (Rats)

Male Sprague Dawley rats from Harlan, 230-350 g, were used for studies. Fasted rats were dosed via oral gavage, with vehicle (15% Solutol HS 15, 10% EtOH, 75% Water) or compound, at a volume of 10 ml/kg. For PK studies, at fixed time points after dosing, the rats were euthanized with an excess of CO₂. Terminal blood was collected through cardiac puncture, mixed in EDTA tubes, immediately spun (3 min at 11,000 rpm at 4° C.), and snap frozen for plasma collection. A piece of frontal cortex was collected and snap frozen for compound level determination. For A-beta lowering studies, at a determined time point after dosing (Cmax if it is ≧3 hr), rats were euthanized as in the PK studies and plasma was collected as described above. Cerebellum was removed and saved for compound level determination, and the remaining brain was divided into 4 quadrants, snap frozen and saved to examine A-beta peptide levels.

Solutol HS 15 was purchased from Mutchler Inc.

Practitioners will also know that similar methods can also be applied to other species such as mice (including transgenic strains such as Tg2576), guinea pig, dog and monkey.

### Analysis of In Vivo Aβ Lowering Studies

Compounds of the invention can be used to treat AD in mammal such as a human or alternatively in a validated animal model such as the mouse, rat, or guinea pig. The mammal may not be diagnosed with AD, or may not have a genetic predisposition for AD, but may be transgenic such that it overproduces and eventually deposits Aβ in a manner similar to that seen in the human. Alternatively, non-transgenic animals may also be used to determine the biochemical efficacy of the compound, that is, the effect on the Aβ biomarker, with an appropriate assay.

Compounds can be administered in any standard form using any standard method. For example, but not limited to, compounds can be in the form of liquid, tablets or capsules that are taken orally or by injection. Compounds can be administered at any dose that is sufficient to significantly reduce, for example, levels of Aβₜₒₜₐₗ or more specifically Aβ₄₂ in the blood plasma, cerebrospinal fluid (CSF), or brain.

To determine whether acute administration of the compound would reduce Aβ₄₂ levels in-vivo, two-three month old non-transgenic Sprague-Dawley rats were used. Rats treated with the compound would be examined and compared to those untreated or treated with vehicle and brain levels of soluble Aβ₄₂ and Aβₜₒₜₐₗ would be quantitated by standard techniques, for example, using an immunoassay such as an ELISA. Treatments may be acute or sub-chronic and treatment periods may vary from hours to days or longer and can be adjusted based on the results of the biochemical endpoint once a time course of onset of effect can be established.

A typical protocol for measuring Aβ or Aβ₄₂ levels from in-vivo samples is shown but it is only one of many variations that could used to detect the levels of Aβ.

Compounds may be administered as a single oral dose given three to four hours before sacrifice and subsequent analysis or alternatively could be given over a course of days and the animals sacrificed three to four hours after the administration of the final dose

For total Aβ or Aβ₄₂ analysis brain tissue is homogenized in ten volumes of ice cold 0.4% DEA/50 mM NaCl containing protease inhibitors, e.g., for 0.1 g of brain 1 ml of homogenization buffer is added. Homogenization is achieved either by sonciation for 30 seconds at 3-4 W of power or with a polytron homogenizer at three-quarters speed for 10-15 seconds. Homogenates (1.2 ml) are transferred to pre-chilled centrifuge tubes (Beckman 343778 polycarbonate tubes) are placed into a Beckman TLA120.2 rotor. Homogenates are centrifuged for 1 hour at 100,000 rpm (355,040×g) at 4° C. The resulting supernatants are transferred to fresh sample tubes and placed on ice (the pellets are discarded).

The samples are further concentrated and purified by passage over Waters 60 mg HLB Oasis columns according to the methods described (Lanz and Schachter (2006) J. Neurosci Methods. 157 (1):71-81; Lanz and Schachter (2008). J. Neurosci Methods. 169 (1):16-22). Briefly, using a vacuum manifold (Waters# WAT200607) the columns are attached and conditioned with 1 ml of methanol at a flow rate of 1 ml/minute. Columns are then equilibrated with 1 ml of water. Samples are loaded (800 μl) into individual columns (the Aβ will attach to the column resin). The columns are washed sequentially with 1 ml of 5% methanol followed by 1 ml of 30% methanol. After the final wash the eluates are collected in 13×100 mm tubes by passing 800 μl of solution of 90% methanol/2% ammonium hydroxide) over the columns at 1 ml/minute. The samples are transferred to 1.5 ml non-siliconized sample tubes are dried in a speed-vac concentrator at medium heat for at least 2 hours or until dry.

The dried samples are either stored at −80° C. or are used immediately by resuspending the pellets in 80 μl of Ultra-Culture serum-free media (Lonza) supplemented with protease inhibitors by vortexing for 10 seconds. Sixty microliters of each sample is transferred to a pre-coated immunoassay plate coated with an affinity purified rabbit polyclonal antibody specific to Aβ₄₂ (x-42). Sixty microliters of fresh supplemented ultraculture is added to the remaining sample and 60 microliters is transferred to a pre-coated and BSA blocked immunoassay plate coated with an affinity purified rabbit polyclonal antibody specific to total rodent Aβ (1-x). Additional standard samples of rodent Aβ/rodent Aβ₄₂ are also added to the plates with final concentrations of 1000, 500, 250, 125, 62.5, 31.3 and 15.6 pg/ml. The samples are incubated overnight at 4° C. in order to allow formation of the antibody-Amyloid-antibody-complex. The following day the plates are washed 3-4 times with 150 microliters of phosphate buffered saline containing 0.05% Tween 20. After removal of the final wash 100 μl of the monoclonal antibody 4G8 conjugated to biotin (Covance) diluted 1:1000 in PBS-T containing 0.67% BSA was added and the plates incubated at room temperature for 1-2 hours. The plates are again washed 3-4 times with PBS-T and 100 μl of a Streptavidin-Peroxidase-Conjugate diluted 1:10,000 from a 0.5 mg/ml stock in PBS-T contained 0.67% BSA is added and the plates incubated for at least 30 minutes. Following a final set of washes in PBS-T, a TMB/peroxide mixture is added, resulting in the conversion of the substrate into a colored product. This reaction is stopped by the addition of sulfuric acid (1M) and the color intensity is measured by means of photometry with an microplate reader with a 450 nm filter. Quantification of the Aβ content of the samples is obtained by comparing absorbance to a standard curve made with synthetic Aβ. This is one example of a number of possible measurable endpoints for the immunoassay which would give similar results.

### Pharmacokinetic Analysis

### Sample Preparation

Plasma samples and standards were prepared for analysis by treating with a 3× volume of acetonitrile containing 500 ng/mL of internal standard (a selected aryl propionic acid). Typically 150 μL of acetonitrile with internal standard was added to 50 μL of plasma. Acetonitrile was added first to each well of a 96-well Phenomenex Strata Impact protein precipitation filter plate followed by the addition of the plasma sample or standard. The filter plate was allowed to sit for at least 15 minutes at room temperature before a vacuum was applied to filter the samples into a clean 96-well plate.

If sample concentrations were observed or predicted to be greater than 1000 ng/mL, plasma samples were diluted with blank plasma 10-150 fold depending on the anticipated concentration and upper limit of quantitation of the analytical method.

Samples of frontal cortex or cerebellum were homogenized then treated in similar manner. To each brain sample, a 4× volume of PBS (pH 7.4) buffer was added along with a 15× volume of acetonitrile (containing internal standard) in a 2 mL screw-cap plastic tube. The tubes were then filled one third of the way with 1 mm zirconia/silica beads (Biospec) and placed in a Mini Bead Beater for 3 minutes. The samples were inspected and if any visible pieces of brain remained, they were returned to the Bead Beater for another 2-3 minutes of shaking. The resulting suspension was considered to be a 5-fold dilution treated with a 3× volume of acetonitrile (with internal standard). Calibration standards were prepared in 5-fold diluted blank brain homogenate and precipitated with a 3× volume of acetonitrile immediately after the addition of the appropriate spiking solution (see below). All brain standards and samples were allowed to sit for at least 15 minutes prior to filtering them through a Phenomenex Strata Impact protein precipitation filter plate into a clean 96-well plate.

Spiking solutions for plasma and brain calibration standards were prepared at concentrations of 0.02, 0.1, 0.2, 1, 2, 10, 20, 100 and 200 μg/mL in 50:50 acetonitrile/water. Calibration standards were prepared by taking 190 μL of blank matrix (plasma or brain homogenate) and adding 10 μL of spiking solution resulting in final concentrations of 1, 5, 10, 50, 100, 500, 1000, 5000 and 10,000 ng/mL.

### LC-MS/MS Analysis

Precipitated plasma and brain samples were analyzed by LC-MS/MS using a Shimadzu LC system consisting of two LC-10AD pumps and a SIL-HTc autosampler connected to an Applied Biosystems MDS/Sciex API 3200 QTRAP mass spectrometer.

For chromatographic separation, a Phenomenex Luna C-18 3 μM (2×20 mm) column was used with an acetonitrile-based gradient mobile phase. The two mobile phase components were:

Mobile phase A: water with 0.05% (v/v) formic acid and 0.05% (v/v) 5 N ammonium hydroxide.

Mobile phase B: 95:5 acetonitrile/water with 0.05% (v/v) formic acid and 0.05% (v/v) 5 N ammonium hydroxide.

The gradient for each analysis was optimized for the specific compound, but generally, the run started with between 0% and 40% of mobile phase B, ramped up to 100% of mobile phase B over 1-2 minutes, then held there for 2-3 minutes before returning to the initial conditions for 4 minutes to re-equilibrate.

The API 3200 QTRAP mass spectrometer was used in MRM mode with negative electrospray ionization. MRM transitions and mass spec settings were optimized for each compound.

Standard curves were created by quadratic or linear regression with 1/x*x weighting. Calibration standards were prepared 1-10,000 ng/mL, but the highest (and sometimes lowest) standards were often not acceptable for quantitation and only those standards with reasonable back-calculated accuracies were included in the calibration curve. Ideally, only standards with +/−15% of nominal concentration would be included in the fitted standard curve, but occasionally larger deviations were accepted after careful consideration.

Sample concentrations below the quantitation range were reported as “BQL”. Concentrations above the curve were usually re-run with larger sample dilutions.

### Administration

The present disclosure includes pharmaceutical composition for treating a subject having a neurological disorder comprising a therapeutically effective amount of a compound of the Formula I, a derivative or a pharmaceutically acceptable salt thereof, and a pharmaceutically acceptable excipient, carrier or diluent.

The pharmaceutical compositions can be administered in a variety of dosage forms including, but not limited to, a solid dosage form or in a liquid dosage form, an oral dosage form, a parenteral dosage form, an intranasal dosage form, a suppository, a lozenge, a troche, buccal, a controlled release dosage form, a pulsed release dosage form, an immediate release dosage form, an intravenous solution, a suspension or combinations thereof. The dosage can be an oral dosage form that is a controlled release dosage form. The oral dosage form can be a tablet or a caplet. The compounds can be administered, for example, by oral or parenteral routes, including intravenous, intramuscular, intraperitoneal, subcutaneous, transdermal, airway (aerosol), rectal, vaginal and topical (including buccal and sublingual) administration. In one embodiment, the compounds or pharmaceutical compositions comprising the compounds are delivered to a desired site, such as the brain, by continuous injection via a shunt.

In another embodiment, the compound can be administered parenterally, such as intravenous (IV) administration. The formulations for administration will commonly comprise a solution of the compound of the Formula I dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of compound of Formula I in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In one embodiment, the compound of Formula I or Formula II can be administered by introduction into the central nervous system of the subject, e.g., into the cerbrospinal fluid of the subject. The formulations for administration will commonly comprise a solution of the compound of the Formula I dissolved in a pharmaceutically acceptable carrier. In certain aspects, the compound of the Formula I is introduced intrathecally, e.g., into a cerebral ventricle, the lumbar area, or the cisterna magna. In another aspect, the compound of the Formula I or Formula II is introduced intraocullarly, to thereby contact retinal ganglion cells.

The pharmaceutically acceptable formulations can easily be suspended in aqueous vehicles and introduced through conventional hypodermic needles or using infusion pumps. Prior to introduction, the formulations can be sterilized with, preferably, gamma radiation or electron beam sterilization.

In one embodiment, the pharmaceutical composition comprising a compound of Formula I is administered into a subject intrathecally. As used herein, the term “intrathecal administration” is intended to include delivering a pharmaceutical composition comprising a compound of Formula I or Formula II directly into the cerebrospinal fluid of a subject, by techniques including lateral cerebroventricular injection through a burrhole or cisternal or lumbar puncture or the like (described in Lazorthes et al. Advances in Drug Delivery Systems and Applications in Neurosurgery, 143-192 and Omaya et al., Cancer Drug Delivery, 1: 169-179, the contents of which are incorporated herein by reference). The term “lumbar region” is intended to include the area between the third and fourth lumbar (lower back) vertebrae. The term “cisterna magna” is intended to include the area where the skull ends and the spinal cord begins at the back of the head. The term “cerebral ventricle” is intended to include the cavities in the brain that are continuous with the central canal of the spinal cord. Administration of a compound of Formula I or Formula II to any of the above mentioned sites can be achieved by direct injection of the pharmaceutical composition comprising the compound of Formula I or Formula II by the use of infusion pumps. For injection, the pharmaceutical compositions can be formulated in liquid solutions, preferably in physiologically compatible buffers such as Hank's solution or Ringer's solution. In addition, the pharmaceutical compositions may be formulated in solid form and re-dissolved or suspended immediately prior to use. Lyophilized forms are also included. The injection can be, for example, in the form of a bolus injection or continuous infusion (e.g., using infusion pumps) of pharmaceutical composition.

In one embodiment, the pharmaceutical composition comprising a compound of Formula I or Formula II is administered by lateral cerebro ventricular injection into the brain of a subject. The injection can be made, for example, through a burr hole made in the subject's skull. In another embodiment, the encapsulated therapeutic agent is administered through a surgically inserted shunt into the cerebral ventricle of a subject. For example, the injection can be made into the lateral ventricles, which are larger, even though injection into the third and fourth smaller ventricles can also be made.

In yet another embodiment, the pharmaceutical composition is administered by injection into the cisterna magna, or lumbar area of a subject.

For oral administration, the compounds will generally be provided in unit dosage forms of a tablet, pill, dragee, lozenge or capsule; as a powder or granules; or as an aqueous solution, suspension, liquid, gels, syrup, slurry, etc. suitable for ingestion by the patient. Tablets for oral use may include the active ingredients mixed with pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

Pharmaceutical preparations for oral use can be obtained through combination of a compound of Formula I with a solid excipient, optionally grinding a resulting mixture, and processing the mixture of granules, after adding suitable additional compounds, if desired, to obtain tablets or dragee cores. Suitable solid excipients in addition to those previously mentioned are carbohydrate or protein fillers that include, but are not limited to, sugars, including lactose, sucrose, mannitol, or sorbitol; starch from corn, wheat, rice, potato, or other plants; cellulose such as methyl cellulose, hydroxypropylmethyl-cellulose or sodium carboxymethylcellulose; and gums including arabic and tragacanth; as well as proteins such as gelatin and collagen. If desired, disintegrating or solubilizing agents may be added, such as the cross-linked polyvinyl pyrrolidone, agar, alginic acid, or a salt thereof, such as sodium alginate.

Capsules for oral use include hard gelatin capsules in which the active ingredient is mixed with a solid diluent, and soft gelatin capsules wherein the active ingredients is mixed with water or an oil such as peanut oil, liquid paraffin or olive oil.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

For transmucosal administration (e.g., buccal, rectal, nasal, ocular, etc.), penetrants appropriate to the barrier to be permeated are used in the formulation. Such penetrants are generally known in the art.

Formulations for rectal administration may be presented as a suppository with a suitable base comprising for example cocoa butter or a salicylate. Formulations suitable for vaginal administration may be presented as pessaries, tampons, creams, gels, pastes, foams or spray formulations containing in addition to the active ingredient such carriers as are known in the art to be appropriate. For intramuscular, intraperitoneal, subcutaneous and intravenous use, the compounds will generally be provided in sterile aqueous solutions or suspensions, buffered to an appropriate pH and isotonicity. Suitable aqueous vehicles include Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

The suppositories for rectal administration of the drug can be prepared by mixing the drug with a suitable non-irritating excipient which is solid at ordinary temperatures but liquid at the rectal temperatures and will therefore melt in the rectum to release the drug. Such materials are cocoa butter and polyethylene glycols.

The compounds can be delivered transdermally, by a topical route, formulated as applicator sticks, solutions, suspensions, emulsions, gels, creams, ointments, pastes, jellies, paints, powders, or aerosols.

The compounds may also be presented as aqueous or liposome formulations. Aqueous suspensions can contain a compound of Formula I or Formula II in admixture with excipients suitable for the manufacture of aqueous suspensions. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan monooleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Oil suspensions can be formulated by suspending a compound of Formula I or Formula II in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

In addition to the formulations described previously, the compounds may also be formulated as a depot preparation. Such long acting formulations may be administered by implantation or transcutaneous delivery (e.g., subcutaneously or intramuscularly), intramuscular injection or a transdermal patch. Thus, for example, the compounds may be formulated with suitable polymeric or hydrophobic materials (e.g., as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

For administration by inhalation, the compounds are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

In general a suitable dose will be in the range of 0.01 to 100 mg per kilogram body weight of the recipient per day, preferably in the range of 0.2 to 10 mg per kilogram body weight per day. The desired dose is preferably presented once daily, but may be dosed as two, three, four, five, six or more sub-doses administered at appropriate intervals throughout the day.

The compounds can be administered as the sole active agent, or in combination with other known therapeutics to be beneficial in the treatment of neurological disorders. In any event, the administering physician can provide a method of treatment that is prophylactic or therapeutic by adjusting the amount and timing of drug administration on the basis of observations of one or more symptoms (e.g., motor or cognitive function as measured by standard clinical scales or assessments) of the disorder being treated.

Details on techniques for formulation and administration are well described in the scientific and patent literature, see, e.g., the latest edition of Remington's Pharmaceutical Sciences, Maack Publishing Co, Easton Pa. (“Remington's”). After a pharmaceutical composition has been formulated in an acceptable carrier, it can be placed in an appropriate container and labeled for treatment of an indicated condition.

What is claimed is:

1. A compound of Formula I or II: 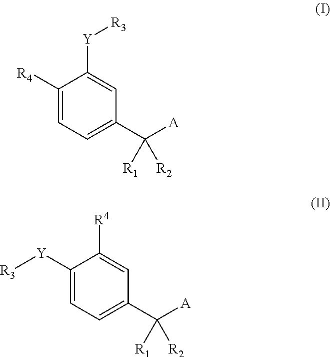
wherein
A is selected from CO₂H, C(O)NHOH, C(O)NHOCH₃, C(O)NH₂, C(O)NH(R₅), C(O)N(R₅)₂, C(O)NH(SO₂CH₃), COCF₃ and a tetrazole group;
R₁ and R₂ are independently selected from: H, F, R₆, OH, OR₆, NH₂, NHR₅, N(R₅)₂, NHC(O)R₅, NHCO₂R₅, SR₆, S(O)R₆, S(O)₂R₆;
each R₅ is independently chosen from alkyl; cycloalkyl; (CH₂)ₙAr, wherein n=0-2 and wherein Ar is a phenyl, napthyl; and a heteroaryl ring and wherein Ar is optionally substituted with up to 3 groups selected from alkyl, halogen, CF₃, OH, OCF3, alkoxy, OCH₂CH₂OCH₃, NH₂, alkylamino, dialkylamino, morpholino, CN, NO₂, alkylthio, alkylsulfonyl; or in the case when two R₅ are attached to the same N and are both alkyl, they can be taken together to form a 5-membered or 6-membered ring optionally containing O, S, N(H) or N-alkyl;
R₆ is C1-C5 alkyl optionally interrupted by —O—, —S—, —S(O)—, or —S(O)₂— groups; cycloalkyl or (CH₂)ₙAr wherein n=0-2 and wherein Ar is a phenyl, napthyl or heteroaryl ring and wherein Ar is optionally substituted with up to 3 groups selected from alkyl, halogen, CF₃, OH, OCF₃, alkoxy, OCH₂CH₂OCH₃, NH₂, alkylamino, dialkylamino, morpholino, CN, NO₂, alkylthio, alkylsulfonyl; or R₁ and R₂ can be taken together to form a 3-7 membered cycloalkyl or heterocycloalkyl ring which optionally bears a C1-C4 alkyl substituent;
Y is a bond or a divalent linking group selected from —O—, —OCH₂—, —OCH₂CH₂—, —CH₂, —C(O)—, —CH═CH—, —CH₂CH₂—, —CH₂O—, —CH₂OCH₂—, —CH₂CH₂O—, —S—, —SCH₂—, CH₂S—, —CH₂SCH₂—, —C(O)NH—, —C(O)N(R₅)—, —NHC(O)—, —N(R₅)C(O)—, —S(O)—, —S(O₂)—, —S(O)₂N(H)—, —S(O)₂N(R₅)—, —N(H)S(O)₂—, and —N(R₅)S(O)₂—, wherein the point of attachment of divalent linking groups, Y, to R₃ in the Formulas I and II is to the right;
R₃ is a C3-C7 alkyl group optionally interrupted by —O—, —S—, —S(O)—, or —S(O)₂— groups or a mono- or bi-cyclic ring system comprising 5 to 10 ring atoms selected from C, N, O and S, provided that not more than 3 ring atoms in any single ring are other than C, said ring system optionally bearing up to 3 substituents selected from halogen, R₆, CF₃, CN, NO₂, OR₆, SR₆CO₂R₆, OCOR₆ and COR₆; wherein the attachment site may be either at a carbon atom or a nitrogen atom of the ring system provided that only three bonds are made to nitrogen.
R₄ is C2-C4 alkyl, OCH₂CF₃, C2-C4 alkoxy, cyclopropyloxymethyl, trialkylsilyl, SO₂NH₂, SO₂N(R₇)(R₈), or mono or bicyclic aromatic or heteroaromatic system ring comprising 5 to 10 ring atoms chosen from C, N, O and S, provided that not more than 3 ring atoms in any single ring are other than C, wherein the ring system must be substituted with an R₉ group and is optionally substituted with an R₁₀ groups; wherein the ring attachment may be either at a carbon atom or a nitrogen atom of the ring system provided that only three bonds are made to the attachment nitrogen atom.
R₇ and R₈ are independently selected from H, alkyl, cycloalkyl, (CH₂)ₙAr wherein n=0-2 and wherein Ar is a phenyl, napthyl or heteroaryl ring optionally substituted with up to 3 groups selected from alkyl, halogen, CF₃, OH, OCF₃, alkoxy, OCH₂CH₂OCH₃, NH₂, alkylamino, dialkylamino, morpholino, CN, NO₂, alkylthio, or alkylsulfonyl; and when taken together may form a mono or bicyclic ring system comprising from 5-10 atoms and which may contain up to 3 heteroatoms selected from O, N, or S and which is optionally substituted with halogen, alkyl, alkoxy, CF₃, CN, OH, NO₂, NH₂, alkylamino, dialkylamino, alkylsulfonyl;
R₉ is selected from OH, NH₂, NH(R₅), N(R₅)₂, alkylsulfonyl, arylsulfonyl, SO₂NH₂, SO₂N(R₅)₂, a heteroaryl group optionally substituted with halogen, OH, alkyl, alkoxy, NH₂, CN, NO₂ and a phenyl ring which is substituted with at least one group selected from ethyl, cyclopropyl, OH, alkoxy, CN, NO₂, NH₂, N(R₅)₂, alkylsulfonyl, arylsulfonyl, SO₂NH₂, and is optionally additionally substituted with a halogen group;
R₁₀ is hydrogen, halogen, alkyl, CF₃, alkoxy, CN, or NO₂; and
pharmaceutically acceptable salts thereof.

2-94. (canceled)
